(19) United States
(12) Patent Application Publication
(10) Pub. No.: US 2013/0171164 A1
(43) Pub. Date: Jul. 4, 2013

(54) COMPOSITIONS AND METHODS OF MODULATING RECEPTOR PROTEIN TYROSINE PHOSPHATASES

(71) Applicants: Nunzio Bottini, San Diego, CA (US); Stephanie Stanford, San Diego, CA (US)

(72) Inventors: Nunzio Bottini, San Diego, CA (US); Stephanie Stanford, San Diego, CA (US)

(73) Assignee: LA JOLLA INSTITUTE FOR ALLERGY & IMMUNOLOGY, La Jolla, CA (US)

(21) Appl. No.: 13/565,511

(22) Filed: Aug. 2, 2012

Related U.S. Application Data
(63) Continuation of application No. PCT/US2011/023517, filed on Feb. 2, 2011.
(60) Provisional application No. 61/300,742, filed on Feb. 2, 2010.
(60) Provisional application No. 61/387,404, filed on Sep. 28, 2010.

(51) Int. Cl. C12Q 1/68

(52) U.S. Cl. CPC C12Q 1/6883
USPC 424/158.1; 435/6.12; 514/44 A

(57) ABSTRACT

Provided herein, inter alia, are compositions and methods of measuring levels of RPTP protein and RNA and treating subjects with certain diseases, such as autimmune diseases.

### CROSS-REFERENCES TO RELATED APPLICATIONS

This application claims priority to U.S. Provisional Application No. 61/300,742, filed Feb. 2, 2010 and U.S. Provisional Application No. 61/374,404, filed Sep. 28, 2010, the disclosures of which are incorporated herein in their entireties.

### BACKGROUND OF THE INVENTION

The balance between the activities of protein kinases and protein phosphatases regulates, at least partially, vital cellular functions such as cell proliferation and signal transduction. These enzymes modify proteins by adding or removing a phosphate group from serine, threonine, or tyrosine residues in specific proteins.

In particular, protein tyrosine kinases (PTKs) and protein tyrosine phosphatases (PTPs) mediate phosphorylation and dephosphorylation of proteins on tyrosine residues. The PTPs are a large and diverse family of enzymes found ubiquitously in eukaryotes.

Protein tyrosine phosphorylation is a common post-translational modification, which is a fundamental mechanism for proper protein stability, interactions, cellular localization, and for regulating enzyme activity in numerous important aspects of eukaryote physiology.

Tyrosine phosphorylation is involved in cell proliferation and differentiation, gene transcription, mRNA processing, transport of molecules in and out of a cell, embryogenesis, organ development, tissue homeostasis, and the immune system. Abnormalities in tyrosine phosphorylation play a role in the pathogenesis of numerous inherited or acquired human diseases from cancer to autoimmune diseases to immune deficiencies.

PTPs act by removing phosphate groups from phosphorylated tyrosine residues on proteins. Receptor protein tyrosine phosphatases (RPTPs or PTPRs) are membrane bound forms of PTPs that generally have a variable length extracellular domain followed by a transmembrane region and a C-terminal catalytic cytoplasmic domain.

Currently there is incomplete knowledge of the biological relevance of RPTPs within a human or animal subject, especially from the immunological standpoint, except that RPTPs are highly expressed in the central nervous system and are believed to be important neurologically. In fact, at the present time only CD45/PTPRC and CD148/PTPRJ are considered immunologically relevant.

Approximately 5 to 8% of people in the United States suffer from an autoimmune disease. Researchers have identified more than 80 different autoimmune diseases and suspect that many more diseases may have an autoimmune component. Rheumatoid arthritis (RA) alone afflicts roughly 2.5 million people in the United States RA affects the joints and bones but may also affect different organs and biological systems. Provided herein are methods and compositions addressing these and other needs in the art.

### BRIEF SUMMARY OF THE INVENTION

In one aspect, a method is provided for determining whether a subject has or is at risk of developing an autoimmune disease. The method includes determining whether a subject expresses a modulated RNA level of an RPTP or a modulated protein level of an RPTP relative to a standard control. The presence of the modulated RNA level or the modulated protein level indicates the subject has or is at risk of developing an autoimmune disease. The RPTP is PTPRA, PTPRB, PTPRC, PTPRD, PTPRE, PTPRF, PTPRG, PTPRH, PTPRJ, PTPRK, PTPRM, PTPRN, PTPRN2, PTPRO, PTPRQ, PTPRR, PTPRS, PTPRT, PTPRU, PTPRV or PTPRZ1.

In a second aspect, a method is provided for treating a subject who has or is at risk of developing an autoimmune disease The method includes administering to the subject a therapeutically effective amount of an autoimmune therapeutic agent. The autoimmune therapeutic agent is an agonist of an RPTP or an antagonist of an RPTP. The RPTP is PTPRA, PTPRB, PTPRC, PTPRD, PTPRE, PTPRF, PTPRG, PTPRH, PTPRJ, PTPRK, PTPRM, PTPRN, PTPRN2, PTPRO, PTPRQ, PTPRR, PTPRS, PTPRT, PTPRU, PTPRV or PTPRZ1.

In a third aspect, a method is provided for identifying a candidate RPTP binding agent. The method includes a first step of contacting a test agent with an RPTP. The RPTP is PTPRA, PTPRB, PTPRC, PTPRD, PTPRE, PTPRF, PTPRG, PTPRH, PTPRJ, PTPRK, PTPRM, PTPRN, PTPRN2, PTPRO, PTPRQ, PTPRR, PTPRS, PTPRT, PTPRU, PTPRV or PTPRZ1. The method includes a second step of detecting binding of the test agent to the RPTP and thereby identifying a candidate RPTP binding agent.

In another aspect, a pharmaceutical composition is provided. The pharmaceutical composition includes an autoimmune therapeutic agent and a pharmaceutically acceptable excipient. The autoimmune therapeutic agent is an agonist or antagonist of an RPTP. The RPTP is PTPRA, PTPRB, PTPRC, PTPRD, PTPRE, PTPRF, PTPRG, PTPRH, PTPRJ, PTPRK, PTPRM, PTPRN, PTPRN2, PTPRO, PTPRQ, PTPRR, PTPRS, PTPRT, PTPRU, PTPRV or PTPRZ1.

### BRIEF DESCRIPTION OF THE DRAWINGS

The enclosed drawings are a part of the present specification and illustrate exemplary aspects of the invention, which may be embodied in various forms.

FIG. 1 illustrates the expression of RPTPs in effector T cells (Teffs) and regulatory T cells (Tregs), normalized to the housekeeping genes RNA Polymerase II (POLR2) or G6PDH. In particular, expression levels are shown relative to PTPRJ in Teffs. In the histogram, the entries appear in the order (left to right) Teff normalized to POLR2, Teff normalized to G6PDH, Treg normalized to POLR2, Treg normalized to G6PDH.

FIG. 2 illustrates the data of FIG. 1, in which the Y-axis has been expanded for a clearer understanding of the invention. In the histogram, the entries appear in the order (left to right) Teff normalized to POLR2, Teff normalized to G6PDH, Treg normalized to POLR2, Treg normalized to G6PDH.

FIG. 3 illustrates single-cell analysis of FoxP3 (a marker of Tregs) expression of cells that were used in the analysis reproduced in FIGS. 1 and 2. In the graph, the entries appear in the order (left to right) Unstained Teff (dark gray), Teffs (middle gray), Tregs (lightest gray).

FIG. 4 illustrates the expression of RPTPs in control (dark gray) vs. arthritic (light gray) wrist joints of SKG mice as assessed by quantitative PCR. In the histogram, the entries appear in the order (left to right) Control, Arthritis.

FIG. 5 illustrates a replicate experiment in FIG. 4. In the histogram, the entries appear in the order (left to right) Control, Arthritis.

FIG. 6 illustrates expression of RPTPs in control (dark gray) versus arthritic (light gray) lymph nodes of SKG mice as assessed by quantitative PCR. In the histogram, the entries appear in the order (left to right) Control, Arthritis.

FIG. 7 illustrates mRNA expression levels of RPTPs in primary mouse fibroblast-like synoviocytes (CD90.2+) and macrophage-like synoviocytes (CD11b+) as assessed by QPCR. In the histogram, the entries appear in the order (left to right) CD90.2+, CD11b+.

FIG. 8 illustrates mRNA expression levels of RPTPs in a mouse fibroblast-like synovial cell line. In the histogram, the entries appear in the order (left to right) Unstimulated, TNFalpha, IL1beta.

FIG. 9 illustrates mRNA expression levels of RPTPs in a first human fibroblast-like synoviocyte cell line. In the histogram, the entries appear in the order (left to right) Unstimulated, TNFalpha, IL1beta.

FIG. 10 illustrates mRNA expression levels of RPTPs in a second human fibroblast-like synoviocyte cell line. In the histogram, the entries appear in the order (left to right) Unstimulated, TNFalpha, IL1beta.

FIG. 11 illustrates mRNA expression levels of RPTPs in a third human fibroblast-like synoviocyte cell line. In the histogram, the entries appear in the order (left to right) Unstimulated, TNFalpha, IL1beta.

### DETAILED DESCRIPTION OF THE INVENTION

### I. Definitions

The terms “subject,” “patient,” “individual,” etc. are not intended to be limiting and can be generally interchanged. That is, an individual described as a “patient” does not necessarily have a given disease, but may be merely seeking medical advice.

A “standard control” refers to a sample, measurement, or value that serves as a reference, usually a known reference, for comparison to a test sample, measurement, or value. For example, a test sample can be taken from a patient suspected of having a given disease (e.g. an autoimmune disease, inflammatory autoimmune disease, cancer, infectious disease, immune disease, or other disease) and compared to a known normal (non-diseased) individual (e.g. a standard control subject). A standard control can also represent an average measurement or value gathered from a population of similar individuals (e.g. standard control subjects) that do not have a given disease (i.e. standard control population), e.g., healthy individuals with a similar medical background, same age, weight, etc. A standard control value can also be obtained from the same individual, e.g. from an earlier-obtained sample from the patient prior to disease onset. One of skill will recognize that standard controls can be designed for assessment of any number of parameters (e.g. RNA levels, protein levels, individual RPTP levels, individual IAD RPTP levels, specific cell types, specific bodily fluids, specific tissues, synoviocytes, synovial fluid, synovial tissue, fibroblast-like synoviocytes, macrophage-like synoviocytes, etc).

One of skill in the art will understand which standard controls are most appropriate in a given situation and be able to analyze data based on comparisons to standard control values. Standard controls are also valuable for determining the significance (e.g. statistical significance) of data. For example, if values for a given parameter are widely variant in standard controls, variation in test samples will not be considered as significant.

The terms “dose” and “dosage” are used interchangeably herein. A dose refers to the amount of active ingredient given to an individual at each administration. For the present methods and compositions provided herein, the dose may generally refer to the amount of disease (e.g. an autoimmune, inflammatory autoimmune, cancer, infectious, immune, or other disease) treatment, RPTP binding agent, inflammatory autoimmune disease (IAD) binding agent, autoimmune therapeutic agent, inflammatory autoimmune disease (IAD) treatment, inflammatory autoimmune disease (IAD) therapeutic agent, RPTP agonist, RPTP antagonist, anti-PTPR antibody, anti-PTPR inhibitory nucleic acid, anti-PTPR RNAi molecule, or PTPR ligand mimetic. The dose will vary depending on a number of factors, including the range of normal doses for a given therapy, frequency of administration; size and tolerance of the individual; severity of the condition; risk of side effects; and the route of administration. One of skill will recognize that the dose can be modified depending on the above factors or based on therapeutic progress. The term “dosage form” refers to the particular format of the pharmaceutical or pharmaceutical composition, and depends on the route of administration. For example, a dosage form can be in a liquid form for nebulization, e.g., for inhalants, in a tablet or liquid, e.g., for oral delivery, or a saline solution, e.g., for injection.

As used herein, the terms “treat” and “prevent” may refer to any delay in onset, reduction in the frequency or severity of symptoms, amelioration of symptoms, improvement in patient comfort or function (e.g. joint function), decrease in severity of the disease state, etc. The effect of treatment can be compared to an individual or pool of individuals not receiving a given treatment, or to the same patient prior to, or after cessation of, treatment. The term “prevent” generally refers to a decrease in the occurrence of a given disease (e.g. an autoimmune, inflammatory autoimmune, cancer, infectious, immune, or other disease) or disease symptoms in a patient. As indicated above, the prevention may be complete (no detectable symptoms) or partial, such that fewer symptoms are observed than would likely occur absent treatment.

By “therapeutically effective dose or amount” as used herein is meant a dose that produces effects for which it is administered (e.g. treating or preventing a disease). The exact dose and formulation will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Remington: The Science and Practice of Pharmacy, 20th Edition, Gennaro, Editor (2003), and Pickar, Dosage Calculations (1999)). For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a standard control. A therapeutically effective dose or amount may ameliorate one or more symptoms of a disease. A therapeutically effective dose or amount may prevent or delay the onset of a disease or one or more symptoms of a disease when the effect for which it is being administered is to treat a person who is at risk of developing the disease.

The term “diagnosis” refers to a relative probability that a disease (e.g. an autoimmune, inflammatory autoimmune, cancer, infectious, immune, or other disease) is present in the subject. Similarly, the term “prognosis” refers to a relative probability that a certain future outcome may occur in the subject with respect to a disease state. For example, in the context of the present invention, prognosis can refer to the likelihood that an individual will develop a disease (e.g. an autoimmune, inflammatory autoimmune, cancer, infectious, immune, or other disease), or the likely severity of the disease (e.g., duration of disease). The terms are not intended to be absolute, as will be appreciated by any one of skill in the field of medical diagnostics.

“Nucleic acid” or “oligonucleotide” or “polynucleotide” or grammatical equivalents used herein means at least two nucleotides covalently linked together. The term “nucleic acid” includes single-, double-, or multiple-stranded DNA, RNA and analogs (derivatives) thereof. Oligonucleotides are typically from about 5, 6, 7, 8, 9, 10, 12, 15, 25, 30, 40, 50 or more nucleotides in length, up to about 100 nucleotides in length. Nucleic acids and polynucleotides are a polymers of any length, including longer lengths, e.g., 200, 300, 500, 1000, 2000, 3000, 5000, 7000, 10,000, etc. In certain embodiments. the nucleic acids herein contain phosphodiester bonds. In other embodiments, nucleic acid analogs are included that may have alternate backbones, comprising, e.g., phosphoramidate, phosphorothioate, phosphorodithioate, or O-methylphosphoroamidite linkages (see Eckstein, Oligonucleotides and Analogues: A Practical Approach, Oxford University Press); and peptide nucleic acid backbones and linkages. Other analog nucleic acids include those with positive backbones; non-ionic backbones, and non-ribose backbones, including those described in U.S. Pat. Nos. 5,235,033 and 5,034,506, and Chapters 6 and 7, ASC Symposium Series 580, Carbohydrate Modifications in Antisense Research, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made.

A particular nucleic acid sequence also encompasses “splice variants.” Similarly, a particular protein encoded by a nucleic acid encompasses any protein encoded by a splice variant of that nucleic acid. “Splice variants,” as the name suggests, are products of alternative splicing of a gene. After transcription, an initial nucleic acid transcript may be spliced such that different (alternate) nucleic acid splice products encode different polypeptides. Mechanisms for the production of splice variants vary, but include alternate splicing of exons. Alternate polypeptides derived from the same nucleic acid by read-through transcription are also encompassed by this definition. Any products of a splicing reaction, including recombinant forms of the splice products, are included in this definition. An example of potassium channel splice variants is discussed in Leicher, et al., J. Biol. Chem. 273(52):35095-35101 (1998).

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are near each other, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

The term “probe” or “primer”, as used herein, is defined to be one or more nucleic acid fragments whose specific hybridization to a sample can be detected. A probe or primer can be of any length depending on the particular technique it will be used for. For example, PCR primers are generally between 10 and 40 nucleotides in length, while nucleic acid probes for, e.g., a Southern blot, can be more than a hundred nucleotides in length. The probe may be unlabeled or labeled as described below so that its binding to the target or sample can be detected. The probe can be produced from a source of nucleic acids from one or more particular (preselected) portions of a chromosome, e.g., one or more clones, an isolated whole chromosome or chromosome fragment, or a collection of polymerase chain reaction (PCR) amplification products. The length and complexity of the nucleic acid fixed onto the target element is not critical to the invention. One of skill can adjust these factors to provide optimum hybridization and signal production for a given hybridization procedure, and to provide the required resolution among different genes or genomic locations.

The probe may also be isolated nucleic acids immobilized on a solid surface (e.g., nitrocellulose, glass, quartz, fused silica slides), as in an array. In some embodiments, the probe may be a member of an array of nucleic acids as described, for instance, in WO 96/17958. Techniques capable of producing high density arrays can also be used for this purpose (see, e.g., Fodor (1991) Science 767-773; Johnston (1998) Curr. Biol. 8: R171-R174; Schummer (1997) Biotechniques 23: 1087-1092; Kern (1997) Biotechniques 23: 120-124; U.S. Pat. No. 5,143,854).

A “labeled nucleic acid probe or oligonucleotide” is one that is bound, either covalently, through a linker or a chemical bond, or noncovalently, through ionic, van der Waals, electrostatic, or hydrogen bonds to a label such that the presence of the probe may be detected by detecting the presence of the label bound to the probe. Alternatively, a method using high affinity interactions may achieve the same results where one of a pair of binding partners binds to the other, e.g., biotin, streptavidin.

The terms “identical” or percent sequence “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site at ncbi.nlm.nih.gov/BLAST/ or the like). Such sequences are then said to be “substantially identical.” This definition also refers to, or may be applied to, the compliment of a test sequence. The definition also includes sequences that have deletions and/or additions, as well as those that have substitutions. Employed algorithms can account for gaps and the like.

For sequence comparisons, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

A preferred example of algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively.

The phrase “selectively (or specifically) hybridizes to” refers to the binding, duplexing, or hybridizing of a molecule only to a particular nucleotide sequence with a higher affinity, e.g., under more stringent conditions, than to other nucleotide sequences (e.g., total cellular or library DNA or RNA).

The phrase “stringent hybridization conditions” refers to conditions under which a nucleic acid will hybridize to its target sequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent hybridization conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent hybridization conditions may also be achieved with the addition of destabilizing agents such as formamide. For selective or specific hybridization, a positive signal is at least two times background, preferably 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency. Additional guidelines for determining hybridization parameters are provided in numerous reference, e.g., and Current Protocols in Molecular Biology, ed. Ausubel, et al., John Wiley & Sons.

Nucleic acids may be substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions.

An “inhibitory nucleic acid” is a nucleic acid (e.g. DNA, RNA, polymer of nucleotide analogs) that is capable of binding to a target nucleic acid (e.g. an mRNA translatable into an RPTP or IAD PTPR) and reducing transcription of the target nucleic acid (e.g. mRNA from DNA) or reducing the translation of the target nucleic acid (e.g. mRNA) or altering transcript splicing (e.g. single stranded morpholino oligo). A “morpholino oligo” may be alternatively referred to as a “morphlino nucleic acid” and refers to morpholine-containing nucleic acid nucleic acids commonly known in the art (e.g. phosphoramidate morpholinio oligo or a “PMO”). See Marcos, P., Biochemical and Biophysical Research Communications 358 (2007) 521-527. In some embodiments, the “inhibitory nucleic acid” is a nucleic acid that is capable of binding (e.g. hybridizing) to a target nucleic acid (e.g. an mRNA translatable into an RPTP or IAD PTPR) and reducing translation of the target nucleic acid. The target nucleic acid is or includes one or more target nucleic acid sequences to which the inhibitory nucleic acid binds (e.g. hybridizes). Thus, an inhibitory nucleic acid typically is or includes a sequence (also referred to herein as an “antisense nucleic acid sequence”) that is capable of hybridizing to at least a portion of a target nucleic acid at a target nucleic acid sequence, An example of an inhibitory nucleic acid is an antisense nucleic acid. Another example of an inhibitory nucleic acid is siRNA or RNAi (including their derivatives or pre-cursors, such as nucleotide analogs). Further examples include shRNA, miRNA, shmiRNA, or certain of their derivatives or pre-cursors. In some embodiments, the inhibitory nucleic acid is single stranded. In other embodiments, the inhibitory nucleic acid is double stranded.

An “antisense nucleic acid” is a nucleic acid (e.g. DNA, RNA or analogs thereof) that is at least partially complementary to at least a portion of a specific target nucleic acid (e.g. a target nucleic acid sequence), such as an mRNA molecule (e.g. a target mRNA molecule) (see, e.g., Weintraub, Scientific American, 262:40 (1990)), for example antisense, siRNA, shRNA, shmiRNA, miRNA (microRNA). Thus, antisense nucleic acids are capable of hybridizing to (e.g. selectively hybridizing to) a target nucleic acid (e.g. target mRNA). In some embodiments, the antisense nucleic acid hybridizes to the target nucleic acid sequence (e.g. mRNA) under stringent hybridization conditions. In some embodiments, the antisense nucleic acid hybridizes to the target nucleic acid (e.g. mRNA) under moderately stringent hybridization conditions. Antisense nucleic acids may comprise naturally occurring nucleotides or modified nucleotides such as, e.g., phosphorothioate, methylphosphonate, and -anomeric sugar-phosphate, backbone-modified nucleotides. An “anti-PTPR antisense nucleic acid” is an antisense nucleic acid that is at least partially complementary to at least a portion of a target nucleic acid sequence, such as an mRNA molecule, that codes at least a portion of the PTPR. An “anti-IAD PTPR antisense nucleic acid” is an antisense nucleic acid that is at least partially complementary to at least a portion of a target nucleic acid sequence, such as an mRNA molecule, that codes at least a portion of the PTPR. In some embodiments, an antisense nucleic acid is a morpholino oligo. In some embodiments, a morpholino oligo is a single stranded antisense nucleic acid, as is know in the art. In some embodiments, a morpholino oligo decreases protein expression of a target, reduces translation of the target mRNA, reduces translation initiation of the target mRNA, or modifies transcript splicing. In some embodiments, the morpholino oligo is conjugated to a cell permeable moiety (e.g. peptide). Antisense nucleic acids may be single or double stranded nucleic acids.

In the cell, the antisense nucleic acids may hybridize to the target mRNA, forming a double-stranded molecule. The antisense nucleic acids, interfere with the translation of the mRNA, since the cell will not translate a mRNA that is double-stranded. The use of antisense methods to inhibit the in vitro translation of genes is well known in the art (Marcus-Sakura, Anal. Biochem., 172:289, (1988)). Antisense molecules which bind directly to the DNA may be used.

Inhibitory nucleic acids can be delivered to the subject using any appropriate means known in the art, including by injection, inhalation, or oral ingestion. Another suitable delivery system is a colloidal dispersion system such as, for example, macromolecule complexes, nanocapsules, microspheres, beads, and lipid-based systems including oil-in-water emulsions, micelles, mixed micelles, and liposomes. An example of a colloidal system of this invention is a liposome. Liposomes are artificial membrane vesicles which are useful as delivery vehicles in vitro and in vivo. Nucleic acids, including RNA and DNA within liposomes and be delivered to cells in a biologically active form (Fraley, et al., Trends Biochem. Sci., 6:77, 1981). Liposomes can be targeted to specific cell types or tissues using any means known in the art Inhibitory nucleic acids (e.g. antisense nucleic acids, morpholino oligos) may be delivered to a cell using cell permeable delivery systems (e.g. cell permeable peptides). In some embodiments, inhibitory nucleic acids are delivered to specific cells or tissues using viral vectors or viruses.

An “siRNA” refers to a nucleic acid that forms a double stranded RNA, which double stranded RNA has the ability to reduce or inhibit expression of a gene or target gene when the siRNA is present (e.g. expressed) in the same cell as the gene or target gene. The siRNA is typically about 5 to about 100 nucleotides in length, more typically about 10 to about 50 nucleotides in length, more typically about 15 to about 30 nucleotides in length, most typically about 20-30 base nucleotides, or about 20-25 or about 24-29 nucleotides in length, e.g., 20, 21, 22, 23, 24, 25, 26, 27, 28, 29, or 30 nucleotides in length. siRNA molecules and methods of generating them are described in, e.g., Bass, 2001, Nature, 411, 428-429; Elbashir et al., 2001, Nature, 411, 494-498; WO 00/44895; WO 01/36646; WO 99/32619; WO 00/01846; WO 01/29058; WO 99/07409; and WO 00/44914. A DNA molecule that transcribes dsRNA or siRNA (for instance, as a hairpin duplex) also provides RNAi. DNA molecules for transcribing dsRNA are disclosed in U.S. Pat. No. 6,573,099, and in U.S. Patent Application Publication Nos. 2002/0160393 and 2003/0027783, and Tuschl and Borkhardt, Molecular Interventions, 2:158 (2002).

The siRNA can be administered directly or siRNA expression vectors can be used to induce RNAi that have different design criteria. A vector can have inserted two inverted repeats separated by a short spacer sequence and ending with a string of T's which serve to terminate transcription.

Construction of suitable vectors containing the desired therapeutic gene coding and control sequences employs standard ligation and restriction techniques, which are well understood in the art (see Maniatis et al., in Molecular Cloning: A Laboratory Manual, Cold Spring Harbor Laboratory, New York (1982)). Isolated plasmids, DNA sequences, or synthesized oligonucleotides are cleaved, tailored, and re-ligated in the form desired.

“Biological sample” or “sample” refer to materials obtained from or derived from a subject or patient. A biological sample includes sections of tissues such as biopsy and autopsy samples, and frozen sections taken for histological purposes. Such samples include bodily fluids such as blood and blood fractions or products (e.g., serum, plasma, platelets, red blood cells, and the like), sputum, tissue, cultured cells (e.g., primary cultures, explants, and transformed cells) stool, urine, synovial fluid, joint tissue, synovial tissue, synoviocytes, fibroblast-like synoviocytes, macrophage-like synoviocytes, immune cells, hematopoietic cells, fibroblasts, macrophages, T cells, etc. A biological sample is typically obtained from a eukaryotic organism, such as a mammal such as a primate e.g., chimpanzee or human; cow; dog; cat; a rodent, e.g., guinea pig, rat, mouse; rabbit; or a bird; reptile; or fish.

A “biopsy” refers to the process of removing a tissue sample for diagnostic or prognostic evaluation, and to the tissue specimen itself. Any biopsy technique known in the art can be applied to the diagnostic and prognostic methods of the present invention. The biopsy technique applied will depend on the tissue type to be evaluated (i.e., prostate, lymph node, liver, bone marrow, blood cell, joint tissue, synovial tissue, synoviocytes, fibroblast-like synoviocytes, macrophage-like synoviocytes, immune cells, hematopoietic cells, fibroblasts, macrophages, T cells, etc.), the size and type of a tumor (i.e., solid or suspended (i.e., blood or ascites)), among other factors. Representative biopsy techniques include excisional biopsy, incisional biopsy, needle biopsy, surgical biopsy, and bone marrow biopsy. Biopsy techniques are discussed, for example, in Harrison's Principles of Internal Medicine, Kasper, et al., eds., 16th ed., 2005, Chapter 70, and throughout Part V.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

A “label” or a “detectable moiety” is a composition detectable by spectroscopic, photochemical, biochemical, immunochemical, chemical, or other physical means. For example, useful labels include ³²P, fluorescent dyes, electron-dense reagents, enzymes (e.g., as commonly used in an ELISA), biotin, digoxigenin, or haptens and proteins or other entities which can be made detectable, e.g., by incorporating a radiolabel into a peptide or antibody specifically reactive with a target peptide. Any method known in the art for conjugating an antibody to the label may be employed, e.g., using methods described in Hermanson, Bioconjugate Techniques 1996, Academic Press, Inc., San Diego.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Typically, the antigen-binding region of an antibody will be most critical in specificity and affinity of binding. In some embodiments, antibodies or fragments of antibodies may be derived from different organisms, including humans, mice, rats, hamsters, camels, etc. Antibodies of the invention may include antibodies that have been modified or mutated at one or more amino acid positions to improve or modulate a desired function of the antibody (e.g. glycosylation, expression, antigen recognition, effector functions, antigen binding, specificity, etc.).

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}—C_{H}1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

For preparation of suitable antibodies of the invention and for use according to the invention, e.g., recombinant, monoclonal, or polyclonal antibodies, many techniques known in the art can be used (see, e.g., Kohler & Milstein, Nature 256:495-497 (1975); Kozbor et al., Immunology Today 4: 72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); and Goding, Monoclonal Antibodies: Principles and Practice (2d ed. 1986)). The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Random combinations of the heavy and light chain gene products generate a large pool of antibodies with different antigenic specificity (see, e.g., Kuby, Immunology (3^{rd} ed. 1997)). Techniques for the production of single chain antibodies or recombinant antibodies (U.S. Pat. No. 4,946,778, U.S. Pat. No. 4,816,567) can be adapted to produce antibodies to polypeptides of this invention. Also, transgenic mice, or other organisms such as other mammals, may be used to express humanized or human antibodies (see, e.g., U.S. Pat. Nos. 5,545,807; 5,545,806; 5,569,825; 5,625,126; 5,633,425; 5,661,016, Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); and Lonberg & Huszar, Intern. Rev. Immunol. 13:65-93 (1995)). Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)). Antibodies can also be made bispecific, i.e., able to recognize two different antigens (see, e.g., WO 93/08829, Traunecker et al., EMBO J. 10:3655-3659 (1991); and Suresh et al., Methods in Enzymology 121:210 (1986)). Antibodies can also be heteroconjugates, e.g., two covalently joined antibodies, or immunotoxins (see, e.g., U.S. Pat. No. 4,676,980, WO 91/00360; WO 92/200373; and EP 03089).

Methods for humanizing or primatizing non-human antibodies are well known in the art (e.g., U.S. Pat. Nos. 4,816,567; 5,530,101; 5,859,205; 5,585,089; 5,693,761; 5,693,762; 5,777,085; 6,180,370; 6,210,671; and 6,329,511; WO 87/02671; EP Patent Application 0173494; Jones et al. (1986) Nature 321:522; and Verhoyen et al. (1988) Science 239:1534). Humanized antibodies are further described in, e.g., Winter and Milstein (1991) Nature 349:293. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers (see, e.g., Morrison et al., PNAS USA, 81:6851-6855 (1984), Jones et al., Nature 321:522-525 (1986); Riechmann et al., Nature 332:323-327 (1988); Morrison and Oi, Adv. Immunol., 44:65-92 (1988), Verhoeyen et al., Science 239:1534-1536 (1988) and Presta, Curr. Op. Struct. Biol. 2:593-596 (1992), Padlan, Molec. Immun., 28:489-498 (1991); Padlan, Molec. Immun., 31(3):169-217 (1994)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies. For example, polynucleotides comprising a first sequence coding for humanized immunoglobulin framework regions and a second sequence set coding for the desired immunoglobulin complementarity determining regions can be produced synthetically or by combining appropriate cDNA and genomic DNA segments. Human constant region DNA sequences can be isolated in accordance with well known procedures from a variety of human cells.

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity. The preferred antibodies of, and for use according to the invention include humanized and/or chimeric monoclonal antibodies.

In one embodiment, the antibody is conjugated to an “effector” moiety. The effector moiety can be any number of molecules, including labeling moieties such as radioactive labels or fluorescent labels, or can be a therapeutic moiety. In one aspect the antibody modulates the activity of the protein. Such effector moieties include, but are not limited to, an anti-tumor drug, a toxin, a radioactive agent, a cytokine, a second antibody or an enzyme. Further, the invention provides an embodiment wherein the antibody of the invention is linked to an enzyme that converts a prodrug into a cytotoxic agent.

The immunoconjugate can be used for targeting the effector moiety to an RPTP or IAD PTPR positive cell, particularly cells, which express a RPTP or IAD PTPR protein. Such differences can be readily apparent when viewing the bands of gels with approximately similarly loaded with test and controls samples. Examples of cytotoxic agents include, but are not limited to ricin, doxorubicin, daunorubicin, taxol, ethidium bromide, mitomycin, etoposide, tenoposide, vincristine, vinblastine, colchicine, dihydroxy anthracin dione, actinomycin D, diphteria toxin, Pseudomonas exotoxin (PE) A, PE40, abrin, and glucocorticoid and other chemotherapeutic agents, as well as radioisotopes. Suitable detectable markers include, but are not limited to, a radioisotope, a fluorescent compound, a bioluminescent compound, chemiluminescent compound, a metal chelator or an enzyme.

Additionally, the recombinant protein of the invention comprising the antigen-binding region of any of the monoclonal antibodies of the invention can be used to treat cancer. In such a situation, the antigen-binding region of the recombinant protein is joined to at least a functionally active portion of a second protein having therapeutic activity. The second protein can include, but is not limited to, an enzyme, lymphokine, oncostatin or toxin. Suitable toxins include doxorubicin, daunorubicin, taxol, ethidium bromide, mitomycin, etoposide, tenoposide, vincristine, vinblastine, colchicine, dihydroxy anthracin dione, actinomycin D, diphteria toxin, Pseudomonas exotoxin (PE) A, PE40, ricin, abrin, glucocorticoid and radioisotopes.

Techniques for conjugating therapeutic agents to antibodies are well known (see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery” in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review” in Monoclonal Antibodies '84: Biological And Clinical Applications, Pinchera et al. (eds.), pp. 475-506 (1985); and Thorpe et al., “The Preparation And Cytotoxic Properties Of Antibody-Toxin Conjugates”, Immunol. Rev., 62:119-58 (1982)).

The phrase “specifically (or selectively) binds” to an antibody or “specifically (or selectively) immunoreactive with,” when referring to a protein or peptide, refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies can be selected to obtain only those polyclonal antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Using Antibodies, A Laboratory Manual (1998) for a description of immunoassay formats and conditions that can be used to determine specific immunoreactivity).

As used herein, the term “pharmaceutically acceptable” is used synonymously with “physiologically acceptable” and “pharmacologically acceptable”. A pharmaceutical composition will generally comprise agents for buffering and preservation in storage, and can include buffers and carriers for appropriate delivery, depending on the route of administration.

Certain compounds of the present invention can exist in unsolvated forms as well as solvated forms, including hydrated forms. In general, the solvated forms are equivalent to unsolvated forms and are intended to be encompassed within the scope of the present invention. Certain compounds of the present invention may exist in multiple crystalline or amorphous forms. In general, all physical forms are equivalent for the uses contemplated by the present invention and are intended to be within the scope of the present invention.

“PTPR” or “RPTP” or “rPTP” (all terms are equal) refer to receptor protein tyrosine phosphatases, which are found in nature as membrane bound protein tyrosine phosphatases. In some embodiments, the RPTP is a mammalian RPTP (e.g. human, mouse, rat, or other mammal). In some embodiments, the RPTP is a human RPTP. In some embodiments, the RPTP refers to PTPRA, PTPRB, PTPRC, PTPRD, PTPRE, PTPRF, PTPRG, PTPRH, PTPRJ, PTPRK, PTPRM, PTPRN, PTPRN2, PTPRO, PTPRQ, PTPRR, PTPRS, PTPRT, PTPRU, PTPRV or PTPRZ1. In some embodiments, RPTP means the full length RPTP (e.g. the protein translated from the complete coding region of the gene, which may also include post-translational modifications). In some embodiments RPTP includes a fragment of the RPTP full length protein or a functional fragment of the full length RPTP protein. In some embodiments this definition includes one or all splice variants of an RPTP. An RPTP may include all homologs of the RPTP. In some embodiments, PTPRA, PTPRB, PTPRC, PTPRD, PTPRE, PTPRF, PTPRG, PTPRH, PTPRJ, PTPRK, PTPRM, PTPRN, PTPRN2, PTPRO, PTPRQ, PTPRR, PTPRS, PTPRT, PTPRU, PTPRV or PTPRZ1 respectively, refers to mammalian PTPRA, PTPRB, PTPRC, PTPRD, PTPRE, PTPRF, PTPRG, PTPRH, PTPRJ, PTPRK, PTPRM, PTPRN, PTPRN2, PTPRO, PTPRQ, PTPRR, PTPRS, PTPRT, PTPRU, PTPRV or PTPRZ1, respectively. In some embodiments, a PTPRA, PTPRB, PTPRC, PTPRD, PTPRE, PTPRF, PTPRG, PTPRH, PTPRJ, PTPRK, PTPRM, PTPRN, PTPRN2, PTPRO, PTPRQ, PTPRR, PTPRS, PTPRT, PTPRU, PTPRV or PTPRZ1 respectively, refers to a human PTPRA, PTPRB, PTPRC, PTPRD, PTPRE, PTPRF, PTPRG, PTPRH, PTPRJ, PTPRK, PTPRM, PTPRN, PTPRN2, PTPRO, PTPRQ, PTPRR, PTPRS, PTPRT, PTPRU, PTPRV or PTPRZ1, respectively. In some embodiments, an RPTP includes all splice variants of the RPTP. In other embodiments, an RPTP may refer to 1, 2, 3, 4, 5, 6, 7, 8, 9, 10, or more splice variants. RPTPZ1 may also be called “PTPRZ”

A “protein level of an RPTP” refers to an amount (relative or absolute) of RPTP in its protein form (as distinguished from its precursor RNA form) A protein of an RPTP may include a full-length protein (e.g. the protein translated from the complete coding region of the gene, which may also include post-translational modifications), functional fragments of the full length protein (e.g. sub-domains of the full length protein that possess an activity or function in an assay), or protein fragments of the RPTP, which may be any peptide or oligopeptide of the full length protein.

An “RNA level of an RPTP” refers to an amount (relative or absolute) of RNA present that may be translated to form an RPTP. The RNA of an RPTP may be a full-length RNA sufficient to form a full-length RPTP. The RNA of an RPTP may also be a fragment of the full length RNA thereby forming a fragment of the full length RPTP. The fragment of the full length RNA may form a functional fragment of the RPTP. In some embodiments, the RNA of an RPTP includes all splice variants of an RPTPR gene.

“IAD PTPR” or “inflammatory autoimmune disease PTPR” refers to PTPRA, PTPRB, PTPRC, PTPRE, PTPRG, PTPRJ, PTPRM, PTPRO and PTPRS. In some embodiments, IAD PTPR means the full length IAD PTPR (e.g. the protein translated from the complete coding region of the gene, which may also include post-translational modifications). In some embodiments, IAD PTPR includes a fragment of the IAD PTPR full length protein or a functional fragment of the full length IAD PTPR protein. In some embodiments and IAD PTPR includes one or all splice variants of an IAD PTPR. IAD PTPR may include all homologs of the IAD PTPR. In some embodiments, PTPRA, PTPRB, PTPRC, PTPRE, PTPRG, PTPRJ, PTPRM, PTPRO, or PTPRS respectively, refers to mammalian PTPRA, PTPRB, PTPRC, PTPRE, PTPRG, PTPRJ, PTPRM, PTPRO, or PTPRS, respectively. In some embodiments, a PTPRA, PTPRB, PTPRC, PTPRE, PTPRG, PTPRJ, PTPRM, PTPRO, or PTPRS respectively, refers to a human PTPRA, PTPRB, PTPRC, PTPRE, PTPRG, PTPRJ, PTPRM, PTPRO, or PTPRS, respectively. In some embodiments, an IAD PTPR includes all splice variants of the IAD PTPR. In other embodiments, an IAD PTPR may refer to 1, 2, 3, 4, 5, 6, 7, 8, 9, 10, or more splice variants collectively. In some embodiments, IAD PTPRs are relevant to methods and compositions described herein relating to any autoimmune disease.

A “protein level of an IAD PTPR” refers to an amount (relative or absolute) of an IAD PTPR in its protein form (as distinguished from its precursor RNA form). A protein of an IAD PTPR may include a full-length protein (e.g. the protein translated from the complete coding region of the gene, which may also include post-translational modifications), functional fragments of the full length protein (e.g. sub-domains of the full length protein that possess an activity or function in an assay), or protein fragments of the one IAD PTPR, which may be any peptide or oligopeptide of the full length protein.

An “RNA level of an IAD PTPR” refers to an amount (relative or absolute) of RNA present that may be translated to form an IAD PTPR. The RNA of an IAD PTPR may be a full-length RNA sufficient to form a full-length IAD PTPR. The RNA of an IAD PTPR may also be a fragment of the full length RNA thereby forming a fragment of the full length IAD PTPR. The fragment of the full length RNA may form a functional fragment of the IAD PTPR.

An “autoimmune therapeutic agent” is a molecule (e.g. antibody, nucleic acid, inhibitory nucleic acid, synthetic chemical, small chemical molecule) that treats or prevents an autoimmune disease when administered to a subject in a therapeutically effective dose or amount. In some embodiments, an autoimmune therapeutic agent is an RPTP binding agent. In some embodiments, the therapeutic agent can bind to more than one RPTP.

An “IAD therapeutic agent” is a molecule that treats or prevents an inflammatory autoimmune disease when administered to a subject in a therapeutically effective dose or amount where the autoimmune disease is mediated by an IAD PTPR. Some non-limiting examples of an IAD therapeutic agent include an IAD PTPR binding agent, anti-IAD PTPR antibody (e.g. an anti-IAD PTPRS antibody or an anti-IAD PTPRE antibody), anti-IAD PTPR inhibitory nucleic acid (e.g. an anti-IAD PTPR antisense nucleic acid such as an anti-IAD PTPRS antisense nucleic acid or an anti-IAD PTPRE antisense nucleic acid), anti-IAD PTPR RNAi molecule, and an IAD PTPR ligand mimetic. In some embodiments, IAD therapeutic agents are useful in methods and compositions described herein relating to any autoimmune disease. In some embodiments, the IAD therapeutic agent can bind to more than one RPTP.

An “RPTP binding agent” is a molecule that binds (e.g. preferentially binds) to one or more RPTPs, RNA that is translatable to an RPTP, or DNA that is transcribable to an RNA that is translatable to an RPTP. Where the molecule preferentially binds, the binding is preferential as compared to other macromolecular biomolecules present in an organism or cell. A compound preferentially binds to as compared to other macromolecular biomolecules present in an organism or cell, for example, when the preferential binding is 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, 2000-fold, 3000-fold, 4000-fold, 5000-fold, 6000-fold, 7000-fold, 8000-fold, 9000-fold, 10000 fold, 100,000-fold, 1,000,000-fold greater. In some embodiments, the RPTP binding agent preferentially binds to one or more RPTPs. In some embodiments, the RPTP binding agent preferentially binds to one RPTP (e.g. PTPRA, PTPRB, PTPRC, PTPRD, PTPRE, PTPRF, PTPRG, PTPRH, PTPRJ, PTPRK, PTPRM, PTPRN, PTPRN2, PTPRO, PTPRQ, PTPRR, PTPRS, PTPRT, PTPRU, PTPRV, or PTPRZ1) in comparison to one or more other RPTPs. In some embodiments, the RPTP binding agent preferentially binds to an RNA that is translatable to an RPTP (e.g. PTPRA, PTPRB, PTPRC, PTPRD, PTPRE, PTPRF, PTPRG, PTPRH, PTPRJ, PTPRK, PTPRM, PTPRN, PTPRN2, PTPRO, PTPRQ, PTPRR, PTPRS, PTPRT, PTPRU, PTPRV, or PTPRZ1 nucleic acid) compared to an RNA that is translatable to another RPTP nucleic acids. In some embodiments, the RNA is mRNA. In some embodiments, the RPTP binding agent is a protein, nucleic acid, ligand, ligand mimetic, or a small chemical molecule. In some embodiments, an RPTP binding agent disrupts the interaction between an RPTP and a physiological or natural ligand. In some embodiments, an RPTP binding agent binds a physiological or natural ligand of the RPTP. In some embodiments, an RPTP binding agent binds the complex of an RPTP bound to a ligand. In some embodiments, the binding agent can bind to more than one RPTP.

An “IAD PTPR binding agent” is a molecule that binds (e.g. preferentially binds) to one or more IAD PTPRs, a target nucleic acid sequence (e.g. an RNA sequence) that is translatable to an IAD PTPR, or a target nucleic acid sequence (e.g. a DNA sequence) that is transcribable to an RNA that is translatable to an IAD PTPR. Where the molecule preferentially binds, the binding is preferential as compared to other macromolecular biomolecules present in an organism or cell. In some embodiments, the IAD PTPR binding agent preferentially binds to one or more IAD PTPRs. In some embodiments, the IAD PTPR binding agent preferentially binds to one IAD PTPR (e.g. PTPRA, PTPRB, PTPRC, PTPRE, PTPRG, PTPRJ, PTPRM, PTPRO, or PTPRS) in comparison to one or more other IAD PTPRs. In some embodiments, the IAD PTPR binding agent preferentially binds to an RNA that is translatable to an IAD PTPR (e.g. PTPRA, PTPRB, PTPRC, PTPRE, PTPRG, PTPRJ, PTPRM, PTPRO, or PTPRS nucleic acid) compared to an RNA that is translatable to another IAD PTPR nucleic acids. In some embodiments, the RNA is mRNA. In some embodiments, the IAD PTPR binding agent is a protein, nucleic acid, ligand, ligand mimetic, or a small chemical molecule. In some embodiments the IAD PTPR binding agent binds the full length IAD PTPR (e.g. the protein translated from the complete coding region of the gene), the complete RNA that is translatable to an full length IAD PTPR, or complete coding DNA of the IAD PTPR, which may also include post-translational modifications or post-transcriptional modifications. In some embodiments an IAD PTPR binding agent binds a fragment of the IAD PTPR full length protein, RNA that is translatable to a fragment of the IAD PTPR, or DNA that is transcribable to RNA that is translatable to a fragment of the IAD PTPR or a functional fragment of the full length IAD PTPR protein, RNA that is translatable to a functional fragment of the IAD PTPR, or DNA that is transcribable RNA that is transcribable to a functional fragment of the IAD PTPR. In some embodiments the IAD PTPR binding agent binds one or all splice variants of an IAD PTPR. In some embodiments, an IAD PTPR binding agent disrupts the interaction between an IAD PTPR and a physiological or natural ligand. In some embodiments, an IAD PTPR binding agent binds a physiological or natural ligand of the IAD PTPR. In some embodiments, an IAD PTPR binding agent binds the complex an IAD PTPR bound to a ligand. In some embodiments, IAD binding agents are useful in methods and compositions described herein relating to any autoimmune disease. In some embodiments, the IAD PTPR binding agent can bind to more than one RPTP.

An “anti-IAD PTPR inhibitory nucleic acid” is an inhibitory nucleic acid that is capable of hybridizing to target nucleic acid sequence (e.g. an mRNA sequence) that is translatable to an IAD PTPR or a target nucleic acid sequence (e.g. a DNA sequence) that is transcribable to an RNA that is translatable to an IAD PTPR. The anti-IAD PTPR inhibitory nucleic acid is typically capable of decreasing the amount of IAD PTPR that is translated in a cell.

An “anti-IAD PTPR RNAi molecule” is an siRNA, shRNA, miRNA, shmiRNA, or other nucleic acid that is capable of inducing RNAi and hybridizing to an RNA that is translatable to an IAD PTPR. The anti-IAD PTPR RNAi molecule is typically capable of decreasing the amount of IAD PTPR that is translated in a cell.

An “IAD ligand mimetic” is an IAD PTPR binding agent that is designed to mimic, in structure or in binding mode, a known IAD ligand or is capable of inhibiting the binding of a natural or physiological ligand to an IAD PTPR. In some embodiments, an IAD ligand mimetic is a synthetic chemical compound, peptide, protein, fusion protein (e.g. PTPR-Fc), peptidomimetic, or modified natural ligand. For example, an IAD ligand mimetic may bind the same amino acids or a subset of the same amino acids on the IAD PTPR that a natural ligand of the IAD PTPR binds during the physiological functioning of the IAD PTPR. IAD ligand mimetics include biopolymers (e.g. proteins, nucleic acids, or sugars), lipids, chemical molecules with molecular weights less than five hundred (500) Daltons, one thousand (1000) Daltons, five thousand (5000) Daltons, less than ten thousand (10,000) Daltons, less than twenty five thousand (25,000) Daltons, less than fifty thousand (50,000) Daltons, less than seventy five thousand (75,000), less than one hundred thousand (100,000), or less than two hundred fifty thousand (250,000) Daltons. In some embodiments, the synthetic chemical compound is greater than two hundred fifty thousand (250,000) Daltons. In certain embodiments, the IAD PTPR binding agent is less than five hundred (500) Daltons. In some embodiments, an IAD PTPR ligand mimetic is a protein.

In some embodiments, an IAD ligand mimetic is a small chemical molecule. A “small chemical molecule” is a molecule that has a molecular weight of less than two thousand (2000) Daltons. In some embodiments, a small chemical molecule is a molecule that has a molecular weight of less than one thousand (1000) Daltons. In other embodiments, a small chemical molecule is a molecule that has a molecular weight of less than five hundred (500) Daltons. In other embodiments, a small chemical molecule is a molecule that has a molecular weight of less than five hundred (500) Daltons. In other embodiments, a small chemical molecule is a molecule that has a molecular weight of less than one hundred (100) Daltons.

An agent may “target” an RPTP, an IAD PTPR, a nucleic acid (e.g. RNA or DNA) of an RPTP, nucleic acid (e.g. RNA or DNA) of an IAD PTPR, a protein of an RPTP, or a protein of an IAD PTPR by binding (e.g. preferentially binding) to the RPTP, IAD PTPR, nucleic acid (e.g. RNA or DNA) of an RPTP, nucleic acid (e.g. RNA or DNA) of an IAD PTPR, protein of an RPTP, or protein of an IAD PTPR. Where preferentially binding, the agent binds preferentially compared to its binding to other molecules of a similar form (e.g. other proteins if the agent targets a protein of an IAD PTPR, other RPTPs if the agent targets an RPTP). An agent preferentially binds to a molecule, for example, when the binding to the targeted molecule is greater than the binding to other molecules of a similar form. In some embodiments, the preferential binding is 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, 2000-fold, 3000-fold, 4000-fold, 5000-fold, 6000-fold, 7000-fold, 8000-fold, 9000-fold, 10000 fold, 100,000-fold, 1,000,000-fold greater. In some embodiments, an agent targets an RPTP, an IAD PTPR, a nucleic acid (e.g. RNA or DNA) of an RPTP, a nucleic acid (e.g. RNA or DNA) of an IAD PTPR, a protein of an RPTP, or a protein of an IAD PTPR when a binding assay or experiment (e.g. gel electrophoresis, chromatography, immunoassay, radioactive or non-radioactive labeling, immunoprecipitation, activity assay, etc.) reveals only an interaction or primarily an interaction with a single RPTP, a single IAD PTPR, a nucleic acid (e.g. RNA or DNA) of a single RPTP, a nucleic acid (e.g. RNA or DNA) of a single IAD PTPR, a protein of a single RPTP, or a protein of a single IAD PTPR. An agent may also “target” an RPTP, an IAD PTPR, a nucleic acid (e.g. RNA or DNA) of an RPTP, a nucleic acid (e.g. RNA or DNA) of an IAD PTPR, a protein of an RPTP, or a protein of an IAD PTPR by binding to the RPTP, IAD PTPR, nucleic acid (e.g. RNA or DNA) of an RPTP, RNA of an IAD PTPR, protein of an RPTP, or protein of an IAD PTPR by decreasing or increasing the amount of RPTP in a cell or organism relative to the absence of the agent, decreasing the interaction between the RPTP or IAD PTPR with a physiological or natural ligand. A person having ordinary skill in the art, using the guidance provided herein, may easily determine whether an agent decreases or increases the amount of an RPTP in a cell or organism.

### II. Methods of Determining Whether a Subject has or is at Risk of Developing a Disease

Provided herein are methods of determining whether an individual has or may be at risk of developing a disease. The disease is associated with a differential expression of a receptor protein tyrosine phosphatase relative to individuals who do not have or are not at risk of developing the disease. In some embodiments, the disease is an autoimmune disease or disorder, cancer, an infectious disease (e.g. viral, bacterial, parasitic, etc.), an obesity associated disease, a metabolic disease or disorder, an inflammatory disease, an immune disease or disorder, or a traumatic injury. In some embodiments, the disease is an inflammatory autoimmune disease (IAD). In some embodiments, the disease is a disease associated with a patient's joints. In certain embodiments, the inflammatory autoimmune disease is rheumatoid arthritis.

In one aspect, a method is provided for determining whether a subject has or is at risk of developing an autoimmune disease (e.g. an inflammatory autoimmune disease such as rheumatoid arthritis). The method includes determining whether a subject expresses a modulated RNA level of an RPTP (an RNA translatable to an RPTP such as an mRNA) or a modulated protein level of an RPTP relative to a standard control. The presence of the modulated RNA level or the modulated protein level indicates the subject has or is at risk of developing an autoimmune disease. In other embodiments, the presence of the modulated RNA level or the modulated protein level indicates the subject has an autoimmune disease. In certain embodiments, the presence of the modulated RNA level or the modulated protein level indicates the subject is at risk of developing an autoimmune disease (e.g. rheumatoid arthritis). The RPTP may be PTPRA, PTPRB, PTPRC, PTPRD, PTPRE, PTPRF, PTPRG, PTPRH, PTPRJ, PTPRK, PTPRM, PTPRN, PTPRN2, PTPRO, PTPRQ, PTPRR, PTPRS, PTPRT, PTPRU, PTPRV or PTPRZ1. In another embodiment, the RPTP is PTPRA, PTPRB, PTPRC, PTPRE, PTPRF, PTPRG, PTPRJ, PTPRM, PTPRO, PTPRS, or PTPRZ1. In other embodiments, the RPTP is PTPRA, PTPRB, PTPRC, PTPRE, PTPRG, PTPRJ, PTPRM, PTPRO, or PTPRS. In still other embodiments, the RPTP is PTPRB, PTPRE, PTPRG, PTPRJ, PTPRM, PTPRO, or PTPRS. In further embodiments, the RPTP is PTPRB, PTPRE, PTPRG, PTPRM, PTPRO, or PTPRS. In other embodiments, the RPTP is PTPRE, PTPRG, PTPRM, PTPRO, or PTPRS. In some embodiments, the RPTP is PTPRE or PTPRS. In other embodiments, the RPTP is PTPRE. In other embodiments, the RPTP is PTPRS. In other embodiments, the RPTP is PTPRF. In other embodiments, the RPTP is PTPRZ1.

In some embodiments, the RNA whose level is modulated (e.g. elevated) has the sequence set forth in SEQ ID NO: 2, SEQ ID NO: 4, SEQ ID NO: 6, SEQ ID NO: 8, SEQ ID NO: 11 or SEQ ID NO: 13 (wherein the “T” is “U” in the actual RNA sequence). In some embodiments, the protein whose level is modulated (e.g. elevated) has the sequence set forth in SEQ ID NO: 3, SEQ ID NO: 5, SEQ ID NO: 7, SEQ ID NO: 9, SEQ ID NO: 12, or SEQ ID NO: 14. In some embodiments, the protein whose level is modulated (e.g. elevated) is encoded by the sequence set forth in SEQ ID NO: 1, SEQ ID NO: 10.

In some embodiments, the method includes determining whether a subject has or is at risk of developing an inflammatory autoimmune disease (IAD). The method includes determining whether a subject expresses an elevated RNA level of an IAD PTPR or an elevated protein level of an IAD PTPR relative to a standard control. The presence of the elevated RNA level or the elevated protein level indicates the subject has or is at risk of developing the inflammatory autoimmune disease. In certain embodiments, the presence of the elevated RNA level or the elevated protein level indicates the subject has the inflammatory autoimmune disease. In other embodiments, the presence of the elevated RNA level or the elevated protein level indicates the subject is at risk of developing the inflammatory autoimmune disease (e.g. rheumatoid arthritis).

In certain embodiments, the IAD PTPR is PTPRA, PTPRB, PTPRC, PTPRE, PTPRG, PTPRJ, PTPRM, PTPRO, or PTPRS. The IAD PTPR may be PTPRB, PTPRE, PTPRG, PTPRJ, PTPRM, PTPRO, or PTPRS. In other embodiments, the IAD PTPR is PTPRB, PTPRE, PTPRG, PTPRM, PTPRO, or PTPRS. In still other embodiments, the IAD PTPR is PTPRE, PTPRG, PTPRM, PTPRO, or PTPRS. The IAD PTPR may be PTPRE or PTPRS. For example, the IAD PTPR may be PTPRS. For example, the IAD PTPR may be PTPRE.

In some embodiments, the method includes determining whether a subject has or is at risk of developing an inflammatory autoimmune disease (IAD). The method includes determining whether a subject expresses a decreased RNA level of a PTPR or a decreased protein level of a PTPR relative to a standard control. The PTPR may be PTPRF or PTPRZ1 (e.g. a combination of PTPRF and PTPRZ1). The presence of the decreased RNA levels or the decreased protein level indicates the subject has or is at risk of developing the inflammatory autoimmune disease. In certain embodiments, the presence of the decreased RNA level or the decreased protein level indicates the subject has the inflammatory autoimmune disease. In other embodiments, the presence of the decreased RNA level or the decreased protein level indicates the subject is at risk of developing the inflammatory autoimmune disease. Thus, the decreased expression of one or more (e.g. 2, 3, 4, 5, 6, 7, 8, 9, 10, 11, 12, 13, 14, 15, 16, 17, 18, 19, 20, 21) RPTPs or IAD PTPRs may be associated with a disease or a risk of developing the disease. In some embodiments, the decreased expression of PTPRF is associated with rheumatoid arthritis. In certain embodiments, the decreased expression of PTPRZ1 is associated with rheumatoid arthritis.

In some embodiments, the increased expression of one or more (e.g. 2, 3, 4, 5, 6, 7, 8, 9, 10, 11, 12, 13, 14, 15, 16, 17, 18, 19, 20, 21) RPTPs or IAD PTPRs is associated with the presence of a disease or a risk of developing the disease. In other embodiments, the increased expression of PTPRA, PTPRB, PTPRC, PTPRE, PTPRG, PTPRJ, PTPRM, PTPRO, PTPRS, or a combination thereof (e.g. 2, 3, 4, 5, 6, 7, 8, 9) is associated with the presence of or risk of developing an inflammatory autoimmune disease. In some embodiments, the increased expression of PTPRB, PTPRE, PTPRG, PTPRM, PTPRO, PTPRS, or a combination thereof (e.g. 2, 3, 4, 5, 6) is associated with an inflammatory autoimmune disease. In some embodiments, the increased expression of PTPRE, PTPRG, PTPRM, PTPRO, PTPRS, or a combination thereof (e.g. 2, 3, 4, 5) is associated with an inflammatory autoimmune disease. In other embodiments, the increased expression of PTPRE, PTPRS, or a combination thereof is associated with an inflammatory autoimmune disease. In some embodiments, the increased expression of PTPRS is associated with an inflammatory autoimmune disease. In certain embodiments, the increased expression of PTPRA, PTPRB, PTPRC, PTPRE, PTPRG, PTPRJ, PTPRM, PTPRO, PTPRS, or a combination thereof (e.g. 2, 3, 4, 5, 6, 7, 8, 9) is associated with rheumatoid arthritis. In some embodiments, the increased expression of PTPRB, PTPRE, PTPRG, PTPRM, PTPRO, PTPRS, or a combination thereof (e.g. 2, 3, 4, 5, 6) is associated with rheumatoid arthritis. In other embodiments, the increased expression of PTPRE, PTPRG, PTPRM, PTPRO, PTPRS, or a combination thereof (e.g. 2, 3, 4, 5) is associated with rheumatoid arthritis. In certain embodiments, the increased expression of PTPRE, PTPRS, or a combination thereof is associated with rheumatoid arthritis. In some embodiments, the increased expression of PTPRS is associated with rheumatoid arthritis. In some embodiments, the increased expression of PTPRE is associated with rheumatoid arthritis.

In some embodiment, the determining of whether a subject expresses a modulated (e.g. increased) RNA level of an RPTP or a modulated (e.g. increased) protein level of an RPTP relative to a standard control includes correlating the levels to a disease or associating the levels with or to a disease. The terms “correlating” and “associating,” in reference to the measurement and/or analysis of RNA or protein levels with a disease (e.g. an autoimmune, inflammatory autoimmune, cancer, infectious, immune, or other disease), includes to comparing the levels with a standard control and drawing a conclusion as to the presence of the disease in a subject or the likelihood of the subject developing a disease. For example, the comparison may be made between levels in the subject to levels in persons known to suffer from, or known to be at risk of, the disease (e.g. an autoimmune, inflammatory autoimmune, cancer, infectious, immune, or other disease), or in persons known to be free of the disease (e.g. an autoimmune, inflammatory autoimmune, cancer, infectious, immune, or other disease), and assigning an increased or decreased probability of having/developing the disease (e.g. an autoimmune, inflammatory autoimmune, cancer, infectious, immune, or other disease) to an individual based on the assay result(s).

In some embodiments, determining whether a subject has or is at risk of having a disease includes one or more of the following actions: collecting a sample from a subject, purifying the sample, measuring a characteristic of the sample, analyzing the results of the measurement; assigning a probability of the subject having or developing the disease. In certain embodiments the assigning of a probability of having or developing the disease is done by comparing the outcome of the measurement to the measurement of the same parameter from a person who does not have or is not at risk of having the disease. In other embodiments, the determination is made by comparing the measurement to a threshold established from previous measurements of patients having or at risk of having a disease and measurements of patients who did not have or were not at risk of having a disease. In some embodiments, determining if a subject has a risk factor (e.g. an elevated or reduced level of an RNA or protein of an RPTP or IAD PTPR) for a disease (e.g. an autoimmune, inflammatory autoimmune, cancer, infectious, immune, or other disease) includes measuring the characteristic or parameter of the subject (e.g. obtaining a sample from the patient, purifying the sample, testing the sample) and analyzing the outcome of the test, using methods and compositions known by persons of ordinary skill in diagnostic methods and as described herein. In other embodiments, determining whether a person has or is at risk of having a disease includes measuring a risk factor from a sample obtained or derived from the subject and comparing the measurement to the same measurement of the risk factor from a standard control, with differences between the two measurements indicating that the subject has or is at risk of having the disease.

As described above, the results may “indicate” the subject has or is at risk of developing the autoimmune disease. This indication may be a correlated measured quantity that strongly or convincingly implies (or points toward) the presence or risk of developing the autoimmune disease. This indication may also mean that a positive outcome of a test equates to a positive answer to the correlation.

The increased expression of a first RPTP and the decreased expression of a second RPTP may also be associated with a disease or a risk of developing the disease. In some embodiments increased expression of PTPRA, PTPRB, PTPRC, PTPRE, PTPRG, PTPRJ, PTPRM, PTPRO, PTPRS, or a combination thereof (e.g. 2, 3, 4, 5, 6, 7, 8, 9) and the decreased expression of PTPRF or PTPRZ1, or a combination thereof, is associated with an inflammatory autoimmune disease. Increased expression of PTPRA, PTPRB, PTPRC, PTPRE, PTPRG, PTPRJ, PTPRM, PTPRO, PTPRS, or a combination thereof (e.g. 2, 3, 4, 5, 6, 7, 8, 9) and the decreased expression of PTPRF or PTPRZ1, or a combination thereof, may be associated with rheumatoid arthritis. In other embodiments increased expression of PTPRE and PTPRS and decreased expression of PTPRF and PTPRZ1 is associated with an inflammatory autoimmune disease. In certain embodiments increased expression of PTPRE and PTPRS and decreased expression of PTPRF and PTPRZ1 is associated with rheumatoid arthritis.

An autoimmune disease is a disease or condition in which a subject's immune system irregularly responds to one or more components (e.g. biomolecule, protein, cell, tissue, organ, etc.) of the subject. In some embodiments, an autoimmune disease is a condition in which the subject's immune system irregularly reacts to one or more components of the subject as if such components were not self. In some embodiments, an autoimmune disease is associated with a differential expression of an RPTP or an IAD PTPR. In some embodiments, an autoimmune disease is associated with a mutation in an RPTP or IAD PTPR. Non-limiting examples of autoimmune diseases that may be associated with a differential expression of an RPTP include Acute Disseminated Encephalomyelitis (ADEM), Acute necrotizing hemorrhagic leukoencephalitis, Addison's disease, Agammaglobulinemia, Asthma, Allergic asthma, Allergic rhinitis, Alopecia greata, Amyloidosis, Ankylosing spondylitis, Anti-GBM/Anti-TBM nephritis, Antiphospholipid syndrome (APS), Arthritis, Autoimmune aplastic anemia, Autoimmune dysautonomia, Autoimmune hepatitis, Autoimmune hyperlipidemia, Autoimmune immunodeficiency, Autoimmune inner ear disease (AIED), Autoimmune myocarditis, Autoimmune pancreatitis, Autoimmune retinopathy, Autoimmune thrombocytopenic purpura (ATP), Autoimmune thyroid disease, Axonal & neuronal neuropathies, Balo disease, Behcet's disease, Bullous pemphigoid, Cardiomyopathy, Castleman disease, Celiac sprue, Chagas disease, Chronic inflammatory demyelinating polyneuropathy (CIDP), Chronic recurrent multifocal ostomyelitis (CRMO), Churg-Strauss syndrome, Cicatricial pemphigoid/benign mucosal pemphigoid, Crohn's disease, Cogans syndrome, Cold agglutinin disease, Congenital heart block, Coxsackie myocarditis, CREST disease, Essential mixed cryoglobulinemia, Demyelinating neuropathies, Dermatitis herpetiformis, Dermatomyositis, Devic's disease (neuromyelitis optica), Discoid lupus, Dressler's syndrome, Endometriosis, Eosinophilic fasciitis, Erythema nodosum, Experimental allergic encephalomyelitis, Evans syndrome, Fibrosing alveolitis, Giant cell arteritis (temporal arteritis), Glomerulonephritis, Goodpasture's syndrome, Graves' disease, Grave's ophthalmopathy, Guillain-Barre syndrome, Hashimoto's encephalitis, Hashimoto's thyroiditis, Hemolytic anemia, Henoch-Schonlein purpura, Herpes gestationis, Hypogammaglobulinemia, Ichthyosis, Idiopathic thrombocytopenic purpura (ITP), IgA nephropathy, IgG4-related sclerosing disease, Immunoregulatory lipoproteins, Inclusion body myositis, Inflammatory bowel disease, Insulin-dependent diabetes (type1), Interstitial cystitis, Juvenile arthritis, Juvenile diabetes, Kawasaki syndrome, Lambert-Eaton syndrome, Leukocytoclastic vasculitis, Lichen planus, Lichen sclerosus, Ligneous conjunctivitis, Linear IgA disease (LAD), Lupus (SLE), Lyme disease, chronic, Meniere's disease, Microscopic polyangiitis, Mixed connective tissue disease (MCTD), Mooren's ulcer, Mucha-Habermann disease, Multiple sclerosis, Myasthenia gravis, Myositis, Narcolepsy, Neuromyelitis optica (Devic's), Neutropenia, Ocular cicatricial pemphigoid, Optic neuritis, Palindromic rheumatism, PANDAS (Pediatric Autoimmune Neuropsychiatric Disorders Associated with Streptococcus), Paraneoplastic cerebellar degeneration, Paroxysmal nocturnal hemoglobinuria (PNH), Parry Romberg syndrome, Parsonnage-Turner syndrome, Pars planitis (peripheral uveitis), Pemphigus, Peripheral neuropathy, Perivenous encephalomyelitis, Pernicious anemia, POEMS syndrome, Polyarteritis nodosa, Type I, II, & III autoimmune polyglandular syndromes, Polymyalgia rheumatic, Polymyositis, Postmyocardial infarction syndrome, Postpericardiotomy syndrome, Progesterone dermatitis, Primary biliary cirrhosis, Primary sclerosing cholangitis, Psoriasis, Psoriatic, arthritis, Idiopathic pulmonary fibrosis, Pyoderma gangrenous, Pure red cell aplasia, Raynauds phenomenon, Reflex sympathetic dystrophy, Reiter's syndrome, Relapsing polychondritis, Restless legs syndrome, Retroperitoneal Fibrosis, Rheumatic fever, Rheumatoid arthritis, Sarcoidosis, Schmidt syndrome, Scleritis, Scleroderma, Sjogren's syndrome, Sperm & testicular autoimmunity, Stiff person syndrome, Subacute bacterial endocarditis (SBE), Susac's syndrome, Sympathetic ophthalmia, Takayasu's arteritis, Temporal arteritis/Giant cell arteritis, Thrombocytopenic purpura (TTP), Tolosa-Hunt syndrome, Transverse myelitis, Ulcerative colitis, Undifferentiated connective tissue disease (UCTD), Uveitis, Vasculitis, Vesiculobullous dermatosis, Vitiligo, Wegener's granulomatosis.

An inflammatory autoimmune disease is a disease or condition in which a subject's immune system irregularly reacts to one or more components (e.g. biomolecule, protein, cell, tissue, organ, etc.) of the subject as if it were not self and the disease or condition is characterized by abnormal (e.g. elevated or reduced) inflammation. In some embodiments, an inflammatory autoimmune disease is associated with a differential expression of an RPTP or IAD PTPR. In some embodiments, an inflammatory autoimmune disease is associated with a mutation in an RPTPR or an IAD PTPR. Non-limiting examples of inflammatory autoimmune diseases that may be associated with a differential expression of an RPTP or an IAD PTPR include arthritis, rheumatoid arthritis, psoriatic arthritis, juvenile idiopathic arthritis, multiple sclerosis, systemic lupus erythematosus (SLE), myasthenia gravis, juvenile onset diabetes, diabetes mellitus type 1, Guillain-Barre syndrome, Hashimoto's encephalitis, Hashimoto's thyroiditis, ankylosing spondylitis, psoriasis, Sjogren's syndrome, vasculitis, glomerulonephritis, auto-immune thyroiditis, Behcet's disease, Crohn's disease, ulcerative colitis, bullous pemphigoid, sarcoidosis, ichthyosis, Graves ophthalmopathy, inflammatory bowel disease, Addison's disease, Vitiligo, asthma, or allergic asthma.

Non-limiting examples of viral infections that cause infectious diseases that may be associated with a differential expression of an RPTP include an infection of Epstein-Barr virus, human immunodeficiency virus, human T leukemia virus, hepatitis B virus, varicella zoster, influenza, avian influenza, herpes simplex virus I, herpes simplex virus II, cytomegalovirus, H1N1, SARS virus, ebola virus, or measles virus.

Non-limiting examples of cancer that may be associated with a differential expression of an RPTP include head and neck cancer, brain cancer, breast cancer, colorectal cancer, esophageal cancer, gastric cancer, hepatic cancer, bladder cancer, cervical cancer, endometrial cancer, lung cancer, ovarian cancer, uterine cancer, pancreatic cancer, prostate cancer, renal cancer, choriocarcinoma, skin cancer, melanoma, basal cell carcinoma, hairy cell leukemia, chronic lymphotic leukemia, acute lymphocytic leukemia, acute myelogenous leukemia, meningeal leukemia, chronic myelogenous leukemia, erythroleukemia, leukemia, B-cell cancers, lymphoma, multiple myeloma, and MDS. More generally, cancer can be defined as any of various malignant neoplasms characterized by the proliferation of anaplastic cells that may or may not invade surrounding tissue and metastasize to new body sites.

A modulated (e.g. elevated) RNA level, protein level, or other measured quantity relative to a control standard is a measured amount (e.g. of the RNA level, protein level, etc.) that differs from the measured or calculated amount of the same quantity in the standard control. In some embodiments, the measured quantity is greater than or elevated above or increased over the standard control. In further embodiments, the measured quantity is greater by or elevated above or increased over the standard control by at least 5%, 10%, 15%, 20%, 25%, 30%, 35%, 40%, 50%, 60%, 75%, 80%, 90%, or at least 100%. In certain embodiments, the measured quantity is less than or decreased compared to the standard control. In other embodiments, the measured quantity is less than or decreased as compared to the standard control by at least 5%, 10%, 15%, 20%, 25%, 30%, 35%, 40%, 50%, 60%, 75%, 80%, 90%, or at least 100%. The modulated level or amount of a measured quantity (e.g. RNA or protein of a specific RPTP or IAD PTPR) can be also be expressed as a “-fold” increase or decrease. For example, a measurable can be at least 1.1-fold, 1.2-fold, 1.5-fold, 2-fold, 5-fold, or larger-fold greater (e.g. elevated over) or less than a standard control.

A subject who is “at risk of developing” a disease has a greater chance of having the disease than a person who is not at risk of developing the disease. In some embodiments, a standard control is derived from the person who is not at risk of developing the disease (i.e. standard control subject)

The amounts or levels of protein or RNA may be measured in known assays or tests to determine if a subject is at risk of developing a disease. In some embodiments, the person at risk of developing the disease has at least a 5%, 10%, 15%, 20%, 25%, 30%, 35%, 40%, 50%, 60%, 75%, 80%, 90%, or at least a 100% greater likelihood of having the disease at some future time relative to a standard control. In some embodiments that future time is one or more weeks, one or more months, or one or more years from the time at which the risk is assessed. In other embodiments, the subject at risk of developing the disease is or is at least 1.1-fold, 1.2-fold, 1.5-fold, 2-fold, 5-fold, 10-fold, 20-fold more likely to develop the disease at some time in the future. As an example, the standard control may be a threshold level of a measurable quantity established by previous testing. The person at risk of developing the disease may be a person that has the measurable quantity (e.g. RNA level or protein level of one or more RPTPs or IAD PTPRs) in excess of or below the established threshold, depending on the measurable quantity (as disclosed herein). The threshold in the previous embodiment may be established by determining the differences between subjects who do not have the disease (e.g. standard control) and subjects who have the disease, and assigning a threshold that separates one group from the other group in terms of one or more measurable quantities.

The method of determining whether a subject has or is at risk of developing an autoimmune disease may also be a method of diagnosing a subject (e.g. a patient) or providing the subject with a prognosis.

The method may further include administering a treatment for the inflammatory autoimmune disease. The treatment includes an IAD therapeutic agent. In some embodiments, the IAD therapeutic agent is an anti-IAD PTPR antibody, an anti-IAD PTPR inhibitory nucleic acid or an IAD PTPR ligand mimetic. In certain embodiments, the IAD therapeutic agent may be an anti-IAD PTPR antibody. The IAD therapeutic agent may be an anti-IAD PTPR inhibitory nucleic acid. In certain embodiments, the anti-IAD PTPR inhibitory nucleic acid may be an anti-IAD PTPR RNAi molecule. The IAD PTPR ligand mimetic may be a peptide or a small chemical molecule. In certain embodiments, the IAD PTPR is PTPRB, PTPRE, PTPRG, PTPRJ, PTPRM, PTPRO, or PTPRS. In other embodiments, the IAD PTPR is PTPRB, PTPRE, PTPRG, PTPRM, PTPRO, or PTPRS. The IAD PTPR may be PTPRE, PTPRG, PTPRM, PTPRO, or PTPRS. The IAD PTPR may be PTPRE or PTPRS. In other embodiments, the IAD PTPR is PTPRS. In other embodiments, the IAD PTPR is PTPRS and the IAD therapeutic agent is an anti-IAD PTPR antibody (i.e. an anti-IAD PTPRS antibody). In other embodiments, the IAD PTPR is PTPRS and the IAD therapeutic agent is an anti-IAD PTPR antisense nucleic acid (i.e. an anti-IAD PTPRS antisense nucleic acid). In other embodiments, the IAD PTPR is PTPRE. In other embodiments, the IAD PTPR is PTPRE and the IAD therapeutic agent is an anti-IAD PTPR antibody (i.e. an anti-IAD PTPRE antibody). In other embodiments, the IAD PTPR is PTPRE and the IAD therapeutic agent is an anti-IAD PTPR antisense nucleic acid (i.e. an anti-IAD PTPRE antisense nucleic acid).

In some embodiments, the method of determining or method of treating includes obtaining a sample from the subject. A “sample” as used herein is defined above and may be any appropriate sample. For example, the sample may be derived from a joint tissue or a bodily fluid. Where a sample is derived from a tissue (e.g. joint) or fluid (e.g. bodily fluid), the sample may be a specimen obtained directly from the subject without further manipulation or a specimen obtained from the subject with further manipulation, such as standard sample fractionation or purification methods. Thus, in some embodiments a particular fraction or component (e.g. a single cell type, a collection of cell types sharing a common feature or characteristic, tissue type, chemical, compound, nucleic acid, protein, etc.) is isolated from the tissue or fluid to form the sample. In other embodiments the component is also modified in preparation for a future use (e.g. flash freezing, addition of preservatives, culturing, etc.).

In certain embodiments, the method of determining or method of treating includes isolating cells from the joint tissue or the bodily fluid thereby forming isolated sample cells.

The terms “isolated” “purified” or “biologically pure” refer to material that is substantially or essentially free from components which normally accompany it as found in its native state. Purity and homogeneity of biological molecules (e.g. nucleic acids or proteins) are typically determined using analytical chemistry techniques such as polyacrylamide gel electrophoresis or high performance liquid chromatography. A protein that is the predominant species present in a preparation is substantially purified. The term “purified” may denote that a nucleic acid or protein gives rise to essentially one band in an electrophoretic gel. In some embodiments, the nucleic acid or protein is at least 50% pure, optionally at least 65% pure, optionally at least 75% pure, optionally at least 85% pure, optionally at least 95% pure, and optionally at least 99% pure. As an example, an isolated cell or isolated sample cells are a single cell type that is substantially free of many of the components which normally accompany the cells when they are in their native state or when they are initially removed from their native state. In certain embodiments, an isolated cell sample retains those components from its natural state that are required to maintain the cell in a desired state. In some embodiments, an isolated (e.g. purified, separated) cell or isolated cells, are cells that are substantially the only cell type in a sample. A purified cell sample may contain at least 60%, 70%, 75%, 80%, 85%, 90%, 95%, 96%, 97%, 98%, 99%, or 100% of one type of cell. An isolated cell sample may be obtained through the use of a cell marker or a combination of cell markers, either of which is unique to one cell type in an unpurified cell sample. In some embodiments, the cells are isolated through the use of a cell sorter. In some embodiments, antibodies against cell proteins are used to isolate cells.

The isolated cells may be of any appropriate type. In some embodiments, the isolated cells are synoviocytes, fibroblast-like synoviocytes, macrophage-like synoviocytes, fibroblasts, hematopoetic cells, macrophages, leukocytes, T cells, or other immune cells. In other embodiments, the bodily fluid is whole blood, plasma, serum, urine, sputum, saliva, a bronchioaviolar lavage sample, synovial fluid, or exhaled breath condensate. In certain embodiments, the bodily fluid is synovial fluid.

Hematopoietic cells include hematopoietic stem cells and any cells that develop from or are derived from hematopoietic stem cells whether directly or through intermediate cell types. Hematopoietic cells include all myeloid cells and lymphoid cells. Hematopoietic cells include monocytes, macrophages, neutrophils, basophils, eosinophils, erythrocytes, megakaryocytes, platelets, dendritic cells, T-cells, B-cells, Natural Killer cells (NK-cells), and NKT cells.

Immune cells include neutrophils, eosinophils, basophils, lymphocytes, B-cells, T-cells, Natural Killer cells (NK-cells), NKT cells, monocytes, macrophages, dendritic cells, or any cells derived from these cells (e.g. macrophage-like synoviocytes, etc.). Included are cells derived from lymphoblasts, monoblasts, myeloblasts, and promyelocytes.

Synoviocytes are cells of the synovial membrane in joint capsules. Synoviocytes include macrophage-like/macrophagic synoviocytes, sometime referred to as type A cells or synoviomacrophages, and fibroblast-like synoviocytes, sometimes referred to as type B cells or synovioblasts. Fibroblast-like synoviocytes produce synovial fluid components. Macrophage-like synoviocytes are resident macrophages.

In some embodiments, the inflammatory autoimmune disease is an arthritis. In some embodiments, the inflammatory autoimmune disease is rheumatoid arthritis, psoriatic arthritis, juvenile idiopathic arthritis, multiple sclerosis, systemic lupus erythematosus (SLE), myasthenia gravis, juvenile onset diabetes, diabetes mellitus type 1, Guillain-Barre syndrome, Hashimoto's encephalitis, Hashimoto's thyroiditis, ankylosing spondylitis, psoriasis, Sjogren's syndrome, vasculitis, glomerulonephritis, auto-immune thyroiditis, Behcet's disease, Crohn's disease, ulcerative colitis, bullous pemphigoid, sarcoidosis, psoriasis, ichthyosis, Graves ophthalmopathy, inflammatory bowel disease, Addison's disease, Vitiligo, asthma, or allergic asthma. In some embodiments, the inflammatory autoimmune disease is rheumatoid arthritis.

Methods for detecting and identifying nucleic acids and proteins and interactions between such molecules involve conventional molecular biology, microbiology, and recombinant DNA techniques within the skill of the art. Such techniques are explained fully in the literature (see, e.g., Sambrook, Fritsch & Maniatis, Molecular Cloning: A Laboratory Manual, Second Edition 1989, Cold Spring Harbor Laboratory Press, Cold Spring Harbor, N.Y.; Animal Cell Culture, R. I. Freshney, ed., 1986).

Provided herein are methods of determining whether an individual has or may be at risk of developing a disease. In certain embodiments, the disease is associated with a mutation of a receptor protein tyrosine phosphatase, relative to the corresponding receptor protein tyrosine phosphatase, in individuals who do not have or are not at risk of developing the disease. In some embodiments, the disease is an autoimmune disease or disorder, cancer, an infectious disease (e.g. viral, bacterial, parasitic, etc.), an obesity associated disease, a metabolic disease or disorder, an inflammatory disease, an immune disease or disorder, or a traumatic injury. In some embodiments, the disease is an inflammatory autoimmune disease (IAD). In other embodiments, the disease is a disease associated with a patient's joints. In certain embodiments, the inflammatory autoimmune disease is rheumatoid arthritis. In certain embodiments, the method is a method of determining whether an individual has a disease. The disease may be associated with a mutation of a receptor protein tyrosine phosphatase, relative to the corresponding receptor protein tyrosine phosphatase, in individuals who do not have the disease. In other embodiments, the method is a method of determining whether an individual may be at risk of developing a disease. The disease may be associated with a mutation of a receptor protein tyrosine phosphatase, relative to the corresponding receptor protein tyrosine phosphatase, in individuals who are not at risk of developing the disease.

Methods for detecting genetic variants are known in the art, e.g., Southern or Northern blot, nucleotide array, amplification methods, etc. Primers or probes are designed to hybridize to a target sequence. For example, genomic DNA can be screened for the presence of an identified genetic element of using a probe based upon one or more sequences. Expressed RNA can also be screened, but may not include all relevant genetic variations. Various degrees of stringency of hybridization may be employed in the assay. As the conditions for hybridization become more stringent, there must be a greater degree of complementarity between the probe and the target for duplex formation to occur.

The degree of stringency can be controlled by temperature, ionic strength, pH and/or the presence of a partially denaturing solvent such as formamide. For example, the stringency of hybridization is conveniently varied by changing the concentration of formamide within the range up to and about 50%. The degree of complementarity (sequence identity) required for detectable binding will vary in accordance with the stringency of the hybridization medium and/or wash medium. In certain embodiments, in particular for detection of a particular SNP, the degree of complementarity is about 100 percent. In other embodiments, sequence variations can result in <100% complementarity, <90% complimentarity probes, <80% complimentarity probes, etc., in particular, in a sequence that does not involve a SNP. In some examples, e.g., detection of species homologs, primers may be compensated for by reducing the stringency of the hybridization and/or wash medium.

High stringency conditions for nucleic acid hybridization are well known in the art as are set forth above. It is understood that the temperature and ionic strength of a desired stringency are determined in part by the length of the particular nucleic acid(s), the length and nucleotide content of the target sequence(s), the charge composition of the nucleic acid(s), and by the presence or concentration of formamide, tetramethylammonium chloride or other solvent(s) in a hybridization mixture. Nucleic acids can be completely complementary to a target sequence or exhibit one or more mismatches.

Nucleic acids of interest may also be amplified using a variety of known amplification techniques. For instance, polymerase chain reaction (PCR) technology may be used to amplify target sequences directly from DNA, RNA, or cDNA. In some embodiments, a stretch of nucleic acids is amplified using primers on either side of a targeted genetic variation, and the amplification product is then sequenced to detect the targeted genetic variation (using, e.g., Sanger sequencing, Pyrosequencing, Next gen sequencing technologies). For example, the primers can be designed to hybridize to either side of the upstream regulatory region of an RPTP or IAD PTPR gene, and the intervening sequence determined to detect a SNP in the promoter region. In some embodiments, one of the primers can be designed to hybridize to the targeted genetic variant.

Amplification techniques can also be useful for cloning nucleic acid sequences, to make nucleic acids to use as probes for detecting the presence of a target nucleic acid in samples, for nucleic acid sequencing, for control samples, or for other purposes. Probes and primers are also readily available from commercial sources, e.g., from Invitrogen, Clonetech, etc.

Expression of a given gene, e.g., an RPTP or IAD PTPR or another disease marker, or standard (control), is typically detected by detecting the amount of RNA (e.g., mRNA) or protein. Sample levels can be compared to a standard control level.

Methods for detecting RNA are largely cumulative with the nucleic acid detection assays described above. RNA to be detected can include mRNA or pRNA (promoter-associated RNA, see, e.g., Schmitz et al. (2010) Genes Dev. 24:2264-69). In some embodiments, a reverse transcriptase reaction is carried out and the targeted sequence is then amplified using standard PCR. Quantitative PCR (qPCR) or real time PCR (RT-PCR) is useful for determining relative expression levels, when compared to a control. Quantitative PCR techniques and platforms are known in the art, and commercially available (see, e.g., the qPCR Symposium website, available at qpersymposium.com). Nucleic acid arrays are also useful for detecting nucleic acid expression. Customizable arrays are available from, e.g., Affimetrix.

Protein levels can be detected using antibodies or antibody fragments specific for that protein, natural ligands, small molecules, aptamers, etc.

Antibody based techniques are known in the art, and described, e.g., in Harlow & Lane (1988) Antibodies: A Laboratory Manual and Harlow (1998) Using Antibodies: A Laboratory Manual; Wild, The Immunoassay Handbook, 3d edition (2005) and Law, Immunoassay: A Practical Guide (1996). The assay can be directed to detection of a molecular target (e.g., protein or antigen), or a cell, tissue, biological sample, liquid sample or surface suspected of carrying an antibody or antibody target.

A non-exhaustive list of immunoassays includes: competitive and non-competitive formats, enzyme linked immunosorption assays (ELISA), microspot assays, Western blots, gel filtration and chromatography, immunochromatography, immunohistochemistry, flow cytometry or fluorescence activated cell sorting (FACS), microarrays, and more. Such techniques can also be used in situ, ex vivo, in vitro, or in vivo, e.g., for diagnostic imaging.

Aptamers are nucleic acids that are designed to bind to a wide variety of targets in a non-Watson Crick manner. An aptamer can thus be used to detect or otherwise target nearly any molecule of interest, including an autoimmune, inflammatory autoimmune, cancer, infectious disease, or other disease associated protein. Methods of constructing and determining the binding characteristics of aptamers are well known in the art. For example, such techniques are described in U.S. Pat. Nos. 5,582,981, 5,595,877 and 5,637,459. Aptamers are typically at least 5 nucleotides, 10, 20, 30 or 40 nucleotides in length, and can be composed of modified nucleic acids to improve stability. Flanking sequences can be added for structural stability, e.g., to form 3-dimensional structures in the aptamer.

Protein detection agents contemplated herein can be used as a treatment and/or diagnosis of autoimmune disease, inflammatory autoimmune disease, cancer, infectious disease, or other disease or predictor of propensity for survival in a subject having or suspected of developing a autoimmune disease, inflammatory autoimmune disease, cancer, infectious disease, or other disease. In certain embodiments, RPTP or IAD PTPR antibodies can be used to assess RPTP or IAD PTPR protein levels in a subject having or suspected of developing a autoimmune disease, inflammatory autoimmune disease, cancer, infectious disease, or other disease. It is contemplated herein that antibodies or antibody fragments may be used to modulate RPTP or IAD PTPR production in a subject having or suspected of developing a autoimmune disease, inflammatory autoimmune disease, cancer, infectious disease, or other disease. In certain embodiments, one or more agents capable of modulating an RPTP or IAD PTPR may be used to treat a subject having or suspected of developing a autoimmune disease, inflammatory autoimmune disease, cancer, infectious disease, or other disease.

In certain embodiments, an RPTP or IAD PTPR diagnostic test may include, but is not limited to, alone or in combination, analysis of individual RPTP genes, mRNA levels, and/or protein levels.

The above methods of detection can be applied to additional autoimmune disease, inflammatory autoimmune disease, cancer, infectious disease, or other disease markers. That is, the expression level or presence of genetic variants of at least one additional autoimmune, inflammatory autoimmune, cancer, infectious disease, or other disease marker gene can be determined, or the activity of the marker protein can be determined, and compared to a standard control for the autoimmune, inflammatory autoimmune, cancer, infectious disease, or other disease marker. The examination of additional autoimmune, inflammatory autoimmune, cancer, infectious disease, or other disease markers can be used to confirm a diagnosis of autoimmune, inflammatory autoimmune, cancer, infectious disease, or other disease, monitor disease progression, or determine the efficacy of a course of treatment in a subject.

Provided herein are methods of determining whether the genome of a subject comprises a genetic variant of at least one gene selected from the genes encoding RPTPs and IAD PTPRs, or an elevated level of RNA or protein. The presence of a genetic variant or elevated level of RNA or protein indicates that the subject has or is at risk of developing rheumatoid arthritis. Said determining can optionally be combined with determining whether the genome of the subject comprises a genetic variant RPTP gene, or determining whether the subject has an elevated level of RPTP RNA or protein to confirm or strengthen the diagnosis or prognosis.

The invention provides kits for detection of inflammatory autoimmune disease markers in a subject. The kit can be for personal use or provided to medical professionals. The kit can be a kit for diagnosing or prognosing an inflammatory autoimmune disorder, or for monitoring the progression of disease or the efficacy of treatment.

The kit may includes components for assessing an IAD PTPR gene expression comprising, e.g., a nucleic acid capable of detecting an IAD PTPR RNA or an IAD PTPR protein binding agent, optionally labeled. One of skill will appreciate that an IAD PTPR gene expression can be determined by measuring an IAD PTPR RNA or protein. The kit can further include assay containers (tubes), buffers, or enzymes necessary for carrying out the detection assay.

In some embodiments, the kit includes components for determining whether the expression level of an IAD PTPR RNA or protein is increased or decreased, e.g., a nucleic acid that specifically hybridizes to an IAD PTPR RNA or an anti-IAD PTPR antibody or an IAD PTPR ligand mimetic that binds to an IAD PTPR protein. Other components in a kit can include, DNA sequencing assay components, Taqman® genotyping assay components, Meta Analysis, one or more detection system(s), one or more control samples or a combination thereof.

The kit may include components to examine more than one inflammatory autoimmune disease marker. For example, the kit can include marker detection agents, such as marker specific primers or probes attached to an addressable array. Exemplary markers include nucleic acids transcribable and/or translatable to an IAD PTPR (e.g. RNA) or IAD PTPR proteins, or other inflammatory autoimmune disease associated nucleic acids or proteins, e.g. IAD PTPR ligands. In some embodiments, the genetic sequence of the markers is detected instead of the expression level of the markers. Additional inflammatory autoimmune disease markers can include the other PTPR nucleic acids or proteins.

The kit will generally include at least one vial, test tube, flask, bottle, syringe or other container means, into which the testing agent, can be suitably reacted or aliquoted. Kits can also include components for comparing results such as a suitable control sample, for example a positive and/or negative control. The kit can also include a collection device for collecting and/or holding the sample from the subject. The collection device can include a sterile swab or needle (for collecting bodily fluids), and/or a sterile tube (e.g., for holding the swab or a bodily fluid sample).

Different biotechnology companies currently offer antibodies that recognize the extracellular domain from various RPTPs.

### III. Methods of Treatment

Provided herein are methods of treating an individual who has or may be at risk of developing a disease by administering to the individual a therapeutically effective amount of a therapeutic agent. The disease is associated with a differential expression of a receptor protein tyrosine phosphatase. In some embodiments, the disease is an autoimmune disease or disorder, cancer, an infectious disease (e.g. viral, bacterial, parasitic, etc.), an obesity associated disease, a metabolic disease or disorder, an inflammatory disease, an immune disease or disorder, or a traumatic injury. In some embodiments, the method is a method of treating an individual who has a disease by administering to the individual a therapeutically effective amount of a therapeutic agent. In other embodiments, the method is a method of treating an individual who may be at risk of developing a disease by administering to the individual a therapeutically effective amount of a therapeutic agent.

In some embodiments, the disease is an inflammatory autoimmune disease (IAD). In other embodiments, the disease is a disease associated with a patient's joints. In another embodiment, the inflammatory autoimmune disease is rheumatoid arthritis.

As described above, the increased expression of one or more RPTPs may be associated with a disease or a risk of developing the disease. In certain embodiments, the decreased expression of one or more RPTPs is associated with a disease or a risk of developing the disease. In an additional embodiment, the increased expression of a first RPTP and the decreased expression of a second RPTP is associated with a disease or a risk of developing the disease. In some embodiments, the increased expression of one or more (e.g. 2, 3, 4, 5, 6, 7, 8, 9, 10, 11, 12, 13, 14, 15, 16, 17, 18, 19, 20, 21) RPTPs or IAD PTPRs is associated with a disease or a risk of developing the disease. In other embodiments, the increased expression of PTPRA, PTPRB, PTPRC, PTPRE, PTPRG, PTPRJ, PTPRM, PTPRO, PTPRS, or a combination thereof (e.g. 2, 3, 4, 5, 6, 7, 8, 9) is associated with an inflammatory autoimmune disease. In certain embodiments, the increased expression of PTPRB, PTPRE, PTPRG, PTPRM, PTPRO, PTPRS, or a combination thereof (e.g. 2, 3, 4, 5, 6) is associated with an inflammatory autoimmune disease. The increased expression of PTPRE, PTPRG, PTPRM, PTPRO, PTPRS, or a combination thereof (e.g. 2, 3, 4, 5) may be associated with an inflammatory autoimmune disease. In some embodiments, the increased expression of PTPRE, PTPRS, or a combination thereof is associated with an inflammatory autoimmune disease. In other embodiments, the increased expression of PTPRS is associated with an inflammatory autoimmune disease. In still other embodiments, the increased expression of PTPRA, PTPRB, PTPRC, PTPRE, PTPRG, PTPRJ, PTPRM, PTPRO, PTPRS, or a combination thereof (e.g. 2, 3, 4, 5, 6, 7, 8, 9) is associated with rheumatoid arthritis. The increased expression of PTPRB, PTPRE, PTPRG, PTPRM, PTPRO, PTPRS, or a combination thereof (e.g. 2, 3, 4, 5, 6) may be associated with rheumatoid arthritis. In some embodiments, the increased expression of PTPRE, PTPRG, PTPRM, PTPRO, PTPRS, or a combination thereof (e.g. 2, 3, 4, 5) is associated with rheumatoid arthritis. In other embodiments, the increased expression of PTPRE, PTPRS, or a combination thereof is associated with rheumatoid arthritis. In certain embodiments, the increased expression of PTPRS is associated with rheumatoid arthritis.

Also as described above, the decreased expression of one or more (e.g. 2, 3, 4, 5, 6, 7, 8, 9, 10, 11, 12, 13, 14, 15, 16, 17, 18, 19, 20, 21) RPTPs or IAD PTPRs may be associated with a disease or a risk of developing the disease. In some embodiments, the decreased expression of PTPRF or PTPRZ1, or a combination thereof, is associated with an inflammatory autoimmune disease. In other embodiments, the decreased expression of PTPRF is associated with an inflammatory autoimmune disease. In certain embodiments, the decreased expression of PTPRZ1 is associated with an inflammatory autoimmune disease. The decreased expression of PTPRF or PTPRZ1, or a combination thereof may be associated with rheumatoid arthritis. In some embodiments, the decreased expression of PTPRF is associated with rheumatoid arthritis. In some embodiments, the decreased expression of PTPRZ1 is associated with rheumatoid arthritis.

Also as described above, the increased expression of a first RPTP and the decreased expression of a second RPTP may be associated with a disease or a risk of developing the disease. In some embodiments increased expression of PTPRA, PTPRB, PTPRC, PTPRE, PTPRG, PTPRJ, PTPRM, PTPRO, PTPRS, or a combination thereof (e.g. 2, 3, 4, 5, 6, 7, 8, 9) and the decreased expression of PTPRF or PTPRZ1, or a combination thereof, is associated with an inflammatory autoimmune disease. In other embodiments increased expression of PTPRA, PTPRB, PTPRC, PTPRE, PTPRG, PTPRJ, PTPRM, PTPRO, PTPRS, or a combination thereof (e.g. 2, 3, 4, 5, 6, 7, 8, 9) and the decreased expression of PTPRF or PTPRZ1, or a combination thereof, is associated with rheumatoid arthritis. In certain embodiments increased expression of PTPRE and PTPRS and decreased expression of PTPRF and PTPRZ1 is associated with an inflammatory autoimmune disease. Increased expression of PTPRE and PTPRS and decreased expression of PTPRF and PTPRZ1 may be associated with rheumatoid arthritis.

In some embodiments, the therapeutic agent is an agonist of one or more RPTPs. In further embodiment, the therapeutic agent is an antagonist of one or more RPTPs. In an additional embodiment, the therapeutic agent is an agonist for a first RPTP and an antagonist for a second RPTP. In some embodiments, the therapeutic agent is an antagonist for one or more (e.g. 2, 3, 4, 5, 6, 7, 8, 9, 10, 11, 12, 13, 14, 15, 16, 17, 18, 19, 20, 21) RPTPs or IAD PTPRs. In other embodiments, the therapeutic agent is an antagonist for PTPRA, PTPRB, PTPRC, PTPRE, PTPRG, PTPRJ, PTPRM, PTPRO, PTPRS, or a combination thereof (e.g. 2, 3, 4, 5, 6, 7, 8, 9). In certain embodiments, the therapeutic agent is an antagonist for PTPRB, PTPRE, PTPRG, PTPRM, PTPRO, PTPRS, or a combination thereof (e.g. 2, 3, 4, 5, 6). In some embodiments, the therapeutic agent is an antagonist for PTPRE, PTPRG, PTPRM, PTPRO, PTPRS, or a combination thereof (e.g. 2, 3, 4, 5). In other embodiments, the therapeutic agent is an antagonist for PTPRE, PTPRS, or a combination thereof. In still other embodiments, the therapeutic agent is an antagonist for PTPRS.

In some embodiments, the therapeutic agent is an agonist for one or more (e.g. 2, 3, 4, 5, 6, 7, 8, 9, 10, 11, 12, 13, 14, 15, 16, 17, 18, 19, 20, 21) RPTPs or IAD PTPRs. In other embodiments, the therapeutic agent is an agonist for PTPRF or PTPRZ1, or a combination thereof. In still other embodiments, the therapeutic agent is an agonist for PTPRF. In some embodiments, the therapeutic agent is an agonist for PTPRZ1.

In some embodiments the therapeutic agent is an antagonist for PTPRA, PTPRB, PTPRC, PTPRE, PTPRG, PTPRJ, PTPRM, PTPRO, PTPRS, or a combination thereof (e.g. 2, 3, 4, 5, 6, 7, 8, 9) and the therapeutic agent is an agonist for PTPRF or PTPRZ1, or a combination thereof, In a further embodiment, the therapeutic agent is an autoimmune therapeutic agent. In another embodiment, the therapeutic agent is an inflammatory autoimmune disease therapeutic agent.

An agonist is an agent that increases one or more functional effects associated with an RPTP or an IAD PTPR activity or function. An antagonist is an agent that decreases one or more functional effects associated with an RPTP or an IAD PTPR activity or function.

The phrase “functional effects” in the context of assays for testing autoimmune therapeutic agents or IAD PTPR therapeutic agents or RPTP binding agents or IAD PTPR binding agents includes the determination of any parameter that is indirectly or directly under the influence of the RPTP or IAD PTPR, e.g., functional, physical and chemical effects. It includes ligand binding, transcription, protein phosphorylation or dephosphorylation, signal transduction, receptor-ligand interactions, second messenger concentrations, in vitro, in vivo, and ex vivo and also includes other physiologic effects such increases or decreases inflammatory signals or inflammatory signaling cascades.

By “determining the functional effect” is meant assays, tests or measurements to assess the functional, physical and chemical effects of an autoimmune therapeutic agent or IAD PTPR therapeutic agent or RPTP binding agent or IAD PTPR binding agent, typically by examining an increase or decrease in a parameter that is indirectly or directly under the influence of an RPTP or IAD PTPR. Such functional effects can be measured by any appropriate means known to those skilled in the art, e.g., changes in spectroscopic characteristics (e.g., fluorescence, absorbance, refractive index), hydrodynamic (e.g., shape), chromatographic, or solubility properties, inducible markers, oocyte RPTP or IAD PTPR gene expression; tissue culture cell RPTP or IAD PTPR expression; transcriptional activation of genes downstream of an RPTP or IAD PTPR; ligand binding assays; changes in intracellular second messengers such as cAMP, cGMP, and inositol triphosphate (IP3); changes inflammatory signals or messengers, or the like.

Provided herein are methods of treating a subject who has or may be at risk of developing an autoimmune disease. The method including administering to the subject a therapeutically effective amount of an autoimmune therapeutic agent. In some embodiments, the method is a method of treating an individual who has a disease by administering to the individual a therapeutically effective amount of an autoimmune therapeutic agent. In other embodiments, the method is a method of treating an individual who may be at risk of developing a disease by administering to the individual a therapeutically effective amount of an autoimmune therapeutic agent. In certain embodiments, the autoimmune therapeutic agent is an agonist of an RPTP or an antagonist of an RPTP. In a further embodiment the RPTP is PTPRA, PTPRB, PTPRC, PTPRD, PTPRE, PTPRF, PTPRG, PTPRH, PTPRJ, PTPRK, PTPRM, PTPRN, PTPRN2, PTPRO, PTPRQ, PTPRR, PTPRS, PTPRT, PTPRU, PTPRV or PTPRZ1. In some embodiments, the RPTP is PTPRA, PTPRB, PTPRC, PTPRE, PTPRF, PTPRG, PTPRJ, PTPRM, PTPRO, PTPRS, or PTPRZ1. In other embodiments, the RPTP is PTPRA, PTPRB, PTPRC, PTPRE, PTPRG, PTPRJ, PTPRM, PTPRO, or PTPRS. In still other embodiments, the RPTP is PTPRB, PTPRE, PTPRG, PTPRJ, PTPRM, PTPRO, or PTPRS. The RPTP may be PTPRB, PTPRE, PTPRG, PTPRM, PTPRO, or PTPRS. In other embodiments, the RPTP is PTPRE, PTPRG, PTPRM, PTPRO, or PTPRS. The RPTP may be PTPRE or PTPRS. In some embodiments, the RPTP is PTPRE. In other embodiments, the RPTP is PTPRS. In certain embodiments, the RPTP is PTPRF. In other embodiments, the RPTP is PTPRZ1.

The autoimmune disease may be an inflammatory autoimmune disease and the autoimmune therapeutic agent may be an IAD therapeutic agent. Thus, in some embodiments the therapeutic agent may be an IAD therapeutic agent. The IAD therapeutic agent may be selected from an anti-IAD PTPR antibody, an anti-IAD PTPR inhibitory nucleic acid and an IAD PTPR ligand mimetic. The IAD therapeutic agent targets an IAD PTPR. In some embodiments, the inflammatory autoimmune disease is mediated by cells expressing an IAD PTPR. In certain embodiments, the cells mediating the inflammatory autoimmune disease are synoviocytes, fibroblast-like synoviocytes, macrophage-like synoviocytes, fibroblasts, hematopoetic cells, macrophages, leukocytes, T cells, or other immune cells.

The method may include treating a subject who has or may be at risk of developing an inflammatory autoimmune disease. The method may include administering to the subject a therapeutically effective amount of an IAD therapeutic agent. In some embodiments, the IAD therapeutic agent targets an IAD PTPR. In other embodiments, the method includes, prior to said administering, determining whether a subject expresses an elevated RNA level of the IAD PTPR or an elevated protein level of the IAD PTPR, relative to a standard control. The presence of the elevated RNA level or the elevated protein level indicates the subject has or may be at risk of developing said inflammatory autoimmune disease.

In some embodiments, the method includes treating a subject who has an inflammatory autoimmune disease and the presence of the elevated RNA level or the elevated protein level indicates the subject has said inflammatory autoimmune disease.

In some embodiments, the method includes treating a subject who may be at risk of developing an inflammatory autoimmune disease and the presence of the elevated RNA level or the elevated protein level indicates the subject may be at risk of developing said inflammatory autoimmune disease.

The method of determining or method of treating may include obtaining a sample from the subject. The sample may be derived from a joint tissue or a bodily fluid. In some embodiments, the method of determining or method of treating includes isolating cells from the joint tissue or the bodily fluid thereby forming isolated sample cells. In certain embodiments, the method of determining or method of treating includes isolated sample cells that are synoviocytes, fibroblast-like synoviocytes, macrophage-like synoviocytes, fibroblasts, hematopoetic cells, macrophages, leukocytes, T cells, or other immune cells. In other embodiments, the method of determining or method of treating includes bodily fluid that is whole blood, plasma, serum, urine, sputum, saliva, a bronchioaviolar lavage sample, synovial fluid, or exhaled breath condensate. In some embodiments, the bodily fluid is synovial fluid.

In some embodiments, the inflammatory autoimmune disease is an arthritis. In certain embodiments, the inflammatory autoimmune disease is rheumatoid arthritis, psoriatic arthritis, juvenile idiopathic arthritis, multiple sclerosis, systemic lupus erythematosus (SLE), myasthenia gravis, juvenile onset diabetes, diabetes mellitus type 1, Guillain-Barre syndrome, Hashimoto's encephalitis, Hashimoto's thyroiditis, ankylosing spondylitis, psoriasis, Sjogren's syndrome, vasculitis, glomerulonephritis, auto-immune thyroiditis, Behcet's disease, Crohn's disease, ulcerative colitis, bullous pemphigoid, sarcoidosis, psoriasis, ichthyosis, Graves ophthalmopathy, inflammatory bowel disease, Addison's disease, Vitiligo, asthma, or allergic asthma. In some embodiments, the inflammatory autoimmune disease is rheumatoid arthritis.

The IAD therapeutic agent may be an anti-IAD PTPR antibody. In some embodiments, the IAD therapeutic agent is an anti-IAD PTPR inhibitory nucleic acid. In other embodiments, the anti-IAD PTPR inhibitory nucleic acid is an anti-IAD PTPR RNAi molecule. In other embodiments, the anti-IAD PTPR inhibitory nucleic acid is an antisense nucleic acid such as anti-IAD PTPR antisense nucleic acid. In still other embodiments, the IAD PTPR ligand mimetic is a peptide or a small chemical molecule. The IAD PTPR may be PTPRB, PTPRE, PTPRG, PTPRJ, PTPRM, PTPRO, or PTPRS. In some embodiments, the IAD PTPR is PTPRB, PTPRE, PTPRG, PTPRM, PTPRO, or PTPRS. In certain embodiments, the IAD PTPR is PTPRE, PTPRG, PTPRM, PTPRO, or PTPRS. The IAD PTPR may be PTPRE or PTPRS. In some embodiments, the IAD PTPR is PTPRS.

In some embodiments the IAD therapeutic agent is an anti-IAD PTPR antibody (e.g. an anti-IAD PTPRS antibody or an anti-IAD PTPRE antibody) or an anti-IAD PTPR antisense nucleic acid (e.g. an anti-IAD PTPRS antisense nucleic acid or an anti-IAD PTPRE antisense nucleic acid). In some embodiments, the IAD therapeutic agent is an anti-IAD PTPR antibody (e.g. an anti-IAD PTPRS antibody or an anti-IAD PTPRE antibody). The IAD therapeutic agent may also be an anti-IAD PTPR antisense nucleic acid (e.g. an anti-IAD PTPRS antisense nucleic acid or an anti-IAD PTPRE antisense nucleic acid).

In certain embodiments, the method includes treating a subject who has or may be at risk of developing an autoimmune disease. The method includes administering to the subject a therapeutically effective amount of an autoimmune therapeutic agent. The autoimmune therapeutic agent may be an agonist of an RPTP or an antagonist of an RPTP. The RPTP may be PTPRA, PTPRB, PTPRC, PTPRD, PTPRE, PTPRF, PTPRG, PTPRH, PTPRJ, PTPRK, PTPRM, PTPRN, PTPRN2, PTPRO, PTPRQ, PTPRR, PTPRS, PTPRT, PTPRU, PTPRV or PTPRZ1. In some embodiments, the RPTP is PTPRA, PTPRB, PTPRC, PTPRE, PTPRF, PTPRG, PTPRJ, PTPRM, PTPRO, PTPRS, or PTPRZ1. In other embodiments, the RPTP is PTPRA, PTPRB, PTPRC, PTPRE, PTPRG, PTPRJ, PTPRM, PTPRO, or PTPRS. In still other embodiments, the RPTP is PTPRB, PTPRE, PTPRG, PTPRJ, PTPRM, PTPRO, or PTPRS. In some embodiments, the RPTP is PTPRB, PTPRE, PTPRG, PTPRM, PTPRO, or PTPRS. In other embodiments, the RPTP is PTPRE, PTPRG, PTPRM, PTPRO, or PTPRS. In certain embodiments, the RPTP is PTPRE or PTPRS. In some embodiments, the RPTP is PTPRE. In other embodiments, the RPTP is PTPRS. In still other embodiments, the RPTP is PTPRF. In some embodiments, the RPTP is PTPRZ1. In additional embodiments, the method further includes administering to the subject a second therapeutic agent. In some embodiments, a method is included wherein the autoimmune disease is an inflammatory autoimmune disease and the autoimmune therapeutic agent is an IAD therapeutic agent. The IAD therapeutic agent may be selected from an anti-IAD PTPR antibody, an anti-IAD PTPR inhibitory nucleic acid and an IAD PTPR ligand mimetic. In some embodiments, the IAD therapeutic agent targets an IAD PTPR. In some embodiments, the second therapeutic agent is selected from a disease-modifying antirheumatic drug (DMARD) (e.g. azathiopurine, ciclosporin, D-penicillamine, gold salts, hydroxychloroquine, leflunomide, methotrexate, minocycline, sulfasalazine, etc.), a non-steroidal anti-inflammatory drug, a steroid, glucocorticoids, a corticosteroid, a TNF alpha inhibitor (e.g. etanercept, infliximab, adalimumab, certolizumab pegol, golimumab, etc.), an immunosuppressant, an interleukin 1 blocker (e.g. anakinra (Kineret)), abatacept (Orencia), belatacept, rituximab (Rituxan), interleukin 6 blocker e.g. tocilizumab) examples of which are well known in the art.

Inflammatory autoimmune disease treatment (e.g. treatments that would be known by one of skill in the art, IAD therapeutic agents, etc.), such as the agents described above, can be used alone, sequentially, or in combination according to the methods described herein. In some embodiments, an inflammatory autoimmune disease treatment is used in combination with a more targeted agonist or antagonist of an RPTP or an IAD PTPR expression or activity.

The results disclosed herein indicate that elevated expression of an RPTP or an IAD PTPR are associated with an inflammatory autoimmune disease. The invention thus includes methods and compositions for inhibiting the expression and/or activity of an RPTP or IAD PTPR DNA, RNA, or protein. Exemplary inhibitors include anti-IAD PTPR inhibitory nucleic acids, anti-IAD PTPR RNAi molecules, anti-IAD PTPR antibodies and IAD PTPR ligand mimetics. In some embodiments, an RPTP or IAD PTPR activity can be inhibited, e.g., using anti-IAD RPTP inhibitory nucleic acids, anti-IAD PTPR antibodies, or IAD PTPR ligand mimetics. The terms “inhibitor” and “antagonist” and like terms are used synonymously herein.

The results disclosed herein indicate that reduced expression of an RPTP is associated with an inflammatory autoimmune disease. The invention thus includes methods and compositions for increasing the expression and/or activity of an RPTP DNA, RNA, or protein. Exemplary activators include anti-RPTP antibodies and RPTP ligand mimetics. In some embodiments, an RPTP activity can be increased e.g., using anti-RPTP antibodies, or RPTP ligand mimetics. The terms “activator” and “agonist” and like terms are used synonymously herein.

Thus, a nucleotide sequence that specifically interferes with expression of an RPTP or IAD PTPR gene at the transcriptional or translational level can be used to treat or prevent inflammatory autoimmune disease. This approach may utilize, for example, inhibitory nucleic acids (e.g. siRNA, miRNA, shRNA, shmiRNA) to block transcription or translation of a specific mRNA (e.g., an mRNA translatable into a specific overexpressed RPTP or IAD PTPR), either by inducing degradation of the mRNA or by masking the mRNA, either with an inhibitory nucleic acid. In some embodiments, the inhibitory nucleic acid (e.g. siRNA, miRNA, shRNA, shmiRNA) does not significantly block expression of other RPTP or IAD PTPR genes.

Double stranded inhibitory nucleic acids (e.g. siRNA, shRNA, miRNA, shmiRNA) that correspond to a single RPTP or single IAD PTPR gene can be used to silence the transcription and/or translation by inducing degradation of such single RPTP or IAD PTPR mRNA transcripts, and thus treat or prevent inflammatory autoimmune disease (e.g., inflammatory autoimmune disease associated with increased expression of an RPTP or IAD PTPR gene). In some embodiments, dsRNA oligonucleotides that specifically hybridize to RPTP or IAD PTPR nucleic acid sequences described herein can be used in the methods of the present invention. A decrease in the severity of inflammatory autoimmune disease symptoms in comparison to symptoms detected in the absence of the inhibitory nucleic acid can be used to monitor the efficacy of the inhibitory nucleic acid.

In some embodiments, an inhibitory nucleic acid (e.g. siRNA, miRNA, shRNA, shmiRNA, antisense nucleic acid) is or contains an antisense nucleic acid sequence that is perfectly complementary to at least a portion of the targeted mRNA translatable to an RPTP or IAD PTPR (or subgroup thereof) described herein. In certain embodiments, the inhibitory nucleic acid includes an antisense nucleic acid sequence having at least 65%, at least 70%, at least 75%, at least 80%, at least 85%, at least 90%, at least 95%, at least 96%, at least 97%, at least 98%, or at least 99%, or 100% complementarity with a sequence of an mRNA translatable to an RPTP or IAD PTPR (or subgroup thereof) described herein. The sequence of an mRNA translatable to an RPTP or IAD PTPR (target sequence) may be 5, 6, 7, 8, 9, 10, 11, 12, 13, 14, 15, 16, 17, 18, 19, 20, 21, 22, 23, 24, 25, 26, 27, 28, 29, 30, 32, 34, 36, 38, 40, 45, 50, 55, 60, 70, 80, 90, 100, 125, 150, 175, 200 or more nucleotides in length. In some embodiments, the inhibitory nucleic acid includes an antisense nucleic acid sequence that is substantially complementary to a a sequence of an mRNA translatable to an RPTP or IAD PTPR (or subgroup thereof) described herein. In some embodiments, the mRNA that is translatable to an RPTP has the sequence or portion of a sequence set forth in SEQ ID NO: 2, SEQ ID NO: 4, SEQ ID NO: 6 or SEQ ID NO: 8, SEQ ID NO: 11 or SEQ ID NO: 13 (wherein the “T” set forth in these sequences are “U” in the actual RNA sequence). In other embodiments, the inhibitory nucleic acid is a sequence having at least 65%, at least 70%, at least 75%, at least 80%, at least 85%, at least 90%, at least 95%, at least 96%, at least 97%, at least 98%, or at least 99% complementarity with a sequence of an mRNA translatable to an RPTP or IAD PTPR (or subgroup thereof) described herein. In some embodiments, the antisense nucleic acid sequence is 5, 6, 7, 8, 9, 10, 11, 12, 13, 14, 15, 16, 17, 18, 19, 20, 21, 22, 23, 24, 25, 26, 27, 28, 29, 30, 32, 34, 36, 38, 40, 45, 50, 55, 60, 70, 80, 90, or 100 nucleotides in length. In other embodiments, the antisense nucleic acid sequence is at least 5, 6, 7, 8, 9, 10, 11, 12, 13, 14, 15, 16, 17, 18, 19, 20, 21, 22, 23, 24, 25, 26, 27, 28, 29, 30, 32, 34, 36, 38, 40, 45, 50, 55, 60, 70, 80, 90 or 100 nucleotides in length. In certain embodiments, the antisense nucleic acid sequence is from 8 to 21, 9 to 21, 10 to 21, 11 to 21, 13 to 21, 14 to 21, 15 to 21, 16 to 21, 17 to 21, or 18 to 21 nucleotides in length. In other embodiments, the antisense nucleic acid sequence is about 22 nucleotides in length. In some embodiments, the antisense nucleic acid sequence has at least 65%, at least 70%, at least 75%, at least 80%, at least 85%, at least 90%, at least 95%, at least 96%, at least 97%, at least 98%, or at least 99% identity or 100% complementarity with a 5, 6, 7, 8, 9, 10, 11, 12, 13, 14, 15, 16, 17, 18, 19, 20, 21 or 22 nucleotide sequence within an mRNA translatable to an RPTP or IAD PTPR (or subgroup thereof) described herein. In certain embodiments, the antisense nucleic acid sequence only lacks complementarity with a sequence of an mRNA translatable to an RPTP or IAD PTPR (or subgroup thereof) described herein by 1, 2, 3, 4, 5, 6, 7, 8, 9, 10, 11, 12, 13, 14, 15, 16, 17, 18, 19, 20 nucleotides. In some embodiments, a nucleotide is nucleotide analog (e.g. adenine, guanine, thymine, cytosine, uracil, hypoxanthine, xanthine, 7-methylguanine, 5,6-dihydrouracil, 5-methylcytosine, or derivatives thereof) in each member of the set of minimum repeating units (e.g. nucleic acid analogs) and complementarity is measured by the nucleotide interactions between the inhibitory nucleic acid and its target nucleic acid. In some embodiments, the antisense nucleic acid sequence has at least 65%, at least 70%, at least 75%, at least 80%, at least 85%, at least 90%, at least 95%, at least 96%, at least 97%, at least 98%, or at least 99% complementarity with a segment of the mRNA translatable into an RPTP or IAD PTPR (or subgroup thereof) described herein, that is also 5, 6, 7, 8, 9, 10, 11, 12, 13, 14, 15, 16, 17, 18, 19, 20, 21, 22, 23, 24, 25, 26, 27, 28, 29, 30, 32, 34, 36, 38, 40, 45, 50, 55, 60, 70, 80, 90, or 100 nucleotides in length.

“Complementary” or “complementarity” as used herein, refers to the capacity for precise pairing of two nucleotides (e.g. A to T (or U), and G to C) regardless of where in the nucleic acid the two are located. For example, if a nucleotide at a certain position of first nucleic acid is capable of hydrogen bonding with a nucleotide at a certain position of an second nucleic acid (e.g. inhibitory nucleic acid, antisense nucleic acid, siRNA, miRNA, shRNA, shmiRNA, precursor miRNA), then the position of hydrogen bonding between the first nucleic acid and the second nucleic acid is considered to be a complementary position or position with complementarity. The first and second nucleic acids are “substantially complementary” to each other when a sufficient number of complementary positions in each molecule are occupied by nucleobases that can hydrogen bond with each other. Thus, the term “substantially complementary” is used to indicate a sufficient degree of precise pairing over a sufficient number of nucleotides such that stable and specific binding occurs between the two nucleic acids. The phrase “substantially complementary” thus means that there may be one or more mismatches between the two nucleic acids when they are aligned, provided that stable and specific binding occurs. The term “mismatch” refers to a site at which a nucleotide in the first nucleic acid and a nucleotide in the second nucleic acid with which it is aligned are not complementary or do not have complementarity. The two nucleic acids are “perfectly complementary” to each other when the first nucleic acid is fully complementary to the second nucleic acid across the entire aligned length of the nucleic acids.

Antisense nucleic acids that specifically hybridize to nucleic acid sequences encoding an RPTP or an IAD PTPR polypeptide can also be used to silence transcription and/or translation, and thus treat or prevent inflammatory autoimmune disease. For example, antisense oligonucleotides that specifically hybridize to a single IAD PTPR polynucleotide sequence can be used. A decrease in the severity of inflammatory autoimmune disease symptoms in comparison to symptoms detected in the absence of the inhibitory nucleic acids can be used to monitor the efficacy of the inhibitory nucleic acids.

The invention also provides antibodies that specifically bind to a single RPTP or a single IAD PTPR protein.

An antibody that specifically detects a single RPTP or a single IAD PTPR, and not other RPTP or IAD PTPR proteins, can be isolated using standard techniques described herein. The protein sequences for RPTP and IAD PTPR proteins in a number of species, e.g., humans, non-human primates, rats, dogs, cats, horses, bovines, etc., are publically available.

Table I summarizes the percent identity and similarity between human and mouse RPTPs.

TABLE I
Percent Identity and Similarity between
human and mouse RPTPs

| Protein | % Identity | % Similarity |
|---|---|---|
| PTPRA | 78.9 | 86.2 |
| PTPRB | 83.8 | 91.3 |
| PTPRC | 40.7 | 53.5 |
| PTPRD | 94.1 | 95.8 |
| PTPRE | 60.7 | 78.6 |
| PTPRF | 93.8 | 96.6 |
| PTPRG | 91.1 | 95.0 |
| PTPRH | 42.7 | 51.6 |
| PTPRJ | 56.4 | 67.0 |
| PTPRK | 98.5 | 99.7 |
| PTPRM | 97.1 | 98.6 |
| PTPRN | 78.8 | 84.5 |
| PTPRN2 | 54.3 | 68.2 |
| PTPRO | 89.5 | 94.8 |
| PTPRQ | 84.2 | 91.5 |
| PTPRR | 81.1 | 88.3 |
| PTPRS | 91.6 | 94.6 |
| PTPRT | 98.9 | 99.5 |
| PTPRU | 94.9 | 96.9 |
| PTPRZ1 | 75.8 | 84.4 |

In some embodiments, the antibody (e.g. the anti-IAD PTPR antibody) is a monoclonal antibody. Monoclonal antibodies are obtained by various techniques familiar to those skilled in the art. Briefly, spleen cells from an animal immunized with a desired antigen are immortalized, commonly by fusion with a myeloma cell (see, for example, Kohler & Milstein, Eur. J. Immunol. 6: 511-519 (1976)). Alternative methods of immortalization include transformation with Epstein Barr Virus, oncogenes, or retroviruses, or other methods well known in the art. Colonies arising from single immortalized cells are screened for production of antibodies of the desired specificity and affinity for the antigen, and yield of the monoclonal antibodies produced by such cells may be enhanced by various techniques, including injection into the peritoneal cavity of a vertebrate host. Alternatively, one may isolate DNA sequences which encode a monoclonal antibody or a binding fragment thereof by screening a DNA library from human B cells according to the general protocol outlined by Huse et al., Science 246: 1275-1281 (1989).

Monoclonal antibodies are collected and titered against an RPTP or an IAD PTPR protein in an immunoassay, for example, a solid phase immunoassay with the immunogen immobilized on a solid support. Monoclonal antibodies will usually bind with a K_{d} of at least about 0.1 mM, more usually at least about 1 μM, and can often be designed to bind with a K_{d} of 1 nM or less.

The antibodies, including RPTP-binding fragments or IAD PTPR-binding fragments and derivatives thereof, can be produced readily by a variety of recombinant DNA techniques, including by expression in transfected cells (e.g., immortalized eukaryotic cells, such as myeloma or hybridoma cells) or in mice, rats, rabbits, or other vertebrate capable of producing antibodies by well known methods. Suitable source cells for the DNA sequences and host cells for immunoglobulin expression and secretion can be obtained from a number of sources, such as the American Type Culture Collection (Catalogue of Cell Lines and Hybridomas, Fifth edition (1985) Rockville, Md.).

In some embodiments, the antibody (e.g. the anti-IAD PTPR antibody) is a humanized antibody, i.e., an antibody that retains the reactivity of a non-human antibody while being less immunogenic in humans. This can be achieved, for instance, by retaining the non-human CDR regions that are specific for an RPTP or an IAD PTPR, and replacing the remaining parts of the antibody with their human counterparts.

The activity of an RPTP or an IAD PTPR protein can be inhibited using IAD therapeutic agents that bind to the RPTP or IAD PTPR. IAD therapeutic agents are described herein.

### IV. Methods of Identifying RPTP Binding Agents

Provided herein are methods for identifying new RPTP or new IAD PTPR binding agents. In some embodiments, the method includes a first step of contacting a test agent (also referred to herein as a candidate agent) with an RPTP. The RPTP may be PTPRA, PTPRB, PTPRC, PTPRD, PTPRE, PTPRF, PTPRG, PTPRH, PTPRJ, PTPRK, PTPRM, PTPRN, PTPRN2, PTPRO, PTPRQ, PTPRR, PTPRS, PTPRT, PTPRU, PTPRV or PTPRZ1. In some embodiments, the RPTP is PTPRA, PTPRB, PTPRC, PTPRE, PTPRF, PTPRG, PTPRJ, PTPRM, PTPRO, PTPRS, or PTPRZ1. In other embodiments, the RPTP is PTPRA, PTPRB, PTPRC, PTPRE, PTPRG, PTPRJ, PTPRM, PTPRO, or PTPRS. In other embodiments, the RPTP is PTPRB, PTPRE, PTPRG, PTPRJ, PTPRM, PTPRO, or PTPRS. In some embodiments, the RPTP is PTPRB, PTPRE, PTPRG, PTPRM, PTPRO, or PTPRS. In other embodiments, the RPTP is PTPRE, PTPRG, PTPRM, PTPRO, or PTPRS. In certain embodiments, the RPTP is PTPRE or PTPRS. In other embodiments, the RPTP is PTPRE. In still other embodiments, the RPTP is PTPRS. In some embodiments, the RPTP is PTPRF. In other embodiments, the RPTP is PTPRZ1. Some embodiments include a second step of detecting binding of the test agent to the RPTP, thereby identifying a candidate RPTP binding agent. In certain embodiments, the RPTP is PTPRA, PTPRB, PTPRC, PTPRE, PTPRF, PTPRG, PTPRJ, PTPRM, PTPRO, PTPRS, or PTPRZ1. In other embodiments, the method includes identifying a candidate IAD binding agent. The method further includes the first step of contacting a test agent with an IAD PTPR. In some embodiments, the IAD PTPR is PTPRA, PTPRB, PTPRC, PTPRE, PTPRG, PTPRJ, PTPRM, PTPRO, or PTPRS. The method further includes a second step of detecting binding of the test agent to the IAD PTPR.

In some embodiments, the test agent is a candidate IAD binding agent. The candidate IAD binding agent may be a candidate IAD therapeutic agent. In certain embodiments, the candidate IAD therapeutic agent is selected from a candidate anti-IAD PTPR antibody, a candidate anti-IAD PTPR inhibitory nucleic acid or a candidate IAD PTPR ligand mimetic. In some embodiments, the candidate IAD therapeutic agent is a candidate anti-IAD PTPR antibody (e.g. a candidate anti-IAD PTPRS antibody or a candidate anti-IAD PTPRE antibody). In other embodiments, the candidate IAD therapeutic agent is a candidate anti-IAD PTPR inhibitory nucleic acid. In still other embodiments, the candidate anti-IAD PTPR inhibitory nucleic acid is a candidate anti-IAD PTPR RNAi molecule. The candidate anti-IAD PTPR inhibitory nucleic acid may also be a candidate anti-IAD PTPR antisense nucleic acid (e.g. a candidate anti-IAD PTPRS antisense nucleic acid or a candidate anti-IAD PTPRE antisense nucleic acid). In some embodiments, the candidate IAD PTPR ligand mimetic is a peptide or a small chemical molecule. In some embodiments, the IAD PTPR is PTPRB, PTPRE, PTPRG, PTPRJ, PTPRM, PTPRO, or PTPRS. The IAD PTPR may be PTPRB, PTPRE, PTPRG, PTPRM, PTPRO, or PTPRS. The IAD PTPR may be PTPRE, PTPRG, PTPRM, PTPRO, or PTPRS. The IAD PTPR may be PTPRE or PTPRS. In some embodiments, the IAD PTPR is PTPRS.

The invention further provides methods for identifying antagonists or agonists of an RPTP or an IAD PTPR expression and/or activity. Methods for screening for antagonists or agonists may include measuring the ability of the potential antagonists or agonist to reduce or increase an identifiable RPTP or IAD PTPR activity or compete for binding with a known binding agent (e.g., RPTP-specific or IAD PTPR-specific antibody). For example, candidate agents can be screened for their ability to reduce an RPTP or an IAD PTPR activity or substrate dephosphorylation. Some embodiments include identifying agonists of PTPRF or PTPRZ1. Other embodiments include identifying antagonists of PTPRB, PTPRE, PTPRG, PTPRM, PTPRO, or PTPRS. Some embodiments include identifying agonists of PTPRF. Other embodiments include identifying agonists of PTPRZ1. Some embodiments include identifying antagonists of PTPRE. Certain embodiments include identifying antagonists of PTPRS.

The screening methods of the invention can be performed as in vitro or cell-based assays or in suitable animal models (e.g. SKG mouse model of arthritis, type II collagen mouse or rat model of rheumatoid arthritis, NOD mice, MRL lupus-prone mice MRL/MpJ-Fas^{lpr}/J. Thus, in some embodiments, the “contacting a test agent with an RPTP” includes contacting the RPTP wherein the RPTP forms part of a cell. The cell, in turn, may form part of a tissue and/or an organism (i.e. an animal model). Cell based assays can be performed in any cells in which an RPTP or IAD PTPR is expressed, either endogenously or through recombinant methods. Cell-based assays may involve whole cells or cell fractions containing an RPTP or IAD PTPR to screen for agent binding or modulation of an RPTP or IAD PTPR activity by the agent. Suitable cell-based assays are described in, e.g., DePaola et al., Annals of Biomedical Engineering 29: 1-9 (2001).

Agents that are initially identified as inhibiting an RPTP or IAD PTPR can be further tested to validate the apparent activity. Preferably such studies are conducted with suitable cell-based or animal models of disease (e.g. autoimmune disease, inflammatory autoimmune disease, cancer, infectious disease, immune disease, obesity, metabolic disease, inflammatory disease, traumatic injury, etc.). The basic format of such methods involves administering a lead compound identified during an initial screen to an animal that serves as a model and then determining if in fact the disease (e.g. autoimmune disease, inflammatory autoimmune disease, cancer, infectious disease, immune disease, obesity, metabolic disease, inflammatory disease, traumatic injury, etc.) or one or more of the disease symptoms are ameliorated. The animal models utilized in validation studies generally are mammals of any kind. Specific examples of suitable animals include, but are not limited to, primates (e.g., chimpanzees, monkeys, and the like) and rodents (e.g., mice, rats, guinea pigs, rabbits, and the like).

The agents tested as potential agonists or antagonists of an RPTP or IAD PTPR can be any appropriate small chemical compound, or a biological entity, such as a polypeptide, sugar, nucleic acid or lipid. Alternatively, modulators (e.g. autoimmune therapeutic agents, IAD therapeutic agents, agonists or antagonists) can be genetically altered versions of an RPTP or IAD PTPR, e.g., forms that are not glycosylated. Essentially any appropriate chemical compound can be used as a potential modulator or ligand in the assays of the invention. The assays may be designed to screen large chemical libraries by automating the assay steps and providing compounds from any convenient source to assays, which are typically run in parallel (e.g., in microtiter formats on microtiter plates in robotic assays).

In one embodiment, high throughput screening methods are employed by providing a combinatorial chemical or peptide library containing a large number of candidate RPTP binding agents (e.g. candidate IAD binding agents, candidate autoimmune therapeutic agents, candidate IAD therapeutic agents, etc.). Such “combinatorial chemical libraries” or “ligand libraries” are then screened in one or more assays, to identify those library members (particular chemical species or subclasses) that display a desired characteristic activity. The candidate compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Int. J. Pept. Prot. Res. 37:487-493 (1991) and Houghton et al., Nature 354:84-88 (1991)). Other chemistries for generating chemical diversity libraries can also be used. Such chemistries include, but are not limited to: peptoids (e.g., PCT Publication No. WO 91/19735), encoded peptides (e.g., PCT Publication WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (see Ausubel, Berger and Sambrook, all supra), peptide nucleic acid libraries (see, e.g., U.S. Pat. No. 5,539,083), antibody libraries (see, e.g., Vaughn et al., Nature Biotechnology, 14(3):309-314 (1996) and PCT/US96/10287), carbohydrate libraries (see, e.g., Liang et al., Science, 274:1520-1522 (1996) and U.S. Pat. No. 5,593,853), small organic molecule libraries (see, e.g., benzodiazepines, Baum C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, and U.S. Pat. No. 5,288,514).

### V. Pharmaceutical Compositions

Provided herein are pharmaceutical compositions for treating an individual who has or may be at risk of developing a disease by administering to the individual a pharmaceutical composition including a therapeutically effective amount of a therapeutic agent and a pharmaceutically acceptable excipient. In some embodiments, the pharmaceutical compositions are for treating an individual who has a disease by administering to the individual a pharmaceutical composition including a therapeutically effective amount of a therapeutic agent and a pharmaceutically acceptable excipient. In some embodiments, the pharmaceutical compositions are for treating an individual who may be at risk of developing a disease by administering to the individual a pharmaceutical composition including a therapeutically effective amount of a therapeutic agent and a pharmaceutically acceptable excipient. In some embodiments, the disease is an autoimmune disease or disorder, cancer, an infectious disease (e.g. viral, bacterial, parasitic, etc.), an obesity associated disease, a metabolic disease or disorder, an inflammatory disease, an immune disease or disorder, or a traumatic injury. In some embodiments, the disease is an inflammatory autoimmune disease (IAD). In some embodiments, the disease is a disease associated with a patient's joints. In a certain embodiment, the inflammatory autoimmune disease is rheumatoid arthritis. In some embodiments, the increased expression of one or more RPTPs is associated with a disease or a risk of developing the disease. In another embodiment, the decreased expression of one or more RPTPs is associated with a disease or a risk of developing the disease. In yet another embodiment, the increased expression of a first RPTP and the decreased expression of a second RPTP is associated with a disease or a risk of developing the disease. In some embodiments, the therapeutic agent is an agonist of one or more RPTPs In other embodiments, the therapeutic agent is an antagonist of one or more RPTPs. In still other embodiments, the therapeutic agent is an agonist for a first RPTP and an antagonist for a second RPTP.

In some embodiments, the therapeutic agent is an autoimmune therapeutic agent. In some embodiments, the therapeutic agent is an antagonist for one or more (e.g. 2, 3, 4, 5, 6, 7, 8, 9, 10, 11, 12, 13, 14, 15, 16, 17, 18, 19, 20, 21) RPTPs or IAD PTPRs. In some embodiments, the therapeutic agent is an antagonist for PTPRA, PTPRB, PTPRC, PTPRE, PTPRG, PTPRJ, PTPRM, PTPRO, PTPRS, or a combination thereof (e.g. 2, 3, 4, 5, 6, 7, 8, 9). In other embodiments, the therapeutic agent is an antagonist for PTPRB, PTPRE, PTPRG, PTPRM, PTPRO, PTPRS, or a combination thereof (e.g. 2, 3, 4, 5, 6). In further embodiments, the therapeutic agent is an antagonist for PTPRE, PTPRG, PTPRM, PTPRO, PTPRS, or a combination thereof (e.g. 2, 3, 4, 5). In certain embodiments, the therapeutic agent is an antagonist for PTPRE, PTPRS, or a combination thereof. In still other embodiments, the therapeutic agent is an antagonist for PTPRS.

The IAD therapeutic agent may be an anti-IAD PTPR antibody. In some embodiments, the IAD therapeutic agent is an anti-IAD PTPR inhibitory nucleic acid. In other embodiments, the anti-IAD PTPR inhibitory nucleic acid is an anti-IAD PTPR RNAi molecule. In other embodiments, the anti-IAD PTPR inhibitory nucleic acid is an antisense nucleic acid such as anti-IAD PTPR antisense nucleic acid. In still other embodiments, the IAD PTPR ligand mimetic is a peptide or a small chemical molecule. The IAD PTPR may be PTPRB, PTPRE, PTPRG, PTPRJ, PTPRM, PTPRO, or PTPRS. In some embodiments, the IAD PTPR is PTPRB, PTPRE, PTPRG, PTPRM, PTPRO, or PTPRS. In certain embodiments, the IAD PTPR is PTPRE, PTPRG, PTPRM, PTPRO, or PTPRS. The IAD PTPR may be PTPRE or PTPRS. In some embodiments, the IAD PTPR is PTPRS. In some embodiments, the IAD PTPR is PTPRE.

In other embodiments, the therapeutic agent is an anti-IAD PTPRS antisense nucleic acid. In other embodiments, the therapeutic agent is anti-IAD PTPRS antisense nucleic acid. In other embodiments, the therapeutic agent is an anti-IAD PTPRE antisense nucleic acid. In other embodiments, the therapeutic agent is anti-IAD PTPRE antisense nucleic acid.

In some embodiments, the therapeutic agent is an agonist for one or more (e.g. 2, 3, 4, 5, 6, 7, 8, 9, 10, 11, 12, 13, 14, 15, 16, 17, 18, 19, 20, 21) RPTPs or IAD PTPRs. In other embodiments, the therapeutic agent is an agonist for PTPRF or PTPRZ1, or a combination thereof. In certain embodiments, the therapeutic agent is an agonist for PTPRF. In further embodiments, the therapeutic agent is an agonist for PTPRZ1.

In some embodiments the therapeutic agent is an antagonist for PTPRA, PTPRB, PTPRC, PTPRE, PTPRG, PTPRJ, PTPRM, PTPRO, PTPRS, or a combination thereof (e.g. 2, 3, 4, 5, 6, 7, 8, 9) and the therapeutic agent is an agonist for PTPRF or PTPRZ1, or a combination thereof, In other embodiments, the pharmaceutical composition includes a therapeutic agent that is an inflammatory autoimmune disease therapeutic agent.

In some embodiments, the pharmaceutical composition includes an autoimmune therapeutic agent and a pharmaceutically acceptable excipient. In certain embodiments, the autoimmune therapeutic agent is an agonist or antagonist of an RPTP. In some embodiments, the RPTP is PTPRA, PTPRB, PTPRC, PTPRD, PTPRE, PTPRF, PTPRG, PTPRH, PTPRJ, PTPRK, PTPRM, PTPRN, PTPRN2, PTPRO, PTPRQ, PTPRR, PTPRS, PTPRT, PTPRU, PTPRV or PTPRZ1. In certain embodiments, the RPTP is PTPRA, PTPRB, PTPRC, PTPRE, PTPRF, PTPRG, PTPRJ, PTPRM, PTPRO, PTPRS, or PTPRZ1. The RPTP may be PTPRA, PTPRB, PTPRC, PTPRE, PTPRG, PTPRJ, PTPRM, PTPRO, or PTPRS. The RPTP may be PTPRB, PTPRE, PTPRG, PTPRJ, PTPRM, PTPRO, or PTPRS. The RPTP may be PTPRB, PTPRE, PTPRG, PTPRM, PTPRO, or PTPRS. The RPTP may be PTPRE, PTPRG, PTPRM, PTPRO, or PTPRS. The RPTP may be PTPRE or PTPRS. The RPTP may be PTPRE. The RPTP may be PTPRS. In other embodiments, the RPTP is PTPRF. In other embodiments, the RPTP is PTPRZ1.

In some embodiments, the pharmaceutical composition includes an IAD therapeutic agent and a pharmaceutically acceptable excipient. In some embodiments, the IAD therapeutic agent is selected from an anti-IAD PTPR antibody, an anti-IAD PTPR inhibitory nucleic acid or an IAD PTPR ligand mimetic. In some embodiments, the pharmaceutical composition includes an anti-IAD PTPR antibody. In other embodiments, the pharmaceutical composition includes an anti-IAD PTPR inhibitory nucleic acid. In certain embodiments, the pharmaceutical composition includes an anti-IAD PTPR inhibitory nucleic acid that is an anti-IAD PTPR RNAi molecule. In certain embodiments, the pharmaceutical composition includes an anti-IAD PTPR inhibitory nucleic acid that is an anti-IAD PTPR antisense nucleic acid. In some embodiments, the pharmaceutical composition includes an IAD PTPR ligand mimetic that may be a peptide or a small chemical molecule.

In some embodiments, the pharmaceutical composition includes an IAD therapeutic agent and a pharmaceutically acceptable excipient. In some embodiments, the IAD therapeutic agent is an antagonist of an IAD PTPR. In other embodiments, the IAD PTPR is PTPRB, PTPRE, PTPRG, PTPRJ, PTPRM, PTPRO, or PTPRS. In certain embodiments, the IAD PTPR is PTPRB, PTPRE, PTPRG, PTPRM, PTPRO, or PTPRS. In some embodiments, the IAD PTPR is PTPRE, PTPRG, PTPRM, PTPRO, or PTPRS. In certain embodiments, the IAD PTPR is PTPRE, PTPRG, PTPRM, PTPRO, or PTPRS. In some embodiments, the IAD PTPR is PTPRE or PTPRS. In some embodiments, IAD PTPR is PTPRS.

In some embodiments, the pharmaceutical composition is useful for treating an individual who has or may be at risk of developing an inflammatory autoimmune disease. In some embodiments, the pharmaceutical compositions are useful for treating an individual who has an inflammatory autoimmune disease by administering to the individual a pharmaceutical composition including a therapeutically effective amount of a IAD therapeutic agent and a pharmaceutically acceptable excipient. In certain embodiments, the pharmaceutical compositions are for treating an individual who may be at risk of developing an inflammatory autoimmune disease by administering to the individual a pharmaceutical composition including a therapeutically effective amount of an IAD therapeutic agent and a pharmaceutically acceptable excipient. In some embodiments, the inflammatory autoimmune disease is an arthritis. In some embodiments, inflammatory autoimmune disease is rheumatoid arthritis, psoriatic arthritis, juvenile idiopathic arthritis, multiple sclerosis, systemic lupus erythematosus (SLE), myasthenia gravis, juvenile onset diabetes, diabetes mellitus type 1, Guillain-Barre syndrome, Hashimoto's encephalitis, Hashimoto's thyroiditis, ankylosing spondylitis, psoriasis, Sjogren's syndrome, vasculitis, glomerulonephritis, auto-immune thyroiditis, Behcet's disease, Crohn's disease, ulcerative colitis, bullous pemphigoid, sarcoidosis, psoriasis, ichthyosis, Graves ophthalmopathy, inflammatory bowel disease, Addison's disease, Vitiligo, asthma, or allergic asthma . . . . In some embodiments, the inflammatory autoimmune disease is rheumatoid arthritis.

The compositions disclosed herein can be administered by any means known in the art. For example, compositions may include administration to a subject intravenously, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, intrathecally, subcutaneously, subconjunctival, intravesicularlly, mucosally, intrapericardially, intraumbilically, intraocularly, orally, locally, by inhalation, by injection, by infusion, by continuous infusion, by localized perfusion, via a catheter, via a lavage, in a creme, or in a lipid composition. Administration can be local, e.g., to the joint or systemic.

Solutions of the active compounds as free base or pharmacologically acceptable salt can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations can contain a preservative to prevent the growth of microorganisms.

Pharmaceutical compositions can be delivered via intranasal or inhalable solutions or sprays, aerosols or inhalants. Nasal solutions can be aqueous solutions designed to be administered to the nasal passages in drops or sprays. Nasal solutions can be prepared so that they are similar in many respects to nasal secretions. Thus, the aqueous nasal solutions usually are isotonic and slightly buffered to maintain a pH of 5.5 to 6.5. In addition, antimicrobial preservatives, similar to those used in ophthalmic preparations, and appropriate drug stabilizers, if required, may be included in the formulation. Various commercial nasal preparations are known and can include, for example, antibiotics and antihistamines.

Oral formulations can include excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders. In some embodiments, oral pharmaceutical compositions will comprise an inert diluent or assimilable edible carrier, or they may be enclosed in hard or soft shell gelatin capsule, or they may be compressed into tablets, or they may be incorporated directly with the food of the diet. For oral therapeutic administration, the active compounds may be incorporated with excipients and used in the form of ingestible tablets, buccal tablets, troches, capsules, elixirs, suspensions, syrups, wafers, and the like. The percentage of the compositions and preparations may, of course, be varied and may conveniently be between about 2 to about 75% of the weight of the unit, or preferably between 25-60%. The amount of active compounds in such compositions is such that a suitable dosage can be obtained

For parenteral administration in an aqueous solution, for example, the solution should be suitably buffered and the liquid diluent first rendered isotonic with sufficient saline or glucose. Aqueous solutions, in particular, sterile aqueous media, are especially suitable for intravenous, intramuscular, subcutaneous and intraperitoneal administration. For example, one dosage could be dissolved in 1 ml of isotonic NaCl solution and either added to 1000 ml of hypodermoclysis fluid or injected at the proposed site of infusion

Sterile injectable solutions can be prepared by incorporating the active compounds or constructs in the required amount in the appropriate solvent followed by filtered sterilization. Generally, dispersions are prepared by incorporating the various sterilized active ingredients into a sterile vehicle which contains the basic dispersion medium. Vacuum-drying and freeze-drying techniques, which yield a powder of the active ingredient plus any additional desired ingredients, can be used to prepare sterile powders for reconstitution of sterile injectable solutions. The preparation of more, or highly, concentrated solutions for direct injection is also contemplated. DMSO can be used as solvent for extremely rapid penetration, delivering high concentrations of the active agents to a small area.

The invention provides methods of treating, preventing, and/or ameliorating an inflammatory autoimmune disorder in a subject in need thereof, optionally based on the diagnostic and predictive methods described herein. The course of treatment is best determined on an individual basis depending on the particular characteristics of the subject and the type of treatment selected. The treatment, such as those disclosed herein, can be administered to the subject on a daily, twice daily, bi-weekly, monthly or any applicable basis that is therapeutically effective. The treatment can be administered alone or in combination with any other treatment disclosed herein or known in the art. The additional treatment can be administered simultaneously with the first treatment, at a different time, or on an entirely different therapeutic schedule (e.g., the first treatment can be daily, while the additional treatment is weekly).

Administration of a composition for ameliorating the inflammatory autoimmune disease, e.g., by treating elevated or decreased expression of the RPTP or IAD PTPR gene, can be a systemic or localized administration. For example, treating a subject having an inflammatory autoimmune disorder can include administering an oral or injectable form of anti-IAD PTPR agent (IAD PTPR antagonist) on a daily basis or otherwise regular schedule. In some embodiments, the treatment is only on an as-needed basis, e.g., upon appearance of inflammatory autoimmune disease symptoms.

A pharmaceutically acceptable excipient is used herein according to its generally recognized meaning in the pharmaceutical arts. It may refer to any substance included in a pharmaceutical composition, which is generally considered to be not an active ingredient, that is commonly employed in order to improve administration or absorption of the active ingredient. In some embodiments, excipients facilitate the manufacture, administration, storage, or efficacy of an active ingredient. Examples of excipients include antiadherents, binders, coatings, disintegrants, fillers, diluents, flavors, colors, lubricants, glidants, preservatives, sorbents, or sweeteners. In some embodiments, the active ingredient may also have one or more properties listed immediately above.

Any appropriate element disclosed in one aspect or embodiment of a method or composition disclosed herein is equally applicable to any other aspect or embodiment of a method or composition. For example, the therapeutic agents set forth in the description of the pharmaceutical compositions provided herein are equally applicable to the methods of treatment and vice versa.

### VI. EXAMPLES

The examples described herein are meant only to illustrate certain embodiments of the invention and not to limit the scope of the invention provided herein.

### Example 1

Experiments were performed to determine expression of RPTPs in T cells using Jurkat cells, a human T cell line. For T cell receptor (TCR) stimulation, Jurkat cells were incubated for 24 hours in the presence of 5 μg/mL of plate-bound anti-CD3 antibody and 2 μg/mL of soluble anti-CD28 antibody. Alternatively, cells were stimulated for 24 hours with 10 ng/mL of PMA and 1 μM ionomycin. Treatment with PMA/ionomycin strongly stimulates the T cell transcriptional machinery, thus showing the activation potential of genes after intense stimulation. RNA was then extracted from the cells (Qiagen kit) and RPTP expression levels were quantified by quantitative PCR (QPCR).

Many RPTPs are significantly expressed in a T cell line. When T cells are stimulated via their T cell receptor (TCR) or with PMA/ionomycin, RPTP expression may be altered. These experiments show that RPTPs have significant roles in T cells.

Table II summarizes experimental data indicating that 10 RPTPs are expressed in T cells in addition to CD45 and CD148.

TABLE II
Expression of 10 RPTPs in T cells in addition to CD45 and CD148
Expression Levels Relative to CD148

| Gene | Resting | TCR Stimulated | PMA/Ionomycin |
|---|---|---|---|
| PTPRA/RPTPα | 30.8 ± 3.5 | 30.9 ± 2.8 | 40.2 ± 14.4 |
| PTPRC/CD45 | 216.1 ± 19.9 | 492.3 ± 44.5 | 565 ± 201.5 |
| PTPRE/RPTPε | 17.1 ± 1.6 | 35.4 ± 3.6 | 47.4 ± 17.0 |
| PTPRF/LAR | 10.5 ± 1.1 | 4.7 ± 0.7 | 3.3 ± 1.2 |
| PTPRJ/CD148 | 1.0 ± 0.1 | 1.7 ± 0.3 | 4.6 ± 1.6 |
| PTPRK/RPTPκ | 46.6 ± 4.3 | 22.3 ± 2.0 | 26.4 ± 10.8 |
| PTPRM/RPTPμ | 0.1 ± 0.0 | 0.1 ± 0.0 | 0.5 ± 0.3 |
| PTPRN2/IA-2β | 10.9 ± 1.0 | 8.1 ± 1.0 | 9.2 ± 3.3 |
| PTPRR/PTP-SL | Not Expressed | 0.1 ± 0.5 | 0.3 ± 0.1 |
| PTPRS/RPTPσ | 44.7 ± 4.0 | 32.0 ± 3.0 | 70.9 ± 25.3 |
| PTPRU/RPTPψ | 12.7 ± 1.2 | 11.9 ± 1.1 | 10.5 ± 3.8 |
| PTPRV/OST-PTP | 0.1 ± 0.0 | 0.2 ± 0.0 | 0.3 ± 0.1 |

It was also determined that animal data, in particular, mouse-derived data, may be a reliable tool to predict a modulated response in humans. In particular, because of the high percentage identity and similarity between human and mouse RPTPs, mouse antibodies are likely to cross react with human RPTPs.

### Example 2

The role of RPTPs in the immune system was investigated, in particular, the role of RPTP in regulatory T cells (Tregs). Little information is available about RPTP expression in Tregs and the expression RPTPs in effector T cells (Teffs).

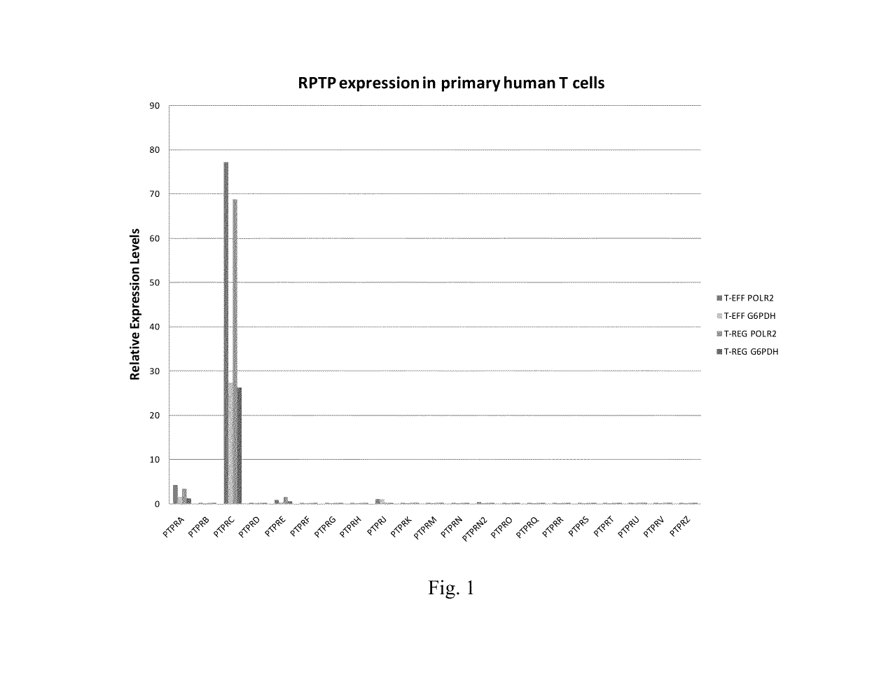

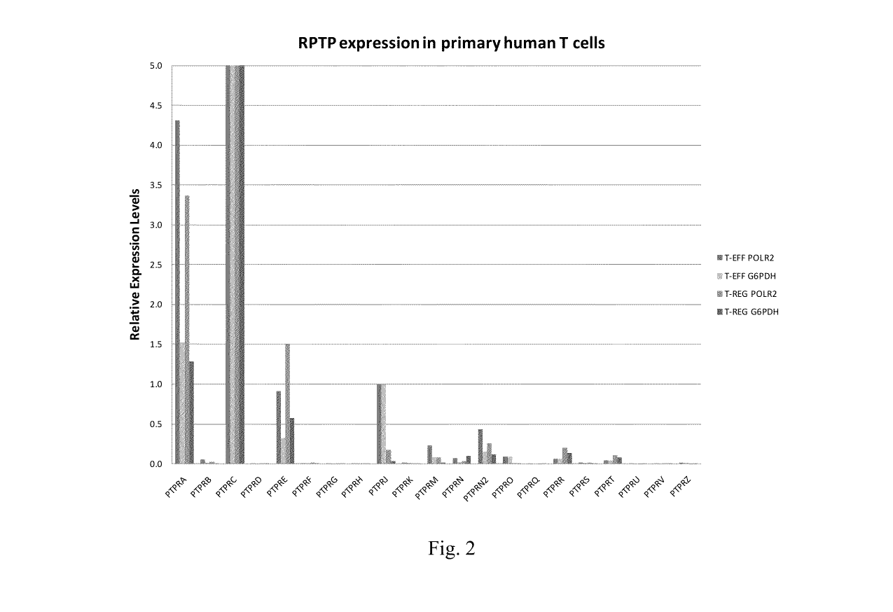

Peripheral blood mononuclear cells from human blood were isolated using Lymphoprep density centrifugation, labeled with antibodies against CD4, CD25 and CD127, and then sorted using fluorescence activated cell sorting to isolate Teffs (CD4+CD25lowCD127+) and Tregs (CD4+CD25hiCD127−). A portion of these Teffs and Tregs were used for analysis of FoxP3 levels (FIG. 2), while RNA was extracted from the remainder of Teffs and Tregs. After RNA extraction (Qiagen kit), the expression of all RPTPs was compared in both primary human T cell populations using QPCR. Expression of the RPTPs was normalized to expression of the housekeeping genes RNA polymerase II (POLR2) or G6PDH, and calculated relative to PTPRJ in Teffs. RPTPs showed expression in Tregs or differential expression between Teffs and Tregs. The results of this investigation are summarized in Table III and in FIGS. 1 and 2.

TABLE III

| | T-EFF | T-EFF | T-REG | T-REG |
|---|---|---|---|---|
| | POLR2 | G6PDH | POLR2 | G6PDH |
| PTPRA | 4.3091 | 1.5299 | 3.3682 | 1.2898 |
| PTPRB | 0.0498 | 0.0177 | 0.0237 | 0.0091 |
| PTPRC | 77.1317 | 27.3848 | 68.7215 | 26.3169 |
| PTPRD | 0.0101 | 0.0036 | 0.0080 | 0.0030 |
| PTPRE | 0.9095 | 0.3229 | 1.5123 | 0.5791 |
| PTPRF | 0.0046 | 0.0016 | 0.0126 | 0.0048 |
| PTPRG | 0.0046 | 0.0016 | 0.0028 | 0.0011 |
| PTPRH | 0.0046 | 0.0016 | 0.0028 | 0.0011 |
| PTPRJ | 1.0000 | 1.0000 | 0.1742 | 0.0313 |
| PTPRK | 0.0125 | 0.0114 | 0.0019 | 0.0021 |
| PTPRM | 0.2307 | 0.0819 | 0.0818 | 0.0127 |
| PTPRN | 0.0741 | 0.0263 | 0.0332 | 0.0976 |
| PTPRN2 | 0.4322 | 0.1535 | 0.2550 | 0.1199 |
| PTPRO | 0.0939 | 0.0939 | 0.0044 | 0.0030 |
| PTPRQ | 0.0031 | 0.0031 | 0.0044 | 0.0030 |
| PTPRR | 0.0654 | 0.0654 | 0.1989 | 0.1368 |
| PTPRS | 0.0173 | 0.0173 | 0.0142 | 0.0097 |
| PTPRT | 0.0419 | 0.0419 | 0.1126 | 0.0775 |
| PTPRU | 0.0078 | 0.0078 | 0.0044 | 0.0030 |
| PTPRV | 0.0031 | 0.0031 | 0.0102 | 0.0070 |
| PTPRZ | 0.0116 | 0.0116 | 0.0044 | 0.0030 |

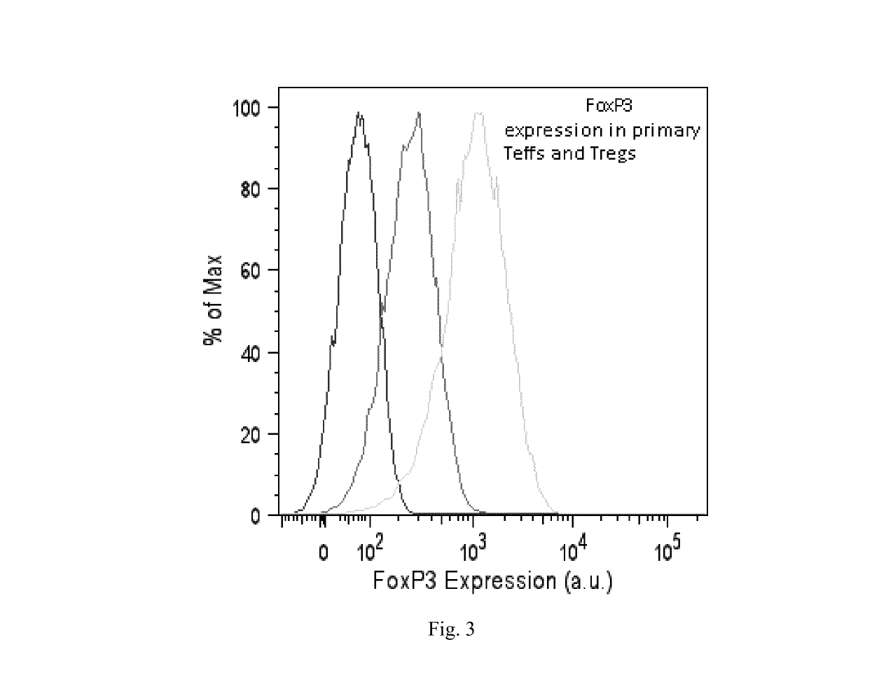

In the experiment illustrated in FIG. 3, cells were fixed, permeabilized, and stained with an anti-FoxP3-AlexaFluor488 antibody. The expression of FoxP3, a marker for Tregs, was then analyzed by flow cytometry. In FIG. 3, Tregs are represented with a green line, Teffs with a red line, and unstained Teffs with a blue line. This assay confirms the purity of the populations of Teffs (which are negative for FoxP3) and Tregs (which are positive for FoxP3) used in FIGS. 1 and 2.

These RPTPs are potentially involved in the differentiation or function of Teffs or Tregs, and thus aid in the identification of novel targets for treatment of the above described diseases, disorders, or conditions by targeting a particular subpopulation of T cells.

RPTPs can be targeted by biologicals (for example, peptides, peptidomimetics, proteins, antibodies, antibody fragments, nucleic acids, RNAi or inhibitory nucleic acids) or small chemical molecules to modulate their activity in a positive or negative way.

### Example 3

The role of RPTPs in auto-immune disease was investigated using the SKG mouse as a model of rheumatoid arthritis. Mouse wrist joints from arthritic (SKG mice) or control mice were homogenized, followed by extraction of RNA with Trizol (Invitrogen). For lymph node analysis, lymph nodes were extracted from arthritic or control mice and strained into a single-cell suspension. Cells were then lysed and RNA was extracted. After extraction of RNA (Qiagen kit), all samples were analyzed by QPCR for the expression of different RPTPs in arthritic versus control tissue.

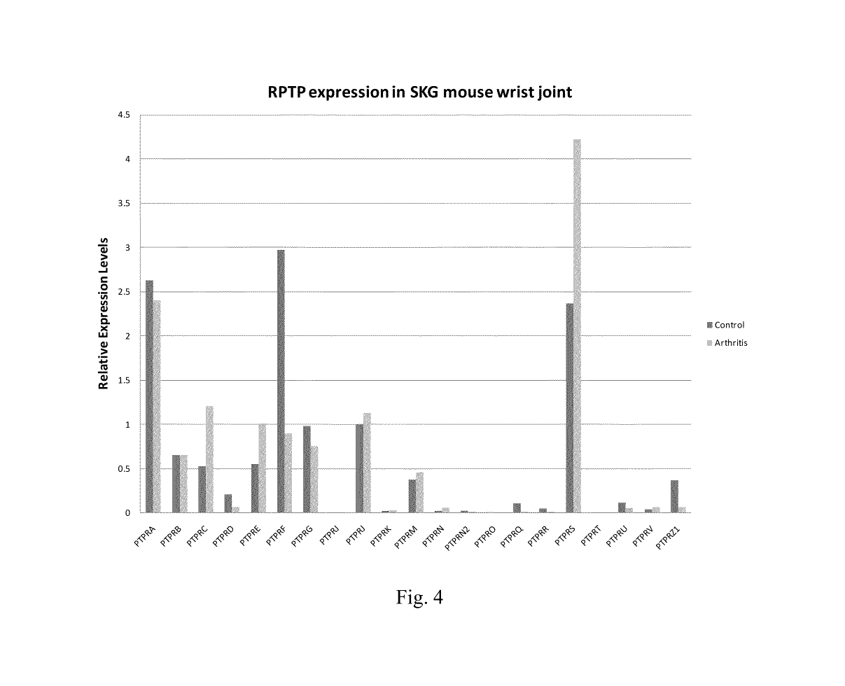

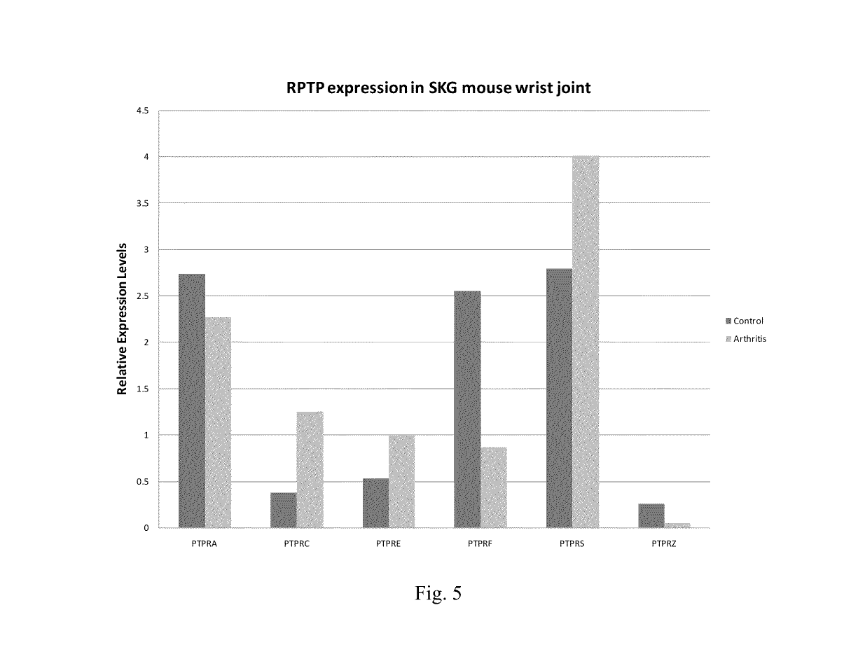

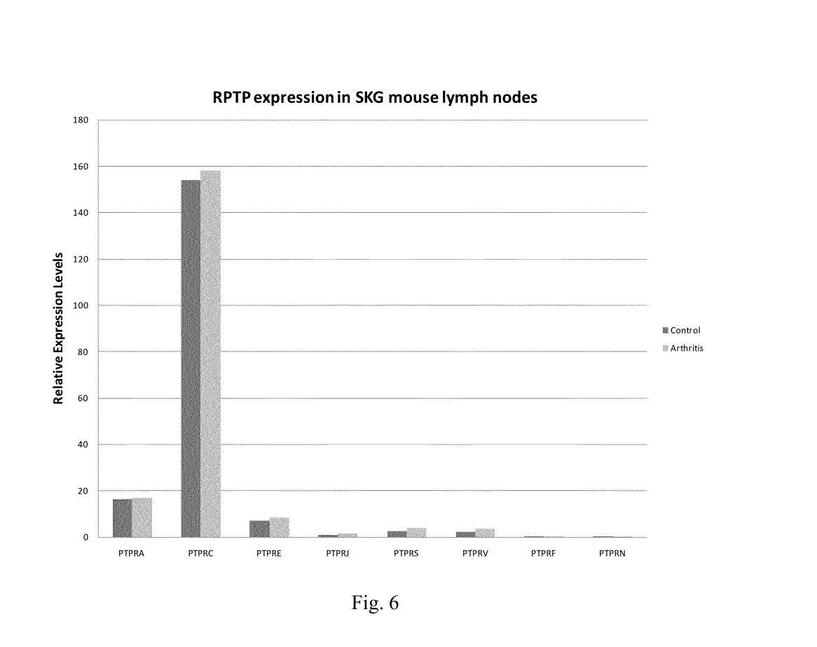

FIGS. 4 and 5 show differential expressions between arthritic and control tissue in mouse wrist joint cells, while FIG. 6 shows differential expression in mouse lymph node cells. Expression levels are shown relative to the levels of PTPRJ in control mice.

In particular, it was found that at least two RPTPs showed dramatic changes in expression in arthritic SKG mice compared to control mice: PTPRS has increased expression in arthritic joints and lymph nodes, and PTPRF has decreased expression in arthritic joints.

These RPTPs are involved in immunity, auto-immunity, and inflammation, and thus this technology is a viable tool in the identification of novel targets for treatment of immune, auto-immune, and inflammatory diseases, disorders, or conditions.

That is because RPTPs can be targeted by biologicals (for example, peptides, peptidomimetics, proteins, antibodies, antibody fragments, nucleic acids, RNAi, or inhibitory nucleic acids) or small chemical molecules to modulate their activity or expression in a positive or negative way.

This is the first analysis of RPTP expression in an auto-immune model. This is also the first report of the increased expression patterns of PTPRS in lymph nodes and arthritic joints of mice, and the first report of decreased PTPRF in arthritic joints of mice.

### Example 4

### RPTP Expression from Primary Mouse Synoviocytes

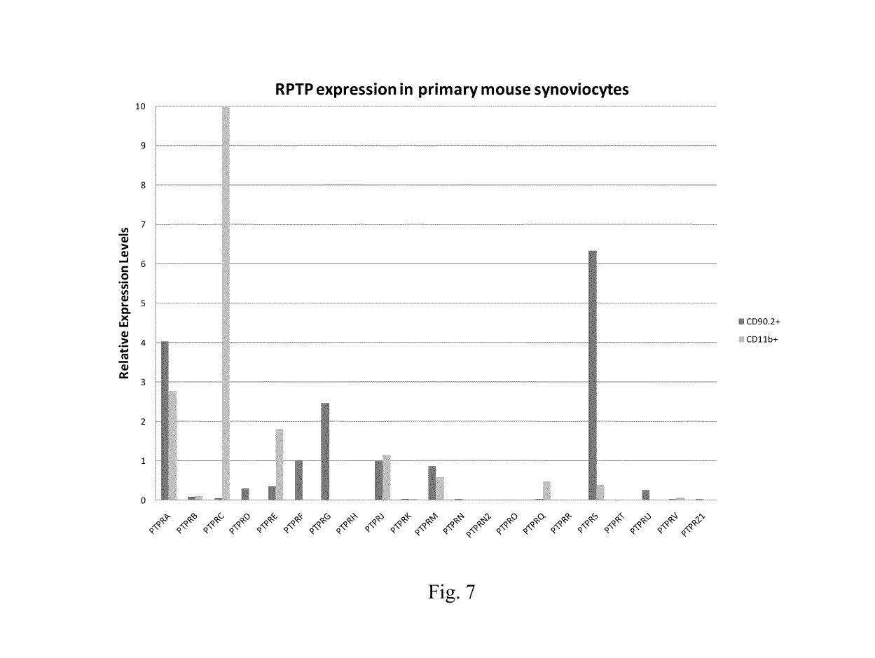

FIG. 7 shows mRNA expression levels of RPTPs in primary mouse fibroblast-like synoviocytes (CD90.2+) (dark gray) and macrophage-like synoviocytes (CD11b+) (light gray) as assessed by qPCR. Primary synovial cells were isolated from the joints of Balb/c mice by collagenase digestion. Cells were stained with antibodies against CD3, CD90.2, CD11b, and CD54. Fibroblast-like synoviocytes were sorted by FACS based upon high expression levels of CD90.2 and low expression levels of CD3 and CD11b. Macrophage-like synoviocytes were sorted by FACS based upon high expression levels of CD11b and low expression levels of CD90.2 and CD3. RNA was extracted from the sorted cells and reverse transcribed into cDNA for QPCR analysis. Expression levels are shown relative to the expression of PTPRJ in CD90.2 positive cells.

### Example 5

### RPTP Expression from Mouse Synovial Fibroblast Cell Line

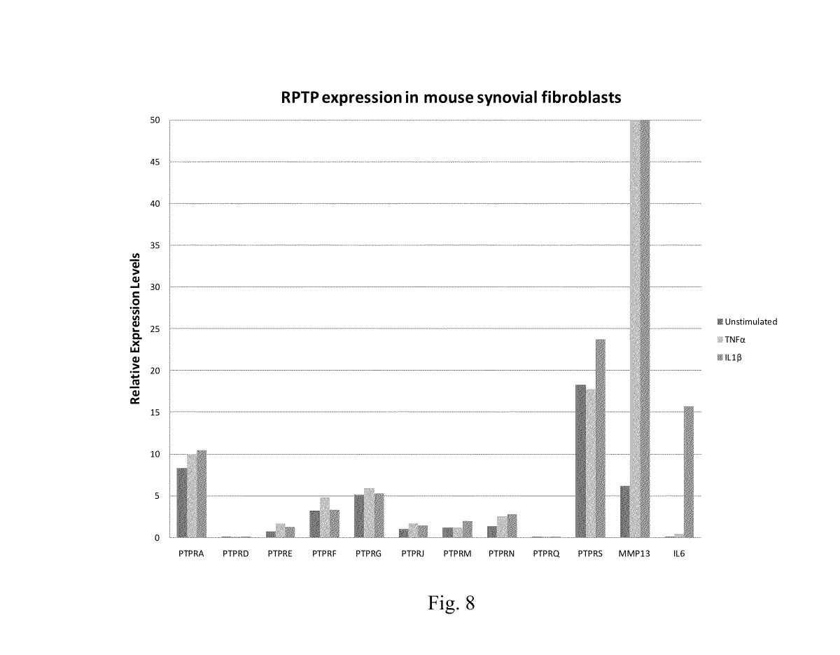

FIG. 8 shows mRNA expression levels of RPTPs in a mouse fibroblast-like synovial cell line. Synoviocytes were isolated from Balb/c mouse joints and were cultured for several passages to remove non-adherent cells and obtain a fibroblast cell line. Cells were stimulated for 24 hours with 50 ng/ml TNFα, 2 ng/ml IL1β, or were left unstimulated. RNA was extracted from the cells and reverse transcribed into cDNA for QPCR analysis. Expression levels are shown relative to the expression of PTPRJ in unstimulated cells. Expression levels of Mmp13 and I16 are shown as positive controls for the stimulation.

### Example 6

### RPTP Expression from Human Synovial Fibroblast Cell Lines

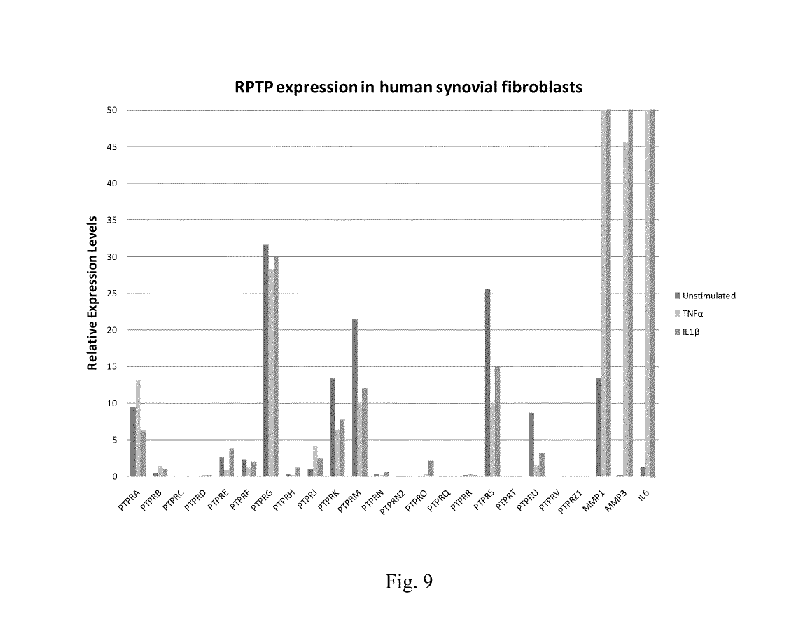

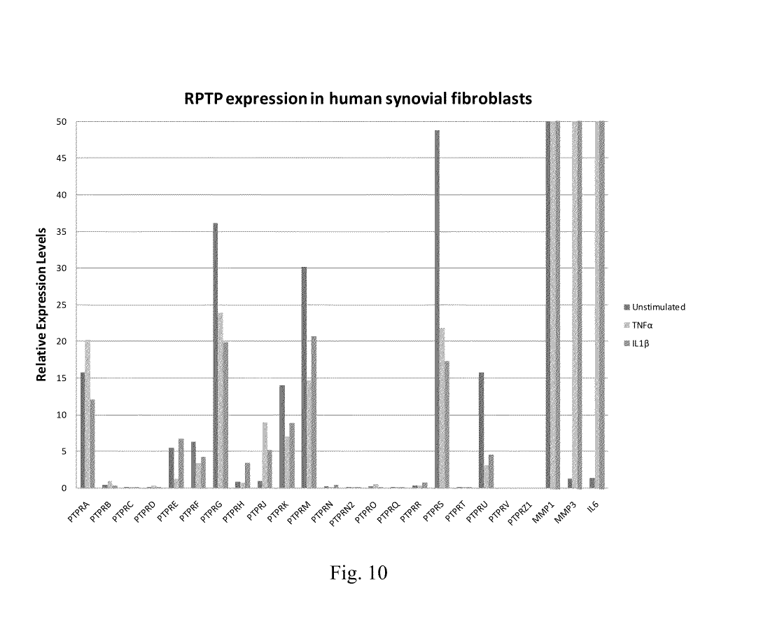

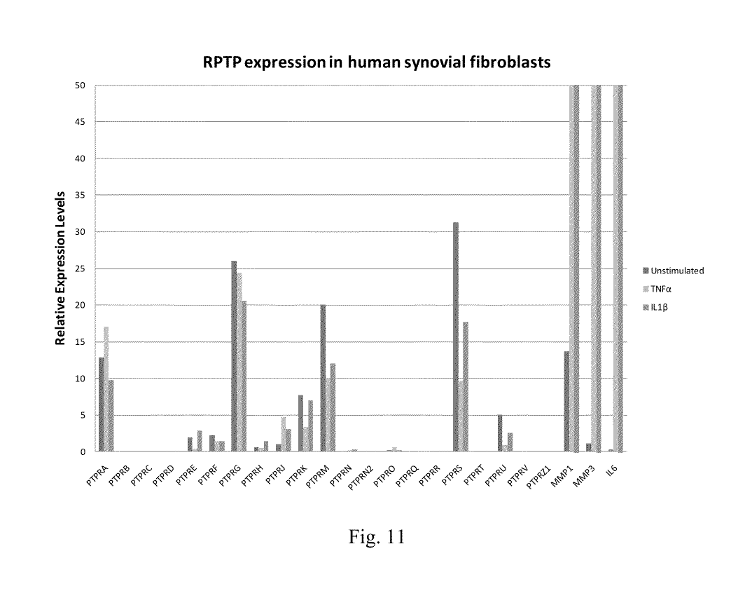

FIGS. 9, 10, 11 show mRNA expression levels of RPTPs in 3 human fibroblast-like synovial cell line. Synoviocytes were isolated from joints of human donors and were cultured for several passages to remove non-adherent cells and obtain a fibroblast cell line. Cells were stimulated for 24 hours with 50 ng/ml TNFα, 2 ng/ml IL1β, or were left unstimulated. RNA was extracted from the cells and reverse transcribed into cDNA for QPCR analysis. Expression levels are shown relative to the expression of PTPRJ in unstimulated cells. Expression levels of MMP1, MMP3 and IL6 are shown as positive controls for the stimulation.

RNA extraction, cDNA synthesis and QPCR performed in Examples 3-6 were performed with Qiagen or Invitrogen reagents. Turbo DNase was purchased from Ambion. All mRNA levels were normalized to the housekeeping genes RNA Polymerase II or G6PDH.

1. A method of determining whether a subject has or is at risk of developing an autoimmune disease, said method comprising determining whether a subject expresses a modulated RNA level of an RPTP or a modulated protein level of an RPTP relative to a standard control, wherein the presence of said modulated RNA level or said modulated protein level indicates said subject has or is at risk of developing an autoimmune disease, wherein said RPTP is PTPRA, PTPRB, PTPRC, PTPRD, PTPRE, PTPRF, PTPRG, PTPRH, PTPRJ, PTPRK, PTPRM, PTPRN, PTPRN2, PTPRO, PTPRQ, PTPRR, PTPRS, PTPRT, PTPRU, PTPRV or PTPRZ1.

2. (canceled)

3. The method of claim 1, wherein said method comprises determining whether a subject has or is at risk of developing an inflammatory autoimmune disease (IAD), said method comprising determining whether a subject expresses an elevated RNA level of an IAD PTPR or an elevated protein level of an IAD PTPR relative to said standard control, wherein the presence of said elevated RNA level or said elevated protein level indicates said subject has or is at risk of developing said inflammatory autoimmune disease.

4. The method of claim 3, further comprising administering a treatment for said inflammatory autoimmune disease, wherein said treatment comprises an IAD therapeutic agent selected from an anti-IAD PTPR antibody, an anti-IAD PTPR inhibitory nucleic acid and an IAD PTPR ligand mimetic, wherein said IAD therapeutic agent targets an IAD PTPR, or a fragment thereof.

5. A method of treating a subject who has or is at risk of developing an autoimmune disease, said method comprising administering to said subject a therapeutically effective amount of an autoimmune therapeutic agent, wherein said autoimmune therapeutic agent is an agonist of an RPTP or an antagonist of an RPTP, wherein said RPTP is PTPRA, PTPRB, PTPRC, PTPRD, PTPRE, PTPRF, PTPRG, PTPRH, PTPRJ, PTPRK, PTPRM, PTPRN, PTPRN2, PTPRO, PTPRQ, PTPRR, PTPRS, PTPRT, PTPRU, PTPRV or PTPRZ1.

6. (canceled)

7. The method of claim 5, wherein said autoimmune disease is an inflammatory autoimmune disease and said autoimmune therapeutic agent is an IAD therapeutic agent, said IAD therapeutic agent selected from an anti-IAD PTPR antibody, an anti-IAD PTPR inhibitory nucleic acid and an IAD PTPR ligand mimetic, wherein said IAD therapeutic agent targets an IAD PTPR or a fragment thereof.

8. The method of claim 7, wherein said method further comprises, prior to said administering, determining whether a subject expresses an elevated RNA level of said IAD PTPR or an elevated protein level of said IAD PTPR, relative to a standard control, wherein the presence of said elevated RNA level or said elevated protein level indicates said subject has or is at risk of developing said inflammatory autoimmune disease.

9. The method of claim 1, wherein said determining comprises obtaining a biological sample from said subject, wherein said biological sample is derived from a joint tissue or a bodily fluid.

10. The method of claim 9, further comprising isolating cells from said joint tissue or said bodily fluid thereby forming isolated sample cells wherein said isolated sample cells are synoviocytes, fibroblast-like synoviocytes, macrophage-like synoviocytes, fibroblasts, hematopoetic cells, macrophages, leukocytes, T cells, or other immune cells.

11. (canceled)

12. The method of claim 9, wherein said bodily fluid is whole blood, plasma, serum, urine, sputum, saliva, a bronchioaviolar lavage sample, synovial fluid, or exhaled breath condensate.

13. (canceled)

14. The method of claim 5, wherein said autoimmune disease is mediated by cells expressing an IAD PTPR.

15. The method of claim 14, wherein said cells are synoviocytes, fibroblast-like synoviocytes, macrophage-like synoviocytes, fibroblasts, hematopoetic cells, macrophages, leukocytes, T cells, or other immune cells.

16. (canceled)

17. The method of claim 5, wherein said autoimmune disease is arthritis, rheumatoid arthritis, psoriatic arthritis, juvenile idiopathic arthritis, multiple sclerosis, systemic lupus erythematosus (SLE), myasthenia gravis, juvenile onset diabetes, diabetes mellitus type 1, Guillain-Barre syndrome, Hashimoto's encephalitis, Hashimoto's thyroiditis, ankylosing spondylitis, psoriasis, Sjogren's syndrome, vasculitis, glomerulonephritis, auto-immune thyroiditis, Behcet's disease, Crohn's disease, ulcerative colitis, bullous pemphigoid, sarcoidosis, psoriasis, ichthyosis, Graves ophthalmopathy, inflammatory bowel disease, Addison's disease, Vitiligo, asthma, or allergic asthma.

18.-30. (canceled)

31. The method of claim 7, wherein said IAD PTPR is PTPRS.

32. A pharmaceutical composition comprising an autoimmune therapeutic agent and a pharmaceutically acceptable excipient, wherein said autoimmune therapeutic agent is an agonist or antagonist of an RPTP, wherein said RPTP is PTPRA, PTPRB, PTPRC, PTPRD, PTPRE, PTPRF, PTPRG, PTPRH, PTPRJ, PTPRK, PTPRM, PTPRN, PTPRN2, PTPRO, PTPRQ, PTPRR, PTPRS, PTPRT, PTPRU, PTPRV or PTPRZ1.

33. (canceled)

34. The pharmaceutical composition of claim 32, wherein said pharmaceutical composition comprises an IAD therapeutic agent and a pharmaceutically acceptable excipient, wherein said IAD therapeutic agent is an IAD therapeutic agent selected from an anti-IAD PTPR antibody, an anti-IAD PTPR inhibitory nucleic acid or an IAD PTPR ligand mimetic.

35. (canceled)

36. (canceled)

37. The pharmaceutical composition of claim 34, wherein said anti-IAD PTPR therapeutic agent is an anti-PTPR antisense nucleic acid.

38. The pharmaceutical composition of claim 34, wherein said IAD PTPR ligand mimetic is a peptide or a small chemical molecule.

39.-42. (canceled)

43. The pharmaceutical composition of claim 34, wherein said IAD PTPR is PTPRS.

44. The method of claim 1, wherein said autoimmune disease is arthritis, rheumatoid arthritis, psoriatic arthritis, juvenile idiopathic arthritis, multiple sclerosis, systemic lupus erythematosus (SLE), myasthenia gravis, juvenile onset diabetes, diabetes mellitus type 1, Guillain-Barre syndrome, Hashimoto's encephalitis, Hashimoto's thyroiditis, ankylosing spondylitis, psoriasis, Sjogren's syndrome, vasculitis, glomerulonephritis, auto-immune thyroiditis, Behcet's disease, Crohn's disease, ulcerative colitis, bullous pemphigoid, sarcoidosis, psoriasis, ichthyosis, Graves ophthalmopathy, inflammatory bowel disease, Addison's disease, Vitiligo, asthma, or allergic asthma.

45. The method of claim 3, wherein said IAD PTPR is PTPRS.


SEQUENCE LISTING (14 sequences)

SEQ ID NO: 1 - DNA, 135296 bp, Homo sapiens
cctcgcgccgcccgcccggcagcccggccggcgcgcgcacgccgcgagccgctggcgctcgggctccgctcggatcccatgcaacagccacgatgtgaagcggggcagagccgggggagcccagcccagccagcctccaggtaaagccaaaaaaaaaaataaaatcatggattagaggaaaaaaaaatgcaggcgctccaaggggggtctcggaggcagccgagtgggcagcgcgccggctttgtgccggggaagcctgcacggccattgtgctcggaggatgctcggctgcagccagttgcacgcccagccgctgcgccccggtagctgcgcccggacaatgccccgagggacagggccggggccgcgctgggggcgctcggcagggggtgctgggggggcttcgtccgcggcgtgggagggggctgggctggaagcgcccagagcgcggcattggaagctgcccagcgccgccggctgcaccaccccgggctgcgggcggggcagaggcgcggaggggggcgcgcgtgtgtgcggagccggtgccggccccggcggcgcccggcccgggtccccgggcgggtcctggaggcagggggcgctgtgccgggggatggggggcatgcgggcgccccggagcggagaggagccgggacgggtggctccaggcggggtggctccagggctgctgcgcccgccgccttccctctccgggtctccctcccctggagaccccctagtctacgccgtctcccctggaacactccgggccctgtcgttgccttatgggaggtcgcccctccggcactcgggcattgccccacccttctgcggcctcggcgcccccttccagcctccccacccaggggcgagcctcagcccccagccccaggggctctggcaggcagacgcgcacgcccagcaccccagtctcccctccgcgctgggggccccgtggggatcccgagagtggggaagccgcccctggaaccgccagccccgcgggttccgcacgccccgggagcccccgggggctgcggggggaggggaggtgacgtcataccgggggcggggccgtcggcactgggttaaagggggcgcggggcgtgccctgaggggagccccccaggttagagtgcccgcgccgtgggtgcgagggttcggccaggttgcggccaagttcacctctctttatgggaatctctcctaaaattaagatcctccccttcggacctccccctacaaatcctggacatttcccaccaattcccccccttcccaaattggggaactccccttcggccgctttctctccagaatcctcaaatcagggaacctttcccacaaactgggaatctttttttcagccacaccccccgccccaaatctgggacctccttcttcggttgaccctctttccccagtctcaaatcgtcttctcgtctccctaaatatgcagcttccaagaacgccccttcctctgcaacccttcctcaaatccagccacctcctgggacctcattcccccaaattaacaaacctcccagagcctcattgggggtgccttgcctcccccttctgggctcagctgggattcccctgtccccccccgtcacaatctaggggagcccccttctgagcatggggtcctcctgcctctggccgagaccctaaaaacacatcccggcagccgcggctagtgaagcgggtgtccgaggtgtcggagtcccggctgagcagcagggatgatacctagaagccggaggaggcaggaagagacccctagggtcccaaggacagggacccacacgctccgggctcccgtccgccaccacccggactttggctgctgggattctggcctcctctgggtggccccctatcgctgctgtccttggggtgcagagggtgacggcgcctctttgtctggggcgcccttcttccctctttctgccgcctttcgcccgggagaaggaagccatcccgctcctctgctcctgcgataccagcggcagctgctggagcgtttattaatggcccagggacttggtcgaatctgccatttgcataagtcccctccccctcccagggtcccttcctttgtgcccccctccccctgccaatcgcgcctgccttctgtccctctgctcttctttctttgccagctgatattttggcggtggaaggcaggcagctggggctgggggcggctgcttcccgccctcaaatcccacagccccccagcttgggcctgactgagtccccacctgggcatccctcccctacccccccgctgccctccacaaagccttccgtggggcatctctttctggctggcaggctggcggagtcttcattatttccctccgggaagcagggtcctgccctgttccccaggcccgggggacaccctcatcatgacggtgcacttctgcatgggggctaggcctcccagccccctgggggccaccgcccgccatactccatcatcctgagagttgttctaattgggctcaggcaaacaaaacgtcgcaggagacccaggctgcctggtgagctcacgcacttttctatttctgccaccctggccgatgacattgcccctaaccccccagggggtgacagagactggtactgtcagtggccgtccagagatgccgtcccaagaggcggtggactggcccgctccccactccccgcgggagatgggtgacaggtcattttggcgtggcccggctgaggcgctggagggtgctactggttggcaggtcagctccggggacgatttccccctctccaggaaaccttgagagcctctgaagtctgcgtgaggtggcgacagagaaggggacattcaaaccccaggttcctgcctgctgccagcagagaaaaaaaaatcactccttataccccggttgagcgttcagtgcgccttttttccctgcctcccaggggatttcaggcatcgtccagtgtccatggaccctgcttggagacacaagtccattttcccccaatttccaccatggaacagggggaagcttcgtggatgccagggatttcgtggtagcatctgggtggggagacctggagccatcactgtgcacccatttttttcttctttgggtcgcatagagcctggggctctgcctgggtttccccagacctggtaccagtctggaggcaaatctatctctgctcatcaaacgtctttctgggaggtgacttggtttccccttctgccttcggggttggcgttgaacagcggttcacagatgtctgagggcagcgcacacctccacttgccaagatggggagacctgtggcttcgctgattattccagcggctgcatggccccaatacaatagttagtgggatggccggagagggacgggcagggacggtgtgacttggtggtgcgacgtggcatttctacccgagcgccccgtggccccgcgttcacctgctttctgtgacggtgcagccgccgtgtgcgtgggggaagaagcggggcgtggagagagttcgggatgggggcgagcgggagggaaaaccctaaaaataaaaagggatatttataaacccagcgggtttgggggataattgacaaagcttaatgtttttcggctgagagcaattccttccccctgctacccattaagctccatttgcagagtcggtcggagccacgggaagatcttgagatgagctttaggtttggcgggcgcagtgctgaccgctgtagcccccttctgggagggtctcgtactttctggaaggagacaggggtcccagaaaggggcactccccagctctcctctgcatccagggtaggctcctggctcaccttccaatgggtgggtgtcctcttagaatttgcaaggaggatgagaacccttctcgggcaaagcctgtgcaggttgcccttcctccgtccttccctgcggcaaaagatgcccagggaggggaatttgaagagagaccctcccccccaccacccgccgccgccccaccctccaaccgctcaccaggcagagtggctcagcctccggtggggacaggaggagaggagattaagacataactgctgttcctcgcaggattcgtgtggcaagctgatacaaaacaaaacaaaaaaaccaaaaattcaccatggattcacggaccaaggcacgtatccggggcttgaaggtttcaagggctttttaaactgtgaattttgtcattttcacaccttaccttgctctggaatcggaaagctgccagcttgtggcttgcggggcagaggagaggggatgctggaaggggattgaggctcaaatgcatgtcatcttatcaccaggtgacattctgaattgaggaagggggacacggagagaggaagtgaagtgtaggggatgtgatcgagggaaagggtggctgttgtctcacgcagagagtggcagccccaaaatgtctaggaaacaggtgacacctgggtctctctaaccctgaggttcaagagtgtggagtgtgtggtttcccccccgccccccccccttttcctttaaggaaagaaaagactctcaaccaaaaagggaagaaaaaaaaaaacccgacgtaccatttctgtcttgtctctggctgatatttgagaagcgctggggagggggttgtgtgtggggaatttgaaaaatcaattggcttacacttctccaaaggcgaaaattaacgggtaggagtgattcacggacaggccctccactttcacatcgctttatttttgtctctctcttttttttttttcctcctcctcctcctccatggagctgccggtcccccaacatcgtggcctggccagactggaccttggggctggtgctattctgggctagccgtggaatcccctcttctgttccccggaatcaaggcaccctctgcgccccgcagaggccgaggggatattccacatctgaaaataaacccaggtttctgtgcggcttcctgtttccttctgattcccgcggatcccaccctggttgggggttcagagcctggatttttggatcatggggactccctcctccggtccccatcgccgtaactggggctggatcacaaatgtcccgtttcatggggacagatttcatctcgtactctttatgttatctgggtggccttgctccttttaagttgtttttgatctttgccataagccccttataggaagggccagctgtctcctgttccccctacctggagaactgaaaattcccctttttctcccagatctcacctgcctgcctgccttccttccttcctggatgacttcccctcttccctttcagttctccgtgcaggagcttcctcacttacagaatgaggttacccctctgggttccaggtcagagccctgacttgagccaccctcctcacttgagctgtgcaaactagggcgggttgcctgacctctctgtgttctctcatctctaagatcagtacagtcgcagtctgcgtcgcggcgagccttgacatgagatgctgtgggtaaacatgcctcccctggctctcaacgcgtccgcagtcagtcgcatgcatccttccttccgctcctggacacccagaggggtatccggcgggcggagaagccatggctctggaagcctgggaccctcggatgaggcaggctgtggggagagaggctgggaggatggcccatcgtcgcgctgtcacgtgggtaattgagagtttgcggaaattcaggggttctggggaagcgaggttggcgcgtctgtgtgtgtataatttaatgccgcttataattaacccccgtgatcagcaaatgaggggatgtcggggggataataacagctgctccgactatttagagttggcgccccgtgccaggccctctgctaagtaagcaagtcagtagcatccttgtctcctccaacctgccgtcccctgtcagcggagttgctgtgatctgagccctattttgcagagagggaaactgaggcacagggaggggccttcactggcctgaggtcacttcacacagcccacaagctgttggctgccacaacctggattcctaacgcctggaaggagctctgcccgattgtcagggatgtttgtttggacagcagtccatctgctgcgggggccacctgaatcgctacagagacaccccccgcacacacacaccccagggctcttgactgccttttctcagaggttaagagcaaggatgctggactgaggctgcatggggttcaaatcccggctctgccgacttgttggctgtgtgacctggggcaagttgctcaacctctctgtgcttccgtcttcctcatctgtctatggagaagagtaacagtgcccaccttgagggattgttaggaggaagaaatgagttaacgtgtgtaagcacagattcagaacggcacctagaatgtagctgccacctctattctgtttttgttattatttttgacaacatgggagctacactgatatgttgaaggacctgcccaagaggggctctccagtgtccacccgcgccccgctaccccgccctcagccccccgatggggtccagcaccagggagatgtcaggatgactcagccactgcaaactggggcgttccccttcactggctgcttaatcaaggccgccccacttctgcggcaggactgggcgactggcgggagctgaaccacagggggcctggatggtggacagggaggacacgtggctgccttgggagctccaggcactccagggactccacatgtggctcccaacttccagagcccctggtgggcccgagtgatcaccaaaggggggagtttgggggggatttgggggtttaacagcagtcatttgatgggcactcaggaaagtgatcggttgacaagcatggtgtgcgagcgagcatgcatgcattcattcgaatattcaccagttcattaattcattcattccatcaggattgctggaaacctgctgctaaggaacttggaagccagaatctaagcagctgtggggcttgggctagtaataaagctgttctgtgcctcagtttcctgatctgtaaaatgggtgctttgacattgcctctatcataagtggagacagatgaactggtgtggggggcaccaaggacagagactgagagtggcacctgctgctttgtggctggaggtgaggggggcggaaacaagaagagaagagttgactttagctggggaagaccacacaggaccccccaccccaaggagttcgaatcgcattgtgtgtgtgtgtgtgtgtgtgtgacaggactcacactgttgctcaggctggagtgcagtgatgccataatggctcactgcagtgtcttaactcctgggctcaagcaatcctcccacctcagcctcccaagtagctggggactataggcatccaccagcacgcccagctgacttttgtattattattattattattattattatttatttattttttttttgtagagatgggtttcaccatattgctcaggctggtctcaaactcctgggttcaagctatctgcctgccttggcctcggaaaatgctgggattccaggtgtgagccaccacacctggcgaattggcattttttatttattttttattttattttatttatttgagacagtcttgctctgtcgccaggctggagtgcaacggcgtgatctcagctcactgcaatctccgcctcccgggttcaagtggttctcctgcctcagcctcccgagtagctgggattacaggcacccgccaccacatctggctaatttctgtatttttggtagagatggggttttaccccattggccaggctggtctcaaactcctgacatcaagtgatccgcctgccttggcctcccaaagtgccgggattacaggcgtgagccaccgcgtctaacaaattgcattttttagatgtgcggtcagagttctctgaaagcccccagagctgggctgccaacatcaggatcaacagaaaacctggaatgttgggaatgcttcttggaacatctgtcaggccaaggcccagagagggagagcaacttgcccaagaccacacagccatgcctgcctgcctgctgctgggccttgcgcaggaggatggacagggacccctttcaccagtccaaagggagaaaccgaggcaggaagccatggcgggtgtgtgattctcaagccataacgcctgtcagggctggagtctccactccagcgggtcgtatgggccaaaaaggcttcttcaacctccaaggaagccctgaatggctccagggccagatctgttggtttttccaaggaaaagcagaaatctagattttaagtaaaattgcccaattttggctgggcatgatggctcacgcctgtaatcccagcactttgggagaccaaggcaggcagatcacctaaggtcagaagtttgagaccagcctggccaacatggtgaaaccccgcctctactaaaaatacaaaaataatctgggcatggtggtgcatacctgtaatcgcagctacttgggaggctgagacaggagaatcgcttgaacctgggaggcggaggttgcagtgagctgagatcatgccacttgcactccagcctgggcgatagagtgagactctgtctaaaaaaaaaaaaaaattgccctattttaacatccttgggttgaaacatcttaacatggcacagtacagaaatccagggccaccgtggactttgaccccttccccaacaattaaaagttgcgtgacttctgacacctccattgccgtccgcgggcctcagtttccaggcctgtgaaatgggtttaagactcccagccacctctgcggttctttagaggcaccagtgtgatagcttacagagagtgctgagaacagggcctgggccagcgtccgcgctcgataaacgttggctctgattatattaatattggtcataatgataaacttgacttaaattaaatactgcgtagaaggaagccggctgtcccgcggggcagggctgcgaattatctactggcggcaggacctgcttctccatccctcgttctttctgtagcgcagtcgtttgctcttggggctggagcacccgggttaccagtattactcgttgagtgtactgctgtggttctcagccggggtcacttttgctcccctgggcacacttggcaacgtctggggacatttttgggtgtcatgtccaggggggtgctgctggtatggagtaggtggaagccagagatgctgctcaactctgtgtaatggccaggatgaccccatcccagataatgaccccaccctcatgtacactgtgctgaggtttagaaaccctgattttgccaggcatggtggctcacgtccataatcccaacactttgggaggttaaagcgggaggattgcttgagcctaggagttcaagatcagactgggcaacacagtgagacctcatctctacaaaaaacaaaaaaaaatcagccgagggttggggtgcacacgtacggtcccagctactggggaggctgaggtgggaggattgcttgagcctaagagtttgaggctgcagtgagctatgattgcatcactccactccagcctgggcaacagagcaaggccttctcttttttttttttttttttttttaaaaaaagaagccctgacttatgctgaagacccaagacagcgagagaaatcaggcagatggtgcctttgtccctgagcgtccacccaacacgctccctgacgtctcgcggggccagaacgcttagattgctaatgggttccctctgcacatctgtcctggttccactgccccattgattattggaagtgggtaattgcccctgtcggatgaattctagagagtgtgaactgccggggtttccggcaacagtgctggcttcaactcccagatatcagcagggtgcagagaggctgccaggtcccaggactttttctcttggccccatctggccctgggagcccagactccggccctgctggggaagggcattaggggtctgaacgccccaggttcggatcctgacaaagaccctccctggctgtgtgagatgacctcattatttccttcccctgagcctcagttttctcatctgtgaaatggggacgagtcttgcctgctgcatcactgggaattcagtggtgtcacattgataacggggccaggagcccaaggtgcacaggaggagctggatgagtctaccctctctccccacagcttcgtgaaccgttaggggtgatgaggttggaacctcaaacccacattctccaaacagctgcaacagaggagaactaacccaagcgggcggaaagtcccccgtggtgggggccgctaatgctccgccaccagtaccagtgtgcctggccctgtgttttctcatttcctcctcagaactgccctgagaggtggcaggtgtccccttgtcttgcggatggggaaactgaggcacaaaggaagggtgagaactgggaatgacaagccctttctcaggtttacagaggcgttgtttctggacaggatgtcgtttctacactcctggaggctgcacccaggcccagcttcacgggggccctgcagtcactcgggggcccacgatcagaaggggctgcatttagatggatgctctgttgatgccgtctggaaattcttaataattttttttttttttttttttgagacagagtcttgctctgtcacccaggctggagtgcaatggcgtgatctcggctcacggcaacttccacctcctggggtcaagagattctgcctcagccttccaggtagctgggattataggcatgtgccaccatgctcggcggctagtttttgtatttttggtagagacagagtttcagcatgttggccagactggtgttgaactcctgagatcaggtgatccacacacctcggcctctcaaagtgctgggatcccaggcgtgagccaccatgcccgacctggaaattcttaatttttttttttttttaggagatggagtgttgctctgttgcccaggctggagtgcagtggcatgatctcagctcactgccagctccacctcccgggttcacgccattctcctgcctcagcttccccaatagctgggactacaggcgcccactattacacccggctaattttttgtatttttagtagagacagagtttcactgtgttagccaggatggtcttgatctcctgacctcgtgatctgcccgcctcagcctcccaaagtgctaggattccaggcgtgagccactgtgcccggccttaataatttttaaacaaggaaaaccccacggtttcattttgcactgggggctgcaattctgtagctggattgttagctacgatgtagccagaggccacttcccacagtggctgggagcaaagatgttatcggccaggatgtgtttttgccccttacgagctgtgtgacttcggggaagtcactttccctctctggatggcaccctcagactcctctctgtgggccgctggcctcatctctcctcctcttcccctcccctttgtccgttccactccatgtagacctatccttctcctgtttccacctccaggcctttgtccttgctctgccgggagctctgtttctggccaggccacccctaccccgccattgcctcccgcaccccgttttgttttccctcacgatatcgctctgtctgcaacccctttaggacctaagctccgccacacctcacccgcccctccagatcagcccgtctggctcctgggagctaccgcctggcctgcgtgtaccagcattgccacgtctaatgaccttgatcaggcaggtaatgatggggaaagccctgcctcggcagctcacagcaagagccccctggtcctgctctgagagtactcggagtgggtgatgttgcagggagaggcagccacagtgacctcaacctcctgagtagctgggattactggcacgcaccaccatggctggctgtatttttagtagagactgggtttcaccatgttggccaggctggtcttgaactcctgtcctcaggtgatccacctgtctcagcctcccaaagtgctgggattaaagctcagctcactctgcccttgggcagggaggccaggagactgggttcccagcggaaatgctgtgtgctctcagctacttgctcaccctctctgagctgatgaatatccgtagtgtctggcaagaagtggtaagggccgggcgcggtggttcacgcctataatcccaacactttgggaggctgaggcaggtggatcacctgaggtcaggagttcgagaccagcctgagcaacatggtgaaaccccttttctactaaaaatacagccagctgtggtggtgcacgcctgtaatcccagcgctttgggaggccgagactggtggatcacctgaggtcaggagtttgagaccaacctggccaacatggtgaaacccagtctctactaaaaatacagctggccgtggtggtgtgtgccagtaatcccagctactcaggaggttaagtcaggagaatcgcttgaacccgggaggcagaggttgcagtgagccaagattgtgccattgcactccagcctgggcaatagagtgagactctgtctcaaaacaaaaacaaaaaaaaacaaaagaagtggtaaggaatggctttgagggtgatgtctctgagtaattggcttcaagccaggggtctgaggaccagtgtcaggaggtcagggaactctaaaacagtctgtgagatttcttgtctcctgaggagaaggtgagaggcccaaagggtctggggacccccagaaagtgtacagtggttgggccagatgattttaagagtcaaccagtacccgggcatggtgactcatggctgtaatcccaatactttgggaggctgagatgggaggattgcttgaggctaggagatcgagtccaacctgggcaacataggaagaccccatctctataaaaagtaaaaaaattagccgggtgtggtggtgtgcacctgtaatcccagctactcaggaggctgaggtgggaggattgcttgagcccaggagtttgaggctgcagtgagctatgatcatgccccattgcactctgggtaacagagcgagaccctgtctctaaaaacaaaaagaactgagccaggcctgatggatgttgggcaccctgtgtgtctggccagacaggtggagaagttactgaggagctcaccaggaatgggccaggggatctttgggggatcgggtttagcatttgaatggtgcaagtttgaatcatgtctgagctgcttgttcagtgtgaccctggggcagtggtttcaccctccagggcctcagttttctcgtctgcaaaatgggggtgaggctgtctcttgggtagaagggtcttggtattggaaggatctatgctaaacacgcttggagtaagccccaaactggctaagatatcataaaatacagcttgatgatcagtgccctctggaagcaaggtcgcaggacctgcatatggggcttgggggcgctggggagtcgctgtgtccggggtattgagttgcgcttggagtgtgcccactgcagtgcggagagcagattggcgggcacctacggcaagccctgcgcagaccgagtcgcagggcaggctgccacgctggcaggagctgggctgggagtgcgagcggggctacccttggaccacctcagcctcagtggggaaactgagtcacgtcaatgtggacagaaccctcagcaggtgtggagggtcagttgtgactctctaggtctgtgccgaggtcactctgcagggtctttggactctatatctggtgttttgctgccccatcgcctaaggactagctagcttgcttgcttcattccttctttccttccttccttcctttttctttctttctctcttgttctttctttacttctatcctccctccctccctccctccttccttccttctcttcccttctttctccttccttctccctcattttctctttctcttctcttcctttcttttcttcgtttcttttttttttctttctttttcttttgacagcatctcactctgtcacccaggctggagtgcagtggcacgatcatagctcactgcagccccaagctcctgggctcaggcgatcctcccgcctcagcctcgcaaagcgctgggattacaggcacgagccaccgtgcccagccagtacttgacacgttttgtttaaacccattctcttttaaaacgtaaaagagaaaactttgtcttgctcttgtgaacggaaacctggtatgtccttgaaggtacaaccttaaaaataaatgtggtgagagcaaaacaagggcattaaattcttgctgcttttgcctctgtctgttccaggctgaaagctgaagcctgagcacccctgaggtgtttaagacatcctagcacccacctgaggcttttagccttaagcccagcacagaatgacccccttgctggttgagtcagtattttgttttgttttcttgagatggagtctcactctgtcgcccaggctggagtgcaatggcccgatctcggctcactgcaacccccacctcccgggttcaagcgattctcctgcctcagcctcccgagatgctgggattacaggcgtgtgtcaccacatacggctaatttttgtatttttggtagtgacagcgtttcaccatgttggccaggctggtttcgaactcctgacctcaagtgatctgcctgccccagcctccccaggtgctgggattacaggcatgagccaccatgcctgtcccagtgttattgaatgtgactacaactgtttggggcatctcctggactttctgggaggctgagccccttctgccttcggcctgaatttcttgaactgagcgttgcttccccatggcctggggagaaggtgatttcttcctgaattcaattaatgagcctattaaaaactatttattttccctgacagttgtttactgaggagaaggagatgaattggccggtgtcctcggcaggcaggaaggaggctgaatgggggtgcttggtggctcagccccagtgcttggggagcacagattgccggtgggggcctgtccttggggacgccttgggcagagcctgtgcttgggtcgtggccactgcttctgaccttggagtcttttgggaggtggatgttggaatgagtgaaaccgtcgtggtgtggaggcagggggccggccccgatgacgtctctcttctctttccaggttaaaatgtctcagatgcgctggtttgagacccaagacctggccccatggcccacatgagatattgtggcgggatctcccaatatcccatcatatcccccgactccgtagcagttgaactttacaatttttatctgggcacatggccacctgggggaaagaaaacatttctcagtgcaccttgcagctgtctgtgccatatgattcaatgctgacccatggcgtaaataagcaggattttctggaaatgcctttcaagagaagtggcacatgctcctctctacctgtcagctggaatatggagatgatggcaggtgctcaggcagccattttggaccatgaggcaggtgtcatagataagaatgggggatcaaagaggtggaaggggcctgggccccagtgattgtcggggaggggaaaataaccctgaatggccaacctggcagcttcccttaggtgaaaagagaaatcattccttatttgtgctttttctggcatttgcaattcaactggatccaaacggtggcagaacatgaaccttgggacaccttctggggatgtggctctcctggggatgtggctcttcttgcccctgtgctccagagaccccagactcgaagttttgcaaacagtgtgaggatggggatgatggtgaacgaccaagcatgatgggtgctgacacggatcccacctctagctgagtgcctcgtggtcctgttgaatctgcacagccctgtgagaagcctgcaaccactgccccactttcgcagataaggaaaccgaggctcagagaggggatgtgacttgtgcagggtcacacagcaagtagagttgagttaggattgggctgaccccacgctttgtggcagggctgcatcctgacacacctgggggcctcccccctttaacccatgggtcactacctccttctcctctcctcctccattgcaaccatcagatctggccctggtctgatcagctgccgccagcctgggacaggccaatagggacatgagagtgaaaactgtgtccccttccatacacaaaatgctcccaggcacaggggtcactgtcttctcctgggtcctgccaggggaagcgggtttcaggttcagagtacagagcctaagaccctaaaaataatacaagccaatgtatgctgagcacttgctgtgtgccggactgtgcgctgagtactgtacacaggccagcttgctttccctttttttctttctttttttttttttttttttttttttgaggcagggtttcgctctgtctcccaggctggagtgcagtggcataatcttggctcaccacagcctctgtctcaggttcaagcgattctcctgcctcagcctcccgagtagctggaatgacaggcacgtgccaccatgctggctgatttttgtattttttgtagagatggggtttcaccatgttggccaggctggttttgaacttctgagcttaagcattccacccaccttggcctcccaaagtgctggtattacaggcatgagccactgcacgtggccttcattttttcatttttgttctatcctctgcctccacctggtccagctcccatcatcacctggaccaacacggtcacctctgctctggtgtgaccccagccctcattcccacagtctgtcttcccacaatgacatctttcagttcttgagtcaggtcccatccctcctctgcctatagccctctatggctctcacctcttgggggaaaaccccaagtcctccctgtagcccaccaggccttgcacaacctgctccatcccttccctgccgtcccctccttcctctctccccgtcctcactctgttccagccacacgggcctcctcgctgttcctccaatacaccaggcatggtcccgccccagggcctttgcacagtctgtgccctctgcctggaatgcttttccccaaacagcaacctccctgacttccttcacgtctcaggtcacttttaaatgaggcctccccgatcaccctatttaaacttgcagcctgccccaccacctagtttcctgtcttcccccttctgctctctttgactcaaagcccttgccaccatttctctgttgaatgcttttttgtttgtttttttgagacggagtttcactctgtcacccaggcgggagtgcagtggtgtgatcttggttcatgcaacctctgcctcccggattgaagcaattctcctgcctcagcctcctgagtagctgggattacaggcatgtgccaccatgcccagctaatttttctgtatttttagtagaaatggggttttgccatgttggtcaggctggtctcaaactcctgacctcaaatgatctgccctcctcggcctcccaaagtgccaggattagaggcgtgagccacctctcccagcctcgaatgcttcatactcattatctgcttctctcctcctctaccatgtccatggcagcatctgcacacagtagggctccaggcatacattttgcctaagcagctcttcccaggggaagaaatgaggctctgaaatgcatgaggtctgttttttctttttcttcttcttcttcttctttttttttttttttttttttgagacggagttttgctcttgttgcccaggcttgagtgcaatggtacgatcttggctcactgcaacctccacctcccgggttcaagcgattctcctgcctcagcctctcgaatagctgggattataggcgtgcgccaccatgcccggctaattttgtatttttagtagaggcagggtttcaccatgttggtcaggctggtctcgaactccctgcctcaggtgatccgcccgcctcagcctcctaaagtgctgggatgacaggcatgagcaccgcgcccggccgcctgaggtctttctgcgggctggaggcagtgcttgcggggagcaggtggcttccacgttcttgcttttaaccacccccaacccgggagtcggccagtcggggcgggctcgagtcggtccccgcggctgcttcttcctcccacccccgcattctcacctcagcctccagaaatagcccctttaaaatagccacgtcctcgagttccggggcctctgcctgcccacggacgtggtgctgcgctgacgggccagcctcctgagtgctgtggggtttttaaaaatatagctggtgttaggtgatattacctcccttttaagcctgaaaaagcccagccaccaacagggctgtgtgtcactgtggatactccagcaaggcacggccgcgagggtgtctgctgtgcagagctggggagggcttccgctaaaggctcacgccagctcaggcggggaccgtggggcctgcagttccctgtcaggtcaggaaacctcagatttgaggctcagtgctgcctcttgccggctgtgtgacctctggcaactgttgaaacctctctgagcctcagtttacccaaatggaaatggggacaatttcagggtttggataaatggttaaatttctctgagcctcagtttatttgtgaaatggggacatctcagggtttgggcaaatagctgaactcagcctcagcttacctatgtggaaatgaagacagttttggggcttggataaatggttctatttctccaagcctcggtttacctgtttgtaaaatgaggacatctcagggcttgggcaaatagctgaacttcccagcctcagtttacccatctggaaatggagacaattttggcgcttggataaatggttctttctctgagcctcagtttacctgtttgtaaaatggggacatctcagggcttgggcaaatagctgaacttctcagtctcagtttacctgtctggaaatgagacatctgaggcttctccactctgggcctggcacacagtgggtactcacgaggcacgagccaaactccttacagcactgaggcctctacaaaatgaccctgtagtcgatctgggtcagggtttagtgaatttagcattagctctgagcctgccacttctaggcggtagatccttcaggaaatcgcttcccccttgagggactcagttttcctcatccctaaaatgggcccatgtcactgggtcgggagacatcagggagatgaagcatagctgcccttttccccaggtcggaatgctccgtagacgtgactcacatttccctgttaccttgatttattctttttatttttctttgttgagacagggtcttgctctgttgcccaggctggagtacagtggcatgatcatagctcactgcagcctcaacctcccaggctcagttgaccctcctgcctcagccccctgagcagctgggactataggtgcacaccaccatgcctggctaatttttttgttgttgtttaattttttgtagagacaaggtcttgttatgttgtccaggctggtctcgaactcctgggctgaagtggtcctcctgcctcagcctcccacagtgctgggattacaggcatgagccagcaccgggcccttgttttattcttctcattcgttgagactgtgttgattagtggccaaaaccagattctagggccagatggctctgtcccttagtagcaatgactccacctctctgggcctcagtttcctcatctggaaaatgaggataatgataacctcaaccatccaggctcccttgggaggatctagtgagttcatagggtgaagccctcaaatcagggctgggctcagcatgaggacagtgtgacgattgtaaagaaatccaggccggacgtggtggctcacgcctgtaatcccagcactttgggaggctgaggtgagcagatcgcctgaggtcaggagttcaagaccagcctgatcaacatggcgaaaccccatctttactaaaaagatacaaaaatgagagcccagcatggtggcgtgcacctgtaatcccagctacttgggaggctgaggcaggagaatcgtttgaggcagagtttgcagtgagccaagatcacaccactgcactccagcctgggggacagagcgaactccgtctcaattaaaaaagaaaaaatccatttatttgctagatgtttatcaggcttgtccctacactggccacttcccgggagccggataagccaagtggatggtgcttgtgtcagcctgtgtgtttggcttcaaggtccgtgtaccccctctcccactggggcagggattaaacctgccggagggacctgtccagccaaggccacgggcactggctgccgcccctgcttcctgctgtgtcacaacaggtgttgagccgccggatggcagagggcaggggcctggcttgccaacagggacaggaaggccccggctggagtaggcgtggaggcttccgccgtcagcctccggggtctcagcggctgctttggccgcagccactgcctccgattttgcgctttccatcatctggggggatttctcagatgcctcctgtccaccaggctctgtgccgggaccctgaagcaataaaggggatggctttgtggatcagggagagagatgataggcaaaatacgatatatagcacgtcaggggtgatcagagaaagcagtaaaggaacaaaggggactcgggagggaggcatgagggttgtcatttttaatggggctgtagggacggcttcattgaggaggtgatgtttgagcaaagccctgatggaggtgaggaggaaccaagcagataacgtgggggaagagcattccaggcagacggcacagcgtgtgccaaggccccggggcaggaccacgcctggcgtgttggaggaacagcgaggaggcctgtgtggctggagcccagtgagtgatggggaggggagaggaggcaataggtcatgtagggtcttgtgggctgcagggagaaccctgggctttgagccctagagaggtgggagctatggagggctgtgggcagaggagggacaggacctgactcagatactcatggttgatgccagggatggaccaggtgaaggcaaaggtgggtggtggggagaagaagcaagtggatttgggatagctttttaaaaaaaaaaaaaaaaaaaagacaaggtctggctctgtcgtccaggctggactgcagtggtgcagtgcaatcttagctcactgcaacctccacctcctgggctcaagtgatccttctacctcagcctcccaagtagctggaactacaggcgcacaccaccacgcatggctaattttttaatttttagtagtgatgggttttgccatgttggtcaggctggtctcgagctcctgacctcgggtgatctgcccaccttggcttcccaaagtgctgggatcccagatgtgagccactgcgcccggccttgggatggcttttgaaggtagagtatccctcctttttttctttccatgtctggggttagtggccaggctgagctgatgctttctcttcgatccttccattagaagtttccagaactcagaatttcagggacaaatatggaggggagggcaggccaggcccttgccctccagggactcactgtctgttaaatggagaatacaaacagatcaagagattgcagtgtgggcctctcacttagcaattcaaggggaatagggtgctagacaccctgacagcctaggccaggaaaaacttttttaaaaataacatcagctgggcccaatggctcatgcctgtaatcccagcactttgggaggttgagacgggaggatcacttgagcccaggagtttgaggctgcagtgagctgtgatctcaccattgtactccagtctgggtgacagagcgagaccctgtctttcaaaataaaaacaaataataacaaccaccaccaagataatatctgcagtctatcagctgcttctcaggtggtgtgaacagctctgtgtgcctttcgattcagtcttcacagcagccatgtgaggggaggaagagacttttatacccattttacagatgaggaaactgaggcctagagagtcgagtcacctgcccagggtcacacagcatggccagactgccttgctgttggacctagctggggtgcagcctgttctgtcctgtcctcattccgtctcgcagcatttcacggtcatcattccatcatttcttcttcttcttttttttttttttgagacagtctcaccttgttgcccaggctggaatgcagtggcacaatcttgctcactgcaacctccgcctcctgggttcaagcgattttcctgcctcagcctctagagtagctgggactacaggcaagcgccaccacacttggctaattttcattttttttttttttggagacggagtctcgctctgttgcctaggctggagtgcagtggcgtgatctcggctcactgcaacctccacctcctaggttcacgtcactctcctgcctcagcctcctgagtagctgggactacaggcacctgccaccatgcccagctaattttttgtatttttagtagagacggggtttcaccgtgtgaagcaggatggtctcgatctcctgacctcatggtccgcccgcctcggcatcccaacgtgctgggattgcaggcgtgagccactgcgcctggcctaattttcatatttttaatggagacggggtttcaccatgttggccagggtggtcccgaactcctgagtgcaggtgatccacctgtctcagcctcagtgctgggattacaggcatgaaccactgcacctggccccttactaatttctaggtgggccacacaccccaccctcttactttaaagatctatgtgagtttgggttctcttatttgagatgtttgcttgggccctgccctgccctgtcctattttaggaaaatttgtgctgtcagtagccacctggaccagtggaaaatctaggattctgttagtcctggattcaaatcctgccgtcacctctttcctagctgtgtgattttgcggccattgcccaacctctctgattgctcatttcctgtgcaatgttttgtgttttttgtttttgagacagtcttgctctgtctcccagtctggggtgcagtggcacaatctcgactcactgcaacctccacctcccaggttcaagtgattctcctgcctcagcctcccgagtagttgggattacaggcgcgtgccaccacgcctggctaatttttgtatttttactagagatggggtttccccatgttggccgggctggtctcgaactcttgacctcaagtaatccacccacctctccctcccaaagtgccgagattataggcatgagccactgcgcctggccaggctcagctatttatttgtttacccaagtaatattttctcagtgcctgctatgcatgaggcattgttttgggtagagtggttcttcctaatgtcccgccatggccactccagccctcaaagtgtgagttgacaaaccctgatgtttgggccatgggagggtgagcaggagccccagctatagttggtatccttcgtggatccctcagaacagacgtctcagcgtggctaggaggaaaatcccggggcagtggggcagccgcccgtgggagaagccaggtccccggtgcctgggagtgcaagcaacagtgctatgtgtgcagaaccccaaatcacagccgggtgccagaaccccaaatcacagccgggtgtggtggctcatgcctgtaatcccagcactttgggagactgaggcaggtggatcacctgagctcaggagttcgagaccagcctggccaacctggtgaaaccccgcctccattaaaaatacgaaaattaggccaggcgcggtggctcatgcctgtaatcccagcactttgggaggccaaggcgggtggatcagttgaggtcaggagttggagaccggcctggccaacatggtgaaaactcttctctactaaaaataaaaaaattagccgggcgtggtggcgtgagtctgtggtcccaactacttggaaggctcaggcaggagaattgcttgaacccgggaggccgaggttgcggtgagccaagatcacagcaccacactccagcctcggtgacagagcgagactccgtctcaaaagaaaaaagaaagaaagaaaaaaaaacccaatcagcaaatctggggcaagaggcattcaggagagctggactcgggacacaggacttttcaggaaacatgaaccatttatttgacttacattttccatttacgtatgtagttcaggaaagaaaagtgttcaaaacctgtgtccaaaagagtattaaaaagccctttctcacagtggctaatctgtaatcccagcactttgggaggccgaggctggaggattgcttggggccaggagttccacatcaggtggggtagtatagcaagaccctgtctctacagaaaattttaaaaattagccaggtgtgatggtgtgtgcctgtggccccagctactgaggaggctgaggtgggagggatcgcttgaggccaggagctctagactaggcaggataagataggaccaccccccaccccggccccagatctgtgcaaattttttaaaaaattaactaggtgtaatggcgtacagctgtggtcccagctacgcaggaggctgaggtgggaggatcgcttggggccaggagttccagatcaggcagggcagcatagcaagaccccatctctgcaaaaagttaaaaaaaaaattagctaggtgtgatggcgtgtgcctgtgttccgagctcctcagaaggctgaaatgggaggattgcttgggtccaggagttccagatcaggtggggcaacatagcaagacgccatctctgcaaaaaactagccaggtgtgatggcatttgcctattgtcccagctactcaggaggctgaggtgggaggatcgcttggggcctggagttccagatcaggcagggcaacatagcaagaccccatctctgaaaaaaattttaaaaattagccaagtatgatggtgtacacctgtggccacagctactcgggaggctgaggcaagagaattgcttgaggccaggaggtcaaggctgtggtgagccatgattgtgtcactgcattccagcctgggtgatggagtgagactctgtctcaaaaaaaaaaaaaaaaaaaaaaaaaaaaaacccttttcaaataaaaattctccatgaaaagaaaattcagaccaatttaagtggcagggttttttatttctacttttgcaataaaatatggaaattgctggagattgataagggaggggaatagagaatgggaggctctgggcgtgagtgatgggaggaaaagtcagggaaggcttcctggaggaggtggcggtggctgatctaagaagtgaaggtagagaaggagttaactgggcagtgaggggagggaagtgtgttctaggcagatggaacagcatgggcaaaagcttcccacacaaggtctttgtgaagcatcagttgtttcactttgtggcctgcactgggcctagtgcatagtaggtgctcgctaaatgcatcatggtagatattcagttggtgggaactgtcctcagtcccaaatctgaatgacgccatgtgggtgttgctttccagcaaccacgttctgaatgatccaagggtcagaggtttgaggttcatctgaagcccctgatctaagcccctgatctgaagcccctgttgagcagggataacctgtgatgtctcgaatcttcagctctctgccctggctactaacaaggctctgtcctcaggcacaaagtggacttgaaaggtggagaaacaacttgaaatgtggagaaacagctgggtgtggtgattcatgcctgtaatcccaggcactttgggaggccaaggcaggaggatcacatgagcctaggagttcaagaccagcctgggcaacatagtgaaatgctgtttcaaaaaaaaaaaatttttttttaaataaataaataaataagaaaggtggagaaacccacatcaaactgacattagtgaaaagctggggagttgattggctcttgaaccaaaaaggctgatgttagaatggaccaccttcaggcatagttggatccaggggctcaaacaagataagcagatccagagagattgagccatttatacctgaggctgcacagctaagaggaggccaaatcatgatgcagactctgggttgctttgaatccaaagttcgtgctgctcagcatgactcagaaggttgatccaagtcagggaggtcacgactctatcttactgaggttcatggaagggaaatgacatgcttaaggtcacacagtgttcaaggagcagagactggcactgaactcagctgaagctgtggtctcgattgattgatagagacaggatcttgctctgttgcccaggctggaatgcagtggctgtgattgtagctcactgcagcctcgacctcctgggctcaagcgatccttccaccttagcccactgagtagctgggactacagacacacgccaccatgcctggctaatttttttacttttatttattattattattattattattattattatttgtggagagagagagagagtcttgctgtgttgcccaggctggtcttgaactgctgggctcaagcagtccgcctgcctcaggctcccaaaatgctgggattacaggtgtgagccactgcgtgcccagcaaacctgtggtcttagaaccacgttgcagggtttcttcttaagcatggattgaacggtgggttggttccgctgaggcaggaaggagaaaaggctctccaggggccaccttgaccttgggccaggccctccccactccatctaagaagcgcccacaccagcccctcctgccgcctgcccttgatggtcgtgacatttcccgtcctcttgataaacacatcacacggagacaggtagggaggtccccgttgactctggctcagtgccagccgtcggccccggccaggcctgcccatctctgtcacaggaggaaatgagactggcagcgatttgttttccgacgagccagccgcctcgctccccagcacccggtttcctatgcgtgtgtgctgactgcgtggattccccagagagcattgtccaacccatccctgtgcaatggaccactctcttaagtctcaccatctcctcctgggtacacccctgtctctgctcactttgcccttgagcatctccagggcacttattaggcgtctgctgtttttaccagggatcaggcgtaggctgtcagcgagcgttgtcaccaaatcccaggccaggaccgggcacggtggctcatgcctgtcatcccagcactttgggaggctgagtcgggcggatcacctgaggccaggagtttgagaccagcctggccaacatggtgaaaccctgtctctgctaaaaatacaaaaaaattagctggagttggtggcgtgcgcctgttattccagctacttgggaggctgaggcaggagaatcgcttgaacccaggaggtggaggttgcagtcagccgagatcgtgcctctgcactccagcctgggcgacagagtgagactctgtctaaataaataaataaatcctaggccaggttttggggctcagcagcctggactcatgtcccccctctgctgccgccatgctgtgtgaccttggacaagtgtcttgacctctccgatgctcagttgcctcatctgtaaaatggagacaattcgaattcaatgagatattatgtgtaaaagactttgaaccatgcctggcacgcagtatgtgacctataaatattagcagttattgttactattgctattattttatttttatttttaatttttttagagacagggtcttgctctgtcgcccagactgaagtgcagtggtgtgatcatagctcactgccgcctccatctcctaggctcaagcagtcctcctgccttggcctctcaagtggctgggactacaggcatgcgccaccacgcctggctaatttttgtatttttttgtagaggtaaggtctcattatgttggccaggctggtctcgaactcctgggctcaagcaatcctcctgcctcaacctcccaaagcactagggtacaggtgtgagccaccatgcctggccctgctgctattattatagcatttgcaaatattggcatattgtgctcaccttctcgtcccacacatcctatgcccgctgtccctgccatgctctttcactgacagtaccaggacgcacacagccagggatgtctttttcagggctgcgtactatgcattctttttcagggctgtgtagtatgccatctacatgcccagcccctcctgacggacatgcccatttgtctctctcttttgctttcctatgagatgtgttgcagggctcgttcacacacaggcctcacgcacaaccaggagatgtcatgtgggatgcgtatttctttcttttttttttttttgagacggagtctcgctgtgtctcccaggctggaatgcagtggcgcacgatctcggctcactgcaagctccgcctcccgggttcacgccattctcctgcctcagcctccccagtagctgggactacaggtgcctgccaccccgcccggctaattttttgtatttttagtagagacggggtttcaccatgttagccaggatggtctcgatctcctgacctcgtgatccacccacctcagcctcccaaagtgctgggattacaggcgtgagccaccgcacccggcccaagggatgcacatttcaatattcactccctctcgcccagttaccctgcagagagcatctgccatctccaccccagccgccagccagggagctcgggtctcccctgaacagctttgatgagacaggaatgttctttttctttccttggctgctcccaagatacttatatccccacccccagcctccacccaggggatggtttcaggtctaaaattttccttcagaccccaaggaaagcttctgtgtgttcagaaaacaaccgtcgtgtcctttctgggcccaggaccctgctgggggcctagagctccatcactggatacttattgagcacctcctatataccaggccctgatcctcacagggtcaggtgttgggaacagagtgacggagaagacagagggaaaaacccttccctcgtggaatgcatcagaaggacaatgaacaaataggggccgggcgcagtggctcagcctataatcccagcactttaggaggccaagacgggcagatcgcctgaggtcaggagttcgagaccagcctggccaacatggtgaaatctcatctctactaaaaatacaaaaggctgggcacagtggctcatgcctgtaatcccagcacttcgggaggccgagtcaggtggatcacgaggtcaggagatcgagaccatcctggccaacagggtaaaaccccgtctctacttaaataaaaaaaattagctgggcgtggtggcatgcacctatagtcccagctactcgggaggctgaggcaggggaatcgcttgaaccagggaggcagaggttgcagtgagatcacgccactgcacgccagtctggagacaaagtgagacttggtttcataataataataataaataaaaatacaaaaattagccgggtgtggtggtacaagcctatagtcccagctactcaggaggctgaggtgggaggatcacttgagctgaggagttcaaggctgcagtgagctgagattgcgtcactgcactccagcctgggtgacagagtgagaccctgcctcaaaaaacaaaaacaagaaacacccaaaaaacaaaagccataaagccaatagattaaatatgtgttaaggattgactgtatgccatgcctttaagaaaaataaagttggccaggcgcagtggctcatgcctgcaattttagcactttgggaggccgaggtgggcagatcacttgaggtcaggagtttgaaaccagcctggccaacatggtgaaaccctgtctctactaaaaatacaaaaattagctgggtatggtggtgggcgcctgtaatcccagctactcggtaggctgaggcaggagaattgcttgaacgggggaagcggaggttgtagtgagccgagattgcgccactgcagtccagcctgggtgatagagtaagactccatctcaaaaaaaaaaaaaaaaagaaaagaaaaaagctagaggaattggttggtggcagggaggggctgttttagctacagaatcaagacaggcctcacagaagaggtgacatttcagtaagaccaacaggaataagagaatgatgtttgctgacatctgaggggagggtgttccaggcagagggcacagcccgtgcaaaggccctgaggcaggaccgcacctggcatgttggaggaacagcgaggaggcctgtgtggctggagcagagtgaggagggggaggaggggaggggagagcagggagaggacagagcaggttgtgcaggcccttgtggggcaccaggaggacttgggcgtttactctgagggaggtgggagccagggagggctatgggcaaaggaggaaccagccctgaccgacgtgttcacaggcgacccctggctgttgcagaaaggacagactgtgggggatgagtgcagacgctgggggaccagagcagaggggacggcacagatccaggccagggatgatgggggtggaccaggcaggggcagaggaggggttgtattctagaaggattttgcagaagggttggctggcttctccaagtggttggctgcagggtagggttgagatgagtcaagaacgactcccaggcctctgtcctgaacatcggtaaagacggagaagatgagctgaggtggcgaagatggtgagagaggcaggcatggaaacgcccccattagcaggacgcctgcggaggttcccaggaagtggtgggctggttctccctggagacatggagttggcagctcctgacttagcccatagcgggttttcaggtgctacaagcaagccattggctctggacaaggcacatcctatcctcagcctcagtttccccatctgagataaataactggcacaacaaaacgagttaatatgcgtcaagcacttagagagtggcctgcatagacagggccctgggaggggctgtgggctacacacttggggctggatggggctagaggttaggacaggaagggaggtctaatttgcagaccagggggctgaagcccagagaggttaaggcacttgcccaaggccacacagcacgtggcacaggctcaaccccactaaggggcctgcacactctgctcaccaccagagccagccaacccccccttcctcccttctctctccacccccggagctgccagctccagtttttggtggtttccaggctcccctcagttcccacttggcactcacctcccacctccacacactctgagcccagggcctagtgccaggcaggctgtagggccagggggtcttggtgagcatcctccacttctacacccagggttccttttgcctccctgccccgtaccccacccgtgctcctggtaatgaagggagaggaacgggagggagcgagcaaaattatatacacacgaacatacaaaaattagtttaggatccgggccaagaaccacttgcgcttggccaggcattgtgggtaattcattgctgattaccataaaagccatagcctggctgacaggcggctgcggggcttttgtttaagattctgatgaatatataatctctctccccgcctggagcccacagttcccctacccccaaggctgccaggtccctgcccaccctgctctcttcccccactgctgaacgtgagtctgcaggttccccgaggggctggcagccacctctgccgcatggagtctgcccctgtgactgcgtcggtgaagcttggcttggcactcgatgctcaccaccagccacaactagcccaggccttgcactgtggctgctgccctgggcctgattccctctcttccgtgtggatgtactgggcttcagctgtttgcttggccccatgctgggactggggacactatagtgactgatgcagacaatccttgcccttaaggagtctggggggacgatggatgattagacgggcatttatctatagttaaagagttcccatctcctgcccaggagatgcaaattaaaacaagatatcatttctcacccatccatttagcaaaactgtttgaagtgtggtattatccagggtggcagcgtcgaggggaaccaggccctccgggcgtggctggcaggagaagaaatcggcatctctgtgacaccacgtcttttatttttaatatgttgttctggccagagaatgcgtacacatgggatacaattgaggatgcagagaaaaatgcatggtggagaatctccagatcctctcccctctctctttccccagtagcagtctcttcccagaacagggtcagtagagtttttccataaaggaccaccttgtaaatctttccagctttgccggccggatgatctctgtctactactcaaccccgccattggagcgtgaaagcagccatagacaaacacgtaaactggtgggcgtggctatgtgccaataaagctttatttataaaaacaaacggtgggccggatttggcccccgatatggcgatgataaaaatggtctccgtctacgcaccgtggtacagtcgccctgagccgaatgtggctattgagcgtttgaaatgtgtccagtgcaaccgaggaactgaattttaaattttattccattttattccgtttaaatttaaacagccacatacggctcgtggctacggttttgaagagcgcagtttatctagaaatcttctgtgaacatttagtaggcggctggatttatattcccttgtttaaccaaatggcagcatcttctacatgctgttctgtaccttgttttcttcccccccacttaaaaatatagtatcttaaaagtaattttataattgtacatctggtattctcattccttttcacagctgtgatactgatctcataacactaagagatactgatctcataacactaagatactaatctcttcgatcattatttgacctcttgtactgttatttttatttatttattcattcattcatttatgtatttatatttttgaaatggagtttcgcttttattggccaggctagagtgcaatggcatgatctcagctcactatagcctctgcctcctgagttcaagcaattctcctgcctcagcatcccgagttgctgggattacaggtgccggccaccacacctggctaattttttttgtatttttagtagagacagagttttgccatgttggccaggctggtctcgaattcttgacctcaggtgatcttcctgcctcagcctcccaaagtgctgggattgcaggcgtgagccactgtgcctggcccccttgtactgttctttacttaaccaatctcctatcgatagacattcaggttgcttcaagccttttgccgtgagtactttctccacgtgtcatttcacgggaaagtgtctgccgggtagatttctagaggtggtgttatcaggcctaaagggaatgtataaacttaacttcggaagataatagccaattgtctcctcggggactattaggacctactcatttccgactgtgtaggaggggaacctgcttgtttagaacaagctgtaacccgaccttccaccccagctgcccttcactggacaccagtgagagacaaaaatgcttccccaaggggacctgggagatgtcagcatggagattcaccatggcattgggtgaggttgggaaaaaatggaggcaaccgaagtgtccacagggaattgggtgataaacgcccaaggcacagccttgccacggaagatcctaccacaggcgagaagaggcctgggctgcgtgtgctcagtagttacaagtcatgtgaggaacagccatgattcgaacagaatatcattttcacatacaaaaaaaaaatcaccctgggccaggtgtggtagttcatgcctgtaatcccagcactttgggaggccgaggccagcgtatcacttgaggtcaggagcatgagaccagcctggccaacatggtgaaaccccgtctctactaaaaatacaaaaattagccaggcgtggtggcgcacgcctgtgatcccagctactcgggaggctgaggcaggagaatgactggaacccaggaagcggaggttgcagtgagctgagattgtgccgctgtactccagcctgggtgacagagtgagactccgtctcaaaaaaaaaaaaaatcaccctgtattctagagttacctttgatcaataacttcatgagaaaaagacacaaagatagatggtaatggcagaggttgcttccaggggagcattagagggactccctcccttcctcaagtatttgtacctattgtgctgctggttctctctgttccaggcatgaggaattcgccctttttttttttttttttttttttttttttttgagacggagtcttgctctgtcgcccaggctggagtgcagtggcgcgatctcggcttactgcaacctccgccttccgggttcacgccattctcctgcctcagcctcccaagtagctgggaccacaggtgcctgccaccacgcccggctaattggaatttgcctttaaaaaaaaaatatatatatatatatatatttatttatttatttttcagagacggggtctccctctgtcacccaggctggagtgcagtggcaggatcatcactcactgcagcctcaaactcctgggctcaagctgtcctaccacttcagcctcccaaagtgccgggattagaggcatgagccaccgtgtccagcttctaggcacaaggatttttttgtttgtttgtctgagatagggtctcattctctgtcacccaggctggagtgcagtggcactattatagctcactgcagcctcgacctccaaggctcaagtgatcctcctgcttcagcctcctgagtagctacaaccacaggcatgcctcaccctgcctggctaatttttgtatttttttgtagagacagggtctcgctgtgttgcccaagttggtcttcaactcctggcctcaagtggtcctcctttctcagcctcccaaagtgcagggatcacaggcatgagccaccatgctcggcaaataatagctgttctgaggagaatttagtaagtgtgcctggatggtgggaaaagcctggaataatagctgatgtgctcactgagcacctgctgtatgcccagctcagttctgagtgttttgctcctgctaagtgcttgataggggtttttatgtcttggggagatggggttgggtgtagatcctaacacctgttcagagagaccaccttctcctacccaggacctgagagcttggggagccacctagagaggtcatgcccccacctgagctctgggggggtagtgggaagttctgccaccccaccccccctcaccagccccccatgtccctcagttgcctctcccatgggatgggacctggcccagggtccccaccctctccttttctgttttgttcagggctagttaggtttcctcttccacacctctcttgcctccagaccttggcatatgctgttccttctacctgggacacccttcccactccccaacctccctgccccagcccagcgcaatttctcccatccttctcatcttagacatcacctcttcctggaagccttgcgacatcccccattacagccctgatctctttttgtttgctttttgtttttagaaacggagtctccccctatcacccagacaggagagcagtggcatgatcgcagctcactgcaacctccacctcccgggttcaagtgattctctcaccttagcttcctgagtagctgggactacaggcgtgcaccaccatgcctggctaatttttgtatttttattttatttttttgagacagagtttcttttcgcccaggctggagtgcaatggtgtgatctcagctcgcaacctctacctcctggattcaagcgatcctcctgcctcagcttcccaagtagctgggattacaggcatatgccaccacgcctggctatttttatttttttatttcttattttttttagtagagatggaggtttcaccatgttggccaggctggtctcaaactcctgaccttgtgatctgcctgcctcggcctcccaatgtgttaggattacaagtgtgagctaccacgcctggccaagacctggcctttaacttccagggccccctgtctgcctcccacagtcatcttgtgtccttagtgctcagcccagagcctggtatacagtaggtgccttataaatgctagagggatgctgagagacgaatcattactggggtgtgacagtttttcattcctcaaatgctgacatggcatggagggccatagccctccgcgcttctgtttattttattgttattattattttttgagacagagtctcgctgtgtcacccaggctggagtgcagtggtgtgatcttggctcacagtaacctccgtatcccgggttcaagcgattctcctgtctcagcctcctgagtagctgggattacaggcgcataccaccacacccagctaattttttgtatttttagtagagacagggtttcaccatgttggccaggctggtcttgaactactgacctcaagtgatccacctgccttggtctcccaaagtgctgggattccaggtgtgagccaccgtggtcctcactcctattacccctcaaatcctgcccctgctcactcactctgcccgctctctggaccagcccccctctctgcaccagtccccctctctttttccagaacttagtgtgttcgttcccacctcggggcctttacacgtgctgtgtcctctgcccctgtcctctatccccatctgtcgctgtctccccagaacagaagccctgagaggttatctgccttgatctgtactgtgtccccagcactcaagacagctctagcacacagtaggtgctggagtttcagctgtgaccaaaaccagatcccacccttgtgcacggggaccaggaggaattaggagctacaccaagccctgccttggagacctccctggtttgtgcctcggtttccttgatctttacagacttcagacccacaaaaatcagccccgggcaccaagagcccattttccctggtgcttagatggggaaactgagtcccagagagtggtgaggactggttcaggcccacacagctcccttgggaagagaacttgggcttcctgcctcctgtgagccacggttttttgttgttgttgtctttttttgtttttgtttttgtttttgagacggggtctctctctgtcgcccaggctggagtgcagtggtgccatctcggctcactgcaagctctgcctcctgggttcacaccattctcctgcctcagcctcccgagtagctgggactacaggcgcccgccaccacacctggctaatttttttttttttttttttgtatttttagtagagatggggtttcactgtgttagccaggatcgtctcgatctcctgacctcgtgatccgcccgcctcaacctcccaaagtgctgggattacaggcgtgagccaccacgcctggccgagccatggtttttgaagtcaggaacgtacccctaggcatggggctgtttgggggtggcctctggaggaggcatccggcagcccagagctgcacacaccagcaatgactccctttcagctcctaacacgtcagggaagaaagcccagggagattcagggtgatctcagacactaacacctgccaatctctttgtaacaaaatcccatccggctgctggtgttcgcaacagtatccagcgaccatgggataactgggtactattttcattgtaacaatgtttctggtgactttcaaggcagggcagaatcttttgggtcttccttagagatatgcgatactgttgaattagtttcttaagtaaattggtttaaggaaaagacgggagacagtggcatgaaagttgttgtttctagacagggcctccctctgtcacccaagctggtgtttagtggcgggatcttagctcacctgcaacctcaaactcctggtcacaagcaatcctcctgcctcagcctcccaagtagttgggagtacagacatgtactgccatgcccagctaatttttaattttttttagagacgggggtctctctttgttgcccaggttgatcttaagttctggctcaaacgatcctcctgccttggcctcccaaggctggccgtgcccagcctacatgaatgattaaatcggggggccaagtgtggtggctcacacctgtaatcctagcacttcggagggccaaggcaaggcggatcatttgaggtcaggagttcaagaccagccttgccaacatggtgaaaccccgtctctactaaaaatacagaaattagctgggtgtggtggcatgtacctgtaaccccagctactcaggaggctgaagtgggagaatcacttgaacccaggaagcggaggttgcagtgagctgagattgccgcgctacactccagcctgagcgacagagtgagactccatccatcatccaatcaatggggggtattgggacatggcagagtcatgagcaaggaggggaggattggggggagactgtgatcacccttcctggggactggtgggtgtagagggggaggatgcgcttgaaggagagaccagcaggcatcttgcagagaggatgtggccagatccattaaatgcatttacagccaggtgtggaggctcatgcatgtaatcctggcactttgggaggctgaggcgagtggatcacctgaggtcaggagttcaagaccagcctgaccaatatggtgaaaccctgtctctactaaaaatacaaaaaatagccaagcatggtggtgggcacctgtaatcccagctaatcgggaggctgaggcaggagaatcgattgaacctgggaggcagagggtgcagggagccgagatggtgccactgtactccagcctgggtgacagagcgagactctgtctcaaaaaaaaaaaaaaaacagcaacaactttaatacacttgacgctggctcaaggtgggagacggcctggcaggggcccaggctggcaatggggcctggcggggggaccggccattctgtccctcccccgtcctccacttttggctgagtcagcatgttggcgctgcagagctgactccggctgtggcctggaagccttttccaagtttctgcccttggggctggagcaagagctcccagaggatgctttgctggccagccagggagatccaggttcaaagcccagcaatgtggcttttgctttctggctttccatgtcactatgttggaaatgtagagtttggataccctaacgtggctgcccagggagtgagagtggccagggtactgtggctgcagatggcacctgctgacctctgtgctattggccagcacctctagtctctgcaaggcctctgagccattgatgtcttcactctttcatgtctatattttttatggaaatttttttgttatatatattttaccgctttttctttcttttcttttcttttttttttttttttttgagacggagtcttgttctatcacccaggctggagtgcagtggcacaatttcagcttactgcaacctctgcctcttgggctcaagcaatcctgcctcagcctcccaagtagctgggattacaggcatgcaccaccatgcccggctaattttggtattttttggtgagatggggtctcactatattgcccaggctggtctcaaaaacccaggctcaagtcatctgtccacctcggcctcccaaagtgttgggattacaggtgtaagccaccacacccgccccacttcttcatttagaaagcatttattgagcatatactgtgtactcgaccctggcagtgaccaagactgacctggcccctttgttgctgggagagctggccacagaactttctgcagtgatggaagtgtgctatgtgggtttcagtactctgaccatgagccacactggaaatgtggccggtgtgacttgaggagcagaattttaaattttatttaatttttctttttcttttcttttttttttttttgagatggagtctcgccccgttgcccaggctgtagtgctgtggcatgatctcacgtcactgcagtgtccacctcctgggttctagcaattctcatgcctcagcctcccatgtagctaaggctacaggtgtgcaccaccacactcagctaatttttgtgtttttagtagagatggggttttgccatgttgtccaggctggtctcaaactcctggcatcaagcaatccacccactttggcctcccaaaatgctgggattacaggtgggagccatggcacccggcctatttaatttttcttcatttaagtttaatgagtcacatgtggctagtggtcctttttctttttcttttcctttattttattttatttcgaagcagagtttcactcttgttgcccaggctggagtgcaatggcgcgatctcagctcactgcaacctccgcctcccaggttcaagcgattctcctgcctcagccccgagtagctgggattacaggtgcgcaccaccatgcccggctaatttttgtatttttagtagagatggggtttcaccgtgttggccaggttggtctcaaactcctgacctcagaagatccacccatcttggcctcccaaagtgctaggattacaggcgtgagccaccacacccagccactagtggttcttgtatttgataactcagacccagacagtgagtaaagacagagaatttcaggtgacagtaaaataatgggtaatgggtgatgggtgctgcttgagccaggtggtcagccagggcatttctgaggaggtgatccttgagggagaatggaagggtgagaagaaaagtgacaagaaaatgtagaggaacagtgttccaggcagaggcatagcctgtgcaaaggccctgaggcaggactgcacttggggtgctggaggaacagcgaggaggcccatgtagctggagcagagtgagggggagagagggaggaatggagggcagggaggagatggagcaggtcatgcagggccttgtgggccaccaggaggacttgggctttgaccctgagggaggtgggagccatggagggctgtgggcagaggagggacgggacctgactcaggtgcacaccggcaccatgtagtgggtgctgtggggaggacagactggagggcgagggtgggaggctggggaccagggcagaggtgactgtgctggtccgagcaggcaatgatcaggctggatgggttgggaagggaatatggagaaagatggacaaagtgagtagaaatttaggagacaaacgtgagagtatttccagaccaacaccatatggggtgagggaggaaaagcagtgagggccagcttccaggttctagcctgagcccctggaaggacagagctgctatcagcagagttggggaagacatgggaggaatggctttgctggatccagccatgcctgaaggtgtcacacattttccaggaaggggaaggtagcctggcctgcgggggcggacaggtgaaaaaggcagacatggagctggggcgtgcctgtggtgggcatcatgcccgctgcggtgcggctggcgggtagacccggcgccagggctgtgatgtgaacatgggtgaggcgggctcctggctggttggcacccgcccatgccccgggtcatgaatgctgtgctaggtaggaggcgggagccttgggcatgggcaagaccagggcagggaagctgagaggaagctgcccaaccagtttctctctggctctcacagccaagagtacagagtttgtacctaaatgaaatgtctgggggttttggtgccaaatatgaaggaggtgggacagacaggctgggggagggggtcctgccccggtcaagggtatggttttatctgagggttccccatcagggaaggtttaaaggcttgagaacatcccaatgcagtcacccctgctttggaagtttcttcaggagacgaagggacacagctgtgtctaagaatgtccctcacagtgttgtttataagggacataatgggataaacaagtgggaattgattaaattctgtgacccggacactggaacatggtctgctaatggtgatccccatcgaacgaaagaaaagcacccattcgtatgcatagaagaaagactggaaggaggccgggcgctgtggctcacgcctgtaatcccaacactttgggaggccgaggcgggcggatcatgaggtcaggagtttgagaccagtctggccaacatagtgaaaccctgtctctactaaaaatacccaaaaaattagccgggcgtggtggtgtgcgcctgtaatcccagctactcaggaggctgaggcaggagaattgtgtgaacccgggaggcagaggttgcagtgagccgagatcgtgccactgcactccagcctgggcgatggaacgagactctgtctcaaaaaaaaaaaaaaaaaaagaaaagaaaagaaagactggaaggaaatggttagcctcagggccatcgtatttgctgttcctcctgcctgcaactctgttgtcataggtgtttgtatgtctgatttgctctcttcctccaggtgtaaatgcaattatcaccacttctaagaggctcccccagggaccccccagtttttgcagccttgcagtgtggtttatttaattatttttggcatctcttagcactgcccggccttatgttattgacagtgtttgtttttctgtgggtgtccatcccccctgcaactagtctgtgccccccaacaggggcaggagtgctggttccatcatgcttaccactgtgtcctcagcacagcacacagtaggtgctcagcacatgtacagggaaagaatgaatgagaacggccgcatgaaacagtttctctctaggcgagggatggattgtgggtaacttttattttcttctttatgctgattttacctaattttctgctgcacgcgcctattttataattagaagaaaagttattttgaaaaaaattaccccccaacgtcccagctgtgggagctgacgtgatccagccccgtgttcctccccaagggcagccgcctcctgggtgtatacagaggacatatcaatggaacgtttttggaggggtccaggggtcagcccccaagggcacggagtaggcggggcaatcagggagggcttcctggaggtggccacccttaacctttagggtgctcaggccaaaaacccagaagtcatccttgagtcccctcttttctgtacatccccaaatgagccgtttagaaaattctattgggtctgcctagaatgtgcctttctccctcatcctctacccagtccagccccagcctagtcatcccccacagtctgccctccccacagcagctgagcatgtctgtgagtacctgagtcagggccttcctctacccacagccctccatggctcccaccttcctcagggtcaaagcccaattcctcctggaggcctctgaggccgtgcgcatcttgtccccattccttccctcccctccctcccctcccctcctctttgtctccccttcctcactctgctccagcaacactggcctcctcgccattcctccaacacgccaggtgcagtgctgccccggggcctttgcatggactgagctctctgcctggaatcctctttcactttctctcaccttctcctggtctttgttcgcatgtcacctcccctctctcctatcctgtgtttattttattttacatttttttagagatagagtctcgctctgtcgcccaggctggactgcagtggcaccatcctagctcactgcagcctcaacttcctggcctcaagcgatgcttccgccctcagcctcccaagtagctggaaccacagtcatgcgccaccaggcccagctaatttttaaaagtttttgtagagatgatgggctatattgcccaggctggtctcaaactcctggcctcaggcgatcctccctcctcatcctacaaccctgaacccaatcatcctcagggcccttaccaccatctgacccacagtggatttgatttcttttgtttctcatgtgtcaccctctttagagcatccacaccctgagggcagggatctttgtcagtcttggtcactgtgtgtctaggattgtgcctggcatcggggcttggaaatgtttgtttagaaaagggtaggctgggcttaaccttattacgggctcatgtctgtgatcccagcactttgggaggctgaggcaggaggttcacttgagtctaggagttcgagaccagtcggggcaacatggggagatcccatgtctacaaaaaaatccaaaaattagccagacgtggtggcatgtgcctgtagtccaagctactcaggaggctgaggtgggaggatcacttgagcccaggaggttgagcccaggaggttgaggctagagtgagccatgatggtgccactgcactccagcctgagcaacagagcgagaccctgtcttgaaaaaagggtaggtgaagatgcctgctttatgacaagggcacacacagtgtggcctttggaggagaataaggtcggaggggctctcctgggtgaggctgggcctgattgtgcaaatgtttgctgagaacctcctttgtactgtggacctgtgctaggtgctggggacactgggtgaccgagacaaatcctgctctgcctcctgaggtcacagtctggtgggagaggcagacccggggactaggcgcatgtagggctctgtgctgatggggccacccagccaagggggagggcacctggcccacctcccccagcccctcgggggtggctgtgtcccctgccaactgcagtcaccactgtggaataggggcaagagtctgcctccacagagacatggacaagaggcccagagagggcaggactggctgggaagcggctggcaagcccttgtctgccgtcccgagtcctgggcagcccagagccaggcctgtatgctgcaggtcatgcagccccaagcctcctgacagtgtggattcattgagcacctgttcagtgcttggcttatgtggcagatggggaaactgtcggcccgagaggtacgggtgctttcttgacacacggggcatccctggattcaaactcgagtctgtatgagccccgtgccttgccacagatacaagcaaggtgcctactctgctcctgagggcccactgtgtgcccagccaagttggggaggtcacgctggttgctcctacgcctacattgacacagggaacctcctgtgtgcctttggtgatgctggggcaccaggcagcctcctccatgaagcacctactgtgtacctcgtcgaggatggggctgcgtgggacgggggacgtcacggcatcctgtgtttagcacctcctgtgtgctctgtgtcaaaggggcaggcagggagcctgtgtgcagacactcacagaccacctactgtgcatggtgctttgtggactaggccacctattgttggagatggggcggggcgattccatgctccattttgcagaggagggaaactgaggcatggtaagggaagggggcagtgtggtatcccccagtgagggaggcatttattcgatgtttgaaggagatgggagccagccactgaggtagcagtgggagggcatggacgggagccgtgggagcggaattgtttctgctgggctctcacagccgcctgtgtgtctcccatcaaagcccacgtcctttgccccactgagtaatggcttctagagagtggggcgtctgtcttcaggatggccctgtacctagataaacccaaggggcttgaaattattcatgatatatcctcagttccgggggccccaggcctccagcagcgctgtggtgagagctcctcctctggacccaaaccctggttccaattcctgctcttcccggccgtgggcagccgtttctctctctttctcggctccgttctttgcagaatgggggtgatggtggggtctgcctcctagagccgtggccggtggcctgaggaagcacagctgatggcagctgagcgcccaggacatcccgttggcgttggcgttcgggcttcgtcctcagcgcgaatgctgggaagagaggcgttttttccagggatgggagcagacgcgggagcccttgggtctccatcccggcctctgcccctgtcccgtccccagacccccaggtcccgctgggcttctacattccgggcccctccggtggagttttcaaacccgcaccccgggcgcaacgctggctcggattggccgcccggacaggtctgcccctccctctccctcctcctctccctctgctccggtgtcgcgggcctggcacggcggccccatctgtttccaatctggccctgtgctctccgccccctcctcaggcttgtggcgagggtgggggcctctgccacggtgtccccccattcattcccctggacccccagcgggagggaagcctctgcgcccgcacctctcccctaccctctggtctaagagggggaagaggggacgccccccgcccccatcaccgtgcctccggtcccacccagactgggaggggaactctgccccttctccgacctcctgtagttgggtgtgtttggggagccctccgccgccctgtccggcatccagcctcctcatgcgttgccatggctacgatcagagctggggaagcgaggagcagtgtagaggctccccgtgagacgtcttcgggagggaggggtcttgggtcgtgccctccctggggcgccccttcgctcagtgtttcatggactcctgcggaggctctccgcgcctccagatggggctcccctctggtccccccgccccacttccggccggggccccgccctgccccccaatccccctcctcggggcccgtcccctgcagccccgcccactgcaaccccgccccctacagccccgccccttgccccgcccccaggccgagctccccaggccgaggcctctcccgagtcttcgctgcgcgccgcgctccgggcggaccgagggacgcggggagcggctgcggcgggccctgtggaggcctcgcggggcctgggcccgatctccgagccgcggggtgagtgcgcccgggacccccttccaccccgaggcccgggccgccccgggctttgtctctcttgtcgcgccccctccaggctccgaggctggggaccacgggggtccccacagcccagcgcgggcctgaccgtgccggccgccctccagggccagcttgggcctgctgctgcctggcgcccccccattcaggcccgtcgcttgcactttggaaaactccagaccgttatgctccgcagggtcccctcccccgcctggcccccagcccgacctcctccccccaccccctctcctcccccacctcccgggggcggagccaggcgcggggcagccttcgctgccagcccgggggagctgtgactggggacttcgcagaaggcggaggaggcaacaggaccgcagagggggcaggtagcggtggggggtgcatggcaggaatccctgctcttagccacccccgaaaatcgcaggaagtttacctccatccatttgtttccgctcctctcgggcctgtggaccccacgttgcctctgtgctttgttggggggctttaagaaactggggggcttggcctcagggaggggcctgtggtgttgtcttctgagacggtcagggcagctcgtacctttcttttacccattcccgtccctgggctctaagccgttccaggacccaagtactgagctgacagagtttggtgaccctccccaaggggcttcctgcagaacccaggctggcctcagctgtctgttccttccagagcctcacccccaccctttaagtctctgccagaccccctagggtggatgcgaggctgtccccgtctcggtctccattgcctggggtggtctctgtctatggtctcagtttctacctctgaaacccaggtgacgccagggatgctgtggataaagtgccaggtggagctggttatgcagcaggcatccagtgcatgcaggggctttgccacgttcctggggtgggaaaagggcttccatccagggggttgacagacccacagtgggagcacagctgtgatggggtagctcagaatgggtcttgatccaccctggggcatccaggagggcttcttggaagaggtgacagctcagctgagccctgaggacaagtatgagggctgagatttgatgaggatgtgaaagggtatgctgggtggaaggaacagtgtgtgtaaatacccctgtgggagccaggggccacaagtgggggggatcaaggtgtcaagggaagcctggcatggggggttgtggcgtgatgaggtcagcagaggccgggctggcagctctggaggctgaggtagcagtctgagttttaatacgtgttgatgggagccatggagggttttagagcagaggaggaacggggctcagcttggagtggaggcatctcgtggctgcagcttacaggatgaactgaccatgggccagctggggaagggtgtgccaggtgggggacctgcatgcgaagaggctcagagacctgaggcagtgtattctgagaactgcaggacctttcgggaagagggggtagggaagacgatggatgcagtcacatcaagaagggcccagagcagcgagcacaggagctgggagcccccgtgggtgtgtgagcagaagcaggggtacccccagatcttggcgtctcaagttgttccatgaagccagcatgggggacacactgacgggcaattttggcagcctgggcgcccagaggatgtggactctgggctcgactgagagccgggctctggcctcaaatcctaacccagcccagccccctcctccatgcaggacccttggtgagcctcggtttcctaatccgttaaacagcagaaaacacagagaacatttaatggggctcagcaccccagaggcacccagtggactttaatgatttccctctccagggctgggcctggttccctgtctgccccacccatccactcctggccaccaggacccctggggacagtgccctgagccttcacgtcgtcccaatacaaatcaatagtcaattaaagccaggtccccggacttcagttccgggaagggccagccccacccagcccctccctgagcccatttcctgatagggagataatggcgccctgggagggacagtggcttggccagagctgacttgagcccaccagtggccacagagcctgtgcttgctgcccaggggggccctcgctgcctgtccctgcttgagtggatgtgggagtgacccaggtgtccagggaggctggaggccagggtccagtcctggaaatgacgttccaggcggtggcacgggagagctgtgggtggggggtggatgtgaaggtggagagattgtgcagggtcaggggtctttcccctgagtcccactgctggcataatcagcctcagcttctcctgccctggggccatgggatggcccctgagggttaggactaggcccccagactcccgccccaggacagggcagcatggactctacagctggagcctgggctgggagaggcaccgggagcctcctctggcccgaggagcccccgcctcgtgcgggtgacacagcctgtttgctgcccggcctgggtcataatcacccctttatgcttcctggaccgcgtgggcacacaaagccggctcccagatcgcgtgacccatgccccggcgcctggttggaatgagtgcccactcgccccccattagcaccgcgggggagcccccagaggcggccagcgggctctaccagtcccagagagagttgtccagagatctcgcagtggccagagaaagacacagtcacaagattaaacgtgggctctagacccaagtcccaccccagctgggggccttggctgagtccctgtttttctgtgagcctcagtttcctcatctgtaaaagtcaagcgcctgcctctgaccagctgccgtgagctagtggtgggcagtgcagcccctgagagccaggtggggcgggcagagaccctccctgagccccacatgggttcagcgtccagcggtcactccctctccttctccctagccctggctcagcctcgctggccaccatgggctgccatgcttttcctaacagccgtgtcatttactgggagcttgcagattactgcacctgacttaatttccctttcttggcagtggcaaggctggtggcccaggctgccttttgcaagaggacatggtggagggttggcctggcaccaacctggccatttccctccctgcccagggccacgtagggctcctggctgctctgtgggtctagtctatgtgcacctctctgtcctcatctctccccctctccctcctaggacctctcagtgctgcccccttgctcttcctcatgctgtttgtagtgcagagacacccttgcccccaattcccattcaccttttggtcctcagccaatattaaatccaggaatggcccacgcaaaggcccgggggcaggactggactgcacttggggtgttggagaaatagcaaggaggcccatgcggctggagcagagggaggagggggagaaagggaggaggggagggcacggaggggacagggcaggtcgtgcaggacctggtgggcctcaggaaggacttgggctttgaccccgaggcaggtgggagccatggagggctgtgggcagaggaaggacgagccctgactcgggtgcttgcagatgctttctggtttctgtgagtggaacagatcaagggacaagggcgggagctgggagaccacgatggaggtgacgacatctgtccaggtcggtgacaatggggctggaagaagtggaggcagaagaatgagtaagaagtgggcaggttggggacagattctgaaggcagagctgatgggacctagggatcttctctcccaaatcctcgcagaggacctcacgcctggggttttcgtggaagcaggagtcggtgggaggattgtgggtgtgcagggtcctatccccatcatgggaggttctcctagggaggattggcacctaagggaccccgggatgttttgtctttggtagtgtctgaaccccactccctgggcccctcccaccaccctattcctgatacatcatccaatgccccaaatgctcctgttactgtagcccctcgtccctgcccctgacaggctggagtggcggatctggggggagtccatggccaggccctgcctgtctgtgcctgactcagccccatcctgcgcagacatcagggaggtgtgggcccaacatggcttctgccgtgggagccagggtgtaattagggctgcctggtttagaaggtggctgatgctgccctgaggtcctcgtgggatgtgggagagccagctgtggctggggccctccatatatgtgccccacctgggctctctgtgcaagtgactcctacccatgccctctgagcttcaggtttgctatgctgcctcagtttccccagaggctctctgcaggcctgcccagctgctcaaagtggtctgggactgacctcgccccctcagtgtggccaagcagatgtgcatttctgggcagggccagccgctgtggtcaccctagccccagctggccacaggctgttttctccatctggggccaccgccttcctcgttggcaacttttgctcttaaagggctagggcagggactcagagtaagggactgtgctctaggctttatttccttcatcttcccagtccccaactggttgtgtgcaaggtgtacctagagtctgactgtgtgtgtgcatgtctgtgtagcctgcctgtgtgcacatatgtgtgtaggaagggaatctgactttgcatatctctatttggctggctggcacatgcacctgtggtctgctgactgactggcctatctatagtctgacagtgtgccggcacctgtgtctgcgtgtgactttgaccggcttgtgcacctgtaggtatctctgatctaattgcatgtttgcagttccctgtaacccaactgtgtatgtgtactgcctgccccaccatgaccccttatgtgtatcttttctggcttggtgtatgtctgtggtctgattttgggcctgtctgtgtttgtggtctgactatgtgcaagttttatttgcctgattgtgtgtgtgtgtgtgtgtgtgtgtgtgtgtgtgtgtgtgtgtgtgcccgtctctgctgcctgactgtgtgaatgcatttgtttctgtgtctcttgcagatatgcaaccgcttgcagtctgactgtgtgtcggtgtgtatcggtggtttgactgtgtgcatctgtgtatgtctgtggcgtgatttagcaggcatcgttctctgtggctggtctgactgtatgcctgtggcctaactatgtacactgaggccccccgaatcccgttcgtcaccctaggctgtgacccggccactacccgcccatccttcccctgggaccgccccagggctgggcgaggtgaccgcagctgggtattttgggagtcagaatcacaggggctgccccgttattatgcgccaccagttcctgtggtcacggcctggacccgcccagggcatgggctggcatgggtgccagcctgactaggcaacaaagatcttatccttgggcctctcccttcccctccctggccctctgacctggctctggacagagtggagattggggagatggggagggggccattccagggttcaaggtatgtgacgagggacccttctgccctggaggagttgaggttcttgtgggagaaacgtgctgaatgattaacatcttttcatttagtttgggaaccagggtgaataccacggaggataaccttagtcatatctgatgctccgggatgtagaatgtagggggaattgacctttgaggggcagagaagggtttcctgaggaagtgacagctaagctgagacctgtcatgtggtggggacagcattccaggcagagggcacagcagaagcaaaggcttggaggtgggactgagcttggctggcctggctgtacctccaagcgagaggaaacagtgaagtcagcaagagtggcttcccctgggggctctgggagccatgggaggtcttagagcagggcagggacaggcaggtgtgtggaagagatttgtcttataacctgggggatcaggttttagagccagacagcagcatagtctggagtcccctccttcctgagaagcagcctgtgagttggagaaaactggggctcagcacccctcagtcctggtgtggggcttggtctgagtcaggggctcaggtcctcagtcctggtgtggggcttggtctgagtcaggggcccagcctgggttggggactcatgctggggttggggtttagctggtggttggaggctgtcttagtccatttgggctgctgtagccaaataccacagaccaagtggcttataaatgacagaaattggtacctcacagttctagaggctgggaagtccaagatcaaggcaacacagatttggtgtctggtgagggctatcttctggttcgtggaaggcatcttctccctgtgtcctcacgtggcagaaagggcaagggagctttctgggagatcctttatgacactaatcccgttcatgaggtctctgctctcatgacctagtcacttcccaaacactccatctcctgttaccatcatagtggggactaggtttcaacatatgattttttgtgggggagacaccttaattctatagcaagggcccagcactttcattccatcccatcaccccatgatccttccccattgtcccatgaccagcccccgcaggaccctcccctgctccatgtgaccctccctctgcctcctccatgaaggtctcctggatttccccactcgcctctcacagccctccattgtctctgcagacgttgccccatctgacgctcggctcgaggcctctctgtgagggaccggggggccatccccctccagggcggagatcggaggtcgctgccaagcatggcgcccacctggggccctggcatggtgtctgtggttggtcccatgggcctccttgtggtcctgctcgttggaggctgtgcagcagaaggtaagaagccggcacgggatgtgcaggggtctgcgtgtttgtttacctgtgtgcatttatggctgtgtgtgtgtgggcacactccccacctcctccctggggccctccattttgccaaatggaacttttatgctccacaaatgaggctgaatgctgaggactgtgtgctgagccctaagagattcagagataaataataatgctggtattcataaagtaataaccgtaatgatgatgacggcgtctgtttccccagcatcaactcagccccaggctccatgctaagcactaattttacctgcttcctctcccttgagcctgacaaaacccagtgtgccagttctgttaccttccacgtttcccaatgagggaaaacgaggcaaggacttatttgagatcactgcttagtgagccacagagttcaccttcccttgctgtgggcagtacctacacctggtggcagacacacaggcagacaaactgtctcaggtggagccaggggaggcttcttgggaaaaagggcattgggactggggttttgtgggtgtataggagttcgccgatagaaaagccattcctcgtcaggatgacagcacccgggaaggcctgcagatgaaagaggttggtatagtttagagatgggcaaggaagcatttctggctgtaatgtgaagaggatgagggtccatctggggcccaatgccaagactcaccccaccatgaaggacctggagaaggcatcactcaggcccatgaggacataggggatgagggcagtagggcaagagactgggttctgcagtcaggctgggtttgaatcctggctctgctgccaacctgctgcacaaccccgggctagtggctcagcctcccttacccactgtcacagagctcatatcattatccacatccagcattcttgtgatgtggacatttcataagcccctgcctgctgctagggacatagcaggccggcaatcctgtgttaccacaaacgtaggtattactgctggttaacaaactggagacatacacccctgcctcattcatttgcgctttttcttattttcatgctgctttttttattgtcgttttttccttttgagatggggtctcactctgtcacccaggctggagtgcagtggcagcatctcagctcactgcaacctctacttctcaggctcaagcaatcctcccacctcagcctcccaggtagctgggactacaggcgtgggccaccatgcctggctaatttttttaattttttgtagagatggggtttcactgtgttgcccaggctggtctcgaactcctgagctcaagcaatccacctgcctctgcctcccaaagtgctagaattacaggtgtgagccagccacggcatccagcctcatgctgttttttagtaaatgcccctatacattcagatcagccaccgcaccctgatgaggtgaatgcgattatcagcatccccgtcgtacaactatggaaactgattgcaccgctgcactcagcctgggcaacagagccagaccttgtctcagaaaaaaaagaaagatgggctgaagcgccttggcctcccaaagtgctgggattgcaggcatgagccactgtgcctgacctggctaatttttatttatttatttatttatttatttatttatttatttattttaattaattaattttttgtgacagagtctcactctgtcgcccaggctggagtgcggtggcgcagtctcggctcactgcaacctccgcctcccagattcaagcgatcctcctgcctcagcctcccgagtagctgggattacaggtgccagccaccacacccggctaattttttgtatttttagtagagacggggtttcaccatgttagccagggtggtctcaatctcctgacctcatgatcctcctgcctcggcctcccaaagtgctgggattacaggcatgagccactgagcccggctggcctggctaattttttaaatttcttgtagagatggagttcttactgtgttgcccggactggtctcaaactcgtgagcttaagcaatcctcccacctcagcctcccaaagtactgggattacaggcgtgagtcccagtgcctggcctggctaagtttttaaatttcttacagagatggagtttttactatgttgcccaggctggtctcaaactcctagatttaagcgatcctcccaactcagcctcccaaagtgctgggattacaagagtgagccactgtgcccaaccagaagtggccatttgaacctgagctgcctggtcccacaggccaagttcccaatgaccacactccctcagggaccagaagccaggtatagctgcactgtgatctgtctggctgcctccagggcctgagtcttcctggcctgtctcacctgcattggggatgtctggccttcagtactgacccctttcagttcttcttttcttttgttttttttttgagacggagtctccttctgtctcctgggctggagtacagtggtgcgatctcggctcactgcaacctccacctcccaggtccaagtgattctcgtgcctcagcctcccgagtagctgggattactggcacccgccaccatgccctgctaatttttgtatttttagtagagatggggtttcaccacattggccaggctggtctcgaactcctgacctcaggtggtctgcccacctcggtctcccagagtgctgggattacaggcgaaagtgctggtgtgacaggcgaaagtgctggggtgacaggagaaagtgctggggttacaggcataggggaacaatttctgctctagaaaccacttcaggtccagtggggccttcccaggatgccgtggggctggccccgggcagggttggaggggacagggtgggtgggtcaccagctctggcactggattcttgtctgcagaatctgaaatggaaaaacagagatggggccaagccaggccacggagcgggaggctcaggcgcgttgcctccagagagggctgggttccctttccagcttttttgatattggcaataccacatgaacttctgtctcttcatctaagagacagggacatccacggtacttgtggtcaataaagacccccaaccccatttatccagcctgcgtgattttcaaggcactggagccaggtaggcctgggttggaatcctgtctctgcccatcacacgccgtgtgaccaaaggcaggtcccttcttttttttttttttttgagatgaagtcttgttctgttgtcctgttgcccaggctcgatctcggctcccactgcagcctctgcctcccaggttcaagcagttctcctgcctcagcctccaagtagctgggattacaggcgtgtaccaccaggcctggctaatttttgtatttttggtagagatggggttttgccatgttggccaggctggtcttgaactcttgacctcaggtgatctgcctgcctcagcttcccaaagtgctgggattataggtgtgagcctctgcgccaggcctcttcacctttctgagcctgttttgctcctcctggccacaggcataaagcttagcacatggcgtgtctttgtgcctctcgagctgcaggcgcggcgccctcaagccccagaggtgtccttcctgtgtctctgcccctttgctgagaaaaaggtggggaagcttcgtccaaaggagagattgcaagcatttgcaagacattttctaacgggggactcagtgggcagggacagctcttccaggctggctcagatcccaaagctgcctggagaagaccgagagggtatctgctctgcagtgatgggggtgcttccgagaggtagaacccctccaaatccgagagcaggcagtgatgttgggagctccgttttctcaagggtcaccacacaccgagaagaagtctgaggtcccagggaaccaaggctgtcccttgacctctgtgctgagagccacataagcaggtatctcagcccagtgttctggggacgcccaggctgcggtgggggcataagggactcctgaccgggcctgcagtaatccaagaaggctccccaggggaggtgctggccgagctgaggttgaagctgccagatgagaggaggaggacgggaccctgggaggaaggaacagcacagacagtggctgattgtagcacccacccgcccggtcttgcttgctggattggagggggcaaggggtgtgttctggctctaggtcagacaggcagctggatgcgtctctaccctcagccagcttctgcccttccttccagcagctccagagggagggatttttaccagaagtaacattcttgacccggccacagtgacttttcctcatgtccttggctgaggacgcatggccccgggtcaccattcctgaggccagaaggagggggcagccccagcctcaatgtggagcaaaacatctgccctaatcaggctcaagttctggatgtggaagggacagtaagatgtcggctttggaggcagaaacccgggttcaaattccagtgctgttatttctgaactctgtgatcttttttgtggtggtttgttttttgagacagagtcttgccctgtcccccaggctggagtgcaatggcgtgatctcagctcactgcaacctctgcctcctggattcaagcaattctcctgcctcagcctcccgagtagctgggattacaggcatgcgccaccatgccgggctaatttttgtatttttagtagagatggggtttcaccatgttgttcaagctggtctcgaactcctgacctcagttgatcctcccgctttggcctcccaaagtgctgggattacaggcgtgagctaccatgcccagcttctgagctctgtgaacccattaacattcctgaatcagtacagtgggattggctgttgctgcaacagagggataaataagagttgaggggccaggcgcggcgcctcatacctgtaatcccagcactttgggaggctgaggtgggtggatcatttgaggtcaggagttcgcgaccagcctggccaacatgctgaaaccccctctctactaaaaatactaaaatcagctgggtgttgtggtgggttcctgtaatcccagctacttgggaggctgagttaggagaaccacttgaacctgggaggcggaggttgcaatgagccaagattacgccactgcattccagcctgggcaacagagcgagactccatctcaaaaaacaaacaaaaaaaaaaagttaagggaggctgtgcattcctgtacatggtgggtctagtgatttttttgttttgttttgttttggagacagagttttgcttttgtcacccaggctggaatgcaatggcaggatgtcagcccactgcaccctctgcctcctgggttcaaacgattctcctgcctcagcctcccaagtagctgggattacaggcacccgccaccacgcctggctgatttttgtatttttttagtagagacggggtttcatcatgttggccaggctggtcctgaactcctgacctcaggtaatccatccgcctcagcctcccaaagtgctgggattacaggcatgagccaccacgcccggccaggtctagtgacttttgggacagatgctcaaagcacacacatacggtcatatcaacaggagcagagccttgagtgagggaggttaggctggtcaggccactggggcaggaactctgttttgtttaaagcccagcctgagccatggatgggtaggcgttggggcccacagggacctacccttggaacctcgggccttcttaaggcctctctgggcttctgaatagctggaactttccagaacttttctggtcaacctgtgtggcccctggactcactctacccttttctagctggtcgtagaggctcaaagtcctagcgttgctcaagtttgagttgttagacctcagcctctcctcagaccccaaacttttacttaaaacaaagttttgctccattttttctttaatcagctcacttttttatttaagcaattttatgttttaaaataacttcaggctgggcgtggtggctcacgcctgtaatcccagcactttgggaggccaaggtgggtggatcacgaggtcaggagatcgagaccatcctggctaacacggtgaaaccccgtctctactaaaaatgcaaaaaaattagccaggcatggtggcgggcgcctatagtcccagctactcgggaggctgaggcaggagaatggcgtgaatccgggaggtgaagcttgcagtgagccgagatcacgccacagcactcccgcctgggtgacacagtgagactctgtctcaaaaaaataaaaataaaaatagataacttcagctgggcacggtggctcacactgctaatcccagagctttgggagaccgagatgggaggttcacttgagcccaggagttccagaccagcctgggaaacatagcgagaccctgtctctgcaaaagattaaaaattagccgcatgtggtagcaagcgcctgtggttccaattactcaggagactgaggccagaggatccctggagcccaggagtttgaggctgcagtgagctatgattgcaccaccgcactccagccttgggtgagagtgagaccctgtctctgtaaaaaaaaataagaagaagaagaaaaaataaaataaaaataaaaataaaaataacttcttcaatttgtctttagttctacatgtaaaaaggtaccaaagtggaaatctctgtccctgtgtccagccacttagctccctggaggcagcactgtttctacacatacatacaatctttccctttttccttttttcttttttttttgagacagagttttggtctgttgccccggctggagtgcagtggtgcggtctgagctcactgcagcctccacctcccgggttcaagcgattctcctgcctcagcctcctgagtagcggggattacaggcatgcaccaccacgcctggctgatttttgtatttttagaagagacggggtttcaccatgttggtcaggcgggcctcaaactcctgatctagtgagcctgccccagcctctcaaagtgctgggattacaggtatgagccaccatgcccaaccccttttttcattttaaaaaatcgtacaacggtaccatttgcgacactttattacctcttgcttatacacacctcctccatttaataatatatcttattgttcctaccctagtagtttattttaaaaagcatcctggctgggcagggtggctcacgcctgtaatcccagcactttgggaggctgaggtggttggatcacctgaggccaagagtttgagaccagcctggccaacatggggaaaccctgtctctactaaaaatacaaaaattagccgggcgtgatgtgtgcctgtaatcccagctacttgggcggctgaggcagaagagtccacttgaacccaggaggcggaggttgcagtgagccaagatcgcgccctgcacttgagcctgggtgacggagtgagaccctgccgcagaaataaataaataaataagtaatcaaaaagcatctcgaggtgagaggatcgcttgaggacaggagtttgagaccagcctgggcaacatggcaaaactcagtacaaaaattagctgggcgtgatggcatgtgactgtagtcccagttactcaggaggctgagttgggaggatcgcttgagcctgggagattgaggctgcagtgaggtatgttcgtgccattgcactccagcctgggcagaagagcgagtccctgtctgaaacaaaaaagcatcctttcccccaccccccactcccttttttttttttttttggcagtttctacatttatttaaacacaaaacatgcacacgagccgtctactcattttcttcactgcgcagcctggcattggggttggtgactctgacggcagctgggcggccctttccacgttggttttgcgtttcttggaggaaacactgtgagcgatctcagcacagggagatttgttgcacatcagcacttccagctccttgacgctggggaccaggaacttccggaagccactgggcagcatgtgctttgttttttgttgctcccgtaaccactgttgggcatcaagatctggcccttgaacgttctgcgaaccctgttgtcaatacctctgggtttcccccagttaacgcttaattttgacatattggtctgactggtgccggatgaacgccttgatcctctttttgacgatcttgggcttcatgaggagtctgagggcggccatgatgctgaggaggagacggctgccacctccttaggcagcgccgaggaagggaggcttttcctccttttttttttttttgagatggagtctcactctgtcgcccaggctggagtgcggtggcgcgatctcggctcactgcaagcttcgcctcctgggttcactccattctcctgcctcagcctcccgagtagctgggactacaggtgcctgccacctcgcccagctaattttttgtatttttagtagagatggggttttcaccatgttagccaggatggtctcgatctcctgacctcgtgatccacccacctcagtctcccagagtgctgggattacaggcatgagccaccgcacccggctttttccccctttttatacttatgtagtattctgctgcatagatagcctggcattcacttgatcagttcctttttattggatatataggctgcttcctaagggctcttgtaagtatgttgcagccctcggggaaaaaaaataaaaaaattagccaggcgtggctgtgggtacctgtagtcccagctacttgggaggctgaggcaggagaatggcatgaacccaggaggcagagcttgccgtgagccaagatcgcgccactgcactccagcctgggcgacagagcgagactcagtctcttaaaaaaaaaaaaaaaaaacacaaaactcagcagtagctccattcatcttaggggatacattgtaacattactgtgtactgtgaccggaaatctagtgtccctctgggagatgcaaaggttactgtaaaaatagtcgataaaaaggaaacaatgttaatgaattctccacagatactgtagcctgtcccaggtgcagcttgatgaggttgctctaggatgttaaaataggcatactcacctgcctggtggctcacgcctgtaatcccagcactttgggagactgaggcgggcggatcacgaggtcaggagatcgagaccatcctggctaacacggtgaaacccccatctctactaaaaatacaaaaaaattagctgggcatggtggcgggcatctgtagtcccagctccttgggaggctgaggaaggagaatggtgtgaacctgggaggcggagcttgaagtgagccgagatcgcaccactgcactccagcctgggcaacagagtgagattctgtctcaaaaaaaacaaaaacaaaaaaaaaacacgtactccaggccaggcgcagtggctcacacctgtaatcccagcactttgggaggccaaggtaggtggatcacttgaggtcaggagttcaagaccaccctggccaacatggtgaaaccccatctctactaaaaatacaaaaattagccaggcgtggtggtgcgtgcctgtaatcccagctacttaggagttagaggtaggagaatcacttgaacctgggaggcggaggttgcattgagccgagatggcaccactgcactctggcctggtgacagagcaacacttcatctcaaaaaacaaaaacaaaaacaaaaacactcctactccaggctttattgcagagactgaatgaatacatcagatcgtcatggagggtgaagaatgtggatgtgaaagccaggctggttcaaaggagcacgttcagctcagccaccgcctggctgtgcatgtccagccaaagcaagtcccttgcctggtttgtgtctcagtttcctcacatgtaaaatggggatggcagagctgggtatggtggttgacgcctataaatccagcagcttgggaggctgaagtgggaggattgctcgaggctaggagtttgagaccaggctggacaacatagctacttgggaggccaaggtgggaggatcacttgaggccaggagttccagaccagcctgggcaacatagtgagaccccaggctggtttttgtttttgtttttgttttttgagacggagtctcactctgtcgcccaggttgaagtgtggtgccatgatcttggctcactgcagcctccatctcccaggttcaagcaattcttctgcctcagcctgccaagtagttgggattacaggtgcccaccactatgtctggctaatgtttgtttccttagtagagacggggtttcaccatgttggtcaggctggtctcgaactgctgacctcaggtgatcctccggcctcagcctcccaaagtgctggggttacagatgtgatggccttcacctgtagtcccagctacttgggaggccaaggtgggaggattgcttgagcctgggaggtctaggctgcagtgagccaagaccgtgccactgcactccagcctgggtgacagagtgaatccctatctctcaggaaaaaaaaaaagaaaagaaaaagaaaagaaggctgggtgcagcggctcatgcctgtaatcccagcactttgggaggcgaaggcaggcagatcatgaggtcaggagaccgagaccatcctggttaacatggtaaagccccatctctactaaaaatacaaaaagttagccgggcatggtggtgggcacctgtagtcccagctactcgggaggctgaggcaggagaatcgcttgaacctgagaggcagaagttgcagtgagccgagatcacaccactgtactccagcttgggcgacagagtgagacactgtctggaaaaaaaaaaaaaagaaaagaaaatgacgatcaggatggcactgcctctccatgctgttggagaggttaaatattaaatattacttcccctttctgagctcctgcctccagctcagagcactctcctgggcactgacccctcgtgtccagccacctcctggatgcctcccaagaagctggagctctgggttcaaatactggctctgacctcatgtcccatggtcaccccccgcccccatgctgggtgcctcagtttccccacatgtctcaccaggaaattagaccaagttattctggcaagtgctttcagggccctccagggtttggctggtcaccatggagggggggttcaggtgctgaatttagggaccccagcatctcacaggtttccccttccatctttcccagtggcactgtgtctgagcaggtgtgcccaggtgaggttgtatccactgtgtctgagcaggtgtgcccaggtgaggttgtatccactgtgtgtgagcaggtgtggctgttgcaggtggaagtggggatatgtgggcacctgggtgcccatgggtgccgtggtggggggcgaggtgggagggtgtttgttgtcatccttgctttcctcctcctctcttcattggccacatggcacttccgtgggcagggcatggaatgcaggttttcgtggctccttggtatcctagccagatgcctggacccatcagcgcccccccttctttccactgtatccccagagccccccaggtttatcaaagaacccaaggaccagatcggcgtgtcggggggtgtggcctctttcgtgtgtcaggccacgggtgaccccaagccacgagtgacctggaacaagaagggcaagaaggtcaactctcagcgctttgaggtgagttggggtagggggaggcagcctggggtcaatgctcccccaaggcagggccacagcacctcagccacagtggacttcgtggacacgttccccaggtggagcatcatctgtggccaagcaagcatggctgcgcagcctccagccagggccctgcccgccttacttgctctaccagctccatgctgtgtggcctcaagcaaagccctgccctctctgagtcttcttcacagtgcagtgtacacagcaccactgcacacagaaggaactttacaactctcaatacacgcccatgatcacacgtgcacattgacacattccgcatgtcttcacatgcctttgcatgcctgtcacatgggcttgcagacatttgtcaccctcacacatgcacacaacaccctgttcttatatgtacccaaatctggtccctgtctgcggcgcatttatactggacaccgacacacgtgcacattcagcctgcagcaattctcccagtcacccccaggatcatggggctgactgccattcccctacagcccagcctagcccagcctagacaggaaccttcctgggaccccagcctcactctgttcttttttcccctgcccatggcagacgattgagtttgatgagagtgcaggggcagtgctgaggatccagccgctgaggacaccgcgggatgaaaacgtgtacgagtgtgtggcccagaactcggttggggagatcacagtccatgccaagcttactgtcctccgaggtattgggctcctcagggcgggcggaggataaactgggccctgggctttggggacaagcacaaaaggaatacatgaaagttaccccaaaccctcctgctttgtgttttttgggaggaccttgatctcccttatccaaccagtaacagtctcagggcccagcgcctactggtggggatttattgtgcctttgcaagaaagaaaggaattgagagactagaatcatcctaatttataatcttcatcatgtgcttctgatgacaaaaggctgatccaactcagatgggttcaagcgagaaaggtaattgatgtgcagaattaaaattccatggagtggctggcttcaggttcagctggatccaggggctcattgaattttctcagagctcactctctgtctcttggctttgtttccttctgtattgatgatatccttagtgagacttcccccactgccaatgaacttggatcccattcccttccctgaactaatcactgtggctcaggaggaacaaacactctgattggctagactcctacctggagccaagagtgagtgtggttcccccaaaggaataaagataaacagggttgggcatggtggctcacacctgcaatcccagcactttgggaggctgaggtgggaggatcacacgtgagactagactgggcaacatagcgagaccccatctctacaaaaaataaaggatgaacggatgctaggtaggcagaaactgctgccccacttccccagacattcacacgggcctgcagtgctgcttgctggtcttgtttaggtgcaattcttttttttttttttttttttttttttttttttttttttttttgagacagtcttgctttgttgcgctggccagagtacagtggcattatctcagctcactgcaacctccacctcccaggttcaagcgattctcctgcctcagcctcccaagtagctgggattacaggcacatgccaccacgcccagctaatttttgtatttttagtagagacagggtttcaccatgttggccagggtggtctcaaactcctgacctcaagtgatccgcctgcctcagcctcccaaagtgctgggattacaggcataagccactgcatgcagcctaaatgcatttcttgaacaagtcgcaaggggtttatagggggctgtctgagtgatgctgttttataatgaggaagcaattttgtgaacaaatttttgttcagcatcatttcaaacagccgcctacaaacccttgcccaaccactctgtgctgccttctccactgcagtttggtcttagcactgggcccagcctgtggagctcaagtcctggctgccacttggttgctgtgtgacctagggcaggttaatgaacttgtctgagctgtaattttctcatctctaaagtcgggctgagtacagggactcaaacatctgtaatcccagcactttgggaggccgaggcgggtgaatcacctgaggtcaaggagttcaagaccagcctggccaacatggtaaaagcccgtctctactaaaaatacaaaaattaggtgggtgaggtgacgtgcacctgtaatcccagctgctcaggaggctgaggcaggagagttgcttgaacccaagaggcggaggttgcagtgagctgagatcacaccattgcactccagcctgggcgacaagagcgaaactctgtctcaaaataaatgaatgaatgaatgaatgaatgaataaaaatgaaatggctgggcgcggtagctcacgccggtaatcccagcactttgggaggccaaggcgggcagatcgcgaggtcaggacttagagaccagcctggccaacatggtgaaaccctgtctgtactaaaaatacaaaaattagctgaacgtgatggtgggcacctgtaatcccagctattcgggaggctgaggcaggagaggatcgtttgaacctgggaggtggaggttgcagtgagccgagatcacgccatcgcactccagtctgggcgaccagggcaagactccgtctcagggaaaaaaaaaaaaaaaaaatcgggtaataatggttctcaccacaaggggtaattccgtgaaccagatgagtgagtgatacagggagcaagattagcacaatgccatatttggtcaattctacttccccgttactgtgttcatcactgttaggattctggtttagtaagatgcttgttaccaatccttctgcagccctcaggtgagtgggttccacctgtcacctgcagagacatcccagtcatgtatatgagccactgccagccattagctgggaggtgacccctgcatgtggcttcccaagatgaggctattccaatgtcctccacggcagcccagctgtgaccagcttggagccctgggaggggttatcacagcggccctagtagtactcgatttcagactctggtcccatgaaactgggactggcatcacaggcgagtgacctgtacagtcatccaggatccctccctcaggccctgtgctttggcctgtaatcccagcagtttgggaggccgaggcaagtggacaacttgaggtcaggagtttgagaccagcctggccaacatggtgaaaccccaaatctactaaaaatacaaaaattagccaggcatggtggtgtgcacctgtcatcctagctcccggattcttgggaggctgaggtaggaggattgcttgaacccgggaggcggaggctgcagtgagctgagattgcgccactgtactccagcctgggtgacacagggagactccatctcaaaagaaaaaaaagaaaggccccgtgctttttaaagctctgctgctggccacatgccgtggctcacacctgtaatgccaacacttcgggaggccaagtcaagaggatcacttgagcccaggagtttgagactagtctgggcaacatagagaccccatgcctcactgtccaagtctctacaaaaagaaaaatgtaaaacttagcctggtgtaatggggcctgcctatagtcccaagtacttaggaggctgaggcaggaggatcgcctgagcctcggagttcaaggctacagtgggctatgatcatgctactgcaccccagtctgggtgacagagcaagaccctgtctctaaaaggggggtgggggggagggggaagaaaagataaaagctcgctattgtggtctggaaagtcttaatacttttctaacaagagaccccacaatttcatttgcgtgggtcctggatcccatcctgctgggggatggaggaaggagcgggggcggagtcaggcccaccgtggcccgccctgctcttcctagcagataattttgccctgacctttcccaggctgggacagaacaacctgccttctcttgccccaagccagcctgcactctgagcactcagggtattaaaaggacaagctgtgaagccatcaactgcccggtcccccggagtctgggaaaaaaatgccccctgcgcccatccacactctttttttttttttttttttttgagatggagttttgctcttgttacccaggctggagtgcaatggcgtgatcttggctcactgcaacctccgcctcctgggttcaggcaattctcctggctcagcctcctgagtagctgggactataggcgcccactagcacgcccagctaatttttgtatttttagtagacacagtgtttcgccatgttggtcagactggtcttgaactcctgacctcaggtgatccacctcctcagcctcccaaagtgctgggattataggcgtcagccaccgtgccgagcccacaccctttttctctctcctctccggctgcctggaagggcctgcttgggctgggagctcattcttcctgctggggcccggggccttggataccctgacccgatcccacccaccttgccaccagccctgagctccgggcacctccggccagtccctggcagttggtgggggaccatctggactgccagggccactccctgccttcctcctcctctgccatctgattgtggaccccacacacgcctcctgcactgttgttttaattaaactccgaggaactgggcagagtgtggttcgcgatgtacgtttttgtttaattaggtgttctcggtagcgtcaggcggccgagacctccccctgcaccctggctggctgctgtccacacacgcttatggggacccctgcggggccacatacacacgggaattgctcacgtgtggacacgtgggcacgggcaagcgtgagcacatacatgcggcacacatgtacccacgcatgaatgaaatacagggttccctcgtaggcacacgagcatgcatagacacacagggggatgccgacagacccagctgtgggcaccaagaccaattgagtgaggcccagtcattttggccctcatctccgatgatgtcatcagtagccaatcagagccactttcaaggtcgtccaggtccctggcccaggaaggatggacaagaggcctgaactgcctcagacggcatccagccatgcccccttcagggccccgatgggttcagacctggttgagtggagggcttttgcagccggtcctgctcagcttcttctgagctcagccaggcctgggttcaaatccttgctacatgctggctgtatgacctgggacaagtgtgtgactgcctgtgcctccgtttccccctctatggaaaaggcttggaggagatcacaggttccgttgcctgcgctgcgcctgggatgctgttagtgctcagggctttccactggggcttttcccatccaagcagtgacccccgagctggaactgccgctctctcgtaatcctggggaggcagggattgattctggcttcccctccctggatcctgcccacctctcccttgccccatcttggcctcctgccctcctgctgcttgcctggggctcagtttcctcccaagatggtggcctcctgcccacctctgtccacggccacccacccctgagccccatgtcccatctgggcctttgtaagcaaatacatgtgtattctttaaacaaacagaaacattgtctacgccaggaagcagcacactttttctccagaccaccaggaagtaaatatttttggctctacaggctggacggtctctgtcgcagcaacaacccaaaactctgccattgtgttggaaaagctgagaaacgaatgggcgtggctgtgtgccaataaaactttatttaattaattaattaatttatttatttatttattttgagatggagtcttgctctgtcgcccaggctggagtgcagtggcgcgatctctgctcactgcaaagtccgcctcccggcttcacgccattctcctgcctcagcctcccgagtagctgggactacaggtgcccgtcaccaagcccggctaatcttctttgtagtttagtagagacggggtttcaccatgttcaccaggatggtctcgatctcctgacctcgtgatccacctgcctcggcctctcaaagtgctgggattataggcgtgacccactgtgcccggctgataaaactttattaataaatacagctacagggctgtttttgcggccctttaccccctccactccagcattcctaccagatgcagaggagctgcctatttcttggtcttaacagcactgcgatttgtcacttttttgtcacttgtccgttggagtggagttgggggggcgggggggcacggttattagtctgccctacgtggctgtttcctaatctcttttttttttttttttgagacagtcttgctctgtcgcccaggctggagtgcagtagcatgatctcagctcactgcaacctccacctcctgggtttaagcgattcttctgcctcagcctcccaaggaactgggattacaggcgtccgccaccatgcctggctaatttctgtatttttagtagagacggggtttcaccatgttggccaggctggtctcgaactcctgacctcaggtgatccacctgcttcgtcctcccaaagtgctgggattacaggtgtgagcccccacggccggcccatcttggtttgtagagcaccctggatgcaaggcagcccgtcacttagctggctcacctcatcacctcagcccagaagcatgttcccaccgctcagggagctctgggaccaggagattccagggacctgagtttctcctccttgtccccgctcccccccgcccccacatcaccaccggtctccatttaccatggaggggagccagaacctcaccacccaagagggataccgagcgctggagacctggtgccactcactcacctggtgctggaggattgagtgctcaaccaggattgagttgtttgttcattgattgagctcctactgtatgcccggttctgcgctgggtgctacggatgctgtggtgacacagatgcaccgttacagatagaattccctgctgcagaggagagtgaggagtgtgggggcatttagagcaagggagttggagctagtcccaggtggtgttccgatcagtagacagatgtgggaacggggattccaggctgagggaacagaacaggtgacccgagggagggggcttgatgactttgagagccgtggaggccagggtgactgggccagagagaaaaagaggagggaaggtggcaggggccagcagcgggcaagcaggcagggttccttctgagggtggccggaccacagaagggtgtacagcgggggagcagtgtggtttatgtttagaaaggcaggtggggctgtggctcacacctgtaatccaggcactttgggaggccacggcaggtggatgatgaggtcaggagttcgagaccagcctggccaacatggcaaaaccccttctctactaaaagtacaaaaattagccgggcgtggagtgcatgccactgccttccagcctggccgacgagtgagactccatctcaaaaaaaataaataaataaaaaataaaaacggtgtcctctgggccaggcacagtggctcatacctgtaaaatacctgtaatcccaatgctttgggaggtcaaggtgggaggattccttgagcccaggaggtcgagaccaatctgggcaacatggtgagaccctatctctatattttatttattttatttttatttttagtttgattttttttgagaccgagtctcactctgtcgcccaggctagagtgcagtggcgtgatcttggctcaccacaacctccgtctcccgggttcaagtgatttttctgcctcagtctcccgggtagctgggattacaagcatgcaccatcatgcccggctagtttttgtttttaatggagacagtgtttcaccatgtgggccaggctgggtcttaaactcctagccttaagtgatccacctgtctcggcctcccaaagtgctgggattacaggcatgagccaccgcgtccagctgatttatatgtatatatctctctgtttaaaaattttttaaaagataaaaagaaaggcgtcctcaggctgctgtaaatgaacccactcagcactgtggaccttggggccaggtttgtgcttatatgtagtagggtgtagagcatctctcagccccacccagtccattccaggagcacccacccccaagtcctgacccccaagttgtgacaacctcagatatccccagacatcgcccagtgtctcccagggcagcagagtcaccccccagtgagaaatatcattctacctggctcagctccctgacaccagcctcccccttgtacagaagagggacccaaccaggtgcagtggctcatgcctgcaatcccagtgctttgggaggcagaagtgggaggattgcttggggccaggagtttgagacccgcctggacaacacagcgagaccccctctctacaaaaattttaaaaattagccaggcgtggtggcgcatgcttgtagtcccggctactcaggaggctgaggcaggaggatcacttgagcccaggttgttgagactgcggtgagctatgatcgagccactgtactccagcctgggcaacagagcaagaccctgtctcaaaaaaagagagagaactggctgtaaaggatggcacccatggctgaggtcacgcagccagggaggagctggacccgtggccaggcagtccctgagccccaggctcaggagtggctgtgcagtgctcagaaccatgcccatttctctcacgtctctgccctcagaggaccagctgccctctggcttccccaacatcgacatgggcccacagttgaaggtggtggagcggacacggacagccaccatgctctgtgcagccagcggcaaccctgaccctgagatcacctggttcaaggacttcctgcctgtggatcctagtgccagcaatggacgcatcaaacagctgcgatcaggtgagtgagggtggctgacaggacgtgggagccttggagggcagcagggggtggggagcatctccagggcccaagttctggggactactggaggacagacagagacagtgtgtggccgtggcaggcgttggctctgggggagtgggggcaaatgtcttcaaagccgcacactgaggatattagggggcacagatgggcatgagtactggagagctatagagggtggtagaagaatagtgcttcaacacctggccaatgaagatataatggatgcagacatgcacaaggatggggagttatatagggccatgggcggggagagtgtctcccatgctcaggcagtgagatcacagctgtcacccacagacagttgtgggtgataggaagccactgagggggctgggcgcagtggcttatgcctgtaatccgagctctttgggaggccaaggcagaaggcttgcttgaggccaggagtttgagaccagcatgggcaatgtagcaagacgctcatctctgaaaagaaagaaagccgccaaggatggtgggtatcggcagtgcccaagcaatgggagacagacaggcagaagtgtcagtgagccatggagggtgacaaggggagaccagcttcaatgggtatgtaatagggagctatggaaggtggcagagagcatcctctacagaagcattaggcacgtccagtgagaatacagtggggcccagacaggcaggagtagtggaagctgtgtaaggcaatggcaggtgtccagaggtgacaagtaaggtgatggggagccatagaaaactacagggagagagtctctccaaggctgagcagtaagaggtgatggcggggcttcaggtctgtaagggggcacagacgggcagtccagcctgtctgttcaggccctggctgggctagggagaccttgggacagagccttggagacgggtggttccaccattgtgtctccacaaagcacccacatggcccatctgtgggtccatctgtctggggggctgtgtctgcgtgtgtgttgtccccccgcttcacgacatgtccacgtgtgcatgtctgggctccctgtgccggcctggcctgcctcccgcctgtgggcacaggtgtgcaggcctggctgctgtcacacacgggcctcccttcccccgcccggagcaaagtgtccgcctgcatctcggccacacgtgcacccacattgcagtcactcccgtgcctgtgcacacagggcagtgatccgcgcacgcgcctctcagcctgcacctccctagtgctcgtgcgtggggacacacgtggtggggatgtgtctccatacattgggagcacacgtgtggctgcacatgcgtgtttgtctgtgtcacggatttactgttttcagttgtgccgggcttgacgccctgccttcgttcaggtcactgttgagatcaccctgcacctccacacacccctctttttgctttctctctccccttcctccgaggatcccgccacggagccccctttgcagcagtacctccccgaatcatccccagcgcccctcactcactgcccatcgttgccccccaccaaagagcctacctacctgttctccgctttctgctcctcctccttagtgaaaaccaagccccaaaaagatcagacgtggagcccagaatgtcttccttggagagactcagacctcacactggggggacaaatcgcctcctatctcccagcctggctgaggatgtttgcagctcataggcctctgcaagcagggggagcggtggcctcagtttccccataaaacatgcctattttccttgtgttctggtgacctctccaggatggaatgtctagcgcctgctaggtgctcagtgaacataagccactttaggaatcataagaaatgttgttattattattctgagcgtccccctcccctcccccgccctcccctcctcgctgaggacataggattctttgtaaccacccgttaacgttcctgttctcttttcttatcctcctgttgtaaacgttcagaaacctttggtaagtctgatttcatcaccacgtcaacaactaacgccccatcccctttgtgccccttctgctccccttctcctcaccaaactaacagccaccgtgatggtaactgtgacccactctgcctggtatgtttactaacgccctctccctcctccccgcgaccggcccctcagccctccccagctggcactagtgtgcggcagagaaagaaacccaaaggaaaagaggaaaagagacaagtccacccaaaggaaaagaggaaaagagacaaggccaggcagacctcaaaatgcaggctgaggggggttcagaggcatcctcgggctcatggaacttcgcacctaacaacatttcctcccgcacttttttcttggccttagccctgctggaaaaggatttaattacagaagtttgggggctggggctgggcgtggagtggagatttttccagcctagctgctactgggcacttctcatccacttttctcagcattcagcctgaatctcctgaaatcagttgtgggactaattcatactaaagtgagagtgactgatatcagcctatgctatcagtcccttcatctctctcagggtgggaggcggaacaccttttaatggctaagggatacattgtttccacttgatagaatagccctgagtgatttggccaacttgaaaagacttctttttttttgagacagtcttaatctgtcacccaggctggagtgcagtggcaccatctcggctcactaaagcctccacgtccccgattcaagcaattctcctgcctcagtctcctgagtagcagggattacaggcacccaccaccacacccggctaatttttgtatttttagtagagacggggtttcaccatgttggccaggctggtaccccaaactcctgacctcaggtgatccacctgcctcggcttcccaaagtgctaggattacaggcgtgagccaccacgcccggcctgaaaagactttctttaaccctgacttccaccatcgtcatcccagtgtcctagcaaaggccagcctggatttccgctgactcggattccagttctccagggtgtaactttaggcaaacttctgtttgctcccctctaaagggtgcagagtggcctcacgagaacacctcgaaagataattcagggactccgaaaagttcaggccagaagctcctcatcccaggaggctggagagctaggctcagtgcccacccctgcctcatgatgggacctcagagattcccctctgagcctcagttggcccgtctgcaaaatgggtttgggccacgcagaaggagctgctttttcacgtgtctggccactcctggcagatcgaggtcttgggatcttcttggccccagccagccgagttatcaagttatcggccatcagcagcagagagaagggggcccaaaacttctccctggatcatgagcagagggagtaacgttccagccccgagccgccttttcctgctttgcagttaaccatccctgcctccaaggcccagataaacaggtctttcctcttttcctcttggtaagaaggctgccaggctctccctgtccctcctactcggcctgtctgtgacacccttccttggctgtattaatgcccggcactggggcactaaccccacccacctcccctgtctctaacaagataattcctgttgctccttgtcccctcctagtctggacttgaaagcagtctcttgctgggctccctaagacggaaagaaggaagggggggtgatgaacctccctccctcagcccagagggaaggccaccaaagggccaggctgacagaccttccctgggcatagagacgcccctcacccatccagagaggcttctagactccatccctccacccccagagattcccgccttctacaggaaaccctcctgctttgacaagccctcagacctcaccctgctcctaggaggcctctcctagcttcctcagcaaagcccgcagggtccgtatgcccagggctggctcgatcccctccctggggcagagtgctgaggcctggcttacggcccagcccagcttccctggggccccccggccccccaacaaccgccttgtgacattcacctggttctctctgcttcccttacagaaagcactccgattcgaggtaagaggctcctcactcacccacccactcacgctcgctcttgctgcccactcagcccctcaggctccacagggcctgcccccaggcccccttccatgcgtgtccaccctgccaccacctttgttccacccagtgttagtccggtctctgcgtctgtgtctgtgtctgtggccagggggctgaatgtggccgaggtgactcctcaggtcacctggctgtggaaggcgggggcgctctctccccctctgggaaggaccctacctggcaagacccccagagtgctgggagccccatgtggtttagccacctctaaaatgagcatgcaggccgggcgcggtggttcacgcctgtaatcccagcactgtgggaggccgaggcaggtgaatcacctgaggtcaggagttcaagaccagcctgggcagcatggtcaaaccccatctctactaaaaacacaaaaattagccgggcatggtggtgggcgcctgtaatcccagctactcaggaggctgaggcaggagaatcacttgaacctgggaggcagaggttgcagtgagccactaagatcatgccactgcactccagcctgggtgacagaatgagactcctcaaaaaaaaaaaaaaaaagaaacaaaacgaacatgcaaagtgggccttccagctccacagttctttctgatctggaggttctaacccaaggacacatagagacatgcactctcaggtcggtcggctccatccctttccccctacccaacttttgcctcagagaagagccaagagaaggcctgagtgggaacctggggaagaatcagtgtccagccctacctttgctacaaatgctgtgtgacctagagcagtctcctgaccctctctgggcctccttagtgatggagatactgcctgcttcccagaacctcgaggtccagggagaataggcagtgctctatggggacggagtctttatccatgacctccagagagaatcccccaatctgtgggtccatgtcatttctgtgggatagtcacaaacaaggctggaaagctcagtctggatttttggacccctggtgccataggctgtgattgaagcctgtgattcaaacacatttggggatattcactcatcttcatttactcatggtcgacaggcacttgggtccaggcctgtgattcaaacatacttagatatgttacactaagttttatttacttatgaaaataaggactttgatccaggcctgtgattacaaacatacgtgtggaaacttttgccttattttttatttgtgggcactcaagtttttatcaaggcctataatttcagatatactcagcttcatttactggtgaaaatgagggacttgtttcaggcatgtggtttcagatatctttggggaaatcttggcctcgttttcattttctcatgggcacagtagtgtttgatcaaggcctgtgatttcagatattcttgagaaggttcattcatttttataggttcatggaaagtgaagtctttgatttgggtctgtgatttcaaatgtattgaggggaaatcagtcctattttcatctactcatgagcaccaagaccttggatttgggcctgtggttttaaatatgcttgaaaaattgccctcttctttgtatctttatttactcatgaccaacaaattctttgatccaggcttggggcattttagcctcaactttacttgtgagtatgagaggctttcaatcagacccctgatttcaaacacttttgaaaatttaatctaatcctgggtacctatgagcatcatagaattgggtctaggaccataatttcaaacacacttgaagaagtccattcagttactcgtgagcaccaggatgttgacttcaggcttgtggttttaaatacaagcaggtaaattcagacttttatttagatgacttaaaatacttggggaaattctgacttctcttcaatcagtttcaggccagaaggcttggatccatggctttgatctcaaacatacttgatacacaccagcatcaacgtttttttactttagctgcctcagggtataacctaatcctgaaatttcaaacacacctgaagaagtacaaccccgtctccatttacttaggggcgttgaggactttgaggactaggattccaaacacaactgaggaacccagacccatgttcatttactcacgggcacctaatgaaacaaagcaaccaccagctccctgaattttcacatgatttgtggtgggccccctcaggtccctggatctatcaaatgatggttatgagttgtgaatgccaacccactctttccttctggccaggagagcatcacttagtgctttctcaaacacacttttctccatctttgtaattcaacttggcagtcatgaggcagcagtctcatgaccagctgcttttccactgtgagtggagaatttgctggggaatctggtgtgtccaggccctggtggggctagatgtgggggtggaggttatacaaggcctggccaagctagggcttccaggttttctcacctcttatctgcctctctggcccagccagcacaggagacaggccagagagcactctggtgggggagctttccctgttcactcctgggcccccctctaacagaccaagctttttcccatcttcccacaggagccctgcagattgaaagcagtgaggaaaccgaccagggcaaatatgagtgtgtggccaccaacagcgccggcgtgcgctactcctcacctgccaacctctacgtgcgaggtagggaacgccgcgtccaggtcaaaggcagggacccgccccgtcccatccccctccctcctcgggctcaccgcctccgggctcccccgtcccctgcacacccaagcaggaaggcccctccccggtcgcggtgggaccttggccgaggcggcgtccctctctgggccttagtttccttgcctgtagaatgggggcagcttaaattcccttcctcgtgccttgcgagagatgccccgtgggcagcaggctctggggaggcagtgatggtggggtaggaaggcgtggggcagggtgagggggtcagtcttgggcctacagggccaggggagcctgcaggcctctgccccgccccaccccacctgcctgcccacccgagttggcttctgtggtgtgtcgtgccttcccgtgctgtgtgctggctttgcttctcccaactttgtggcctcgtagctccccgcttcactgccccccctaatccagggccctcaatctagaagttgaggaggggtcttcgaggctgcccctcccaccccccaccagctggtccggagcccggcgccttctgcctcttcctaatgactctccgagaagggcagtccaggccgatgggaggctggagccacagcgggcacaggcacccggggggctcggtagttgcaccgagctggggcaggcacggcacccagcgaagcttgttactctcagtttccaattcccactcagtcatgcccagcagcgtgggggcctcgccccagcacctccccgctcccctgccccaaggaagcttacttccttgtgggtcccttggtgctgcctcaaaggtgccagggggctcccttgcccccagatgcctccgttggcctcctcttatctcgtgtgttggctcttgggtcttttcagaagttgtagtcttcctcctcctcctccctttcacctcctagtcgctgtttctgatcagatttgtggtagaaacagcttctgccaggatgccagggcgcccttcaatccctagggctgccccagttagcgctgtgtaattgactcaatcacatccttctctgggcctcagtttcccccatgcgaagtaccaagccctagttgttctccatccaggaggccgtgtggctcatcccttacccacagaccctcactcaccaccttcgtccctaccatccacagcacccgctggccctgaagtgattccttggtcctggacctggggttttgcattttgtctctgaggaggaggtcgcatggagggggaggggatggggatgtgggtgtatggacgggtggtgggtaggggcacgtctgatccactgtcacggcacctctggtgagttgtaatgcgttcgtgactccaactgactggtcttcacctaacctgcttatttctctgccaacacctgggatgggggtttgtaaatccagcagggctcaaaagagtgtggacagcgggtgtggggcagggagccgggtggggcctttagtgagggtgcaagaagggaggggggcttctgtgtcctcatcccccagaggtggggtccccagcatgagcctccatcctcttggggacagttgagggggtgaggttctgtttccccatctttgcctgggtcttagatgctccagaatcaagcttagaaaagaatggagaaaggcaaatgtagtagatcttgtatgttgagggcagagtggagagaagacaaaaaggtcattgctgttctttttattttttaattttaatttttttttttggtttaaagaaaaaaaaaacaacaaactattgcagcttttagccttgggaagtcctctttttactctctgtatctcccctattttttctcttcttcccatgtcattgtgttctgcatcaaaccccctttacatccctcagagcttcgagaaggttggtgtgtttttttcttttttactttctttacccacatttttacattctgaaaatgacacgcacacacacaaaacacatgtagacaccacacggacacacgctgacacacacacggaaaaaaaatagaaaaatgaacaacaattttaaaagatggaatatgaatcataagcacaaagcaaactaaacaaaaaaccgccaagcgagcgcaaaacccagaaagacacgaaaatctcagccgcgcccagcccggcctagggccggccgcacacatgcgatgcttgcattatggtcgcctctgcatccgttggtaatgttgttgctaagttttttgcactcctcgtcgtcatggtaacttgggcctgatgcatgctgggagaatgtaaaaatttctctgcatgcctctcttgtcggtttggtttggtgaattcccaacgccttctgttggagtgtttctcgtcttctgttctgtttttctcttttggttttcttttttcctttttcttttttttttcttttttctttttttctttggttttgtttcgttttggggggaaaaaatcaaatggttcttttttcccctcccattcccttcacctggtcctcttctcccccaaagcgactcgtctttggttctgtcttgtgtctgtgctatttggttttggtttcttgggttgagctgttctgtggttgttctccaaacgcattgtgatttaaaagccagtgttcctgtcccagctatcatccaggagggcagaaggggtcccacagctgggatgccagggcagaaagtgaccacttaggcacaagggcacaagggaggtggcacagacagatggcaggaggtcagaacatgggtggggtgtgtggagagagcagcccaattttcctgacctcactggccagctcctctttccccagaggtagtaactcagctccctgcctccaggcagccatgttggaaggaagcttaagcaggcatgttcccatccttctacctcagaatgagactgcctcctgcgctcccaaataagcccaaacccaggcatttgaagtactgctgaatgtgcactagcattttgagttcttattcattttagtgacatatccaaacatggattatggtcagttttaaatgaggggagaggacaataacagcaccattttcacaggaccatgcatactcggtgtgtaattggcactcaaaatggcagctgctgtgatgactgtatgcattctggctgggtaccaatccagaagtgcccattaggaaatgtagcctgacagactttggaagaaagacttggccaattatctgacctggaacactatgcctctgcctcccaggcagcactggctgcttcccccatcccctctcagactctgagcccggtcacaggcgtcttttgtccagcaacacagcatggtcactttctgagtacctgagcatcagagctgtggaattctggaaaaagcccaggggaaccttggacacagcctctcccctgctctctctgcctccccaactctccctctgcttctctttctctttttcaactctctttctctctgtgtgtctctcactcttgcaaatcttcagagatgcacacccccatcttctgctcccctccccaagaaaaggcacgcccttgagagtcttactccaacccccaataaaaaacaccccccaataaaatatccgtgtagatttttaaccaccaaccccagctgactaccccgtttagacgatgttgaggacagaagccagactttggaatcaccttttccccctcttgtccctcactcaccttctccaaacttaccaattattaaatcctggaccaagttggcaagaggactttagatgcagtaagatttgcttattaacccagcatgcgagctgccaggctgaccctcagagcagtgccaaaagatggcagaaaggggtctcccagcccctccaccaaagctaggtgctggtgcattggaactgagtgaacagagggtctctgaagaccacttggactttctgggcagtacagcccagaaagtgatgagagcataggctctggactctggcagacctggattccagttttgtctgtgcccctccctggctacgtgaccttgaactagtcatttgaccacttggagtccccacttcctcatctgaacagtagagatcataaatgaaccttgatcacagggtaggtgaaaggatttcattgaactaagtgctctgtgtaacaagcattcccagtaggattgttgtaaataaaggattcaagcaaatgatctgggcaagtcacttcccttctttgtgggtcttagtttccctgttatgtacttcacatgtccactgtttgaataagatggcatatgggaaacttttaaaaataattgtagggcagaagctaattcatttgctcagacctcatttttcgctagtgctttcctgagctcatctcatgatctcattttctcctggctccagccccatccccatgagatcagcggactacccagagccatagctctcattttccccactgtcatttcctaggaagttctgtggccacagaaagggacaacaaaggcccatattaagtacaaggctacccttgcagataagtaccagcccaccagaagagatgggctacctggcatgttctgaaagctacaagtatttacattgttgccagagcagccagtcacaggaattaattgcaaaaaggggaaaaaatgcaacccctaatctcactcttatttattttctaaactgacagatctccagctttggctgactctgggtggtacagggggtaaaatattcaggcttgcccttctcctcctgaaatttatctctgggatccaaagggcttaagtcaaaatgtgtccatttttaaaaatacagctggcagctccactcccagctgtagttgaattctccctcctccgacttttgacaaggtgggaagatgatcagattatattctccctgactatgaatgttcttttctggttctttaaaactctaagcctttagagtatgagttagtggctaaaggtagtgtctttactgcctggtcttgcaacatagcattattgaaacgtagtgcacaggacccatgtaaatggtttgccaccctctgatgccaggggaacttggaaaaagctggagacatcaggaggaattttctttttcttttactttttttttttttttttttttttgagatggagtttcactcttgtcactcaggctggagtgcattgacgtgatctcggctcactgcaacctcctcctcctgggttcaatgattctcctgtctcagcctcccaagtagctgggattacagttgccctccaccacgcctggctaatttttgtatttttattagagacagggttttgccatgttggccaggctggtctcctgacctcaggtaatctacctgcctcggcctccaaaagtgctgggattacaggtgtgagccaccacactcggcctttttttttttttttttttttttgacagaatctcactgtgttgcccaggctgaagtacagtggcgcaatctcggctcactgcaacctccgcctcctgggttcaagtgattctcctgcctcagtcccctgagtagctgggattacaggcacctggcaccacgctcagataatttttgtatttctggtagagacagggtttcaccatgttggccaggctggtctcaaactcctgacctcaggtgattcacccacctcagcctcccaaagtgctgggattgcaggagtgagccaccgtgccctgccggttgagcaatgttctaccttgaagggtctgaggatatcttcaaggttttggaagaggccgatgggactggcataatttaacgcagagtcttgagtgctaagctaagaaaccagtacctgctgcgtgacaccagcaaggcctagattgcccgattcccaggtcttctttttcagtatttgtgacctgtccccaggccttttcagatcaatccatctatgcaaatttatggggaaaaattgttttttaaattaaatttccaatacccaagccctaaaattgatggatgtgaccccaggtgttccccttacctcttggccccccaaaacagggacagacatagatggtgggctggaacacccctcacctcctgtattcccagaaagcctcgcgttgaggtgtgttggccagctccctagtttgtgcttactatacctggccacgcctccctacctaaggccgctggcttaaccctaggggcaggcagtgttagatcagacccagaccttctcatcccaccctcatcacatcggggagaggggactccaggggcgggaaggcaggcgtccctccatttggccagggtgggcggcgaggagggggtcactctgcaggaacactgagctctgaacacctctcgcctgctgcctgcctcacaccctctgcattcgctgtttcctctgttgggggagggggtttgtgaggggaatattagattacaccttgtcatttggaaagccccgtgtctccggcggccacagcgaggttgggggggtggtgagggaagtccatggattggccagaactgggggaaaaacaaaaagaaatgagagaaagagagagcgggtaccaaaaaaaaaaaaaaaagtatctagagcgcatccaaaggtccagctcaaaataccaatgacttgccaaggccgctggtcagcccgtgggccagggagccagccaggtccctccgggccttgcacgctacctcagtggcggagggagctggcggtgggggggagaggcgggagggagggaaggaggcacagcaaaatgatggagaagggtagcagaaaatacgcaggtgggctgaggggcggtgcccaccctccttgggctacaggctggagttcaaaggagaggacaagaagtgggctatggaattaggaatgtttccaacccctcccttcccccccgacaggtgtaatctaagcttaaataaaccccccggaggctgcacaatttcttggcatctctcccctgccctctccatccgcatattcattttggagtttggagaagtatctagaatcttctcccaccccaaaatgcccagcagagcccccccccccccccccgccgcccccgcaccccttggagctgcggcttgctgaatcgttgagatgtctgacactgtcggggttccctacctagtgcttcaaccagatcacctcacttttgagtttccttcctcccctttaccctcgcccccactcccaacccccttttttggtttttgtgtttctgcctcttcctgcctgggggaggggtcgtctcctggttgctttgacggtttttgagttgtttttccatgtgaatcggggtttgaagcgaccaccccccacaacctgccacccccccaccccaccgcccaccctcccgaccccctcgcccaagcgggtccccccgctgggaccccccggctactcggtgacgagaagagctggaaaccagagccagctggagcgtgctgaggaccaaggctggtgtggtccccttataacctgcctggaccctccattcaggtcggtaattgggggcgggggtcccgcggggcctctccgtcccgtgggcaccgtgcccaccttactttctaacctgcacctcaagcttgcagccgggggtggaaagatgacatgggtgggtgccagcaccaaaccgggcagaacacagcgtcagtcacccgcccttgccagctgggaggatgcccagaggccttacaaggccgccagggagggggagatctgaaccacctagtgccaatgggcatgatcctcccttaaactcgtggcaaaccagccctcttcctagagcacaccctttgaaacaaatgaggcgggtgagaaagcaagtgggcaagtttcttaggtgtcagaggagccattataatttattggcaggtccatttaactgatggttgccggccatgttggggattgtgaaccctaaaattactgctgttgataaatgccctcccacattaaactgatttatagttaaccctgtactgcaagtcttttacatattcaacaggtatccccttgtctctacttctaaaaaaagttaaaggtaccttgcaaattatgacatccctatggagagatattttaagataagatcaaacaggaacagggtcttttggataagatgttgacccttacagaatagataaattggcctggcacttaacctttagatcctaaagctagatcttactccctttgacgttccaacaattcctttgagtataagccttttactgatggcttctggacatttattatgatggctactggatattattcaaccagtggcaactggccattacatggagtttggtataggtgatgggtacttttttcctctttggtgtccccagtgggtcaggacacacaagtttgaaagagctagatgccaaggattgatacttggccgtaatcctcgttactcactatggtgagaaaagccttgttttctctgagaggcagcaaccaggctggcctggctttgagctctggctacactacttcctttctgtgagttccatctagtcttgtcacctctctgagtctctgttccctcatctgcaaaatgagcataaggacgtttgcctctcaaaattgttagaaggaagaaataagatgggaattcagtagaaattcttactgtttccatttcgtttgtttgtttgtttgtttatttatttatttatttggagatggagtctcgctctgtcacccaggctggagtgcggtggcacgatctcagttcactgcaacctctgcctcccgggttccggcagttctcctgcctcagcctccagagtaactgggattacaggcgcccgccgccacgcctggctaatttttgtatttttagtagagatgggattttgccatgttggccaggctggtctccaactcttgatctcaggtgatccacctgccacagcctcccaaagtgctgggatcacaggcatgagccactgcgcctgcctccatttcctttttataattcatccctgaactcccttaaggtagagaagctgtttgatcgtcccagcccctgggaggctgaaaggtaacttcaccagctccatgcctgagtttagcacctgctgtgccaggcactgcccgagtcaggttaatgcctttctcggcaggatacccaattccctccattttacagatgaggaaactgaggctccaggaggatgggggaccttcccaaggccactgggctcattcagccacatcagctcctccacgtctgcctggagaggtttactatgggattcagagaccttgaggagttcaccttgctttgtgatttcagctttgggacctccaacttgctcagccctaagagaggcagggtgtcgagaagtggactgagaggggctttagagagagagacttgagtcgggctgggttggtttgttccgaaggatttttgtccacgaggctcccacccctgtggccgccccaaacccgtggccaagactcttgggtgctggatggggaggccggatcctgtgttctgccagggggagggcgtctccctggggaagccacagtcctcctcctttcagaccccgattttcagccccacacccattccatgatttttttttttttcttcttgcttttgggtgttgtgttttgttttggagggagttttttcgtttgtttgttttttagattctctctttctttttttaaattgcttttgctttgttatttcatttgttcttcctggttttgttttggacgtcgataccgtttttctgtccttgaataattagctccccccacgctcatttccccattgtgttctgtttccatgacaactcagccttggcatcttacctgtgcctcgctgtgggtgccggcggctggcctggaccgcgtccgctggctggccggaccccattctccccccacacccccaccctccacccagccctaccagaaagccacccgccccccccatctcacggcacctcgcccctcacctccactctgtgtgcatggcccttctgtctccccatgccccgactcctcccctggagcagtaccctccactccctccatcctgtctccagtgcgacttctggacttttcgtggggcttctttttccgaagggctgccagcttccggaccccactcccacgcctgccgacgtccccccttctctttgcctcccgcccggattcaggaaggcaggggttgggatttccagctaggaaccgaggctggaagggagggagttggggccacctccctttatccccttgaacctcctcctcagcatcgtagaccagaccccctgagaccaccccctgaggctgcacctcccaatcccgtatgaaaatcagaggcaggtgttgatggggatcctaactgagcctccagtccgtcatctgtgcagttgaaacaagactccttcacccatccaaagctggggacgcacccatgtgtcttctttaggacctgagactgtcacccagggacaaccccttttcctgctagaaggatttattaggttcacccgagtccttcccacagcagttaaacagcccccgtgggccagcagtgctctttgcaagttcaggtgcccactggcctcctccccttccccctgtcctggaacccatctgacttccgaaaacggagcccccgaaatgcatcctgctgccttcatggtctttgagagaaggttgcatccacttccccgctaagaaacgcacagaacatgtccctgttctctcaacgttcaagttcattcccttgtttattattattaattattattattattattattattaatatactatttattattctgccagggctgtgatgtgacaatgtggcatgtcacattggatcacatctgctagaattaatatgcagcattgaagtgggcccccccttctttctttctttctctgcccctcccgctctctgtccctattttctattcttctctctttctccccattcattcgtctcctctctctggtgtcacgtaagtgtgcactagagatccccaaaatccagatcttccccctgcagttcctccctcctttctctctctgtctctcgctcgctctctctttctctctctctctctctctcaatctattttgcatgatgtttgtgactcagagctgcatctattgatggaaacttgtcagatctgacagaagcttttagaagggtttctatacccaaaaaccacaaaaatttcattagagtttctcatcttttcctggagggcccggtcggagggggcgggtaagtgggcagggccccaggtcactggggagtgggggactgacatctcaggctccctctgtctcctggcctcaacaagtgtcctatgaccgccacatccccagagaaccagggagggttggaagttgccccctctcctaacccactcacatcccgaaacttggcagccagaatcttccagcgctccccgaaggggatgcccccattcgggagtgatcggaggccacctgctccctgaagccactgctgtctgcaaagatgagaaaccgctgataaatttttgtgttcgtatctgtgctgtgaccagccaccctggtctatgaaaccctgttgctatgagataatgcttcatgtctattatatattgatatatctctctatatattgtttgtgaagtaccttcctttgaagttttggtttctttgtgttggaatttggttttttaatttggttcttttgttttcttttcatttgggggaaaaaaatcaacagagggaaaagaaccagccctgtttatatttaaatggttttcctttttttttctttttttctttctttccttctttctttcttccttcctgcttttctttttctcctccgcatcctgtcttcccctcccaacctcaccccacctcacccctcgcatctccctcccgccgccactgccgccccctccccagtccgccgcgtggccccgcgcttctccatcctgcccatgagccacgagatcatgccagggggcaacgtgaacatcacctgcgtggccgtgggctcgcccatgccatacgtgaagtggatgcagggggccgaggacctgacccccgaggatgacatgcccgtgggtcggaacgtgctggaactcacagatgtcaaggactcggccaactacacctgcgtggccatgtccagcctgggcgtcattgaggcggttgctcagatcacggtgaaatgtgagcagggcccatggggctcgtaggtggaggggcaggctggagtcgatccccgacttcaggcacaggtgggaggcagcgtgggagcacctccgagttaccctcttggcttcagtcattcatggctaggaaggggacggttgttctttgaacaggtattatttattggttataaatgatcctggctgggcgcagtgattcacgcctgtaatcccagcactttgggaggccaaggcaggaggtttgcttgagcccaggactttgagaccagcctgggcaacatagccaggccccttctctaccacaaaaaaatgaaacatttagccaagtgcagtggtgcatgcctttagtcccagctactcaggaggctgaggcaggaggactgcttgagcccaggagttcaaggctgcagtgagctatgatcgcactactgcagtccagcctgggcaacagagccggaccctgtctttaaaaataaataaaatgggccaggcgcggtggctcacgcctgtaatcccagcactttgggaggcctaggcgggcagatcacctgaggtcaggagtttgagaccagcctggccaacatggtgaaaccctgtctctactgaaaatacaaaaaaaaaaaaaaattagctgggcgtggtggcgcacatctgtaatcccagctactcgggaggctgaggcaggagaatcacttgaacccgggaggtggaggttgcagtgagccaagatcgagccattgcactccagcctgggtgacagagcaaggctccgtctaaaaaaaaaaaaaaagaaaaaagaaaaaaaaggccgggtgcggtggctcacacctgtaatcccagcacttcgggaggccgaggcgggcggatcacgaggtcaggagatcgagaccatcctggctaacacagtgaaacccgtctcaactgaaaatacaaaaaattagctgagtgtggtggcaggcgcctgtcgtcccagctactcgggaggctgaggcaggagaattgtgtgaacccgggagacggagcttgcagtgagccgagatcgtgccactgtgggtgacagagtgagactccatctcaaagaaaaattaaaaaaagaaaatgggtactgggtggaccctgggccatagtttgttaccccttgctgtagacgatgacaggagtcatgaggcccatgattactgctgggcaaatcaaggctcagagaaggcgactgaatggtgaaaagtcacacagccttcagggtcctcaaccaatgtgcccaatgcgtgacacagctgcctcctgcccacagctctccccaaagctcccgggactcccatggtgactgagaacacagccaccagcatcaccatcacgtgggactcgggcaacccagatcctgtgtcctattacgtcatcgaatataaatccaagagccaagacgggccgtatcagattaaagaggacatcaccaccacacgttacagcatcggcggcctgagccccaactcggagtacgagatctgggtgtcggccgtcaactccatcggccaggggccccccagcgagtccgtggtcacccgcacaggcgagcaggccccggccagcgcgccgcggaacgtgcaagcccggatgctcagcgcgaccaccatgattgtgcagtgggaggagccggtggagcccaacggcctgatccgcggctaccgcgtctactacaccatggaaccggagcaccccgtgggcaactggcagaagcacaacgtggacgacagcctgctgaccaccgtgggcagcctgctggaggacgagacctacaccgtgcgggtgctcgccttcacctccgtcggcgacgggcccctctcggaccccatccaggtcaagacgcagcagggaggtgaggcgtggggtcggcaggactcggggccccggccgtgcggcttggttctttgcatgctccctggaagccgcagagcggtttgtgggttatgcacggtgctttaagatattttcatgctagcatttaggccgggtgtggtggtttacgcctgtaatctcagtactttgggaggccaaggcgggcgggtcaccggaggtcaggagttcgagaccagcatggccaacatggtgaaaccccatctctactaaaaattagccgggcgtggtggcctgtgcttgtaatcctagctactcgggaggctgaggcaggagaatcacttgaacccaggaggtggaggttgcagtgagctgagatcatgccactgcactccagcctgggcgagagagtgagactccacctcaaaaaaacaattaaaaaatatatgtatatattttcatgctagcatttaaaatcggggagctaggccggaggcggtggctcacacctgtaaccccagcattttgggagtcggaggcgagtggattacctgaggtcaggagtttgagaccagcctggccaacaccgtgaaaccccatttctgttaataatacaaaagaaaaatagccggccgtggtggcgggtgcctgtaatcccagctactctggaggctgaggcaggagaatcacttgaacctgggcagagtttgcaatgagccaagatcgcaccattgcatccagcctgggcgacaagtgaaacgccatctcaaaaaaattaaattaaaaaaaaaaaaaaagagagctagccaggcttggtggcatgtgcctgtagtcccagctactcaagaggcagacgcaggaggatggcttaagcccaggagtttcagtccagcctggacaatatagcaataccccatttcttaagaaacatatataggacccagtgtagtggctcatgtctgtaatctcagcactttgggaggccaagataggaggatttattgaggccagcctggacaatgtagctagaccccatctctacacacacaaaaaattaaaaattagctaggcgtgatggtgcacacctgtagtcccagctactcaaggctgagatgagaggatcactggagcccaggagttcaaagctgcagtgagctatgatagcaccactacactccagcctgggcaacaaagcaagaccttgtctctaaaacaaaaacaaacattaaaataaaatcggagagggctgggtgtggtggcttatgcctgtgatcccagcacttcaggaggctgaggcgggtggatcacctgaggtcaggagtttgagaacagcctggccaacatgacaaaacctcatctctactaaaaaaaaaaaaaaaatacaaaaattagccagtcgtggtggcacacgcctgtagttccagctactcaggaggctgaggcaggagaatcacttgaactcgggaggcggtgagccaaaatgtcaccactgccctccagcctgggcaacagagcgagaccctgctcaaaaaaaataaaattggacagttatcctagccaaatttttggcttccctttaaaaaatctgatttgacaagcccatagggctaagatcaaacacccgggtagatcccagtggctgtgtgggagacaaggggggctgtgtgtgcttccttccctcttctctgttaactcctcacccctgttggcatttgagttttaagaacctccctcatctgttctcattagagggaccagcgccactgcttctgaaataatgaaaactgctggctcagcgtggtggcccacgcctgtaatcccagcacgttgggaggctgaggcaggtggatcacgaggtcaggagttcaagaccagcctggccaataatggtgaaaccccatctctaccaaaaatacaaaattagccgggcatgatagcgtgtgtctgtaatcccagctactcaagaggctgaggcaggtgaatcacttggacccaggaagcggaggttgtggtgagctgagatcgtgccactgcactccagtctgggcaacaagagcgaaactctgtctcaaaaataaaagaaaactgcctcacccctaaggcagtgggccagtctccctccctgctcaggtggcaggacttggaagaggcatttaggcatgggggaccccagctctctgggagtgtttgaccctttcctttggggctcaggctttcaaaacctaaagtctgggcagactcaggcacccactgaagcaaagggccgggacggttggcactctgtccatgtctgtgagcggatacagaatggagatccaaatgttaaaataggcaccctttgccaggcactgtggctcacacctgtaatcctgacactttgggaggctgaggcaggaggtttgcctgaagccaggagttcaagaccagcctgggcaacatagcaagacctcatgtgtacaaaaaattttaaaattagctgagtgtggtggtgtgtgcctgtggtcccagctacttaggagggtgaggcgagaggatcacctgagcccagaagttggaggctgtagtgagctatgatcgtgccactacactccagcctaggtgacacagtgagaccccgtttcaaaaactaaaataaaatgaaataaaataggcacacatatctccctgctaatttctctgtcatctaattcgcagccagcgagggcactggggtgatttgaaagggagatttttaggccaggtgtggtggttcgcgcctgtaattccagcactttgggaggccgaggcaggaggatcagttgaggtcaggagtttgagaccagcctggccaattggtgaaatcccgtctctactaaaaatacaaaagttagccgggcgtggtggcgggtgcctgtaatcccagctacttgggaggctaaggcaggtgaatcacttgaaccctggaggcggaggttgcagtgagccgagatcgtgccattgcactccagcctgggtgacagagtgagactccatctcaaaaaaaaaaaaaaaaaatgaaggggatttttattttttttatttttatttatttatttatttatttttgagacagagtctcactctgttgcccaggctggagtgcagtggcgcgatctccgctgactgcaagctccgcctcccgggttcgcaccattctcctgcctcagcctcccgagtagctgggactacaggtgcccgccaccgcgcccggctaattttttgtatttttagtagagacgaggtttcaccatgttagccaggatggtctcgatctcctgatctcgtgatctgcccgcctcggcctcctaaagtgctgggattacaggcgtgagccaccgtgcccggccgaaagggggatttttaaatgggttcttttcctttggaagaagagaggatggaggaacagaggtggacaggtgacaatgggatgggggcgaggtccccaggaaaataaaagaacattctagaagcacccagagctggcatcttttagtggggacactcagcaggtggggccccctttgtggggtggacgctccccgactcctagttgtctccctgcacccccacagtgccgggccagcccatgaacctgcgggccgaggccaggtcggagaccagcatcacgctgtcctggagccccccgcggcaggagagtatcatcaagtacgagctcctcttccgggaaggcgaccatggccgggaggtgaggcagcgcggggacgggctggcctgccctgggctcctccccggcgctctctgacgctgcccgccttgtctcttccccctctttgtccctatgtgtgctgcggattctgctcttcctcaccccttctgcttctcctgcgtctctgtccctttgtgcctttcctccacttttttctcctctgtctcatctcctttgtctttctcctctgctgctggcggcctctctctggttccgtatgtatcggcgtccccatctctcttgatctgtgcttctctctgtgtcttgtcttcaccggtgcctctgacgctcactctccatccctccgggtctgtttctgtctgtttatgtctctgtctctgtttctctttctgtctctgttttctgtgtgttttttttctgtctccatctgtctctgtgtttctatccctctgcctctctttctgtctctctgtctctgtctctgtatctgtttctttctcctctctctctatttctgtctctatctctatttctgtctctctctcctctctttctctgtccctgtctcaatctctctctcctccgtttctgtctgtctctctcccctgtttctctgtgtttctgtctctgtctctatttctctgtctctgtgtgtctatctctgtatctgtctccttctcaatttctctattcctctgtctctgtatctctctcctgtttctgtctgtctctgtatctctctctgtctctgtctctcccatttatctctgtctctgtgtctgtctctctcctttgtttctctgtctctatctctgtcactgtgtccctctgtctctatctctgtctctgtgtccctctgtctctgactctctcccctgtttctctctgtctctgtctctctccgtctctgtctctctccgtaccctctttttctgtctgtatctctgtctctacctctctctcccctctacctctctccctcatccctctctgtccctctctgccgcacccctgtctctatttctctgtctctctctctctctctctctctccccctcccccctctgtttctgtctctctctctctcccctttgtttctgtctctctctctctctcccccatccctctctgtccttctctgccctgcccccaggtgggaaggaccttcgacccgacgacttcctacgtggtggaggacctgaagcccaacacggagtacgccttccgcctggcggcccgctcgccgcagggcctgggcgccttcacccccgtggtgcggcagcgcacgctgcagtccagtaggtgtctcgcggggccgcccttctctgcgggagggaccacggcctgaccgcagacagcccctcggcagacggacccaggcggggcctgctgcagtgggtggggggcgcctcccggcctgtctccgctttgttccccgacctcggatccatctacccgcgctgtgcctcagtatccccatcagcacagcgagggtctcagcatggggttgtctgtgcccctgggcctggtgacccgggttgagacttggggtcaaggagaagtggctgatcttggccctggtgtaggagaggttctgggaccacgccttctttctcctctttccatcctggagtgggggctgattgaagggccacgttgtgcccatttcccaggcattttgagggtaccgtgaggctgtggtcacggctcgcaccccatcattctcagagcaaaggacacagagacgaacccaccttccccgtcggtcttcctcccccgggcagggagccttggctctcaagaccccaggacgaggtcccagttcccgagggtggggagggggtggaatcggagccgaggccaggtttcgtgggggcatgcaggtccccactcggggctcaaatctctggagttctctgctgtgccggcccggcctccctgtttggagcgagtcacccttcacctggcagttggctgtgggtcaccgccaccctctctccctgacttctctaaactcggagctggacaggccccttctgccgtctccccacctcacctgcctccctggctccgctcagcccagcgtggccttccctggtgtggcctccaccggcgtggtctctctgtcgtggcctccccctgcttccccctcttccctcccttcacccagtcagtcacggcgtagccaccccaggctttcttccagaatcagtcatgatggtcaaaccttcaaggcccgccctgggtgtgtgtacacaggggcatctcggatcagtcgaagggttaacatcaactccagccggggcatctgagccgctgggatcgggggggtggggcgcctcggagacacaacccccctgcccccgccggcccccttccctccttcctgctccccacaaaccaagccgagctgagcacctggcggctccaaggcgtctgggcgcgtctgtgtgaggtggagtcagtgagggtggcctgtggcttgggcagccacctgagagactggctgaggtgtcctgaaggtgagtacacgctggtctcaagcaggttcacaaaaacggcccaccccgcgtgcccccccgcctgccccagccactctgggctgaacggacagtccacggcccgaattcggccccagaaactctcccaaaccacgtgccaccaccatgccacgctgcccacgcacgcacatccacgtctttgcacacatcccttcccgagatgtcatgcaggttgggccggattgtgaaggacgtgacctacacggtcacctagaaagcggcttcggcccagccacgcgaacctccggggcggcccaggacagggagttggggagaagggactccccgcttcttgccccgagacgggtggggcccaggagaaccgacagcaaagactcagggccaggcgtatccatccccccggggactgcgggctccttaggctgaacacagggtaggtgcaggtcagaccactcagccgtgggtctccccagacaccgtgccaccttcctgcctccagcctggaacctcagcgtgcgcacgctgggggctggagcagggctgcggggccactggcatcctgagacccaccaagagttcggtctggccttgtagctcagcgctcggccgttccctctctcgtaagaaagtcttttaagttagcagttgaagagtggaaggtaggtaaccagactgggcgttttctggaaggagtctttctttcttttattaagtttttctcatcatcatcaccatcactgttgtttcttattttattatggtttcggttttattttttctccccctttctcagcaaatccattccttccgacacattccttgattgttgttttttttttttttttccccctgctccctgcctgattccccgcaccccccgggcacctttggttggtgtggctggtcaagaaatggggaagggttccctttgtctagttttggaggaggggcctctaatgttttgctttaagtgaggccggagggtctctctagttgaagtaagctgttggggtggcctctgcagtctctggtagcccagtgtctctgaggacaggattcgtctctgggtttcagatgctagcaggatagtgtccatagtaagtactcagtaaacatgtgctcagccaccaagcaagctgctctgatggtgagggtttctaatgctcttgccacgagcaaggccagtaatctctctgggcctccctccaggggacgtagattctaaaatcgtgggcacttgggagagtggcttccgccatacccaaccagcacaaggctgagatctgagctgccaacctcaaaccaagccagcctgtgaatcacggccaagtctgggttccaatcctggctctgccacctcatcactgcatgcccctggacaggtagcttgctgtttctgagcctcagtttcctcacctgtaaaatgggctcacctgtaaaatgggctgcacttctggtttttcggcaagattgagaggtgttatacaaaataatgaaaagcttccagggtggggtctggccacagtcggtacacaaccagtgactgtagaaagcccagcaggcagactttggaatctggtttccccaccgttgacgcggggtggcgggtggggagtgacagttctgaccacctgaggggttgtgagaattaaatgagataatgcatgcattaaatgagatgatacatgcgttaaatgagataatacatgaaacatgggctgagggtgctgcacctgcagtaggcgtgcagaaaatgaagtttattatcactaatgccctcagcacagcgtctggccatagtaggcacgcaacacatagagacttactgattcataagcctttaacacacagacagttccctttaatatgcgggaactctcagtgttattcagcaaagaccttaacatactgcctggcacacagtaggtgccccattaatacagccactgttttaaatctacatatggattggcacagtgctgggcacctagttggcactcagtaaatggaagctattagcagtttataacaagcttagcctagtgccaggcgcacagtaggtactcaaaacatgggtggcaacaattattgttctgcaaacctcttggcacaatggctgacatacagtaagtgctcaataagtggcacttgctataaagaggtggcctaagtgtgaggcacgtagtagatgctcagatgggcaccattatcagtttgaaagggtgtgggatagggctggttacacagtttttactgaaaacattcacttattatctgcaaagcccttggcacagtgtgtgcaaatagtaggtgctcaataagaactattactatttttatataaggtattgagtgcaatgcctggcacacggtgccaggtgctgttacagttatccaataaagcccttagcacgatgcttggcacacagtaggtgcttaagaaatgaaaactattggccgggcgcggcggctcatgcctgtaatcccagcactttgggaggccgaggtgggcggatcacgaggtcaggagatcgagaccatcctggccaacatggtgaaaatccgtctcccctaaaaatacaaaaaattagccgagcgtggtggcgggcgcctgtagtcccagctactcgggaggctgagacaggagaatggcgtgaacccgagcagcagagcttgcagtgagccgagatcgcgccactgcactccagcctgggcgacagagcaagactgtctcaaaaaaaaaaaaaaaaagaaagaaagaaatgaaaactattaacattccgtaaggcccttagtagagttgcctgacacatagtaggtgcttcatatgtggaaacttactgtttttcagtaagccccagcacagcacctggcacacaataggtgcttaatatgtggtgactactatcgttttctgatgaagcctttaacacagtgcctggcacacagtaggggcctaatacatggggactattaacttttctgatacagcccttagcacagtgcctggcacgcagtaggggtttaatatgcagggactactactgttttctgagagagcctttggcacagtgcctggcacatagtaggtgctgcttaaatgacaactgctgtttttctcttcagagacctgttaccgtacccagtccatagtagatgttcaataaatgagacttgctgttgataggttagtagtagatgtagtgtggtgccaggtgcatagttgggactgaacacagaagtgctgtgatctttaaagattttggcacacagtaggtgctcaatatgagctatgaagtcctgagcctggtacctgacaccagtaggtgtccaataaacaggcacttttattattacttgataaagcccttgacattctatctggcacacagtaggtgctcaagacatgggcattattttcaagtgaggccaattggatggtcctagttgaagtaagcttttgggatgggctttgggtccctggtagcacaagtgttcagtaaataaatgcttttactacaagaaaaattttaccatattttccgtaaattattatgaagcccttggcaccatgcctgacctacagtaggtggccagtaaataagtgcatttgctgttattgtatgaagcccttggcatgacacctggcacatggtaggtagctaataattggggcttctattgctgttgcctggcacatagtgtgtgacatatagatttttttttttttttttttttttttttttttggagatggagtcttgccctgttgcccaggctggagtacagtggcacgatcttggctcactgcaacttccgcctcccaggttcaagtgattctcctgcctcagcctcccgagtagctgggattacaggctcccgccaccatgcccggctagttttttgtatcttgtagtagagacggagtttcaccatgttggctaggctggtctctctaactcctgacctcgtgatccacccacctcggcctcccaaagtgctgggattacaggagtgagccactgcgcccggccgagattcttatgctattattctatgaagcccttggcacaatgcttgacacacagtaggtacctgataagtggagcttctattgctgttccccttagcatgatgcgtggcacatactagatattcagtaaatagatgctctattattttgtgaagcccttggtgctggcacatagtaggtgcctaatggagcttctgttgccattccccttggcacagtgcctggcacatagtaggtagttaataaatggagcttctgttgctgttccctttggcatggtacctggcacgtagtaggtgcttaatggagcttctattgccattccccttggcatggtacctggcacatagtaggtgttcaataaatagatgcttttactattatttcgtgaagtgcttggcgctggcacatagtaggtgtctaatagagctctgttgctgttccccttgccacggtgcctggcacataggtgttcgacatagattattttgttaccattctatgaagccttagcacaatgcttgacacgtagtaggtgcctgataattggagcttctgttgccattccccttggcatggttcctggcacatactaggtacccaataattggagcttttgttgctgcttcccttggcgtggtgcctggcatatagtaggtgcttaataaatggagcttctgttgccgctccccttggcatggcgccaggcacatactaggtgttcaataaatggatgcttttactgttattttgtgaagcccttggcgctggcacatcgtaggtgcttaatagagctctgttgctgttccccttgccacggtgcctggcacatagtaagtgtttgacatatagattattttgctatcattctatggagtcctcagcacaatgccttaccacaattattagtaggtgcctaataattggagcttctatggctattccccttggcatggtgcctggcacacagtaggtgcctaatggggcttctgttgccattccccttggcatgatgcctggcacatagtaggtgcttaatgaatggagcttctgttgccattccccttggtatggtggcacatagtaggtgcctaataaatggagcttctgttgctgttccccttggcacggtacctggcgcataataagtgcttaataaatggagcttctattgctgttccccttggcatggtgcctggtacatagtaggtgttgagtaaatagatgctttactatttgtgaagcccttgttgctggcacataggaggtgcctaataaacagagcttctgctgccattccccttggtatggtgcctggcacataataggtgcctaataaatggagcttctgttgctgtttcccttggcacagtacctggcacataataagcctttaataaatggagcttctattgctgttccccttggcatggtgcctggcacatagtaggtgttcagtaaatagatattttactattattttgtgaagcccttggtgctggcacatagtaggtacctaataagtggagcttctgttgccgttccctttggcatggtgcctggcacatagtagatgttcaacaaatagatgcttttactattttgtgaagcccttggtgctggcacatagtaggtgcctaataaatggagctcctgttgctattccccttggcacgatgccaagcacatagtaggtgcctagtaagaagggcttcccttgctattcccccacccagtttctggcatggggaccatagctgtccaatgcctaagacctactgtgcaccacgtaggtgcagcctggtgagcacagggttcttggccagccctcccagccccatcccacctgcccgcccatccacccgcccatcccacctttggttgacacccacttcttttgggttcgacttctctttggtttggttctgttttgtttgttatcatcttcttttttattttctctttcccatcttttgtttccccgcccccctccccacccatcccacctcccttccccacccccccacgttctccccccctccaatagaaccgtcagccccccctcaagacgttaaatgtgtcagcgtgcgctccacggccattttggtaagttggcgcccgccgccgccggaaacgcacaacggggccctggtgggctacagcgtccgctaccgaccgctgggctcagaggacccggaacccaaggaggtgaacggcatccccccgaccaccactcagatcctgctggaggccttggagaagtggacccagtaccgcatcacgactgtcgctcacacagaggtgggaccagggcccgagagctcgcccgtggtcgtccgcaccgacgaggatggtaagtacctgccccaggcccgggaccccccgcaggccccagcccggcccccgcctcgaagccttccatctggaaacctgggctggtggtcaaagggcggaagcctcactcggaggaagtcgggaagcaagcagcctccccgagaaatccggggctccttgactttggaattggcgattctagacatgggcaggcttagctctgcagcatctgctcccagagcctcgtgcctggacagtttcgctggctgccctctattttggtggaacagtggaaacctttggaaatggggtggaggaagtggcaaatagcacctgcaacaacaatgtgctgatcatggttaaccagctccccgggtggatggagaccagtttgtgtagcattggccaatttccatggtgtaaatgttcccgtcctcacccatttcaagttagcagcaagtttgaagctggaaagcaaggcagtggtcctagcttggagtcacgtggtcgaagcctagatcatggagagaaatgacagacacctttcccaataaaattgcaacttcttggccaagcacagtggctcatgcctgtaatcccagcactctgagaggccaaggcaggaggatcatatgagtctcggagtttgagaccagcctaggcaacatagtgagaccccattcctccaaaaacatttttaaaacatagccaagcatggtggcatgcacatgtagtctcagctactcaagacgctgaggtgcggggattgctgggcatagcagtttgaggctgcaatgagctgtgatcatacccctgcactccagcctgggtgacagagtgagaccccatctctacaaaaacgtattagctgagcatggtggcacaggcctgtagttgcagctacttgggaggctgaggtgggaggatcgcttaagcccaggagttcaaagctgcagtgagccgtgatcctgccactgcatgccagcctaggtgacagagtaagagaacctgtctcaaaaatatttcagcctcttggtcctgggaacctgattcgttttctgagccatttgcctttgtttcgttggacagccccattatgtcctaagcccagagaagaaatgaagcagctcttctttatgccaaaccctggggtgggtgaaaaagatcctgagtgccaagtgggccacctgcctgcttgggtatttaggaacatacagctggactgcctggtgcaaatcctggctttgcctcttgctaaggctgtgatcacagaatcaactctctaggattcaatttccccaaatgtaaaatgagaatgacaggagaccctatcttgcagggttatgatgaggatggaagcagtgtagggtgaagaagagggtgtctggaggccctgggaaggtggtttagactcgggggggggcagcctggatccatggggcgcaccttcaggggcccctcccagggctgtggggggtgtgcaggaggccatgtcctcccgcccggtggtgagcgctaagagcgcccagttattcaccggcagccactcggcatcctggaatcggacagtggcactgcccggggcagggccgggcgccctggtaacggctccgcccgcccctcccctcccctcccgcccgccagtgcccagcgcgccgccgcggaaggtggaggcggaggcgctcaacgccacggccatccgcgtgctgtggcgctcgcccgcgcccggccggcagcacggccagatccgcggctaccaggtccactacgtgcgcatggagggcgccgaggcccgcgggccgccgcgcatcaaggacgtcatgctggccgatgcccaggtgggcggggggccggggcggggccggggacgggctccgggcggggacgggcccttgcgctccccccacctctgcttctctttctccatctctggcctcccccgctcaccttcctcctctctcccctgccgcctcctcccgcgctcagtgggagacggatgacacggccgaatatgtaagtagcgcccctggccacccacctactgctgtgcgcttcccgcgggccgtgctgtgctgtgactcctgcacgccccttatcatcagtagtaaaacggtctcctcctctgggccctcccatgcgcccctcgctctcccattacctcttggagcgcccagcctcgatgtttgcaccagggaaatgggatactcggaccactgtgcgccccagggggcagacccgagggtggcttgagcaacaggacctgagggtcgtccctgaatcctctcaccctgatggctcctgccattggtggggcttaagcctcctgggtccttctcctgcccgtagtcatttctcctgtgagtatgtctcctctcctgttgacccagccccaccctttactgtcactcaattcagccctgtctggcctagaaatgcctctctctggatatgctgtcgtcttcattctggcccccgggtgatgagaggtgtcagcggggccagagtggggaaggatgtctgtgatagcctggttacaaccccacaatgctttaaaccagagaggaattgctgtgtgacttggggcaagttgctacctctctctggactacctctctgcccctaaaacgggagtaccagtctctgctgagaaccccaggtctgacattaaattggcactggtcccttggctgggcgtggtggctcatgcctgtagtcccagcactttgggaggttgaggcaggtggctcacctgaggtcaggagttcaagaccagcctggccaacagggcaaaaccccttctctactaataatacgaaacagcctggtgtggtggcaggcacctgtaattccagctactcgggaggctgaggcaggagaatcgtttgaacctgggaggcagaggttgcagtgagccaaaatggcaccactgcggtccagcctggtcagcagagggagaccctgtctcttttttttttttttttctccagacggagtctcacactatcgcccgggctggagtacagtggcgccatctcggctcactgcaacctccacctcctgggttcaaacaattctcctgtctcagcctcccgagtggctgggattacaggcatgagccactggtcctggctgatcctgtctctaataaacaaacaaacaggcactggtcccacatgctctgatgcctcccatctctcacccacactggatctagagggaatgttcccacctgggcttcaactctgctcttctcgcagagaccctgggagggatggactgtgaggctcaggggtaggagaccctgtttgtgcatccccgtcacccctccttatccccttggcctgggtctaccccacagttcccaggcatgccggcctgtgcttgggggggtgcgtgtgtgcttgcagacatgggcacacatgatgctgtgatcgtgtgcacaggtgttgtgcaagtccgcgtgcccgggagtgttctgtgctgtcattcattggacgagcactcacttattgagcacctactatgtgctagatgctgaggatgtggtggtgacaagagcccctcctccctaacagtcatactcagtgagagggatgttaatcggaacaaaacacacatcggcagggcatggtggctcacgcctgtaatcccagcactttgggaggccaagacgggtggatcagctgaagtcaggagttcgagaccaacctgaccaacatggtgaaaccctgtctctactaaaaatacaaaaccaggcgtggtggcgcatgcctgtaatcctagctactcgggaggctgaggcaggagaattgcttgaactagggaggcagaggttgcagtgagctgagaccatgccattgcactccagcctgggcaacaagagcgaaactctatgtccaaaaaaaaaaaaaaaaattgttacagctgtggtggggatggggtttagacagtgaaataaacatttaaaaatcagctctcagccaggcacagtggctcacgcctgtaatcccagcactttgggaggcagaggcaggcggatcacttgagcccaaagtttaagaccagcctgggcaacatggcaagatcccatctctacaaaaaatataaacatcattagccaggcatgtggtggtgcacgtctgtattcccagctacttgggaggctaaggcgggaggatcacttgagctcaggaggtcaaggctgcagtgagctatgattacaccctgcactccagcctgggcgacagggtgagaccctgtctcaataagataaaagagtaaaaatcagctctctgggtgggcaggagggggcagtcctgttttgtagcattggaccgtttccatggtgtaaatacttcccaccatgtcaatttcaagctaccggggaggtgcccagtagcccataatcatgtattcctactctacagatgcaatagacaagatgatctcaaagcagtaaccatacagatacccacagagaacagtaaccctagtgaaataactagatttgctgtatttattgcctttgctttcaatataacttagttgattgtaagtttatacagtttatttatttatttttgagacagtcttgctctgtcacccaggctggagtacagtgccacgatgttggctcactgcaacctccgcctcctgggttcaagtgatcctcctgcctcagccaccccagtagctgggactacaggcatgtgccaccacgcctggcttttttgctttttttttttttttttggtatttttagtagagacgaagtttcaccatgttggccaggctggtctggaactcctgaccttaagtgatcctcctgccttggcctcccaaagtgctgggattacaggcgtcagccactgcgcccagcctcgtacagtttaatttttaataacagctctgtttcataagtggctcacaaaattcctgaaaatttaaccagcggctctcatgagttggtaccgggcacctccagcacaccagtgggtgtagacaggggggaaggccagtgccaaagggcctgaggcaggctgtgcctggtgcattggaggaacagcaaggagagccctttgtgagccacagggaagacttgggctttgaccccaagagaggtgggagccatggagggctgtggggagagcggggacggaccctgactccagtgctcacaggcgccctctggctgctgtggggaggacagactgtcgggggtgatggcaaggagtgaggggagcggagcagagaggaccgagttggtcctggcaagcgacaatgggctcgtccggctgggttgttgcatgtaccctctgagcatggccatgccacgtgtcgtggtctctgcatgagctcatgtgcacgtctccacccctgatccgggccctgcatcctccttgcttgttctcagcacggggagtgggggtgggtggggctgctcccagccccagccgtcgtgctgacccccagccgtgcctgcaggagatggtcatcacaaacttgcagcctgagaccgcgtactccatcacggtagccgcctacaccatgaagggcgatggcgctcgcagcaaacccaaggtggttgtgaccaagggagcaggtatgcaacccgccctcctcccacccaccaacagcccctttgcccccactggcaccagagcgtgactccagagtccttgagctcacaagggcaggcagagggtgcaccaggcagctggaacagtgagacaaaggtgtgagggctgctcaggtgggtgtccagggcccagcttggccccccccgcccctgcctcctcctttgtcactcacttcctgtccccccggctcccaccagacagaaacagttaacagccacctctgacccctttgccctctcaacctttacttgggctgttccctctgccaggataccccttcccctgctggctggctcctcttctgcctttaggtctcagctcaaggtacacctggccctggaaagccttccctgattgcctcatcccataatcttgatccttctgggccccctagtctcctgtgcttcccccatccaggctctggtcgccccggctaggttgttatttcagggtggtgcacctgtcttcccctactgcaccaggaagccacctttctcttggtggccctgcctcagtttccccttctagcccctccattgtcccttccatcgtccccaaggggctggcttttttttccagccaaaatggagctgaattaagaaaaagaggctgccgtgaagatgtaaggggagggtacaccgggcggagggcacagcccagaaaaagacttggggggtggggcgtggtggtggtggtggtgggcggaacaagctcgggtgtttcaggaacaatcgactgacttctgtggccttcatcccagtccccacacccaccctctcctcggggacccttgtagccacaccactgtgtaccccacctttcaggcctcccagctggacacagcccctgcccttgtccacactattcccttcacgcaccttgtctgcgtcctccagcatccagctctaacgccacctcctccaagcagcctcctgtgatctctccctcccttcttctagccagcagggcacttccctcctctctcagggacattgtattagagctgtgtttgctaagcagaaaccactgcatacgctgctctccccaccaccctgggtattcatagcagtccattcaaggccctgataagtcctgcagtagagaagtccattttcatgtatacagcccagctttcccccttctttgcctgatcccagaacctctctcggcacattgattactacccaggctgtgcgaaacaaagagcaggggcggattttggatttctttatgtcccatgggtattggtcaggtgcctgcattgagcaagggccaaatcaacaaggcaggagatggttgccttattaatggaaggaggaggggttctctgagtcctcacttccttgctgtgctttatgcgcccctagcccagtgccggctctacacctttgcatgggccgttccctcttcctggagcacgattcccccagatcttctcgtggttccctctctttcttctttcaagtctttgcttcttaagtgaggctttcttgagacctacttttatttttaagacggagtcttgctctgttgcccgggctggagtgcagtggcacgatcttggctccctgcaacctctgcctcctgggttcaagtgattttcctgccacagcctcctgagtagctgggattacaggcgtgcaccaccacccccggctcatttttgtatttttagtagagacagggtttcaccatgttggccaggctggtctcgaactcctgacctgaaatgatctgcccatctcagcctcccaaagtgctgggattacttacagatgtgagccaccacacccagccttgagatccacttttaaaaatcacaaccccaggctgggcacggtggctcacacctgtaatcccagcactttgggaggctgaggcgggtggatcacctgaggtcaggagttcaagaccagcctggccaatatggcgaaaccccatctctactaaaaatacaaaattacccaggcgtggtggtgtgtacctgtaatcccagctgctcgggaggctgaggcaggagaatcccttgaacccgggaggcagagcttgtagtgagccgagatggggctactgcacttcagcctgggcaacagagcaagattctgtctcaaaaaaaaaaaaaaaaaaaatcacaaccgcagctgggtgcagtggccttataatcctagcactttgggaggccgaggcaggaagctcacttgagcccaggagttcgaggctgcagtgagccgtgatctcaccattgcactccaggctgggcaacagagtaagaccccccccaactcaaaaaaatgttatattgaaggaaaagaattaaaaatacaaccccaccttgtgcctctgggctggcggatggcgctgggaccctgacacctgccccgtatccccgcagtgctgggccgcccaaccctgtcggtgcagcagacccccgagggcagcctgctggcacgctgggagcccccggctggcaccgcggaggaccaggtgctgggctaccgcctgcagtttggccgtgaggactcgacgcccctggccaccctggagttcccgccctccgaggaccgctacacggcatcaggcgtgcacaagggggccacgtatgtgttccggcttgcggcccggagccgcggcggcctgggcgaggaggcagccgaggtcctgagcatcccggaggacacgccccgtggccacccgcagattctggaggcggccggcaacgcctcggccgggaccgtccttctccgctggctgccacccgtgcccgccgagcgcaacggggccatcgtcaaatacacggtggccgtgcgggaggccggtgccctgggccctgcccgagagactgagctgccggcagcggctgagccgggcgcggagaacgcgctcacgctgcagggcctgaagcccgacacggcctatgacctccaagtgcgagcccacacgcgccggggccctggccccttcagcccccccgtccgctaccggacgttcctgcgggaccaaggtaggcgcgcggcgacccctgcaccagagccggaccgggccttgcgcccacccccaccccagccccctccctctcccctgcccaggtaagtgctcacttttctgcttcctcgtggtcactcaaggcaagtccggaccccgaggctccggcgttggcaaaggtgggacgtgcctcggtggcaccaccgccccccaccactgccccctccccttgctcccctcccccctcttcctcctcctccaccttctttctgcagcctctgcacccgtgccgtctcccgaagcagggacttgggacaggacagggcagggccgggccagggccaggcaggaaggccgtgggcatgggcacagcggacgacagggagcgcctgggccgccccaccccaccccgcttcacgccacccagtgccaggcactcatggggcctctcttctgctctctccctgctcggccgcctccccggcagtctcgcccaagaacttcaaggtgaaaatgatcatgaagacatcagttctgctcagctgggagttccctgacaactacaactcacccacaccctacaaggtgcgactgcccagccagcaggcaggcaggcaggcagggtcaggggttgtagttcagcagggcttaaggctcagtttggagttggtgtgaagtgagggatcagggctcagtcagagatgggtctcagctaggattgaggcttagtcaatactgggaatcagacttggggcccagcctaggattggagatcagctggaactgaggttcagtcaatattggggatcaatgttgggctcagccttggattgtggcttagctgggattgaggttcagtctgggattaagatttagtccatattggaaatcagagttggggctcagccttagatcatggcttagctgggactgaggcataatcaggattgggactcagagttggtacttagcctgaggtcagagtttagtctggagttggtgccaagtctgggatcgggggtcagcctgggattgaggttcagtctgagatcaggaatcagtctaggatcaggtctcagaggtgggtttccacctagggtcagcgctcagctggaattcaggatcagtctgggttcagggttcagtgggattggggctcagtcgggactgggaattcagcatgggatcagggctcagtcaaagtgggcttctacctaggatcaggactcagctggaatttaggcacagtctggggtcagggttcagtgaggttggggctcagtcagagttggggactcagcctgggatcagggatcagtcagtgttagatttctgcctagggcaggattcagtgggatcagggctcagtcagggtgggggactcatcctggagctggggctcagtccatgggcatgagcccaccaggccctgctgagctagccctggtccgcagatccagtacaatgggctcacactggatgtggatggccgtaccaccaagaagctcatcacgcacctcaagccccacaccttctacaactttgtgctgaccaatcgcggcagcagcctgggcggcctccagcagacggtcaccgcctggactgccttcaacctgctcaacggcaagcccagcgtcgcccccaagcctgatgctgacggcttcatcatggtgtatcttcctgacggccagagccccgtgcctgtccagtatgctcacccccatgctccgggttccttgtcacccccatcagcctactatatggctggggcaagccacgtgccctctctgtgcctcaatttccttgcctatgaaacacagaccctatcagccctagttcatggggtcatccaagattcaatgcttctcatgggtcattgtcactaaatgtgtcctgtgtctgctcttctagcatttcctgataccccatatgcctgaccctccactgggtattactagggcccctgaaaggatttggttctgggcttcccaactagtggggaggcaaagctgagagtccccagtggggagaggagcactctgcctcagaggcagagaagatttaagggctacaaggttatgtgggcattgaagctggagctttgaaggatgtataggagtttaacaatgagcattacatgaagtaagccttctacgcagaaagtacagcatcatccaaggcctggaggcctgagtcagcaactgtggcgtttgtggatggggcataggggaataggctcattgtatgaacagaagggaagactcagagaaacggcttcccccgtttgcccccatgaagcatcccttggtcatccctatcgacagcccctctgactctttcccatctccctacaggagctatttcattgtgatggtgccactgcgcaagtctcgtggaggccaattcctgaccccgctgggtagcccagaggacatggatctggaagaggtgagggcctggggcccaacccgcagggcccagatgcattccagttttcacgtgctctgttgctcccagctgagctctgagccacccatgactctgttcccgcagctcatccaggacatctcacggctacagaggcgcagcctgcggcactcgcgtcagctggaggtgccccggccctatattgcagctcgcttctctgtgctgccacccacgttccatcccggcgaccagaagcagtatggcggcttcgataaccggggcctggagcccggccaccgctatgtcctcttcgtgcttgccgtgcttcagaagagcgagcctgtaagtcctgaatggtgtccgcccacatccagacacacctctgaatgaggcagaccccattccccagggccctgacctctgtggtcagtcacaggcagctccctgtggtcagagctgggcagaggggaagacttgggagcggagggatcctgaaacgaaggtttcttagaggaagagaccttggagctggggcgttgaggggtgagtaggagttcaacaaagaaaaagcatcccaggatttgcatgtgcaaaggtctggaggtacagaaaggagagtggctggagcagagaagggaatggggagagcaaagattggcagggaccaggtcacataggccaccagggtcaaggttctaggaatataggaggatctagagcgggggagaaacgtaggttcatgtttgagaaagacccatctggccactacaaggagggtggacactgatggagacccccctcctttccctgcagacctttgcagccagtcccttctcagaccccttccagctggataacccggacccccagcccatcgtggatggcgaggaggggcttatctgggtgatcgggcctgtgctggccgtggtcttcataatctgcattgtcattgctatcctgctctacaagaagtaagccccatgtcctccccgacttgacttgacagcagggagggcttcctggaaaaggagggaggagttgtctcagagaatcatcactaaaggtctctgttgtacacatggggaaaccgaagttcagagaggttaagccacttgatcaaggtcacacagcatgggaatggcagagctagggctcaaagcaggccagtggcttggcttctcctcttaacccctatcttcaactcagcaagatttggggtagaggctgcctgtgtccagttcaatgaggatagcaattgtagtggccccatggggcacatcccagggctgagataagcctccttgtggtgtcgataggcagggggacaaacaggaaatatgagcaccattttccagaggagaccaaagctcacagaggttaaggaaggcactgagagtcacagaacaagcccttgatggccggcacaccctcacctttcttctttttttcccccttctcacctgtgtctgctttcgggaacagcaaacccgacaggtaagaaccagccctcccccgtggaggcttcaggcaagaggaagaggacagggaatgggctgatagtgtgcccacaggagaggggatgcccggctgcccccacagcttgtacacgtagctgtacttggtcctggaaaccctagtcatacgggtccatggtccttttcttaccatgcattcctggggcctgaattgtgttcacacatgaacatgtgccactactaactggactgttccgcttgcttctcctaaagtaaacgcaaggactcagaaccccgcaccaaatgcctcctgaacaatgccgacctcgcccctcaccaccccaaggaccctgtggaaatgagacgcattaacttccagactccaggtatgtacttatccctggtaccagggatgccaccaacaggggagatagctggggagaagcagtagctgccaccctgggggcccctcattggccccctctgaactatgcttggtgcagccacattctggattcccaaatacctccaaccctacatggatctgggcttggaactcataggttggttctgcttacaaccaagtgaggcttttttttttttaaagttaacatttaaccaatggctgctgtcctcttcaggagcctaaagacgatgtttaaaaaatggagaaaacacaaataaacccctcatttctttgggcagaaagagctattcttctgtttatgtctctggttttaatgttcctcggtggtgggagttgaggtttgccctcagccattttccacactcccttgggatatatccctagcaaggtacagaaattatacctgcccaatgccctgtacctcttcccaccatggctgccagggttcatgcctctttatagttccctgtagattaaagtcacctgacagtccaggagaactgaatcaaggccttatccttgatcattttcatgtatgattcacaaactctaccatctcctccctcttcatgggctcatgccagctgcttctaaccaaataaggaatttttcattttactgatggctgctattctccttatgggttcacagccaagttacatctgtccttccaactgtacccggtctccacttcgtatctctgaactggttcatatcaactctattttccaaccaaatgaagattttaaacatttgtatttcactgatgtctttagtcctccttaaagcctcatgcatagcctcacatgccattcctgatgtactttctttttttttgtctctggactggcttgtgtcacttctgttttcatccaaatgaggagtttgaccattagtatttaactgatggctgctgtcctcataaagggatcatgcagggcctcatgtatcacccctcgtgtattctcttccctttctttcctctgtactgattcgtgttgctttggtttttcagccaaatgagaattttaaatgtgcatgtaactgacggctgcagtctcctgaagggatcatgcagagtctcacaattcaccgctaatgtgctcccatctcatttctgtctctggactagctgatgtctgttgtgtttccatccaaataaggattttcaacatgtgcatttaactgatggttgctgtcctccttgaagctcatggcatgggcccatatgtcctttccctgctcccccagcttctccttcaccatcctgcatcctccttcctcagaatgccctctgctgacctcaacagaggggacccaaggccaggctggtctgggggaggggatctgtagacagaagaacctagcactttggagtccttccagagtgacgggtcaagaccaggattgactggtgtccagccagaggggtacacagttagcagctatcgtgagtcctcaggtgggggcccaggtggctgtacataccccccgttgtccgcgttgctctgccctttgcatccgccagccagtgaggggtgggggaggcagaggtgggacccccccctaccccctgcatgttttcatttatttattgtttgctgtttgcagattcaggcctcaggagccccctcagggagccggggtttcactttgaaagttagttcttgtgtctgtttttggcttcctttcgcccccgcccccgttcatttcctctttggtttctccatcgtgtctcctgtctctgtccacacgcccacccatcaccggcctttgcccacctgtcttgtgctggctcctggaagagggcagggggttggtccattgggccctcaggtggctgtgtgtgactgctgtgtgtgtggtgtgtatgtgtgtgtcagggatgtgggtgaggcacgtatgtacccaggacgtgcccacaggacaggcaggccccttagatgtgagctcttggggggcacccaggacagctagggacacatggaagtctcagaacattaaatgttaccagctggtctggaaaccacctgagagctgaacatccggtacacaggtcccaggagaggtcagggttctgcagacccagatatcgggaagggagaaccgagcccacacccagcaccttcggcagccatcctagggggcttgaaaggataagagctctcaggccagggccttcttgcacctgctcctgccccaagcaagcgcagggacagatggggacccctcactcctctcacgtacacacacacatactgtttcccctccctagtctgtcgcctctacctggaagcggatttcgtgcctctgatcccagagggacagaaaggggtgtcagtcgcccacccaaggacagtatttccaaagcacagccggctgcgtctgcaatttaatttctgtgccacacacttgcttcttggctcgaggcactgggagcttgacatcctctggggtgacccttctgccagcccctgcacccccagcttggttggaaggaaacaggatgggccaatgctgtgatccaggcccctccaggggtgtcagcctctgcacggagcagagggcctcaaacctgctgcctttagagtcccctaggcctggggtcccagccccttcatgagccaccctgagtcttcccctcctgccaagccccaccccggccatgcccttctaaccacagctcacccatcgcagactcacacgtagatcacccccctcccccgagtccccggctggccccccaagtgaccaaccccggcggggtctgctccaccctgtaggcatgcttagccacccgccaattcccatcgcagacatggcggagcacacggagcggctcaaggccaacgacagcctcaagctctcccaggagtatgaggtgagcccacccccgcctcccaccctcatcacctcggcctcacaggcacctaccctgacacccgcgctctccccttcctctgtagtccatcgaccctggacagcagttcacatgggaacattccaacctggaagtgaacaagccgaagaaccgctatgccaacgtcatcgcctatgaccactcccgtgtcatcctccagcccattgaaggtagttcggagatcccgggtcttaacctgcccttccccactgcatggaaagtgctagatcccatgtctccccttgggccactgaggcctgattctggtcatagacctggtccagctccaactggaggccactgggcatctttagatatgtatttaactgatggctgctctctctaacatggcttcatctgtccttctgcatcagttgtgaccaatcagagcagttttgctgcccaggggacatctggcaatgtccagggacatctttggttgtcacaacttgaagggaagaaatgttgctggcgtggagtgggcggaggccagggatgctgctcagcaccctgcagtgcccagaatggccccgccacagagggcgatctggccccaaatggtgatggcgctgagggggagaaactgtgaagtacacgtcccatggtcaccttggggacaatctgggagcggtcagagtgaacttccaggaagtggttggccacacagagccaaactagcaagacagcccccacagatccctggcctctcgccctggctgcaggcatcatgggcagtgattacatcaatgccaactacgtggacggctaccggtgtcagaacgcgtacattgccacgcaggggccgctgcctgagacctttggggacttctggcgtatggtgtgggagcagcggtcggcgaccatcgtcatgatgacgcggctggaggagaagtcacgggtgagccttggaccccacggccctcaagcccctgccagtcagcctgtccctgctgacacctgttctcttgcccgatacagatcaagtgtgatcagtattggcccaacagaggcacggagacctacggcttcatccaggtcacgttgctagataccatcgagctggccacattctgcgtcaggacattctctctgcacaaggtggggcccaggctggggctgggggctgagggtggaggaatgtgttggaagggaggcagagggccccagcttctacccagggagctccccatgggacagagagaaagcggagcgtgtctacccaggctgactacattctcatggaacccacttccgtggagggggaaactgaggctcagagaggacactgcttgtttccacccacggcaggagaacaaagccggcagagcccaactcagcaatgagcctgatttcttcttgcctttactcatgccccaggtcctctttgcccacttgtcctctggcctcacccagcttgagcctatgatgggggcctcagcagggccccagccctgccttcaaggagtctccagacagagatggacagatattggcagccctgcaaagtcaggactgggatggagaagatcttttagatatgtatttaactgatggctgctgtccatagtgcggttccatctgtttttctccatctgtggttctgaattaggacaattctgccatccaggagacatttgttattgtcatgatggggaggggaggtgctactggcaccgtgatgtatccccaacacccagccaggtcatctcaccgtcaggcacctgcttgacctaagtgccagaaaactgagtacatggacaagagtgagaattcttggttcaatcacacacattaagcatatgttatacattaagcatatgttatatctcactccctaacctggacacaagcacacaacagggacctaaacagacaaaacgttctgcccttgggggatttaacagtctagtagacaagacacagaccacaactatgaaatgtcaagaaagcttacatgtcaaaggagaaaaataaagtggaggagaaaaacagtgttagggacctggaagagggtatgatggggagatttggggggacagcatttcaggtaagagggtgcagcctgggcagaggccctggggcaggaccgtgcctggtcatgcaggatcctgtgggccttggggaagactttggctttgaccctgtgggaggcaggagccatggagggctgtgggcagaggaggtaccagatctgactcaggtgccctctggctgctgcagggaggacagactggggggcttgggtgggagccaaggtctgcggcaaagagggtggtgataggtctagaccagattggcctgcatggagaacatggagcaggtggattgggatagactttgaaggcagagccgacagtcccgggtgaggtgttggatgtgagaggggaagagttaagggtgactcccaagctgtggccagagtgcccagaaggttggagccaccttcagctaagacgggggagcaggtttggggagaaggccagatacagttggccacgttgtgtgtgaggcgtccgcaagactctacttggaaaccgtccagggccagctagagcccaagtcaggcacttggggacatggtgactgggaggcgcagacatgggtgtttttttttttgagatggagtttcactctgtcacccaggctggagtacagtggcgccatctcagctcactgcaatctccacctcccaggttcaagcaattctcctgcctcagcctcccaagtagctgagattacaggcatgcaccaccatgcctagctaattttgtatttttagttgagacgaggtttctccatgttggtcaggctggtctcgaactcctgacctcaggtgatccgcccacctcggcctcccaaagtgctgggataacaggcatgagccaccacacccggccacttgggcatcttcagcacatgggtggcaggttgagccggagtgacaggtgacatccttgtggtccccagaatggctccagtgagaaacgcgaggtccgccagttccagtttacggcgtggccggaccatggcgtgcccgaatacccaacgcccttcctggctttcctgcggagagtcaagacctgcaacccgccagatgccggccccatcgtggttcactgcaggtacgtccaccccactctgccccatccgcttccccccggtccccgcagcccagcagcccctgaacgccacgtggctgtccctgcagtgccggtgtgggccgcacaggctgctttatcgtcatcgacgccatgcttgagcggatcaagccagagaagacagtcgatgtctatggccacgtgacgctcatgaggtcccagcgcaactacatggtgcagacggaggaccagtacagcttcatccacgaggccctgctggaggccgtgggctgtggcaacacagaagtgcccgcacgcagcctctatgcctacatccagaagctggcccaggtggagcctggcgaacacgtcactggcatggaactcgagttcaaggtgccagcggggtggaggctgctgtgggcggggtggggaggaggcccaaagagccgaaaatcaggcccgcctcgagcaatggggactagaaggtggtggcagggtgcccctacctacctatccaccattggtgctccagctgaaagcatagccttcctcatcctgatggtggcctctggcttcctcctccccacagcggctggctaactccaaggcccacacgtcacgcttcatcagtgccaatctgccttgtaacaagttcaagaaccgcctggtgaacatcatgccctatgagagcacacgggtctgtctgcaacccatccggggtgtggagggctctgactacatcaacgccagcttcattgatggctacaggtaggtgccatgcctttcccacctcagggccccagaaggagcccatctctactctcaagaggcctccagtcttgtgggagacagagctggggagatactggtagccctgagctgtttaaatacacatctttagatgtatatttaactgatggctgctgtccgtgaatctttgggtgtgtgtttaactgatggctgctgtccataatgtgattccatctgttctatcagtggttctcaattggggtaattctgacaccctgaggatatttcttattttcatgagtactactggtactgcctgggtggggaccaggcatgctgctcagcatcctacagtacacagaacagcttgtctcagagaacaatccagcgccaaatgtcaatagcactgagattgagaaatcctggcaaaggaggggagggtgttcctggcagagggtgccacatgtgcaaaggcccggggctgagagcttggcccacgcaagcaactgaaagtagggcagtggcccttgaaggagatggagggaagaaatcaggagaggttagtggatagtcccgcagctttgtcaagggatttgaatttgtcatgagactggtggtttccccacatgcaaaatgggatcaccaggaggggaagtggtgcacccagcagccattcctggcaggcagtggggtggtaggtgtagctgtcactggcttggtcctgaccctcccgggcagcatcaggtgagtaccacgccgggtctccctgccggccctgggctcatacccccaacgcctgcaggcagcagaaggcctacatcgcgacacaggggccgctggcggagaccacggaagacttctggcgcatgctgtgggagaacaattcgacgatcgtggtgatgctgaccaagctgcgggagatgggccgggtgagctaggggccacagcagggaagggcgagggccagactgtggctcagacagcgctgaccacggccgccatctcctccacaggagaagtgtcaccagtactggccggccgagcgctctgcccgctaccagtactttgtggtagatccgatggcagaatacaacatgcctcagtatatcctgcgagagttcaaggtcacagatgcccgggtgagtgaagagacagactggcgggaggggctggagccttagtgatctggaaagggctgccctggttggtgatggatacaaaggccaagggttaggcataagcattgggaatcaggagaagtgtcagctgaccagttggccacttgcttctactgccacataggtggaagagagggtcttatctctgtgctgaccctgcagtgtctccactcactagggaacatggccccaggggatgatattcaaggaagtgaatggggctttcctctggaagcagccacctgtggaggatgatgttaaagggctgaaagggacttgagggattgagggtgaggggtggtagacggtggacaaggagagtcagatggtggggactggcaccgttgggtgaggagagatgagtatggatggtgttggctcaatgaaggactgcagtgtcaggtggagggagaaaggccaggtattaggtggcaacagatgaagactcatagtggaaggtggccctgttgggtgacaaaggatcaaagttagtggtggaagatcgacgatggtagagaacgcagtgaggggatgatagaggatggggaatgttggccaagatgatgccaaaggatggtgaaggctgctgaatgaacagcaatcaccgggcattgtgctgtgagcagtgcaagatggtgcccttggaggggtggaatctggaattggagagaagaacattcgcataacaggatggagaatgtcagtggaatgtggtagtttgggtcagtgaagggtgatgctttatcagtgatggatggaggaagacagaattggagaagggcaaatgtttggccagcagggggtggagaaaatcaatgggaaatactaggactaagaaaggacagtaaacaatagtcaatggtataaaccaatgggtggtacaagattgaggaagatggggttgggaaaggagattttagaaaatgtaaaacatcagggaaagatggtacattggatggcaaatggtggcagaggataacaattttggatggcaccattttgatggtggaggatgggattagaacaaggcagatgatggggaatggagaatgtcagtggaagatggtacccttggatggtggagagtgatgaattgttagtgatggatggtggaaaatggcaccattgagttgtgaaggatggaagatggcagagaatgatgtggttggatggcaccactgggtggtaggaaaggagactgcaaagaggaatggttgagtagtggggaatgaagacagtcagtggaagatgccatcactgaatggttaaggatgagttaccaagaggggatggtggtggatgatggaagatgatggtgttgggtggaactcgggacggcagaaggtagtgagttaccagcagtgggtggtagcagtggcactgttattggatggtggaagatgatggtgttggatggtggacgatgaaagatgatggcaccattgggttgtagaagatgagtcatcattgatgagctatggacagtgatggagttgagttgtgctgtagggccccgaaatggcacagagtggaagatgacagaggatcatggtgaataggagctgggaagggcagagctggttgcactatgggatggcaaaggatggaggatggaggtgctggatgtcatacaggaaatgtggggagaggaacagctgggtggcaagaagtggtgaggatgaggttggagcatctcttatggaaagcagtaccattggattgtgaaggacggtaagttgtcagggtaaatgaatggcagagagtgtacaaaatgggggtgagggccgggcctggtggctcatgcctgtaatcccagctaatcgggaggctgaggcaggagaattacttgaatcactggaggtggaggttgcagtgagccgagatcacgccattgcactccaacctgggcgacaagagtgaaactccatctcaaaaacaaacaaacaaacaaaaaatgggggtgaagatagggaggatggagaaaaacccccatggccgctgctgacctgataacaagattgggctctctgcactctaggatggccagtcccggactgtccggcagttccagttcacagactggccggaacagggtgtgccaaagtcgggggagggcttcatcgacttcattggccaagtgcataagactaaggagcagtttggccaggacggccccatctctgtccactgcaggtgagctccctcgagtgtcccagcttttggccctgctggggaaactgaggcctggtggggacagccccaagcttaggctgtcccatctgggctgttagagtcctcggggaatcgggtggtcagatcagggctccccagcacctgcccctgaatggctgtgctcactttactgccaccagtgccggcgtgggcaggacgggcgtcttcatcacgcttagcatcgtgctggagcggatgcggtatgaaggcgtggtggacatctttcagacggtgaagatgctacgaacccagcggccggccatggtgcagacagaggtaaagatgggacagggaggggaggcagcctggcctcacgccccgactgcccctaagctccagtttcctcctctgtgagatgggtacagccacctgggactctctgggtcactgtgggtagacggtccacagacccctcccgcactggaaggggaccagtaacccttttaacacaacagtcagctgaaggtcaaaggcgagaagtggcccttggagtggacagagatgggctcattgcagtccccaagtcacagcaagtctgtggctcagcctggaccctgccaagaaccactgacccccaagtccaaccaatgtacagatggggaaactgaggcccagactcctagcttcaaggggaatgttccagtggagggggttctgaacagcagccatgtcttaaaagatgagtagaggggacccggcgtggcagctcacacctgtaatcccagcactttgggaggctgaggtgggaggatcgcttgagcccatcctgggcaacacagaaagaccccatctctacaaaaaaaatagaaatgaggctgggtgtggtggcttatgcctgtaatcccagcactttgggaggccaaggcaggcagatcacccgaggtcaggagtttgagaccagcctggccaacatggcgaaaccctgtctctactaaaaatacaaaaatcagctgggcatggtggtacacgcctgtcatcccagctacttgggaggctcaggcaggagaatcacttgaacccaggagacagaggttgcagggagccaagatggcgccactgcactccagcctgggcaacacagcgagactccgtctcaaaataaataaaaaagaaaagatgaatagggcctccaggcagacgggaggagagggcgttccgggtgaagagcctggctgtgttaggcagagaggtgtggcatagctcacttcattgtgggagggccgtgggggccaaagagaccttctgcaagagacacgcacaaggctgaggagcttggcctgctcctgagggcgatagggagccctgggaggcgttagagcagcggaggtactaacaggtcacctctgctcccccaggatgagtaccagttctgttaccaggcggcactggagtacctcggaagctttgaccactatgcaacctaaagccatggttccccccaggcccgacaccactggccccggatgcctctgcccctcccgggcggacctcctgaggcctggacccccagtgggcagggcaggaggtggcagcggcagcagctgtgtttctgcaccatttccgaggacgacgcagcccctcgagcccccccaccggccccggccgccccagcgacctccctggcaccggccgccgccttcaaatacttggcacattcctcctttccttccaattccaaaaccagattccggggtggggggtggggggatggtgagcaaataggagtgctccccagaaccagaggagggtggggcacagaccatagacggacccctcgtcctcccccagcggtggtagggggacccggggggctcctccccgctctgcagcctggggacactgggctgggaccagaatccagctttcttttaaaactctcagtgtaactgtatcccgtgacatttcattttttttaaatagtgtatttttttttccatttttttttttaagagaaacaaacaaaagactcgccagtcaatgactttcaaagagaactaactttggcttattcatattctgttcaaagacagtctattttttcactgtagaaagcgtccttgtgtgatagttacgttcgcaaacgcgcacgccaggcccatggctgtaccttggcttttttttttttttttttttttttaatttttcctaccatcagaaagtgtgctttgctcacagaagaatgggatgtccttttttctttcttggctttttttttccccctttttgtttcatttttataaattaaattttcagacatatcaaatacagttctgagggtaaggtcatgggggagctcggacccagtggcgttgggtgcggttgagggggacgctgctgtaagaggagagagatgacagtggtcctcctctgagagcctgagctgtctccccgtctcccgcccccaaggagacagagaggatcctacttcttcggggacagtggctgtatggctgtgctgccccacatcagggaccctttccccctgggactgtggggcagtttgggagcaaaaccagaaggacaggcccccctctacccgcctaccctgagcaagcgagttgttcctctttgtacaagggcaggtctgcggttactttcaacactgtttattccagcggaagcagccgggtggttttcccacccccgtgtatgtagatatatcgactttgtattaaaggaagatcgtctga

SEQ ID NO: 2 - DNA, 7347 bp, Homo sapiens
cctcgcgccgcccgcccggcagcccggccggcgcgcgcacgccgcgagccgctggcgctcgggctccgctcggatcccatgcaacagccacgatgtgaagcggggcagagccgggggagcccagcccagccagcctccagacgttgccccatctgacgctcggctcgaggcctctctgtgagggaccggggggccatccccctccagggcggagatcggaggtcgctgccaagcatggcgcccacctggggccctggcatggtgtctgtggttggtcccatgggcctccttgtggtcctgctcgttggaggctgtgcagcagaagagccccccaggtttatcaaagaacccaaggaccagatcggcgtgtcggggggtgtggcctctttcgtgtgtcaggccacgggtgaccccaagccacgagtgacctggaacaagaagggcaagaaggtcaactctcagcgctttgagacgattgagtttgatgagagtgcaggggcagtgctgaggatccagccgctgaggacaccgcgggatgaaaacgtgtacgagtgtgtggcccagaactcggttggggagatcacagtccatgccaagcttactgtcctccgagaggaccagctgccctctggcttccccaacatcgacatgggcccacagttgaaggtggtggagcggacacggacagccaccatgctctgtgcagccagcggcaaccctgaccctgagatcacctggttcaaggacttcctgcctgtggatcctagtgccagcaatggacgcatcaaacagctgcgatcagaaacctttgaaagcactccgattcgaggagccctgcagattgaaagcagtgaggaaaccgaccagggcaaatatgagtgtgtggccaccaacagcgccggcgtgcgctactcctcacctgccaacctctacgtgcgagagcttcgagaagtccgccgcgtggccccgcgcttctccatcctgcccatgagccacgagatcatgccagggggcaacgtgaacatcacctgcgtggccgtgggctcgcccatgccatacgtgaagtggatgcagggggccgaggacctgacccccgaggatgacatgcccgtgggtcggaacgtgctggaactcacagatgtcaaggactcggccaactacacctgcgtggccatgtccagcctgggcgtcattgaggcggttgctcagatcacggtgaaatctctccccaaagctcccgggactcccatggtgactgagaacacagccaccagcatcaccatcacgtgggactcgggcaacccagatcctgtgtcctattacgtcatcgaatataaatccaagagccaagacgggccgtatcagattaaagaggacatcaccaccacacgttacagcatcggcggcctgagccccaactcggagtacgagatctgggtgtcggccgtcaactccatcggccaggggccccccagcgagtccgtggtcacccgcacaggcgagcaggccccggccagcgcgccgcggaacgtgcaagcccggatgctcagcgcgaccaccatgattgtgcagtgggaggagccggtggagcccaacggcctgatccgcggctaccgcgtctactacaccatggaaccggagcaccccgtgggcaactggcagaagcacaacgtggacgacagcctgctgaccaccgtgggcagcctgctggaggacgagacctacaccgtgcgggtgctcgccttcacctccgtcggcgacgggcccctctcggaccccatccaggtcaagacgcagcagggagtgccgggccagcccatgaacctgcgggccgaggccaggtcggagaccagcatcacgctgtcctggagccccccgcggcaggagagtatcatcaagtacgagctcctcttccgggaaggcgaccatggccgggaggtgggaaggaccttcgacccgacgacttcctacgtggtggaggacctgaagcccaacacggagtacgccttccgcctggcggcccgctcgccgcagggcctgggcgccttcacccccgtggtgcggcagcgcacgctgcagtccaaaccgtcagccccccctcaagacgttaaatgtgtcagcgtgcgctccacggccattttggtaagttggcgcccgccgccgccggaaacgcacaacggggccctggtgggctacagcgtccgctaccgaccgctgggctcagaggacccggaacccaaggaggtgaacggcatccccccgaccaccactcagatcctgctggaggccttggagaagtggacccagtaccgcatcacgactgtcgctcacacagaggtgggaccagggcccgagagctcgcccgtggtcgtccgcaccgacgaggatgtgcccagcgcgccgccgcggaaggtggaggcggaggcgctcaacgccacggccatccgcgtgctgtggcgctcgcccgcgcccggccggcagcacggccagatccgcggctaccaggtccactacgtgcgcatggagggcgccgaggcccgcgggccgccgcgcatcaaggacgtcatgctggccgatgcccagtgggagacggatgacacggccgaatatgagatggtcatcacaaacttgcagcctgagaccgcgtactccatcacggtagccgcctacaccatgaagggcgatggcgctcgcagcaaacccaaggtggttgtgaccaagggagcagtgctgggccgcccaaccctgtcggtgcagcagacccccgagggcagcctgctggcacgctgggagcccccggctggcaccgcggaggaccaggtgctgggctaccgcctgcagtttggccgtgaggactcgacgcccctggccaccctggagttcccgccctccgaggaccgctacacggcatcaggcgtgcacaagggggccacgtatgtgttccggcttgcggcccggagccgcggcggcctgggcgaggaggcagccgaggtcctgagcatcccggaggacacgccccgtggccacccgcagattctggaggcggccggcaacgcctcggccgggaccgtccttctccgctggctgccacccgtgcccgccgagcgcaacggggccatcgtcaaatacacggtggccgtgcgggaggccggtgccctgggccctgcccgagagactgagctgccggcagcggctgagccgggcgcggagaacgcgctcacgctgcagggcctgaagcccgacacggcctatgacctccaagtgcgagcccacacgcgccggggccctggccccttcagcccccccgtccgctaccggacgttcctgcgggaccaagtctcgcccaagaacttcaaggtgaaaatgatcatgaagacatcagttctgctcagctgggagttccctgacaactacaactcacccacaccctacaagatccagtacaatgggctcacactggatgtggatggccgtaccaccaagaagctcatcacgcacctcaagccccacaccttctacaactttgtgctgaccaatcgcggcagcagcctgggcggcctccagcagacggtcaccgcctggactgccttcaacctgctcaacggcaagcccagcgtcgcccccaagcctgatgctgacggcttcatcatggtgtatcttcctgacggccagagccccgtgcctgtccagagctatttcattgtgatggtgccactgcgcaagtctcgtggaggccaattcctgaccccgctgggtagcccagaggacatggatctggaagagctcatccaggacatctcacggctacagaggcgcagcctgcggcactcgcgtcagctggaggtgccccggccctatattgcagctcgcttctctgtgctgccacccacgttccatcccggcgaccagaagcagtatggcggcttcgataaccggggcctggagcccggccaccgctatgtcctcttcgtgcttgccgtgcttcagaagagcgagcctacctttgcagccagtcccttctcagaccccttccagctggataacccggacccccagcccatcgtggatggcgaggaggggcttatctgggtgatcgggcctgtgctggccgtggtcttcataatctgcattgtcattgctatcctgctctacaagaacaaacccgacagtaaacgcaaggactcagaaccccgcaccaaatgcctcctgaacaatgccgacctcgcccctcaccaccccaaggaccctgtggaaatgagacgcattaacttccagactccagattcaggcctcaggagccccctcagggagccggggtttcactttgaaagcatgcttagccacccgccaattcccatcgcagacatggcggagcacacggagcggctcaaggccaacgacagcctcaagctctcccaggagtatgagtccatcgaccctggacagcagttcacatgggaacattccaacctggaagtgaacaagccgaagaaccgctatgccaacgtcatcgcctatgaccactcccgtgtcatcctccagcccattgaaggcatcatgggcagtgattacatcaatgccaactacgtggacggctaccggtgtcagaacgcgtacattgccacgcaggggccgctgcctgagacctttggggacttctggcgtatggtgtgggagcagcggtcggcgaccatcgtcatgatgacgcggctggaggagaagtcacggatcaagtgtgatcagtattggcccaacagaggcacggagacctacggcttcatccaggtcacgttgctagataccatcgagctggccacattctgcgtcaggacattctctctgcacaagaatggctccagtgagaaacgcgaggtccgccagttccagtttacggcgtggccggaccatggcgtgcccgaatacccaacgcccttcctggctttcctgcggagagtcaagacctgcaacccgccagatgccggccccatcgtggttcactgcagtgccggtgtgggccgcacaggctgctttatcgtcatcgacgccatgcttgagcggatcaagccagagaagacagtcgatgtctatggccacgtgacgctcatgaggtcccagcgcaactacatggtgcagacggaggaccagtacagcttcatccacgaggccctgctggaggccgtgggctgtggcaacacagaagtgcccgcacgcagcctctatgcctacatccagaagctggcccaggtggagcctggcgaacacgtcactggcatggaactcgagttcaagcggctggctaactccaaggcccacacgtcacgcttcatcagtgccaatctgccttgtaacaagttcaagaaccgcctggtgaacatcatgccctatgagagcacacgggtctgtctgcaacccatccggggtgtggagggctctgactacatcaacgccagcttcattgatggctacaggcagcagaaggcctacatcgcgacacaggggccgctggcggagaccacggaagacttctggcgcatgctgtgggagaacaattcgacgatcgtggtgatgctgaccaagctgcgggagatgggccgggagaagtgtcaccagtactggccggccgagcgctctgcccgctaccagtactttgtggtagatccgatggcagaatacaacatgcctcagtatatcctgcgagagttcaaggtcacagatgcccgggatggccagtcccggactgtccggcagttccagttcacagactggccggaacagggtgtgccaaagtcgggggagggcttcatcgacttcattggccaagtgcataagactaaggagcagtttggccaggacggccccatctctgtccactgcagtgccggcgtgggcaggacgggcgtcttcatcacgcttagcatcgtgctggagcggatgcggtatgaaggcgtggtggacatctttcagacggtgaagatgctacgaacccagcggccggccatggtgcagacagaggatgagtaccagttctgttaccaggcggcactggagtacctcggaagctttgaccactatgcaacctaaagccatggttccccccaggcccgacaccactggccccggatgcctctgcccctcccgggcggacctcctgaggcctggacccccagtgggcagggcaggaggtggcagcggcagcagctgtgtttctgcaccatttccgaggacgacgcagcccctcgagcccccccaccggccccggccgccccagcgacctccctggcaccggccgccgccttcaaatacttggcacattcctcctttccttccaattccaaaaccagattccggggtggggggtggggggatggtgagcaaataggagtgctccccagaaccagaggagggtggggcacagaccatagacggacccctcgtcctcccccagcggtggtagggggacccggggggctcctccccgctctgcagcctggggacactgggctgggaccagaatccagctttcttttaaaactctcagtgtaactgtatcccgtgacatttcattttttttaaatagtgtatttttttttccatttttttttttaagagaaacaaacaaaagactcgccagtcaatgactttcaaagagaactaactttggcttattcatattctgttcaaagacagtctattttttcactgtagaaagcgtccttgtgtgatagttacgttcgcaaacgcgcacgccaggcccatggctgtaccttggcttttttttttttttttttttttttaatttttcctaccatcagaaagtgtgctttgctcacagaagaatgggatgtccttttttctttcttggctttttttttccccctttttgtttcatttttataaattaaattttcagacatatcaaatacagttctgagggtaaggtcatgggggagctcggacccagtggcgttgggtgcggttgagggggacgctgctgtaagaggagagagatgacagtggtcctcctctgagagcctgagctgtctccccgtctcccgcccccaaggagacagagaggatcctacttcttcggggacagtggctgtatggctgtgctgccccacatcagggaccctttccccctgggactgtggggcagtttgggagcaaaaccagaaggacaggcccccctctacccgcctaccctgagcaagcgagttgttcctctttgtacaagggcaggtctgcggttactttcaacactgtttattccagcggaagcagccgggtggttttcccacccccgtgtatgtagatatatcgactttgtattaaaggaagatcgtctga

SEQ ID NO: 3 - protein, 1948 aa, Homo sapiens
MAPTWGPGMVSVVGPMGLLVVLLVGGCAAEEPPRFIKEPKDQIGVSGGVASFVCQATGDPKPRVTWNKKGKKVNSQRFETIEFDESAGAVLRIQPLRTPRDENVYECVAQNSVGEITVHAKLTVLREDQLPSGFPNIDMGPQLKVVERTRTATMLCAASGNPDPEITWFKDFLPVDPSASNGRIKQLRSETFESTPIRGALQIESSEETDQGKYECVATNSAGVRYSSPANLYVRELREVRRVAPRFSILPMSHEIMPGGNVNITCVAVGSPMPYVKWMQGAEDLTPEDDMPVGRNVLELTDVKDSANYTCVAMSSLGVIEAVAQITVKSLPKAPGTPMVTENTATSITITWDSGNPDPVSYYVIEYKSKSQDGPYQIKEDITTTRYSIGGLSPNSEYEIWVSAVNSIGQGPPSESVVTRTGEQAPASAPRNVQARMLSATTMIVQWEEPVEPNGLIRGYRVYYTMEPEHPVGNWQKHNVDDSLLTTVGSLLEDETYTVRVLAFTSVGDGPLSDPIQVKTQQGVPGQPMNLRAEARSETSITLSWSPPRQESIIKYELLFREGDHGREVGRTFDPTTSYVVEDLKPNTEYAFRLAARSPQGLGAFTPVVRQRTLQSKPSAPPQDVKCVSVRSTAILVSWRPPPPETHNGALVGYSVRYRPLGSEDPEPKEVNGIPPTTTQILLEALEKWTQYRITTVAHTEVGPGPESSPVVVRTDEDVPSAPPRKVEAEALNATAIRVLWRSPAPGRQHGQIRGYQVHYVRMEGAEARGPPRIKDVMLADAQWETDDTAEYEMVITNLQPETAYSITVAAYTMKGDGARSKPKVVVTKGAVLGRPTLSVQQTPEGSLLARWEPPAGTAEDQVLGYRLQFGREDSTPLATLEFPPSEDRYTASGVHKGATYVFRLAARSRGGLGEEAAEVLSIPEDTPRGHPQILEAAGNASAGTVLLRWLPPVPAERNGAIVKYTVAVREAGALGPARETELPAAAEPGAENALTLQGLKPDTAYDLQVRAHTRRGPGPFSPPVRYRTFLRDQVSPKNFKVKMIMKTSVLLSWEFPDNYNSPTPYKIQYNGLTLDVDGRTTKKLITHLKPHTFYNFVLTNRGSSLGGLQQTVTAWTAFNLLNGKPSVAPKPDADGFIMVYLPDGQSPVPVQSYFIVMVPLRKSRGGQFLTPLGSPEDMDLEELIQDISRLQRRSLRHSRQLEVPRPYIAARFSVLPPTFHPGDQKQYGGFDNRGLEPGHRYVLFVLAVLQKSEPTFAASPFSDPFQLDNPDPQPIVDGEEGLIWVIGPVLAVVFIICIVIAILLYKNKPDSKRKDSEPRTKCLLNNADLAPHHPKDPVEMRRINFQTPDSGLRSPLREPGFHFESMLSHPPIPIADMAEHTERLKANDSLKLSQEYESIDPGQQFTWEHSNLEVNKPKNRYANVIAYDHSRVILQPIEGIMGSDYINANYVDGYRCQNAYIATQGPLPETFGDFWRMVWEQRSATIVMMTRLEEKSRIKCDQYWPNRGTETYGFIQVTLLDTIELATFCVRTFSLHKNGSSEKREVRQFQFTAWPDHGVPEYPTPFLAFLRRVKTCNPPDAGPIVVHCSAGVGRTGCFIVIDAMLERIKPEKTVDVYGHVTLMRSQRNYMVQTEDQYSFIHEALLEAVGCGNTEVPARSLYAYIQKLAQVEPGEHVTGMELEFKRLANSKAHTSRFISANLPCNKFKNRLVNIMPYESTRVCLQPIRGVEGSDYINASFIDGYRQQKAYIATQGPLAETTEDFWRMLWENNSTIVVMLTKLREMGREKCHQYWPAERSARYQYFVVDPMAEYNMPQYILREFKVTDARDGQSRTVRQFQFTDWPEQGVPKSGEGFIDFIGQVHKTKEQFGQDGPISVHCSAGVGRTGVFITLSIVLERMRYEGVVDIFQTVKMLRTQRPAMVQTEDEYQFCYQAALEYLGSFDHYAT

SEQ ID NO: 4 - DNA, 7233 bp, Homo sapiens
cctcgcgccgcccgcccggcagcccggccggcgcgcgcacgccgcgagccgctggcgctcgggctccgctcggatcccatgcaacagccacgatgtgaagcggggcagagccgggggagcccagcccagccagcctccagacgttgccccatctgacgctcggctcgaggcctctctgtgagggaccggggggccatccccctccagggcggagatcggaggtcgctgccaagcatggcgcccacctggggccctggcatggtgtctgtggttggtcccatgggcctccttgtggtcctgctcgttggaggctgtgcagcagaagagccccccaggtttatcaaagaacccaaggaccagatcggcgtgtcggggggtgtggcctctttcgtgtgtcaggccacgggtgaccccaagccacgagtgacctggaacaagaagggcaagaaggtcaactctcagcgctttgagacgattgagtttgatgagagtgcaggggcagtgctgaggatccagccgctgaggacaccgcgggatgaaaacgtgtacgagtgtgtggcccagaactcggttggggagatcacagtccatgccaagcttactgtcctccgagaggaccagctgccctctggcttccccaacatcgacatgggcccacagttgaaggtggtggagcggacacggacagccaccatgctctgtgcagccagcggcaaccctgaccctgagatcacctggttcaaggacttcctgcctgtggatcctagtgccagcaatggacgcatcaaacagctgcgatcaggagccctgcagattgaaagcagtgaggaaaccgaccagggcaaatatgagtgtgtggccaccaacagcgccggcgtgcgctactcctcacctgccaacctctacgtgcgagtccgccgcgtggccccgcgcttctccatcctgcccatgagccacgagatcatgccagggggcaacgtgaacatcacctgcgtggccgtgggctcgcccatgccatacgtgaagtggatgcagggggccgaggacctgacccccgaggatgacatgcccgtgggtcggaacgtgctggaactcacagatgtcaaggactcggccaactacacctgcgtggccatgtccagcctgggcgtcattgaggcggttgctcagatcacggtgaaatctctccccaaagctcccgggactcccatggtgactgagaacacagccaccagcatcaccatcacgtgggactcgggcaacccagatcctgtgtcctattacgtcatcgaatataaatccaagagccaagacgggccgtatcagattaaagaggacatcaccaccacacgttacagcatcggcggcctgagccccaactcggagtacgagatctgggtgtcggccgtcaactccatcggccaggggccccccagcgagtccgtggtcacccgcacaggcgagcaggccccggccagcgcgccgcggaacgtgcaagcccggatgctcagcgcgaccaccatgattgtgcagtgggaggagccggtggagcccaacggcctgatccgcggctaccgcgtctactacaccatggaaccggagcaccccgtgggcaactggcagaagcacaacgtggacgacagcctgctgaccaccgtgggcagcctgctggaggacgagacctacaccgtgcgggtgctcgccttcacctccgtcggcgacgggcccctctcggaccccatccaggtcaagacgcagcagggagtgccgggccagcccatgaacctgcgggccgaggccaggtcggagaccagcatcacgctgtcctggagccccccgcggcaggagagtatcatcaagtacgagctcctcttccgggaaggcgaccatggccgggaggtgggaaggaccttcgacccgacgacttcctacgtggtggaggacctgaagcccaacacggagtacgccttccgcctggcggcccgctcgccgcagggcctgggcgccttcacccccgtggtgcggcagcgcacgctgcagtccaaaccgtcagccccccctcaagacgttaaatgtgtcagcgtgcgctccacggccattttggtaagttggcgcccgccgccgccggaaacgcacaacggggccctggtgggctacagcgtccgctaccgaccgctgggctcagaggacccggaacccaaggaggtgaacggcatccccccgaccaccactcagatcctgctggaggccttggagaagtggacccagtaccgcatcacgactgtcgctcacacagaggtgggaccagggcccgagagctcgcccgtggtcgtccgcaccgacgaggatgtgcccagcgcgccgccgcggaaggtggaggcggaggcgctcaacgccacggccatccgcgtgctgtggcgctcgcccgcgcccggccggcagcacggccagatccgcggctaccaggtccactacgtgcgcatggagggcgccgaggcccgcgggccgccgcgcatcaaggacgtcatgctggccgatgcccaggagatggtcatcacaaacttgcagcctgagaccgcgtactccatcacggtagccgcctacaccatgaagggcgatggcgctcgcagcaaacccaaggtggttgtgaccaagggagcagtgctgggccgcccaaccctgtcggtgcagcagacccccgagggcagcctgctggcacgctgggagcccccggctggcaccgcggaggaccaggtgctgggctaccgcctgcagtttggccgtgaggactcgacgcccctggccaccctggagttcccgccctccgaggaccgctacacggcatcaggcgtgcacaagggggccacgtatgtgttccggcttgcggcccggagccgcggcggcctgggcgaggaggcagccgaggtcctgagcatcccggaggacacgccccgtggccacccgcagattctggaggcggccggcaacgcctcggccgggaccgtccttctccgctggctgccacccgtgcccgccgagcgcaacggggccatcgtcaaatacacggtggccgtgcgggaggccggtgccctgggccctgcccgagagactgagctgccggcagcggctgagccgggcgcggagaacgcgctcacgctgcagggcctgaagcccgacacggcctatgacctccaagtgcgagcccacacgcgccggggccctggccccttcagcccccccgtccgctaccggacgttcctgcgggaccaagtctcgcccaagaacttcaaggtgaaaatgatcatgaagacatcagttctgctcagctgggagttccctgacaactacaactcacccacaccctacaagatccagtacaatgggctcacactggatgtggatggccgtaccaccaagaagctcatcacgcacctcaagccccacaccttctacaactttgtgctgaccaatcgcggcagcagcctgggcggcctccagcagacggtcaccgcctggactgccttcaacctgctcaacggcaagcccagcgtcgcccccaagcctgatgctgacggcttcatcatggtgtatcttcctgacggccagagccccgtgcctgtccagagctatttcattgtgatggtgccactgcgcaagtctcgtggaggccaattcctgaccccgctgggtagcccagaggacatggatctggaagagctcatccaggacatctcacggctacagaggcgcagcctgcggcactcgcgtcagctggaggtgccccggccctatattgcagctcgcttctctgtgctgccacccacgttccatcccggcgaccagaagcagtatggcggcttcgataaccggggcctggagcccggccaccgctatgtcctcttcgtgcttgccgtgcttcagaagagcgagcctacctttgcagccagtcccttctcagaccccttccagctggataacccggacccccagcccatcgtggatggcgaggaggggcttatctgggtgatcgggcctgtgctggccgtggtcttcataatctgcattgtcattgctatcctgctctacaagaacaaacccgacagtaaacgcaaggactcagaaccccgcaccaaatgcctcctgaacaatgccgacctcgcccctcaccaccccaaggaccctgtggaaatgagacgcattaacttccagactccaggcatgcttagccacccgccaattcccatcgcagacatggcggagcacacggagcggctcaaggccaacgacagcctcaagctctcccaggagtatgagtccatcgaccctggacagcagttcacatgggaacattccaacctggaagtgaacaagccgaagaaccgctatgccaacgtcatcgcctatgaccactcccgtgtcatcctccagcccattgaaggcatcatgggcagtgattacatcaatgccaactacgtggacggctaccggtgtcagaacgcgtacattgccacgcaggggccgctgcctgagacctttggggacttctggcgtatggtgtgggagcagcggtcggcgaccatcgtcatgatgacgcggctggaggagaagtcacggatcaagtgtgatcagtattggcccaacagaggcacggagacctacggcttcatccaggtcacgttgctagataccatcgagctggccacattctgcgtcaggacattctctctgcacaagaatggctccagtgagaaacgcgaggtccgccagttccagtttacggcgtggccggaccatggcgtgcccgaatacccaacgcccttcctggctttcctgcggagagtcaagacctgcaacccgccagatgccggccccatcgtggttcactgcagtgccggtgtgggccgcacaggctgctttatcgtcatcgacgccatgcttgagcggatcaagccagagaagacagtcgatgtctatggccacgtgacgctcatgaggtcccagcgcaactacatggtgcagacggaggaccagtacagcttcatccacgaggccctgctggaggccgtgggctgtggcaacacagaagtgcccgcacgcagcctctatgcctacatccagaagctggcccaggtggagcctggcgaacacgtcactggcatggaactcgagttcaagcggctggctaactccaaggcccacacgtcacgcttcatcagtgccaatctgccttgtaacaagttcaagaaccgcctggtgaacatcatgccctatgagagcacacgggtctgtctgcaacccatccggggtgtggagggctctgactacatcaacgccagcttcattgatggctacaggcagcagaaggcctacatcgcgacacaggggccgctggcggagaccacggaagacttctggcgcatgctgtgggagaacaattcgacgatcgtggtgatgctgaccaagctgcgggagatgggccgggagaagtgtcaccagtactggccggccgagcgctctgcccgctaccagtactttgtggtagatccgatggcagaatacaacatgcctcagtatatcctgcgagagttcaaggtcacagatgcccgggatggccagtcccggactgtccggcagttccagttcacagactggccggaacagggtgtgccaaagtcgggggagggcttcatcgacttcattggccaagtgcataagactaaggagcagtttggccaggacggccccatctctgtccactgcagtgccggcgtgggcaggacgggcgtcttcatcacgcttagcatcgtgctggagcggatgcggtatgaaggcgtggtggacatctttcagacggtgaagatgctacgaacccagcggccggccatggtgcagacagaggatgagtaccagttctgttaccaggcggcactggagtacctcggaagctttgaccactatgcaacctaaagccatggttccccccaggcccgacaccactggccccggatgcctctgcccctcccgggcggacctcctgaggcctggacccccagtgggcagggcaggaggtggcagcggcagcagctgtgtttctgcaccatttccgaggacgacgcagcccctcgagcccccccaccggccccggccgccccagcgacctccctggcaccggccgccgccttcaaatacttggcacattcctcctttccttccaattccaaaaccagattccggggtggggggtggggggatggtgagcaaataggagtgctccccagaaccagaggagggtggggcacagaccatagacggacccctcgtcctcccccagcggtggtagggggacccggggggctcctccccgctctgcagcctggggacactgggctgggaccagaatccagctttcttttaaaactctcagtgtaactgtatcccgtgacatttcattttttttaaatagtgtatttttttttccatttttttttttaagagaaacaaacaaaagactcgccagtcaatgactttcaaagagaactaactttggcttattcatattctgttcaaagacagtctattttttcactgtagaaagcgtccttgtgtgatagttacgttcgcaaacgcgcacgccaggcccatggctgtaccttggcttttttttttttttttttttttttaatttttcctaccatcagaaagtgtgctttgctcacagaagaatgggatgtccttttttctttcttggctttttttttccccctttttgtttcatttttataaattaaattttcagacatatcaaatacagttctgagggtaaggtcatgggggagctcggacccagtggcgttgggtgcggttgagggggacgctgctgtaagaggagagagatgacagtggtcctcctctgagagcctgagctgtctccccgtctcccgcccccaaggagacagagaggatcctacttcttcggggacagtggctgtatggctgtgctgccccacatcagggaccctttccccctgggactgtggggcagtttgggagcaaaaccagaaggacaggcccccctctacccgcctaccctgagcaagcgagttgttcctctttgtacaagggcaggtctgcggttactttcaacactgtttattccagcggaagcagccgggtggttttcccacccccgtgtatgtagatatatcgactttgtattaaaggaagatcgtctga

SEQ ID NO: 5 - protein, 1910 aa, Homo sapiens
MAPTWGPGMVSVVGPMGLLVVLLVGGCAAEEPPRFIKEPKDQIGVSGGVASFVCQATGDPKPRVTWNKKGKKVNSQRFETIEFDESAGAVLRIQPLRTPRDENVYECVAQNSVGEITVHAKLTVLREDQLPSGFPNIDMGPQLKVVERTRTATMLCAASGNPDPEITWFKDFLPVDPSASNGRIKQLRSGALQIESSEETDQGKYECVATNSAGVRYSSPANLYVRVRRVAPRFSILPMSHEIMPGGNVNITCVAVGSPMPYVKWMQGAEDLTPEDDMPVGRNVLELTDVKDSANYTCVAMSSLGVIEAVAQITVKSLPKAPGTPMVTENTATSITITWDSGNPDPVSYYVIEYKSKSQDGPYQIKEDITTTRYSIGGLSPNSEYEIWVSAVNSIGQGPPSESVVTRTGEQAPASAPRNVQARMLSATTMIVQWEEPVEPNGLIRGYRVYYTMEPEHPVGNWQKHNVDDSLLTTVGSLLEDETYTVRVLAFTSVGDGPLSDPIQVKTQQGVPGQPMNLRAEARSETSITLSWSPPRQESIIKYELLFREGDHGREVGRTFDPTTSYVVEDLKPNTEYAFRLAARSPQGLGAFTPVVRQRTLQSKPSAPPQDVKCVSVRSTAILVSWRPPPPETHNGALVGYSVRYRPLGSEDPEPKEVNGIPPTTTQILLEALEKWTQYRITTVAHTEVGPGPESSPVVVRTDEDVPSAPPRKVEAEALNATAIRVLWRSPAPGRQHGQIRGYQVHYVRMEGAEARGPPRIKDVMLADAQEMVITNLQPETAYSITVAAYTMKGDGARSKPKVVVTKGAVLGRPTLSVQQTPEGSLLARWEPPAGTAEDQVLGYRLQFGREDSTPLATLEFPPSEDRYTASGVHKGATYVFRLAARSRGGLGEEAAEVLSIPEDTPRGHPQILEAAGNASAGTVLLRWLPPVPAERNGAIVKYTVAVREAGALGPARETELPAAAEPGAENALTLQGLKPDTAYDLQVRAHTRRGPGPFSPPVRYRTFLRDQVSPKNFKVKMIMKTSVLLSWEFPDNYNSPTPYKIQYNGLTLDVDGRTTKKLITHLKPHTFYNFVLTNRGSSLGGLQQTVTAWTAFNLLNGKPSVAPKPDADGFIMVYLPDGQSPVPVQSYFIVMVPLRKSRGGQFLTPLGSPEDMDLEELIQDISRLQRRSLRHSRQLEVPRPYIAARFSVLPPTFHPGDQKQYGGFDNRGLEPGHRYVLFVLAVLQKSEPTFAASPFSDPFQLDNPDPQPIVDGEEGLIWVIGPVLAVVFIICIVIAILLYKNKPDSKRKDSEPRTKCLLNNADLAPHHPKDPVEMRRINFQTPGMLSHPPIPIADMAEHTERLKANDSLKLSQEYESIDPGQQFTWEHSNLEVNKPKNRYANVIAYDHSRVILQPIEGIMGSDYINANYVDGYRCQNAYIATQGPLPETFGDFWRMVWEQRSATIVMMTRLEEKSRIKCDQYWPNRGTETYGFIQVTLLDTIELATFCVRTFSLHKNGSSEKREVRQFQFTAWPDHGVPEYPTPFLAFLRRVKTCNPPDAGPIVVHCSAGVGRTGCFIVIDAMLERIKPEKTVDVYGHVTLMRSQRNYMVQTEDQYSFIHEALLEAVGCGNTEVPARSLYAYIQKLAQVEPGEHVTGMELEFKRLANSKAHTSRFISANLPCNKFKNRLVNIMPYESTRVCLQPIRGVEGSDYINASFIDGYRQQKAYIATQGPLAETTEDFWRMLWENNSTIVVMLTKLREMGREKCHQYWPAERSARYQYFVVDPMAEYNMPQYILREFKVTDARDGQSRTVRQFQFTDWPEQGVPKSGEGFIDFIGQVHKTKEQFGQDGPISVHCSAGVGRTGVFITLSIVLERMRYEGVVDIFQTVKMLRTQRPAMVQTEDEYQFCYQAALEYLGSFDHYAT

SEQ ID NO: 6 - DNA, 6006 bp, Homo sapiens
cctcgcgccgcccgcccggcagcccggccggcgcgcgcacgccgcgagccgctggcgctcgggctccgctcggatcccatgcaacagccacgatgtgaagcggggcagagccgggggagcccagcccagccagcctccagacgttgccccatctgacgctcggctcgaggcctctctgtgagggaccggggggccatccccctccagggcggagatcggaggtcgctgccaagcatggcgcccacctggggccctggcatggtgtctgtggttggtcccatgggcctccttgtggtcctgctcgttggaggctgtgcagcagaagagccccccaggtttatcaaagaacccaaggaccagatcggcgtgtcggggggtgtggcctctttcgtgtgtcaggccacgggtgaccccaagccacgagtgacctggaacaagaagggcaagaaggtcaactctcagcgctttgagacgattgagtttgatgagagtgcaggggcagtgctgaggatccagccgctgaggacaccgcgggatgaaaacgtgtacgagtgtgtggcccagaactcggttggggagatcacagtccatgccaagcttactgtcctccgagaggaccagctgccctctggcttccccaacatcgacatgggcccacagttgaaggtggtggagcggacacggacagccaccatgctctgtgcagccagcggcaaccctgaccctgagatcacctggttcaaggacttcctgcctgtggatcctagtgccagcaatggacgcatcaaacagctgcgatcaggagccctgcagattgaaagcagtgaggaaaccgaccagggcaaatatgagtgtgtggccaccaacagcgccggcgtgcgctactcctcacctgccaacctctacgtgcgagtccgccgcgtggccccgcgcttctccatcctgcccatgagccacgagatcatgccagggggcaacgtgaacatcacctgcgtggccgtgggctcgcccatgccatacgtgaagtggatgcagggggccgaggacctgacccccgaggatgacatgcccgtgggtcggaacgtgctggaactcacagatgtcaaggactcggccaactacacctgcgtggccatgtccagcctgggcgtcattgaggcggttgctcagatcacggtgaaatctctccccaaagctcccgggactcccatggtgactgagaacacagccaccagcatcaccatcacgtgggactcgggcaacccagatcctgtgtcctattacgtcatcgaatataaatccaagagccaagacgggccgtatcagattaaagaggacatcaccaccacacgttacagcatcggcggcctgagccccaactcggagtacgagatctgggtgtcggccgtcaactccatcggccaggggccccccagcgagtccgtggtcacccgcacaggcgagcaggccccggccagcgcgccgcggaacgtgcaagcccggatgctcagcgcgaccaccatgattgtgcagtgggaggagccggtggagcccaacggcctgatccgcggctaccgcgtctactacaccatggaaccggagcaccccgtgggcaactggcagaagcacaacgtggacgacagcctgctgaccaccgtgggcagcctgctggaggacgagacctacaccgtgcgggtgctcgccttcacctccgtcggcgacgggcccctctcggaccccatccaggtcaagacgcagcagggagtgccgggccagcccatgaacctgcgggccgaggccaggtcggagaccagcatcacgctgtcctggagccccccgcggcaggagagtatcatcaagtacgagctcctcttccgggaaggcgaccatggccgggaggtgggaaggaccttcgacccgacgacttcctacgtggtggaggacctgaagcccaacacggagtacgccttccgcctggcggcccgctcgccgcagggcctgggcgccttcacccccgtggtgcggcagcgcacgctgcagtccatctcgcccaagaacttcaaggtgaaaatgatcatgaagacatcagttctgctcagctgggagttccctgacaactacaactcacccacaccctacaagatccagtacaatgggctcacactggatgtggatggccgtaccaccaagaagctcatcacgcacctcaagccccacaccttctacaactttgtgctgaccaatcgcggcagcagcctgggcggcctccagcagacggtcaccgcctggactgccttcaacctgctcaacggcaagcccagcgtcgcccccaagcctgatgctgacggcttcatcatggtgtatcttcctgacggccagagccccgtgcctgtccagagctatttcattgtgatggtgccactgcgcaagtctcgtggaggccaattcctgaccccgctgggtagcccagaggacatggatctggaagagctcatccaggacatctcacggctacagaggcgcagcctgcggcactcgcgtcagctggaggtgccccggccctatattgcagctcgcttctctgtgctgccacccacgttccatcccggcgaccagaagcagtatggcggcttcgataaccggggcctggagcccggccaccgctatgtcctcttcgtgcttgccgtgcttcagaagagcgagcctacctttgcagccagtcccttctcagaccccttccagctggataacccggacccccagcccatcgtggatggcgaggaggggcttatctgggtgatcgggcctgtgctggccgtggtcttcataatctgcattgtcattgctatcctgctctacaagaacaaacccgacagtaaacgcaaggactcagaaccccgcaccaaatgcctcctgaacaatgccgacctcgcccctcaccaccccaaggaccctgtggaaatgagacgcattaacttccagactccaggcatgcttagccacccgccaattcccatcgcagacatggcggagcacacggagcggctcaaggccaacgacagcctcaagctctcccaggagtatgagtccatcgaccctggacagcagttcacatgggaacattccaacctggaagtgaacaagccgaagaaccgctatgccaacgtcatcgcctatgaccactcccgtgtcatcctccagcccattgaaggcatcatgggcagtgattacatcaatgccaactacgtggacggctaccggtgtcagaacgcgtacattgccacgcaggggccgctgcctgagacctttggggacttctggcgtatggtgtgggagcagcggtcggcgaccatcgtcatgatgacgcggctggaggagaagtcacggatcaagtgtgatcagtattggcccaacagaggcacggagacctacggcttcatccaggtcacgttgctagataccatcgagctggccacattctgcgtcaggacattctctctgcacaagaatggctccagtgagaaacgcgaggtccgccagttccagtttacggcgtggccggaccatggcgtgcccgaatacccaacgcccttcctggctttcctgcggagagtcaagacctgcaacccgccagatgccggccccatcgtggttcactgcagtgccggtgtgggccgcacaggctgctttatcgtcatcgacgccatgcttgagcggatcaagccagagaagacagtcgatgtctatggccacgtgacgctcatgaggtcccagcgcaactacatggtgcagacggaggaccagtacagcttcatccacgaggccctgctggaggccgtgggctgtggcaacacagaagtgcccgcacgcagcctctatgcctacatccagaagctggcccaggtggagcctggcgaacacgtcactggcatggaactcgagttcaagcggctggctaactccaaggcccacacgtcacgcttcatcagtgccaatctgccttgtaacaagttcaagaaccgcctggtgaacatcatgccctatgagagcacacgggtctgtctgcaacccatccggggtgtggagggctctgactacatcaacgccagcttcattgatggctacaggcagcagaaggcctacatcgcgacacaggggccgctggcggagaccacggaagacttctggcgcatgctgtgggagaacaattcgacgatcgtggtgatgctgaccaagctgcgggagatgggccgggagaagtgtcaccagtactggccggccgagcgctctgcccgctaccagtactttgtggtagatccgatggcagaatacaacatgcctcagtatatcctgcgagagttcaaggtcacagatgcccgggatggccagtcccggactgtccggcagttccagttcacagactggccggaacagggtgtgccaaagtcgggggagggcttcatcgacttcattggccaagtgcataagactaaggagcagtttggccaggacggccccatctctgtccactgcagtgccggcgtgggcaggacgggcgtcttcatcacgcttagcatcgtgctggagcggatgcggtatgaaggcgtggtggacatctttcagacggtgaagatgctacgaacccagcggccggccatggtgcagacagaggatgagtaccagttctgttaccaggcggcactggagtacctcggaagctttgaccactatgcaacctaaagccatggttccccccaggcccgacaccactggccccggatgcctctgcccctcccgggcggacctcctgaggcctggacccccagtgggcagggcaggaggtggcagcggcagcagctgtgtttctgcaccatttccgaggacgacgcagcccctcgagcccccccaccggccccggccgccccagcgacctccctggcaccggccgccgccttcaaatacttggcacattcctcctttccttccaattccaaaaccagattccggggtggggggtggggggatggtgagcaaataggagtgctccccagaaccagaggagggtggggcacagaccatagacggacccctcgtcctcccccagcggtggtagggggacccggggggctcctccccgctctgcagcctggggacactgggctgggaccagaatccagctttcttttaaaactctcagtgtaactgtatcccgtgacatttcattttttttaaatagtgtatttttttttccatttttttttttaagagaaacaaacaaaagactcgccagtcaatgactttcaaagagaactaactttggcttattcatattctgttcaaagacagtctattttttcactgtagaaagcgtccttgtgtgatagttacgttcgcaaacgcgcacgccaggcccatggctgtaccttggcttttttttttttttttttttttttaatttttcctaccatcagaaagtgtgctttgctcacagaagaatgggatgtccttttttctttcttggctttttttttccccctttttgtttcatttttataaattaaattttcagacatatcaaatacagttctgagggtaaggtcatgggggagctcggacccagtggcgttgggtgcggttgagggggacgctgctgtaagaggagagagatgacagtggtcctcctctgagagcctgagctgtctccccgtctcccgcccccaaggagacagagaggatcctacttcttcggggacagtggctgtatggctgtgctgccccacatcagggaccctttccccctgggactgtggggcagtttgggagcaaaaccagaaggacaggcccccctctacccgcctaccctgagcaagcgagttgttcctctttgtacaagggcaggtctgcggttactttcaacactgtttattccagcggaagcagccgggtggttttcccacccccgtgtatgtagatatatcgactttgtattaaaggaagatcgtctga

SEQ ID NO: 7 - protein, 1501 aa, Homo sapiens
MAPTWGPGMVSVVGPMGLLVVLLVGGCAAEEPPRFIKEPKDQIGVSGGVASFVCQATGDPKPRVTWNKKGKKVNSQRFETIEFDESAGAVLRIQPLRTPRDENVYECVAQNSVGEITVHAKLTVLREDQLPSGFPNIDMGPQLKVVERTRTATMLCAASGNPDPEITWFKDFLPVDPSASNGRIKQLRSGALQIESSEETDQGKYECVATNSAGVRYSSPANLYVRVRRVAPRFSILPMSHEIMPGGNVNITCVAVGSPMPYVKWMQGAEDLTPEDDMPVGRNVLELTDVKDSANYTCVAMSSLGVIEAVAQITVKSLPKAPGTPMVTENTATSITITWDSGNPDPVSYYVIEYKSKSQDGPYQIKEDITTTRYSIGGLSPNSEYEIWVSAVNSIGQGPPSESVVTRTGEQAPASAPRNVQARMLSATTMIVQWEEPVEPNGLIRGYRVYYTMEPEHPVGNWQKHNVDDSLLTTVGSLLEDETYTVRVLAFTSVGDGPLSDPIQVKTQQGVPGQPMNLRAEARSETSITLSWSPPRQESIIKYELLFREGDHGREVGRTFDPTTSYVVEDLKPNTEYAFRLAARSPQGLGAFTPVVRQRTLQSISPKNFKVKMIMKTSVLLSWEFPDNYNSPTPYKIQYNGLTLDVDGRTTKKLITHLKPHTFYNFVLTNRGSSLGGLQQTVTAWTAFNLLNGKPSVAPKPDADGFIMVYLPDGQSPVPVQSYFIVMVPLRKSRGGQFLTPLGSPEDMDLEELIQDISRLQRRSLRHSRQLEVPRPYIAARFSVLPPTFHPGDQKQYGGFDNRGLEPGHRYVLFVLAVLQKSEPTFAASPFSDPFQLDNPDPQPIVDGEEGLIWVIGPVLAVVFIICIVIAILLYKNKPDSKRKDSEPRTKCLLNNADLAPHHPKDPVEMRRINFQTPGMLSHPPIPIADMAEHTERLKANDSLKLSQEYESIDPGQQFTWEHSNLEVNKPKNRYANVIAYDHSRVILQPIEGIMGSDYINANYVDGYRCQNAYIATQGPLPETFGDFWRMVWEQRSATIVMMTRLEEKSRIKCDQYWPNRGTETYGFIQVTLLDTIELATFCVRTFSLHKNGSSEKREVRQFQFTAWPDHGVPEYPTPFLAFLRRVKTCNPPDAGPIVVHCSAGVGRTGCFIVIDAMLERIKPEKTVDVYGHVTLMRSQRNYMVQTEDQYSFIHEALLEAVGCGNTEVPARSLYAYIQKLAQVEPGEHVTGMELEFKRLANSKAHTSRFISANLPCNKFKNRLVNIMPYESTRVCLQPIRGVEGSDYINASFIDGYRQQKAYIATQGPLAETTEDFWRMLWENNSTIVVMLTKLREMGREKCHQYWPAERSARYQYFVVDPMAEYNMPQYILREFKVTDARDGQSRTVRQFQFTDWPEQGVPKSGEGFIDFIGQVHKTKEQFGQDGPISVHCSAGVGRTGVFITLSIVLERMRYEGVVDIFQTVKMLRTQRPAMVQTEDEYQFCYQAALEYLGSFDHYAT

SEQ ID NO: 8 - DNA, 6018 bp, Homo sapiens
cctcgcgccgcccgcccggcagcccggccggcgcgcgcacgccgcgagccgctggcgctcgggctccgctcggatcccatgcaacagccacgatgtgaagcggggcagagccgggggagcccagcccagccagcctccagacgttgccccatctgacgctcggctcgaggcctctctgtgagggaccggggggccatccccctccagggcggagatcggaggtcgctgccaagcatggcgcccacctggggccctggcatggtgtctgtggttggtcccatgggcctccttgtggtcctgctcgttggaggctgtgcagcagaagagccccccaggtttatcaaagaacccaaggaccagatcggcgtgtcggggggtgtggcctctttcgtgtgtcaggccacgggtgaccccaagccacgagtgacctggaacaagaagggcaagaaggtcaactctcagcgctttgagacgattgagtttgatgagagtgcaggggcagtgctgaggatccagccgctgaggacaccgcgggatgaaaacgtgtacgagtgtgtggcccagaactcggttggggagatcacagtccatgccaagcttactgtcctccgagaggaccagctgccctctggcttccccaacatcgacatgggcccacagttgaaggtggtggagcggacacggacagccaccatgctctgtgcagccagcggcaaccctgaccctgagatcacctggttcaaggacttcctgcctgtggatcctagtgccagcaatggacgcatcaaacagctgcgatcaggagccctgcagattgaaagcagtgaggaaaccgaccagggcaaatatgagtgtgtggccaccaacagcgccggcgtgcgctactcctcacctgccaacctctacgtgcgagagcttcgagaagtccgccgcgtggccccgcgcttctccatcctgcccatgagccacgagatcatgccagggggcaacgtgaacatcacctgcgtggccgtgggctcgcccatgccatacgtgaagtggatgcagggggccgaggacctgacccccgaggatgacatgcccgtgggtcggaacgtgctggaactcacagatgtcaaggactcggccaactacacctgcgtggccatgtccagcctgggcgtcattgaggcggttgctcagatcacggtgaaatctctccccaaagctcccgggactcccatggtgactgagaacacagccaccagcatcaccatcacgtgggactcgggcaacccagatcctgtgtcctattacgtcatcgaatataaatccaagagccaagacgggccgtatcagattaaagaggacatcaccaccacacgttacagcatcggcggcctgagccccaactcggagtacgagatctgggtgtcggccgtcaactccatcggccaggggccccccagcgagtccgtggtcacccgcacaggcgagcaggccccggccagcgcgccgcggaacgtgcaagcccggatgctcagcgcgaccaccatgattgtgcagtgggaggagccggtggagcccaacggcctgatccgcggctaccgcgtctactacaccatggaaccggagcaccccgtgggcaactggcagaagcacaacgtggacgacagcctgctgaccaccgtgggcagcctgctggaggacgagacctacaccgtgcgggtgctcgccttcacctccgtcggcgacgggcccctctcggaccccatccaggtcaagacgcagcagggagtgccgggccagcccatgaacctgcgggccgaggccaggtcggagaccagcatcacgctgtcctggagccccccgcggcaggagagtatcatcaagtacgagctcctcttccgggaaggcgaccatggccgggaggtgggaaggaccttcgacccgacgacttcctacgtggtggaggacctgaagcccaacacggagtacgccttccgcctggcggcccgctcgccgcagggcctgggcgccttcacccccgtggtgcggcagcgcacgctgcagtccatctcgcccaagaacttcaaggtgaaaatgatcatgaagacatcagttctgctcagctgggagttccctgacaactacaactcacccacaccctacaagatccagtacaatgggctcacactggatgtggatggccgtaccaccaagaagctcatcacgcacctcaagccccacaccttctacaactttgtgctgaccaatcgcggcagcagcctgggcggcctccagcagacggtcaccgcctggactgccttcaacctgctcaacggcaagcccagcgtcgcccccaagcctgatgctgacggcttcatcatggtgtatcttcctgacggccagagccccgtgcctgtccagagctatttcattgtgatggtgccactgcgcaagtctcgtggaggccaattcctgaccccgctgggtagcccagaggacatggatctggaagagctcatccaggacatctcacggctacagaggcgcagcctgcggcactcgcgtcagctggaggtgccccggccctatattgcagctcgcttctctgtgctgccacccacgttccatcccggcgaccagaagcagtatggcggcttcgataaccggggcctggagcccggccaccgctatgtcctcttcgtgcttgccgtgcttcagaagagcgagcctacctttgcagccagtcccttctcagaccccttccagctggataacccggacccccagcccatcgtggatggcgaggaggggcttatctgggtgatcgggcctgtgctggccgtggtcttcataatctgcattgtcattgctatcctgctctacaagaacaaacccgacagtaaacgcaaggactcagaaccccgcaccaaatgcctcctgaacaatgccgacctcgcccctcaccaccccaaggaccctgtggaaatgagacgcattaacttccagactccaggcatgcttagccacccgccaattcccatcgcagacatggcggagcacacggagcggctcaaggccaacgacagcctcaagctctcccaggagtatgagtccatcgaccctggacagcagttcacatgggaacattccaacctggaagtgaacaagccgaagaaccgctatgccaacgtcatcgcctatgaccactcccgtgtcatcctccagcccattgaaggcatcatgggcagtgattacatcaatgccaactacgtggacggctaccggtgtcagaacgcgtacattgccacgcaggggccgctgcctgagacctttggggacttctggcgtatggtgtgggagcagcggtcggcgaccatcgtcatgatgacgcggctggaggagaagtcacggatcaagtgtgatcagtattggcccaacagaggcacggagacctacggcttcatccaggtcacgttgctagataccatcgagctggccacattctgcgtcaggacattctctctgcacaagaatggctccagtgagaaacgcgaggtccgccagttccagtttacggcgtggccggaccatggcgtgcccgaatacccaacgcccttcctggctttcctgcggagagtcaagacctgcaacccgccagatgccggccccatcgtggttcactgcagtgccggtgtgggccgcacaggctgctttatcgtcatcgacgccatgcttgagcggatcaagccagagaagacagtcgatgtctatggccacgtgacgctcatgaggtcccagcgcaactacatggtgcagacggaggaccagtacagcttcatccacgaggccctgctggaggccgtgggctgtggcaacacagaagtgcccgcacgcagcctctatgcctacatccagaagctggcccaggtggagcctggcgaacacgtcactggcatggaactcgagttcaagcggctggctaactccaaggcccacacgtcacgcttcatcagtgccaatctgccttgtaacaagttcaagaaccgcctggtgaacatcatgccctatgagagcacacgggtctgtctgcaacccatccggggtgtggagggctctgactacatcaacgccagcttcattgatggctacaggcagcagaaggcctacatcgcgacacaggggccgctggcggagaccacggaagacttctggcgcatgctgtgggagaacaattcgacgatcgtggtgatgctgaccaagctgcgggagatgggccgggagaagtgtcaccagtactggccggccgagcgctctgcccgctaccagtactttgtggtagatccgatggcagaatacaacatgcctcagtatatcctgcgagagttcaaggtcacagatgcccgggatggccagtcccggactgtccggcagttccagttcacagactggccggaacagggtgtgccaaagtcgggggagggcttcatcgacttcattggccaagtgcataagactaaggagcagtttggccaggacggccccatctctgtccactgcagtgccggcgtgggcaggacgggcgtcttcatcacgcttagcatcgtgctggagcggatgcggtatgaaggcgtggtggacatctttcagacggtgaagatgctacgaacccagcggccggccatggtgcagacagaggatgagtaccagttctgttaccaggcggcactggagtacctcggaagctttgaccactatgcaacctaaagccatggttccccccaggcccgacaccactggccccggatgcctctgcccctcccgggcggacctcctgaggcctggacccccagtgggcagggcaggaggtggcagcggcagcagctgtgtttctgcaccatttccgaggacgacgcagcccctcgagcccccccaccggccccggccgccccagcgacctccctggcaccggccgccgccttcaaatacttggcacattcctcctttccttccaattccaaaaccagattccggggtggggggtggggggatggtgagcaaataggagtgctccccagaaccagaggagggtggggcacagaccatagacggacccctcgtcctcccccagcggtggtagggggacccggggggctcctccccgctctgcagcctggggacactgggctgggaccagaatccagctttcttttaaaactctcagtgtaactgtatcccgtgacatttcattttttttaaatagtgtatttttttttccatttttttttttaagagaaacaaacaaaagactcgccagtcaatgactttcaaagagaactaactttggcttattcatattctgttcaaagacagtctattttttcactgtagaaagcgtccttgtgtgatagttacgttcgcaaacgcgcacgccaggcccatggctgtaccttggcttttttttttttttttttttttttaatttttcctaccatcagaaagtgtgctttgctcacagaagaatgggatgtccttttttctttcttggctttttttttccccctttttgtttcatttttataaattaaattttcagacatatcaaatacagttctgagggtaaggtcatgggggagctcggacccagtggcgttgggtgcggttgagggggacgctgctgtaagaggagagagatgacagtggtcctcctctgagagcctgagctgtctccccgtctcccgcccccaaggagacagagaggatcctacttcttcggggacagtggctgtatggctgtgctgccccacatcagggaccctttccccctgggactgtggggcagtttgggagcaaaaccagaaggacaggcccccctctacccgcctaccctgagcaagcgagttgttcctctttgtacaagggcaggtctgcggttactttcaacactgtttattccagcggaagcagccgggtggttttcccacccccgtgtatgtagatatatcgactttgtattaaaggaagatcgtctga

SEQ ID NO: 9 - protein, 1505 aa, Homo sapiens
MAPTWGPGMVSVVGPMGLLVVLLVGGCAAEEPPRFIKEPKDQIGVSGGVASFVCQATGDPKPRVTWNKKGKKVNSQRFETIEFDESAGAVLRIQPLRTPRDENVYECVAQNSVGEITVHAKLTVLREDQLPSGFPNIDMGPQLKVVERTRTATMLCAASGNPDPEITWFKDFLPVDPSASNGRIKQLRSGALQIESSEETDQGKYECVATNSAGVRYSSPANLYVRELREVRRVAPRFSILPMSHEIMPGGNVNITCVAVGSPMPYVKWMQGAEDLTPEDDMPVGRNVLELTDVKDSANYTCVAMSSLGVIEAVAQITVKSLPKAPGTPMVTENTATSITITWDSGNPDPVSYYVIEYKSKSQDGPYQIKEDITTTRYSIGGLSPNSEYEIWVSAVNSIGQGPPSESVVTRTGEQAPASAPRNVQARMLSATTMIVQWEEPVEPNGLIRGYRVYYTMEPEHPVGNWQKHNVDDSLLTTVGSLLEDETYTVRVLAFTSVGDGPLSDPIQVKTQQGVPGQPMNLRAEARSETSITLSWSPPRQESIIKYELLFREGDHGREVGRTFDPTTSYVVEDLKPNTEYAFRLAARSPQGLGAFTPVVRQRTLQSISPKNFKVKMIMKTSVLLSWEFPDNYNSPTPYKIQYNGLTLDVDGRTTKKLITHLKPHTFYNFVLTNRGSSLGGLQQTVTAWTAFNLLNGKPSVAPKPDADGFIMVYLPDGQSPVPVQSYFIVMVPLRKSRGGQFLTPLGSPEDMDLEELIQDISRLQRRSLRHSRQLEVPRPYIAARFSVLPPTFHPGDQKQYGGFDNRGLEPGHRYVLFVLAVLQKSEPTFAASPFSDPFQLDNPDPQPIVDGEEGLIWVIGPVLAVVFIICIVIAILLYKNKPDSKRKDSEPRTKCLLNNADLAPHHPKDPVEMRRINFQTPGMLSHPPIPIADMAEHTERLKANDSLKLSQEYESIDPGQQFTWEHSNLEVNKPKNRYANVIAYDHSRVILQPIEGIMGSDYINANYVDGYRCQNAYIATQGPLPETFGDFWRMVWEQRSATIVMMTRLEEKSRIKCDQYWPNRGTETYGFIQVTLLDTIELATFCVRTFSLHKNGSSEKREVRQFQFTAWPDHGVPEYPTPFLAFLRRVKTCNPPDAGPIVVHCSAGVGRTGCFIVIDAMLERIKPEKTVDVYGHVTLMRSQRNYMVQTEDQYSFIHEALLEAVGCGNTEVPARSLYAYIQKLAQVEPGEHVTGMELEFKRLANSKAHTSRFISANLPCNKFKNRLVNIMPYESTRVCLQPIRGVEGSDYINASFIDGYRQQKAYIATQGPLAETTEDFWRMLWENNSTIVVMLTKLREMGREKCHQYWPAERSARYQYFVVDPMAEYNMPQYILREFKVTDARDGQSRTVRQFQFTDWPEQGVPKSGEGFIDFIGQVHKTKEQFGQDGPISVHCSAGVGRTGVFITLSIVLERMRYEGVVDIFQTVKMLRTQRPAMVQTEDEYQFCYQAALEYLGSFDHYAT

SEQ ID NO: 10 - DNA, 178856 bp, Homo sapiens
agccggagctggagccgaggcggcggcgggacgcggccggccggacaaatttcctgctaggctgcggacagcgggcggcaggagccggcgcgagcggcttcaggaacccacggcctctgcgcgtccccgcgacccttcttcgcgcccggcgaagacagccgggcgccccggagggcggcgggcaggcgcccgggagatgcggagcctccgctgcagcgcgatctgcgcgaccagaccggcccccccgaggtgagcgcgcgtgcggacagggaccctgcgcccctgtggggaactgtgcaccccgggaggcccaagcaggccggggcgctgcctcggccgctgccgcgggagggaggggccgctccggggctcagagtccggaccccggccccgccgcccccgcggcgcgcccagtaccagcggctggggcccgtacgacccccgaccccggcctgtggcgcgtcccctcacccggagtccccagcccaacttggcgaccacagtggcagtgctcacgctccctctcggtaaacaggaggagggcgcgcgtgtctgcgggtggctacagtgcgcgggggccggcggcagggcggcgtacgtgaaagcgggcgacacagagagggggtgcaggccgcgcgtggggggctgcgcccactcgaggctggaggctggagcctttgaagaaagtggctcgagttgtgccaactctgggaggggccggcgctctgccaggatgctgccccgcagccgggcgggcgcccgcgccttcccagggaggcaggtggagcgggatgcagcagccgccgggggtcggagatgcggcagggagaccctggcaggtggtccaggctggactgccgcgatttgcagggtcgacgacctcacagggctcctgagcacagatgcgagcgcggtgccaattctgagagctcccgggaccatctggggaaggccgggcaacccacaggatggggaggcagagccaagccggatggagaccgcgctggacctgacctaaattccttccaggcatgcaaacctttccagaaagattaggcaattatacttgcagaattatgatattggtttgtcttgagtcgtgaccattccatcagagtttcaatccaggaaactcaaagaaagaatacagctctctgcttgtattttagagtgaatgagcgtataggtgagttgaatgaacaccagcaaagagtgatcacgtgatacagaaattactgtgtccatgtccagtcagctctgcatttggccccaggtaccccttgacccagggcgtctggaggtctttgtgtcccaaagatggatgaagttctgcctctgcacactaacgactcttgtccatttaaactgtgtgtatgtgggtggggagggggggtgtggagggtcgtttccaaaactgggataatggccagaggagggcatgaaatggtgggggccagtggctggggcaagcgtctgaccaccacggtcactcagccagcatggtatgggcagagctctgtggtgtgaacagggttagggtctgtactttccaagcacaggctcttggcatttggggagcaaaaggtcagaggcgaggtggagcaggcccaagaggtgggggctgcgctgtcagtgagagaggcctgacgtaccacccagcccttcccagacaaggctctgcccctgaatctgaccttgggggccaagatttttgttcattgatgttggtgatgcctgtgagtcaccaccgccccctccctgccccaacagaccgttcccatgtggacaagagctaggagctagatttgtctctgtaaaaggtcaggtcactggatgtgtccagatggcttgggacttagtcacacactccctcatcagttccagtcaaaccagttgagggaagtcgatgagggtgttattgtctttacgtgtgatgcttcttggaggccctgtgtggacacaaagtgaggacaatgcggaaagtcacacagaggccacctctcatttctgtgggggacagctggcagagagagatgcgctccctgtgtcagcaccgccacctccacggaagcctggaggaggtcctgaagcactgtgccctgatgggagccccctcttctccagggatgcgtgtgtgcaaaccattgcccctgagagtagatgcggctgtgtgtgtacatccttgcccctgagaagagaggtggctgtgtgtgcaaatcttgcccccaaaccccaagagaagatgcattcacagtgggggtgatggtagatgcatcagtttcaactggtgaaagtatgcttctcccagctgtgcctcagtgggtccactgtgctccatctgcaccaccattagcctagtctaaggtgcaccgctgatcaatattccctccctgaaggccagaggtgtgtttctaggcccggccactcctctgtagttctcctccgcagctgcccacttgacatttctccctgggatatcaaatgccccttgactcaatgcgtcaaaatcaaacttgtgatctcccctccccttccagtactttcccagtgttgtctttatccatccaattccaccagcctccatgttgcttctgaaatgtatgccagactatagtttctgtgaagagagggcaatactgtatctccagtgcccagccatgtgcctaccagtggagccttgaggactactgtccatggataaacaacttggaagtgagaccttcccgaagcgcatgtgctgatggagcaagtgtgtgtctgttgcgtggatgttgtatggttgataggcagccacacatttttgccgatgtggatgggcatggtccatccagggctcatgtggccagcccttctggagatgctggactcacctgctaaaatgactgctggcttggaccagagacaccccaagtgacctcccagggaggcaggcttgggagcagggagggaacaggatcgaagctgccccgtattcccttgctagacaagtctttttcatttataacatttgagtcaccctcacagaagttctgttgccaaagcaggaatgaatgtatccattagggtaaagactggccttcttaatatttgaatatcacttgggaagaaaactacagttctaagaacatctctagtttttcttttggctttcttcctactgacccaatggctttgtcttccataaagctcccaaagctttccagatcccaagactcagctccctgctctcagcctccttcccctttatttccatcttagaaactcctacccatacctcaaggcccacctcaaaagtctcagtctccacaaagccttcctggctcccctacctgagaggaatctctcttactcttggttccatagaacgttatttctgcctgtcctaaagcacttactggacttgactttgatttccagttcacttgctcaagtagtttatctcctccactaggcagccagtgctttaagggtggagggtgtgtctctgagagcatgtactttgggttcagtcagtcaagttctgactctggtgtgaccttggctaaatgactcaaaatgcaggcttcagtgttctctagaatgggagtaatcatcgctacttcccagggctgctgctgtgagggggacataggatacatataattgacaggccgcttggctggtatgcagtaaactgtggttatctttcccctacaacatctaatgctgtgcattgcacatgtaagaatcagtgaagtttgaggccgggtgcaatggttcacacctgtaatcccagcactttgggaggctgaagcacgcagatcacttgaggccaggaattccagaccggcataggcaacatgatggaactctgtaccaaaaacacaaaaaaattagatgggcatggtggtgcacacctgtagtcccagctactcaggaggctgaggtgagagaaacatctgagcctgggaggtcagggctgcagtgagccatgttcacacaactgcactccagcctggttgacagagaaagaccctgtcatggaaaaaaaaagaatcaatgaagtttgaatgaatacatgaaccaagccaaaaaccatacaaatagatagataccttttgtgcatcatccagatcagtaggtcaaatcagtgtggtgtggcatgccagataatttttgcatggcaatctaaatgattattatgtaaattttgcgtgattaagtattttaatcccataatgttatcacttaccatgaaggcattcattcttcaatatgcaccattgactccagtgatcgatgaggcagaaatgcaacttgctgtgcacattaaccaggctcgtggcctgaattaaaccaggctgggtgtgaggcactgcaaaagcaaacgaaacccacccctgagaagcagtgtggcctagaatcaggtgtctggaatcagggtaagagagcagcttctagaccagaacataaagctaggaagctgtggcctaccctagaaactctcagaacttcactttgcttatccaaagaaaggggctgtcctgggccatctctgggcccctcacagctgctgcacttccgcccgatttcaccccttccccaccactgggcaggaagggctgctgactccgtacaaggctggatcccaggcagggaggcctcctgtttcttggctcagcatttgggccacgtgtctctctgctctggacaccaaatctagagagagtgaggagaggcccgtgggtacatgtagcgtgctgccaattttaggtactggccctagacatatcaagagtttattttggaggattaacttctcattattttgtcactaggatttttttttttcaccaaaagcagagtgctggatgatattttagatggctgggattttagatcagccatacctgaggcaataagagttggtcgaatgttaccaatccttgagcagacacagatgagcagttgcggggtcgttcgctgctggatgtgggctaggagacccctggtttggggattatgaaggcagcctccttttcttccctgggctctggctgctgttttcccagtcatacccatcatggtctgccctgtccaggtgttccatgtggggagtattgatgtggtgcccagcctttgaactgttaacaacaccctgctcacagccagaaatgtgggccccacatctttctttcaactgtatgaatacttcagaggctttgattgagcttaagaaatacaaattcttaacaatatatttcagaaaaataaacatatccttttatattgattcccaatgaaataggggccagataaaatattttaaaagggcatctgtttttaaaaatatctttcaaatgcaatcaagaatgttcagttaatccggtccattatgtatattgtaacaagtaacaaaaggggcagagaaatctttctcccaaaaacaaatataaagtccagcagggagtagggaaatgtttgaagaaaaaaaaaagattcttagttatgtgctaaaaaccaaacgtgttttgggcaggtgctttgatgttagtgtttgttgggtggagagtaaagaattgataggaaaacttgtttggatcccaggtctgggtgccagctacactacatttgatccaatggaagggctagcatgggaacaatagcatttggtgtgagcgccctgggcaggtacatgactctgtcgtcagctcccagggatggacagccagtcccacaatgcctggagctcagagctcactctgtgcatgtatgtgacatggagagatggatggatgaaggtaatagaactgagtaggagcctgtccaggtagcagtggaggaggccaagtggcggaggccttccctgccatgattttactctgtctgaataccagcagcaagtggaagccacggggctttattttcaagggaggtgacacagcctggggctcagggaaaggacttaggagcagaatgactgggatcagatgggggcccatgagactgaggctctggacaaggcaagggacttcccctggcccagtgtgagcccaagtctttacggtggggagcatgatgccgccaaggcagctgcacggggatcggtggaggtggtacgcagaggcctggccccatgatcaccaaagggccagcggtctcacacaaggcaagggcccaggggaccggaatcaaaggcctgagatgatgtgagccctgacttctcctctgccacagtcgctttgtcctcaaggcaagtcatttaattcctattcaataaatgcacaaacataatttttggttattaatgtccctaaatctgtttggacatttccagctgaacattagtcaccagttttgtactagagagaaaattagttccatctttcttctcaactgagatttttaaatgtcctatcacatagctacttgattacagtgtattattttaaatgacctctgtactaagtccttttattttggcgtcctctcactccagaaggacttggcattggcatttttcaggctgtgtctccacagtaggactttcataggtctgcggtgcagactggtttgctgtccacaggcttggccctgactcctcggctaacatgcacaagggcgctctgagggtcgggaacgcacccaccaagcacagcccagacgaaatggaagctcctgtccaggcaggttcccggtgaaactctagggagctccagccttcctttcttctccagtttattaagaggctggcctcagaggtggacacctacttccacaatttgatgtccgaccctgggactctcctctttaccctgcccccacctcatccaccagccttttggtctgaaatccttgtcatggggtaaagctttttattctgttgttggaaacacagtatatggtttggctgtgtgtcccctccaaatctcatgttgaaatgtggtccccagcgttggaggtagggcctggtaggaggtattggatcatgggggcaggtacctcatgattactagcgccatccctttagtgatgagtgagttcttgctcagctagttcacatgatatctggtcattttaaagaatgtggcaccccccacccactctctctcttgttcccactctcactgtgtggcatgctggctccctccctctgccttgattggaagcttcctgagcctcacctgaagcaggtgccagcattatgctttgtgcacagcctgcagaaccatcagccaattaaacctcatttccttataaattacccagcgtcaggtattcctttattgcaatgcaagaacagactgacacacacagcacaggtggaaatacaggctttgcggacaccggtggcctgtttttctgacactatcatacttcccaggataggctctactcttaactcttctaaccccatccacttgttataaaacacactttcagaaaaaggctaggttgttactgaggtaacatgggatggatctttggaggtgggaaggcctccagcctcctctggggaagacaaagtaaatcctccctatggaattctccagcttgttttcttgattccagagagacctgctagtgaattcaactaacaggttggttttcagcacagaatatttcctacagatgtcggtgtttctttccaaatccttcctctcttcagggcgctttcccactgttctagacgtaggagagaaagcacttctcagatggtgagcttttatcttccaatcttgtcaaccgaggagacctggaagttccctttcaacctggaatgtgagccctgcttgctgggcacgagataaggaccaaggattgacccaggacaatttcagagagcacaggggtggcatatcctcatgtcagggcgtggagctaatattcagacccaaacactctgaatctctgaagcagaagctctcttaaaattcctggggaggaaatcagtatgaattaataaaaagcctgaatattgtggatttttattaaattcgaggatttatagtaactcaatccaggccaacaaaacgttgccttccggtacttactttaatgaatagaaagcatgtaaatgctcttccaaagagaataacacattttagcatcttaaatatgtaaaaataaaactcctgtacaatcctagttgcactaggaagtctacttatcaaaaaaagaaacttctgtttccagccacattatttcaaatcatcccaaaacttacattgttactagtaaaatttttcattgtgattaacactttttttccagtatggttttcctctttctgaaagcaaccttagtttcataaacagttggacatttaagtactggtcaaaaaagtactctgaaccagtaacctcgtccttaattttgaactgctgttgactaagcgctctcttttcctaagctcatagcagcccttcgttcagtgcttcagtaattcaataaattactttttgccaaggttgcaaacatcagctaaagttcagtcatctactggagatgttatcagtctgtccatttaaattcttttgcgcaaagtccagatttggacccaaagtaatataaaaatgcaagagccagaaatttaataattttgtactgtgttgtgccaagccaggcctattcttagctagccaacaacattctttcaaaggatattttttggttttcatcaaatgtaaatagtgtttaagagtctgtaatatcatttagaaggaacaaatggaagtattaaacctttattgactacctacagtatatagaggatctataacttatgatggttttattatagtttctcaaccttacaatgggtttattgggcattgggtacattttaaacttatgatatattctatttacaaggggttcatcaggatatgacctcactgtaagcccaggagcatccgtacgtacaagcactgtgcttagcgctggagttacagggaaaaaaaaaaaaaaaggcacgatggcttgtgacatcctgtggatgctccaggtccacaggaagacagaactcacttccagggagtgttaaatgtgatgctggatgctcctgtggaggaggggagacatggaagggcctccccaggtagagcagtgacgtggtctggctctgtgtccccacccaaatctcatcttgaattgtaatccaaatagtaatccccacatgttgggggagggaccttgtaggaggtgattagatcatgagggtggtttcccccatgctgttctcatgatagtgagtgagttctcatgagatctgatggttttataaggggctcttcccccttcacttcactgtgcacttcactgtgcacttctccttcctgtcgccttgtgaagaaggtgccttgcttccccttccaccatgattgtcagtttcctgaggcctccccagccatgctgaactgtgagtcaattaaacctctttcctttataaattacccagtcttaggcagttctttatagcagtatgaaaacagactaatacaagcaggcttggtttactgggccatttcctaggcagctgacagggattgagcattccagacagaggggctaccatgtgcttgcctggcagtaggaagcggtgtgtcctgtctggggaagagcaggtgtctgcatggagctggagcccagggacagaggggatgggacaaaaaggtggaaagtttaaggatcacccaaacatttgtacaccatccaggagtctggattctgtgtgacggaccagcagttcttaccgtgtcttaaattatgaccccctttgtgattctagggaagactatggactcattctccagaaaaacgcatgtgtgcacacagtcatttgtatagtttcagcaagtctttcctaacacccctccccccatcctgaggctcacaggttccaggtgtgtcccctttgtgcggcctggccagccccacagagcccagcagcatgctcagctgtgagtttgagaaggggtcccctgctggctgtgtgtggaaagccagaatggaggtgacgggggaaaccgactcagcaggttggatttataggcaacatgaccatgtgggtgagaggatgagagtgagaaccaaggccccgcagttttcggccagggtgactggatgcatccggaccagacacaggcttacgagatgatggtgcgcccagggtgtgagctgagtttgagggtgccagagggggaagccgctccaacatgtggaatccaagcagctgattccagcagggagggtctgtccaaagggaagcgaagagagtttttcagcatgaaactgatggtgccaatgtttgtgcttcactgcgctaaactgtcatctttctgttaaatttggcttacagggccaggtgcagtggctcatgcctgtaatcccaacactttgggaagctgaggttggaggattgcttgaggctaggagtttgagaccagtctgggcgacatagtgatactgtctctaaaaaatattagccaggcatgatggtgcatgcctgtagtcccagttgctccgggggctaaggcaggaggatcacttgagcccaggaggtcgaggctgccatgatcatgccactgtactccagccttggtgacagagcgagaacctgtctcaaaaaaattttttttattttgaaaattgggttacactgtacattcttagggtctagacaatattcctaataaaatgccatgtaggctgggcacggtggctcacacctgtaatcccagcattttgtgaggccaaggtaggtggatcacatgaggtcaggagttcacaactagccgggccaacatagtgaaaccccatctctactaaaaatacaaaaattagctgagcacgctggcacgcacctgtaatcccagctacttgggaggctgagacaggagaatcgcttgaacccatgacacagaggttgcagtgagctgagatcacaccactgcactcagcctgggtgacagagtgagacaccatctcaaaaaaaaagccatataatgatggttgttgtcatcagagaagtcggctcacagggggtgatgtctcctgctcccacttacaaagtaggtgagaatgatctcgagaagctccatgaggtgaccctcagcctctgatggggacaaacaggagtcaccctgctctctctctggctcccatgactcttatctaaagacctcaggctggcttggagcaccccctttaaacttcctttaaaaaaggagaaaatcggcctggtgcagtggctcacgcctgtaataccatcacttttggaggccgaggtggccagatcaggaggtcaggagatcaagaccatcctggctaacacggtgaaaccccatctctactaaaaacacacaaaaaatagcccagcatggtggtgggcgcctgtagtcccagctactcaggaagctgaggcaggagaatggcatgaacccgggaggcggagcttgcagtgagccgagatcacgccactgcactccagcctgggcgacagagcgagactccatctcaaaaaaaaaaaaaaaagagagaaaatctatatggaagaaatgaaaattattgaaggagcttggggaaaaggctgaaagaatcttggctgttcatgcaaaccaagtcccagggctggcttcatggctgttgggcaacccctgcccagtcagcatcctggcagttgtcactttggcagagtcggaggacagacacacaatgacagcaagagcaaaggcagggcctgcccgcgaggaagagccacggctgttgagcgtggatgtgtatttctcaaggagtatgaaggagtctttttgcaaaattgcaaagaactggcctgactgggaagtgcaagagttatcttcccttcagggaagagggaagtttaactggggcctccttgggctggaacacatctcaaagtcatggttcttggaaacccagggtgcatttatggacaagtctattataggagttgtaggtgtatcttgaagatgcaaaaattcattttgtacctttattttacacatcagaccttacccaggtaccattctgtgctgggcagtgcctaggactgaggcagatttggctccattggtgtgcccaggaaatggacttcattgcacactgaaggaccagcccttcaatgcattgtcttcaaataaaaatgaaaatttcactcatttctggtgattttctcaataacctttctcataggtttgaatataactagaaacataatttttgtccaagtctttcagttcaccaaaaaccaaagttaaattctacccttgcgctttagatgttaacaatttgagaagtttggcgaacaattccatctccttatttagagccagagctggaggccagctgcagatgaggaaagggagacttgcccaggggcacgctgcctgctcctgccacccgtgggctgccttcttcctgttctgttctgtgctgcctctagtggactgaccccttatccctccttcctgtgcctgcaacacggcttatggtgctgggcctcaggaaatggaggtccctcatctgcatccataagaagaaagcccgggttattcatctgtttagacagaagtgcactccaacctttagcaggatctgaaggtgagaagctatgacttcattactgtgcatccatcttttgtctgcttggccctagtcctctgggtgtggtttccgcctggcaaattacttccaggagatgcttgagaaggcagtagcgtgtggaaacatcttatgtgcagcatgcttttgtttacaaatgatttattattggttatgtcaaaatacacatcctgaatgaatgggcagtgctgggtagaaatcagtgacatcaatcgtggttattttacttagcagtggattttttttttttccttttcagtgtagtagtaatttgtaaatgctttatggaggttatgctttctttagtggcttggcatcttggaaattggagcagccaggctcggcgtgctgtgtccatttttagccctagcaggcaggaagtgtggccatgctctctcagggtaactgctgcgagtttgtggctagaatgatgttgggtgggagttactcaactgtttgaatgcaatgggcttagctaatggtaagaaagtgcgtggagcctgggaagcaattgatagctggttaccctgagctcagcctctagtaacaaatgacacaaggcatttgtgaaggctggaaagcctccgtgagaagagcaccttgagtcccactgggaaggggcagccccttggagcatgaggggtctcttgctgcctctgctttgggtctggctgagggtccactttgctccctgaccccagccagggtaccagttcccagattgctggtgggcgtgtgacagctcatgtggccaagcaccctgccctgcttcttcaaagctcctaggtgggggagtcccagtggcagggcagagggaccaacccccatggctgactcagctgtagtccacccagattgcatttatgggtaagtctactagaggcactgtacccgagcctggggcaacccttcttctttcgtggacttccctggtctctccaggacgtttcagtgcaaatgcagctgtgtgataccaccccatacactggcggggctgaagggaggttgggttccgtatcagtcagggaccttagggatgcacgagatggaaatatgactcaagttcagtcaagttcataggggcgttgacttacaaaactgaaagctcaggtacaactggatccacatggccagatgttgccaccaaggccctggctctctctcatatgtgctctgacagactcagtcctgtggggcctccagcaaactcacattctgtcttccctgtgtaaggagagggcgtttccctacagatcaacccagacatgagtctccttggctctgacgagccaggcttggataactggcccaacctcaaagcttcctcggtgtctggggcttgccctgttctgataatccaggcttgggccctctgtcttcccaggggccagaggtggaaacacagctcctttggcaagtgtcctattttcagtgggggtccagggcaataaatgaaatactcgttacgataataccagcgatcactggtgttgagggtgcacttgctgagtgccagccagatcctgctctaagcacttggacatgttaactcggtcagtgctcacagctgcaaaaccaggtgagggcagcaattagtgcatttttgacagaaggaactggaggcccagatagggtagcagcagggcagggatttgaaaagggattgagaccaataatccccaagacggaggagtgatgcctatgtgtggaagcaggggccacacggctgctggggatggggtgggaagaggcgtcctgtggcctccaggctcactcagggagcagtcagatggaaggccggggtagacacctggacctgcctgggaagggctttagaatcacaggagggacgtgagctccatcttgaactgggggaggtgatgccagggatgtggacgtctatagggactgggctgggggcacagagctgggggccaggagaggctcactgagctgttgcccagcagggctgtgcctcagagtcacaggggaggttcccgagagcctcctgcttggaccctgctctcatgattggctccaagttctacagtgaggccagggcagctgcattttttacaaagctcccccaggaaattccaagtgtggccagggtggagaatgccagggtcttctgtagggtcccagaatgaggtcaggactttcttcaatgcagagggcaggaggggagtccaggaggaatggacagggtgctgtggctcagggtgccggtggaaggcaggcatcgatgctgagtctgaagccctgtgctgggcaggaactttgctcttgcaggaagtgaaactgctggaggaaattgctaaaggaaagcaatggaatagactaatccatgacaagttgttagtgaattcatgaagtccaattttaagagctgctttttgaaagaaggctactggagaaaggctaactaaataaacaagttgctgatgttgggatgtcaggcagctgagctgacagagatcactcccagcagccctgaagccacaggagccaccacacatggtcatttacacagttccattcattcattcactgtctcaagttcatttaacacatgtgaatggaaggccttctggtgctgagactccaatggtgaacaaaacccataggtgcccttgcattctttgccgccggacacctaacctgaatctgggggtcctgtgggaggcagtcccgtaaggggctgtctgtgcggagctcagccctgctctggagactccgtggggcaagggggccagggggccatcgagggccgctctgaggtccttgtgttgtccgtgtgggattgagaaggcctcatgccaggagctgtgctcttctcttccaggtgggccggggccatgtgtattcattaggggcctgtcatccaagtgggcacttaggacaaagtggttgcccagctctctgctccacagaaccccatctgccacacatgataccacttttgctaggccttcttctaaagagttccactgaacccgtaagagatgtgtgttcctgccactctgttcccagccacaacatagaaaggggactgaactctataccaaggtgggtgggctgagagccccaggcacagcggcttctgtcgggccatttctgcccaggttcctttctctggctccctccctgctcccatcttccctccccctctgccctctctggcttttcccatcctcagcttcatctcttaggacctcgtcttccagaaccccagacccagccaagctcggcctccctcaggccttcagctagtgacctcagcattggtggagggactgctctgaccccagacccagcccttagtggcaacagtggcagctctgtgggcagctgtggctaacttgagcgtctggcggcattcccagcctgcactcctgtcccaggcagaggcctggagagctggtgctcagcccggcatagcggctggcgtgccattcttttgacatgagtaatagcaaagcaagtccaagaaatgtccagtgttttctccctgttgttgcccaatttcaaagagataatccaatcaccttatcaggtgaggggtggggcaggcttcccagaagcagacagagtgtggggacaccctgacctggccccaggagtcttggctatccagagcgagtgctacaagtctgggaagaacctcataatggaatcgcttgcctgttttgatcctgatgtgaaaaccagtttgaatttttttttttttttttttttttgagacggaattttgctctgttgcccaggctgaagtgcagtggcacgaactcggctcaccgcaacctctgcctcccaggttcaagtgattctcccgcctcagcctcctgagtagctgggattacaggcgcccgccaccatgcttggctaatttttgtatttttagtaaggactgggtttcaccacgttggtcaggctggtctcgaactcctgacctcgtgatccacctgcctcagcctcccaaagttttgaattatttttaaaggctccaaggagagcttctgttttgacagaaactctatcaagatatcatccagcccagtgtggagacacaatcttcctttaacacaggattcaaaccacatgcagtttacttccgggtatctctgtcaggatcttgcattcatttcccactgcctgatgtctggttggtggaagctgagacccctgagggatcagaacagatccagggagagctgggagtgctacttttccatgtctggtctgggttttagggagtgatgttcccacagaaggcttaagctggaaaatatccatgatgagtccagggtacatttggtggaggtttaaatactgtgtcaagatgcatacaaataaacttaaccgtggtgcatggcagaagatgtctctgggaaactgagtgaacaagtgaaaggaacacaagagaacaaactcttaagcttttttgtagagaaagagtttgtgctcgaaatagggacaagttacagggtgacaggcatgactgttttcagcagggagggcaagcgaaggtgggtgagcttgtttgttgttgttgttgttttgaaacagagtctccctctgtcacccaggctggagtgcaatggtgcgatctcggctcattgcaaccttcgcctcccggattcaagtgattcttgtgcctcagcctctcgagtagctgggactacaggcgcccaccaccgtgcctggctaatttttgtatttttagtagagacagggttttgccatgttggccaggctgatctcaaattcctgatctcaagatcctacgcctacacctcggcctcccaaagtgctaggaccacaggcatgagccactgcatctggccaggtgagctgttttaagaccacaagatcaggcagacatgggattgtcagcaggatttttaccatcaggtaacagtcaatgtgttcccctccgcccaacttccctcactgtactcaaggaatatctgatgacccaataataaaaacttacaagattaactgagataacccagtttaatgttcaataaggtcccactgggctgcagaatacagaaaaattgacccatataacttggcatttgtatcttttctgcaaagtctcaataatgtataattttaaaatctagaagttatggagcattgaagtagctgagctttctagaaagcggacattgtaatgaataagcaaagcaagaaaacctcagacattggtaccagctctattttaagcagaggcatgtggagctccctgggatacttggaaatgcacattcccaggtactgcagaggctgttcctatgaactatgctaactcaatcttcagcacagtcttctgttttctgagctctagagcaaggttggcaattttgccccatcttacctgcctgaagccagcacacctgagggcttggcttgggctggaagatccacttccatggctggaggatgttggccatggacaggaggccttcagtccttgccagatggacctatccacaggactatgtgtcctgatgatctgggagctggcttcacccagagagagtgatccaggagaaagcaagaaagacgttacagtgtctttaggacctagcctgggaagctgtattctgctgtttctttgacatcctatttgtcacctgatttagccttattccttcaggaaagaatggctggctctttccaaacagtatgtatgggtgagggatcctcattcttttctgaccccgggtggattagttgtggctcctggcaatatcccggatctgactggttaacttagtgagaaagggagcttggcagacggctaccgcagagttctcagaatcaatagtgaagctggagaacaaggctcagaaaacagggaggagcccaggaagctagcagcctgaaccatggtcgaggccaggccacaggaagcttttagtaagaatgtggcctgtggtccacggccactgagcctgctaggcccttccagctgtggcctctgcaacagttccaaagcatccctggggctatcaccctaaagaccaacatcttgggtaggattgatggttggagtctgtgtcaccccctgacaaatcctaactgcttggcggagggaatgtgagtgaacaggaaggacctcccctgggaggtggagcactttggagcagcagctgataggtaaagggcacctcaccagcccacaaaggacaaaaggttaccctgtctatactgagctcaagtgtcatttttgactacaagtatacctggtattacagtattaacatactcctgaaaagttatgttaattggaattgtgctttatatacactgggtaagcttaatacctttcagaagaccatagctaatccttttgtgaagcagctaatctttttagagaacaaatggtcacttcttaactatttctttcaagattaggagtctattgtgttttctcaaggcagagtagccagtgtttaagttttcccttttgctcacggtgaggaaatgttactttatagaaagctgcttgcagcctctcatgcagaatgaaaaaacaatgagtttttttccttcattttaaatctccactgtaatcaactgtcttttgcaaggagtacagattgttgaaaattttaaagagcatgtatgatttcattcttaccagcaaggctgttttaatatgagcatattttaaaactaagaatggcaggatctgaatgagcaaagaagcaggcgctgtgagttgttctgcggcaaagcacatttggacattcacctccaacatttctgtttttcttctgtgaggagctttgagactatatatagtggaggtattttgttcggtttcctagagttgctttcacatttcctggataacagcatgccgggagaggaggctgggaggctggctggtgtgggcgtgaggagcaagcctgagaggtttagactctgctccaccaggtctagcgacgggctctgtgtgtccgaagtagtcatggtggccacaggaaaggagacgcctagcaggggaggaactagacaggggacgagttgggaagctgctgtcacaatcaaagaccagaggggagaaagaggggaagtgagagagaccaagggaaagagagttgtctttttttttttttttttttttttttgagatagagtcttactctgtcacccaggctggagtgcagtggtgtgatctcagctcactgtaacctctgcctcccaggttcaagcaattctcctgcctcagcctcccaagtagctgggattacaggtgtgcaccaccacacctgactaatttttgtatttttagtagagatggggtttcactgtgttggccaggccagtctcgaactcctgacctcatgatctgccagcctcggcctcccaaagtcctaggattacaagcatgagccactgcgccaggtctctcttaatccaagagtgacaatggcaggtaggtacactggagagtgacaatggcaggcaggtacactggatgccccccacccccccgggggttgggttctcgctcaccattagggagatagcatgttgaacagtagcgtatttctccatatgtctaactcggctactttttcctccagcattggaacacaaaactatcattggctgatgccagatgaagtactggcttgtattcctttaaacaccagaatcctactcaatgtggcaagacactccgcatgagagggcatagtgggtaatggtggatccacttagcggggttgagttttctcagttttaatgtatgtaagatgtgggcccatattatggttagcaaaaccaatgctcagagatggaaactaagttgttcagggccatgtaaccagagggtagtagagctgggatcaagccccagtttgtctccccaacccagggcatctctgcagacctgaacagtccccctcctcatggagaggagccttctggagggggtgctgcctgggaattggggcagctagttttgtgccaccttctggaattgcagactgtgatttggaggttgtcacgtctttgagtaaaatacacagctgaagaaaactcagaaagtaacatttttgagtgggtagtttagtcgtatcagtgaaagggaatcactttcttgatttgattgtacttttaaaagagaatactaatacctatttacctggtgcgttttaatgaatgactcattatttattattattgtacagtattgttgaaagcatgatggctttcttttaaacagctttttccatacttcatattgcatttaagtataaaacacagaaatcatcatcgtgttttggctaacacaattgatgcactcttcaaaaactattagagagtatattgatttcctggagataagaaccctgatttgctttttttcttcaagattgtaaattgcattagtggtgaccgtcagatttatagttaaagcaaaacttcaagaggggtaaagtggatgactcacaaacctcccgtcatcagtgttcactgtttacgcttatctggccagcctgtctcatgcagcatccaaacggactttaaaattcctttaatttaacacagaaacgttctctgatgagtttctgttttgtttatctggtcatctctgtgcccttgtttcctgtcctgctccctctttcagtaaatcttgtttgctatcaacaggcctgtgtttgtttccttggagcttggaattcctgagcgggaaggaaatactaggaaacttaatgcagatatgtaattcttttactgttttcaaatgtacagggaccatcggttgaattgatgtatccttccttcttttggaagtgctcagtgccagttcatttccccttttgcatagctttgatggcttaattccacgcacctagacccatgtctcctggatggaggacagattagctgtgctgggttagactcataggcttatctggaatcctgcactcatctacaacgctgattgagtgaacagcaaacatcgtactgtgggtacaaacttggaggtgtttacttggaaatttatatctaccttccaaataacatcttggaaagcacactggtagatgaggcctcattgatgaattcacttagaaaaacatcatagcattcatttttatatcaatcttcattcagcccaatatttgtagctgaacaaaacactgacaacatgtcagacacgaaggccatggtcagttgcctttcacacaggaaaacaaagattagctgtgtagctacggaaagcgaatgtctgttaagtacagaggttgtagttaacagctttatgtattcaatttgtttgcaattgacaacacctcattaattgtaagcccagtgacactgcttgctgtttcaagtcacttttaaattacacacgtgctacttaatcttaaaagcaaaattaaacattggactggtttacatttcaagctacaatatggaaccattgtatttggaggaatgagtttaatatgcattgtaaaataaaattagggggtactttgcattcacagcggcttatgtaattaggttcagtcaactgtaatgtttcaggttaatgtcttccatgatgtatgctgtgtaaatagtgaacttacatatcccttaatacatctgaattattacataaatccttaatattaatataataaagtattttaagcaaattacttggaagtgtgagttgcatggcaagtaggtaaaactgatttatttttaagagatcagcaaagataaacatttcatttatttaacaactacagatatttatcaagtctctacctggtccaggtacagaggacacaaagatctgaaaactatagttaccgccctcaagaatctgctaactgagtgtgacgtttccagagggttccctggaatacggatccccagttccctgttgggcgtaggcaggacagttgggagccacaggctgcagcgggtgggttctcttgagagtttgtgggtagagttcagaaggcctgcaaatctagacggagaaaacatttgcacttttatttccaagtaattggcttcttttgcaatcctacatatcctctatgcctttaaaacattgtttttaggcctggcgtggtggctcacacctgtaatcttagcactttgggaggctgaggcaggcagatcacttgaggtcgggagttggagaccagcctggccaacatggcaaaaccccatctcttctaaaaatataaaaattaaccagatgtgatggtgtatgcctgtaatcccagctacttgggaggctgaggcaggagaattgcttgaacccgagaggcgaggtgaaggttgcagtgagccgagatcacaccattgcactctagcctggacaacagaggcagattccatctcaaaaaaaaaaaaaaaaaaagaaagaaagaaaacaaaagcattgtttttagattttattttttaaattacatgcttggagtaaaagtcactcattttggcgtacagctccatgaattttaacaaatgcagagttgtataaccagtaacactatcgagacatgcacattttaccaccctgcccccgaaaaaaaaaatctgtcatcatgcacctttatgtcagaccctgtaaccccacccctaccctggtatccactgatctattctctatctttgtaattttgccttttctgaaatgtaatatatgtggaattatttatagccttttgagtctggcttctttcacttagcacacggatttaagaaacatccttgtgtgtatcaatagtccattccattttatggctgagtagcattctgttgtatgcatgtagcgcagtttgtttatccatttcccagttgggggacatttgggttgtcttcagtttgaggcacttaatgaataaaatggctataacatgtgcatgtaggtttttgcataaacctaggttttcatttcacatggataagtagataggatgggattgctggctcacatgttaagtgtgtttaactttataagaaacttcccaactactttctgtcatgggttcaccatttgcttttccaccagcagttgctctgcatctttgccaacatttgctattgccagttatttattttttatttatttatttacttttttttttttttgagatggagtctcactctgttgccaggctggagtgcagtggcgcgatatcggctcactgcaacttcagcctcccggattcaagcgattctcctgcctcagcctcccacgtagctgggattacgagcgcgtgccaccacactcagctaatttttgtatttttaatagggacggggtttcgccatgttggtcaggatggtctcaatctcctgacctcgtgatccacccgcctcggccttccaaagtgctgggattgcaggcatgagccaccgtgcccggcccgccagttgttttttcctttgtaagtttttgccatccttacaggtgagtagtagtatttcatgtcagttttaatttgcatttccctagtgactcacagtgtccagtatcttttcaggcccttatttgtatataaagttgtttatctcctttaatagtatctgttcaaatcttttgcctgtttttaaaattgggttgttaatgctatatattttattgtatgcaattaaaagcattattctgatgatatttccctaaatggcccaaaggattcgtgttccagaaaatattaagaacctttgaaagacacactgactttaaaatgttctttacatttttgccttttctcgtgacacgtctactttgctcaaagtccgcctcaaggcagaagctggcctgtgatagaatatttcccaccccatctttctttctttctgtttttcgtatatgaagagggatttccaaatgccaagtgtgggcattcctatgcccccttcccaggagcagcaagccatttctctattctgatttctgaaactctaccgccccctttgcatcgttacccaaatggaaggaatcctaggttgtggggaaaatgtgaactaacatattccagaaaaatgaccccactccaccttaggaattaggagtttttttcttaatgcaggacaaaaatctgattgtgtattttatcatcatttatcatgcacattttattactgtttatctccacaacaccatgggcttgatatatttttctgccttaatctccatttttttcatcaaagcttcccagtagggactcatctcctagcaaaaatggtttttaactatttgggatatttttaaaagacttttgtggacccttttatatggtcagccaagttttgacttagtcatcactcattctttaagtcaagtttcaataccgtcttcaatggttaacgtttcttggaaccataaagcttatttatgatgaatatattaattttattttgcagttcttctattaaggaatataagaaggaatatagggcacagaaagtggttctgtattatttttatttgaattcaacttatgattgtcctcctcgaatcaaaaagaaaaaaacattgaaaaagcaaattacacgcaaagtctatattgaaaattaacatgccgcgttgaagaactctgccatgtgttaattattgatattgttgctttagaattagctttatcgataaatggatgtcctaaattttatatttggcctattgtggccaaatatatgcaaacttacgttaagtgcttatgttttggggtataggagctataaaaaaagattgttcccttccatatgtgtacagttcttctggggaaatgaagacatacacaggtggaacggtaagaacagtggcgaagaagggctgcacggagtttctctccagccttttgcgcatggcacgcaggctcatcattgcagctgcagttttttactctgctttccctgcagcaacagagacaggagggtggagaagggcgcctgtgctcttttgcagctgtgtgtgctcatgccaccctcccactcaacactccctgctcagattcctcacgcagcagtgacaaagtaccactcactcagtggctcaccgtcagagaagcctgggcctggcctggcccagctggttctctgctcatcgtctcacttgacgaagttgacgggctgcagggtcatgtgcctttccagaggctcaggatgaattcactcccaaactcattcaggtattggcagaatgcagttccttgaggttgtaggactgaggtctcagcttccacacaggctgtgggctgggggaggtcctcagagctagaggctgtcccgatgctttagcgggtggctccttcctccatctttaaagcccgccatgtgctcccagtctctctgaccccctttctccctcatctctctggctctcctgctcctcgtctgcctttaagggctcatgtggtaacacagggtgcacccggatgatccaggataatctccctacttacagtcagccgtttagcaaacataatcccatctgcaaagtcccttcacaacggtacctggattactgtttgatggaaaaaaacaggactggaaatcttgggggacatttttggattctgcctaccaccccctccatacacacataaacagcaaaatgacaaaattcaaggctctcctgagtgactgtttgcagcaaagaaagttgtttccatttcagagacaatatgtgctgagataagtttgtgatatttacacatttggtgtttaatcctcgtggactacattatacatatctttaggaatactctttacataaattaagataattttctctttcctgaaaacataacagcatgccacgcgttctggatgaaaatgaggaactgtttacaccatgggcccaattaaactctgcgagaattctgatgtgcgataaaaatgtgctcagatgtatagcatattttaagtttcctaggactttccttgcaagcataatcacaaattaatattaaaaagctatttaattacaatatcaagaatgtatctaaggctccctggcatttaacagagaaagttatgctaaacatacatcattattttcctccatgcagttgtccagggtgacctctcttagctgggattgacaaatccaccccaggtggcagagtggaagagcttggagtttggagttagaaggtctggattgaattttctctttccaggcccctaccgaagtcctgtggtttccctaaacctcgatttccctaatgggcatgataatcctgatttgagatttaatgaagtcatatcaaagaaaatactttgtaacctataaagcacagatgcttgctaaacaactcctgtaaacatacattatcccccttccccccaccccccgcgcacacacacacacacacacacacacacacacaactggtgattgtaactcactcatctctctttcccacagttgttttgaaaacttctgggtttctgaaatgaaggttctttgcagaggacaatcttggaaaaaaagatgaagtttgatttcccttttttctttgcactataggggtgttcactgaggttgcagagcaccaaccatgtctgcagaggagttcacacctcactggtgttcattgtattgcttcaccgttgagagcctgaatgtgaaccttgttgtattaaggtgatattcttgtccttcatgtgttagaatatctatttgctttgaagagagagtcagactccctgccttttgggacatggctttgagatctgcctttcagtaagtaagacgctatcctcaaattgagctgggctacctctctacatgggaaaacctaagtacttcacagaaagctcggtgacggtttgagtttcccagaatgccgcactcatccccaaggataagtcacgtctttgggggatgatggggcttcgacactggacacgtggacactggaccggaagttagaatccagtctcccagccctcaaccccatgctcctttccatggcgaaggaggagaagctcgccgccaccccactggcctgctctgctgaggttgtggagcaccgggaagcaggggccgagccttccccgctctgtgcctgggggctctgggcactgtctacacctgggaggtggtcagcatatgtgcgtgggaacttgctggaaggaaggaatgacccttctagccatggaacggtggtcatccgtgagccacggcctcaggaggtgggttcccttaaggctccggctctgggggcagcgccccctgggagcgtccatttttaagcctgtgaagaaagggctgctgacgtttggggcccccgctatgagcatagcctccactcctccccaatccctgtccgtgcctctacatacctctgtggcatttgcaaacccttttcttagcaggtgctcccatctgtgtttgcccttctctcctcccgtttccatggagaggctggcaccaggacgcgtgactgacagtgctgtctcccccagcagtcccctctgagtctgtcgggtgtgggctccgggctccaccctgcttcttccttagcccgctctctgcccctcccactgcccaggctgccggggcttccagctcccgtggtgcaaacgtggcctttgctggcggcaggatgcggttgcgtcaggagtaagcagagcttttgtgaccaaaatcaaccccggggcatctcaggctctagcgctggtgggcgtgtgagatcatttcagcccaacccacatcccaggcttgaatcccattaccaggggcaaccgatccttcttgtcctttcccagattggtgatggatgtccactgtgtctccatgcagctggccactctgtggacagctgtttgtgactcagctcctcccagcctgagggcatctgggccctgcaactccatcctttgctcccctttcttcccgcggggaccctttcatctccctggcaggaccctgtctccacccgagggcacgttctctgacctcttctgcggagtaccctggcttgatgacgcttcctgttccttcccagggttgtgctgggggcatcagtctttgaacagtccctccctgagggctgccgtgtcccctcagtgcccaggcacatcaagcactgctacaaacccagacattcacagggacccctccacctgaagtccaccataaggtagaagcttccggaaggggtgatgcaaaggggtcaaacatttagactacgcctcagagctgcatgattccttcctctaactgtgaagcaaatacagcgatttctctgggtctgccatcgaggccttcttccctctgctctcacctcaggtgccttcctaatgtgcaggtgacgattgcctctgagctcaggcatgcagcttctgggatgcactctcctttcacggcaaagcattctgcataggaaaaaccgtctctgcgtctccaagctgtggcctagcccttcattcatcaaatgggatacaactttcatttcttattaaaatctggaagtcactctccatttgtaagatatcccaggggttgatgtcatcttgtaaagggggacagtgccattttctatcttacactttaaaaagacaaaaccactagctacaaacatcattcaccacagcatgggagaggcaaccccacagcaaaggttatgtcatgtttcataaatgggtatggatttgggggctggtttttttaataaaaacatattttggtactttggaacttgacataatttgagctgtttatgttgtagatgttatgaatagatgttatggtattaattaatagtgtgtattttctatgtctgggcttgcctggaccgctggtccttgcctttctttctgacacagaatttccagggcctcaacctgcttctgtctggaagtgacagcccactccacaggggtcttcagataggggtagaaggtggtgccctgggctctcctggctctagagaatctggctctagaatcacgaagccttccagcatccccagcttcccccttgcagtaatcccataagggaagcgttgtcccattttacagagagtccgtggggctgagaggtgtcatgaattattctaatggtagtaactctattggagaggctgaaacccggggctcgtggatgttgtgacccaccccccaccagaacctcccatcctgactcactgaatatttagtaccatcgtcccctggaggatggtctagttggcaagtagctgaggatttttaattctaatttagtttaaacatggctgtttgttgctgttgctctctaaagggccttattaaagactcttgctgcagtgaaggtggcctgtctaggaatcgatatggatgttgagatctgagactctacaacacccccaatctcagtccttctcgctccataggcagttccattccctgcagatctaactctgagattctcactcctgaaaatgcctggacaataaataaatccaaggtgaaggtgttgatgccagtgacctagctgcatcattttattcatggcatcaagccttcaagaatgtggcttaacttgtatttattcacatgataatggctttttataaatctgcatctcttcctcccctaggaagaaaaaaagggcatctcttcatgagtcacttgattaaaattcctctccagtactctgtaccatccactcatactggaattgttgaaaatcaggtgatttgcaactccacacttgcccaaattaaaccatgagtggaattgacaatatgaatcctgaattttctgagctggaaggaggttctagtgattacccacaccagggatggcattttacacatgaagaaacagaggccccagggttgtttctgatccaggtaggatcaatatctcggtagagaaaagggggaatttttttgcttatactttttaaaataagctacttgtggctgggcgtggtggctcatgcctgtaatcccagcactttgggaggtcaaggcaggcagatcacttgaggtcaggagtttgagaccagcctggccaacatggtgaaaccccatctctactaaaaatacaaaaattagatgggcgtggtggcacatgcctgtaattacagctacttgggaggctgaggtggaaggatcgcttgaacccaggaggctgaggttgcagtgagctgagatcacaccactgccctcaagcctgggaggcaaagcgagactctgtcttaaaaaaataaaagtaaaataagctactcgtaaggggtgtgtgtgtgtgtgtgtacatgcatgtgtgtaatctctgtggcgtgtgacctggatatattcataaaggtgtaagaataattcaagtaggtaacgctctctcacattttttaaaaaaagaaagaaggaaaatatttattccttatatttcaggatggcagtcaattagggtgtgatgtgacttaaaggtcacaggagaccttgcctcttgccaagtgctgccacgactgtgtggaaaatctcttgtgtactggggacccagtttctttcctgtgaagggagttgacagaaataaaaaagatcactgaatgagataatcgttgtaaaggagccagcatagcagccccttaattagggggagtgtgctgtgtcactgaaacactctttgtttccaatcagggagctctcactgatcagtcaggactctaattgttggatagagtcaacaattagaaaaaaagttatagtctcctaggagacatagacaagtgagctgacagggtgggctcagctcaccctgtcccagcaccttgggaggagtgtcaaggcaaacgacaggcagctgggtgcagtggtgtgagcctgtgattcctgctacttaggaggctgaggtgggaggatcgcttgagcctgtgagtacaaggctagcttgggcaattttggggagacagaataaacaaaaattgactttttttaaaaaataagtttgtattttcttcaattcaaaacatttttagaattgatattactttttatagctttaaggcatatttcacatcgatattacttcttaatatatatatttttttgctacagatttggagtgtgtaaattgataaaattagtatcaatgctattgtaactacctggaattggttaaatagcagcagtgccagtcacctttcttagagtagagattgccagcaggcaatatcttcatcctcaggacccaggtctctcttctctgaccctgcctggaccctgacaatcccagggcgctctgcgttggaatcagtttggatgtgtgttaccagatcttcgctgaagcagggcactctgcattggagtaaatttggatgtgtgttaccagatcttcgctgaagaacgcaaggaacccagcatgcaaagctggccactacagttacatttttccctccctttgaaaaaaattttatgaatgcctttctgaacaaagaatatgaactaaccctcatcattcattacatttaaatagcataacatgccattgaaatccttttccagacgtttcagcaagtgaaatcatttgtggttcatgttatagctttttcagtaacatgacttgagaaatttttcttcttactaataaaagggaatcattcccgcagttaaagaattcatcttggaatcctctggtttttcatcatgctttacttttaaaacttcccgttctgctgtagcaaacttcccattctgctgtggcaaacacgagtgcatattagttttcatctttggaagatgaatgaagtatgtgacttttcaaacagggatctcacattttaagtctagttattccagaataaaaacaagccagaggcccaaacacaggattcttcactgtcgttagcaatttgggcacaaatctgaagggccagtttggtgcaggtgtgctggaggatacatggagaaaaaaaaacatgttccagtctagttctgatacacagaaattgaaagagcaacccaaggagttgggttgctcttccacatggtcaggatgagccaaggcttccgggtagggcttttagggcaagtgggccccagctgagcccagcaggggcagggccaagatgagcagaaggaagagataggctctggaaggagaggcaggaggaggcaggtgtgggcaggtgcaggcagatgtgggcaggtggaggcaggaggagacaggaagaagcaggtgagggcaggtgcaggcagatgtgggcaggtggaggcaggaggagacaggaggaagcaggtgagggcagatgcaggcagatgtaggcaggtggaggcaggaggagacaggaggaggcaggtgagggcaggcgcaggcagatgtgggcgggtggaggcaggaatagacaggaagaggcaggtgcaggcaggtacagacagatgtgggcagatggaggcaggtagaggcaggaggaggcaagatgaggtaagaggagccaggtgcgggcaggtgcaggcagatgtgagcaggtggaggcaggagacaggaagaggcaagaggatgccagctgcctgaggtggctgacagcttcacccagctgaggttcttgcccatcctatacacccatcccaggacctctctctaaacactctgctctgactttggcatttccttgaataaacaccccagaagctcctcgtctccttcatacaataatccagtctgcctagcccagtgaatgagggttctaccagagcccagccaacccctaggacagctcacatcccaccatccctgaggtactgcaggctcacagccttcctgatggaccacacctgtgcaccgggctggtcccctgcatgcacctctcattcaagttgccttcctctgggaagcccccagggacttcttttctgtcctgcactggcagagtccccctgcttcccctgcacccagcacattccttccttcatcaggtgttgctgcacctctgctgctgggcaggcttggtgccaggcactgaacacacagctgagtcccagatggatgtcttagtgtcaggagacagagtaaagttctaggcctattgtccatcgtttcttgagacagggtcttgctctgttgcccaagctagccttgaactcacaggctcaagctatcctcccacctcagcctcccgagtagtagggattacaggcttacaccattgcacccagctgcctgttgtctgccttgacattcgtcccaaggtgctgtgacccagttcatttgtctgtgtcccctaggagactatgactttcctgcaggcagggttgtaccttctcatctgcaccctcagtgcactcaggcaggtagtgtccagaaaatgcttgaagacaagaaaggttcccccagtgagtccatgcagagcagaacggcaagcagggcaggaggcaccaagctggcagggcagggtcttgtcccagatgtactgtgtcctggagaatgatgtgctaaccacatgcaggccgcctgggatggagaagagcagtggagtgagtcttccctgcctggtgggaggtggaggccctgtggcatctgaggtcaaggtcactgccactgggctggcagggctatttctccaaatgttagaatcaagaacttcagattaaacttcccctctgcctactccttccccatgtcagcattgctgaacagctgccatttagcaattgtgggagcaacacctatttattgagggtccattctgttctgggccctgacacagtagccccaggaggtgtcacaaatgcaagagtcaggcaggtgtcatcatacatgagccaggggagccaggtgggcctgagccgaggggagcaacacctatcccggccacaggggtgccctgctcaggctcagcccgctgtgggctcacaggagcacgtcttctgatttttcaagagaagctgagcacctagatttttgtgtgaaatgctacaatttaaaaatataaacaatgaatgtaaacaaatcttacaccaggtaggctaaataggagccatttcctagccgcacttggtttctggacagccagttttccaccttgattgataaaagaaataaccattcttggttttttatttacttccagatctccattcccctgctctttctacaatacaactcccaccccctccatctcactaaagctcccactgatgctcctgaagccttgatctgtacatccaggcttccttggtttgtggccacaaaccccgaatccctcactctgatttgcagagtaggggacaggtgagggctgggcccagggcagagccaccagtgagaagttgggcaacccttgatctcatcagtgtggcccagactgtgtcactggtaagggaaaaggcaaagaaaacaggcaaaatattgatgtttatctgcagagctgaatgtctttttatctcatagaaaaaaaaaaataggtcagcctcccctgaaatgcagggcaagcctcctatagtccagtcaagagcatttcgagtgtcacttgccatggaaggaagctggagaccacgatgtcagagaaatgagaatgagagaaacagaaggagagctgtgatatccaggtagatggtttaagcacagggaaccaaggaatctgtctccatctgctagcaaactgaggctctaatcactatcagaaacggaagaatgttgaggaaaggtgatcagaggtttgggtgctgttcatcaacctgtctaaaatgaaatttttaaagtatgtttttctttaaattgtacgacatcctcgatttataaagcaaatcaactaacaaagctatgcctttgtgggcactcaacttgatcctttgtctttaggttaaaaaccaaatcctacaaaatgtccatcaagggatgaatgaataaccaaaatgtggtcatacatgcaatggaatgctgttccgtcctaaaaaggaaggagctcctctcatttgctgcaacacggatgaacctggaagacgtactgagtggaataagccagttacacacagacaaatactgtatgattccactcacatgaggtccctagagtagtcaaatttgtagagacagaaagttgaatggtgggtgtcaggggctggagagagagggatggagggttatgtttaagagggacagagtttcagtttgggaagaggaagaagtactggggcgaggtagtgatgatggttgcacagcagtgtaaatgcacgtaatgcccctgaactgtacactcaaaaagggttgaaatgttcaatttcattttacacgtacattaccacaatttaaaaaactcatacaagagcaggaatttcagggccgcatctgttatcataaaaccatggcactgttgagactcggaagcaattttaatcacgtgggccaggaaaccggtgactttcttatctctttgatctcatggaaacaccaacactgttttcatgtgttttattcttctcttcccagctctgccagacatccacacatcctgtctaatgaaaacagtaagactgtttaaactccagaggtcatgtaaggaaatcattcctaaatgtgtagttcagagtaaaaagtgctcttgtcgatagcaacttttcataaatttgtttttctgagcgtcacagtggtatttgttcatctctaaaaatctggattgtttcaagatgatttttaaaattatttataaggaacccccaaacttgcctggctgccagtctctcacactccctgtaaccctcgagttgaggagtaggttgctaatcctctcctagagtgtctctgccaccagcctggaaggcactggggctccatgagttcctgcagctctttgtctgtgcatgcgagaactttcccgtcgtcttctcccatgcaggagggagacggctacctgagcctctgagtctaggttgaaggaaggtcggtgaagaccccacccacagtgagacaatgcggtggtcatggggaagctccacccaccaacaccccaagccccagaagttctgctgcctggaatgcgcaggaggcttccagggcagctgaggtgactctgatttcatttgctcaggcagatggtattggagagactcagccactttattctgcagttggtcagcacttcccaaggggattttattggtcttctccaaactctcttaattccagaccttcccagcctcacatttgctagtctcagaacagagctaaggaggcctgttcctcacgagctggacaggcagactggaatgaccgtgcccactgctggggtggcagtattgggcatggtgtatgagtgtgtgtgggttgacctggctgtggactctgggtggaaggaggaggaagtagttcctatgggcattggggctgggaagtctaattagaagcaatagtgttggcccaacaagtggggcatttgtgcacgacagaacctaagggcacacaaagttgctcctgtcagggatgtgagtcagccaatggccaagctttgactttcggccagggtggagagtggcagcaaatcatggttctgctgctggaaggactgaagtccagggaatattactgctcagagggcctgataagaatccagggaccagaactggggtataaagtaggactgagaattaagaggaagaccacagctgttgtgtcacatgcagcatgatagtcagggatggatgagcacctctgggaggtacttgataaatattgattgaatggctaaatggtgggtgggtggatggatggatggacgatggacagatggatggatatgtggatggatggatggatggatggatggatgcatgcatggataagtggatggatggatggacagatggatggatacatggacagacggatggatggatggatggatggatggatggatggatggatggatgggtggatggatggataaatggatggatggataagtggatggatggatgggtggatagatggatggataggttagtgaggttagaaaagtaggcagaaactggatcatgaaggccctataaggattttgcacataatagaaggtcatttgaggagattgagcaggagagtgacaaatggaactgtgtatttgaaagatctctgtggctccacgtggatagcaggctggtgtaggtacaagactggaagcaagagactagctagggggttgttgcagtgaaagaagatagtggcataaaattaggtaggtggcaatgagatggagagagtgggcagattccacacatttttagaaagcctgaagatcaggaatcagtgactgttctttagatgtgtggggaaaaagagggcggcatctagagtgacccccagtgggctggacagctggggggatggtggcaccttctatcaaaataatgggggaaaggaagagaaggctagggatggatcgttgtgactgtcatgggccaggagtgccttgtggcatggatctgtttataagcctatctccttcattggacctcaggaaagaccctccagcttcctcacccttcccctgcacatccagagcttagtgcagtgcccagtacctggttggttttcgaagttattggactaaagagtgaatatggatctgggattagacagtctttaagatcattccctgcttagttgtgctgctgtagcaacattcatacatgggggtgagaggggtgacttgcggaactggcaactcaatgtggagaatcttccagggcaagcaggtagaggagaaaaggcctctgtgcctgacctcatatctgaagcccaaggcaacatgccagccagactttgggctccttgcctgtggacacctgtgtcttactgtctgctctgggctctcctttgaggaggcctctgtctcagaacaaatgtcacaaacaatgaatgccatgcttcaaaggaagctgatgtatggagcgtgtgtcaccttcctctacaggaatggggtttggcagtgatccaaagactcactgttatgacatgttctcagggaagtaggtcatatgtccccagggtattcagagaggcctgtgagtgtgagatgtttgggctgagcagggctttcttcctctcaaggctccaaagggcgggccaacaggtcatttggtttttctcagtgatatgttttcctcctcaagattcatcacccctctctcctcctctgcttgttctgaggcaaatggacacagaaatacattctctgttttatccttcatcgttgctaacccctgtggggtcgctgacatcatggagttctcttctggtcgtgcatattgtagctcatgaagaaaaggttgtaaatcaccatttcaggatgttctgcctttgacctactatgactccatggagtatcccctacagagctctctgtgggtctctgagaggcattggtgtgtggctgcatctggcaccttgaggtgaagatgggccaggagtagatccaggaggagcacagcagagcataggaggtggctggagaagaaggtcagatgctgtggggtcaggaagtgcctgtcagagaagcacctgggagtttgtttctggctgaggccagcaaggggtgcaggctgcatgacttccttcttctgaatcttcctttcagggcccaagtacaaagagaaaacacacatcaaaatgcttggagaatgaatggctattcgtgtatataccatttcactgatattgataacttactatgtggccgatcctgctcaagggcctaggaagaaaaagaactctcagccctcattttggggcaaataagtaaggatatatatacaaggatgtttattgcggcaaaaaataaaacactaaatttataatggcaaggcctgtcaacagggaaagattgaaaagagattgattctgcatctgcatcatggagcagtctgcagcccctgggagatattcatcagacctcgcaggctggcagggagggatttccaccccgcattggtaagagagaagagcaagatgcgcttgcacattgcacctgtgcacacttgctcatctgtaattgtgggtacagggacaaaggtgtgtgcagacacactgcaggtcacaaagactgcatagaggcatagagtgaaagtgggtacagaaaaggggaggagtaggaagaaaagaaacaaggaatcacaggacacggcagctcgtgtctgtaatcccagcactctgagaggctgaggtgggaggatcacttgagcccaggagttcgagaccaacctgggcaacatcgtgagaccctggctctgctaaaaactaaagccagatgtgatggcttgtgactgtagtcctagctactggggaggctgaggcaggaggatccctcgagctcaggagggcaaggccaccatgattgtgccactacactccagcttagatgacagaatgagaccctgtctcaaaaaaaaaaaaaaaagctatatgtttttaaatatcatttatagtgtgatttgtgtaaaattgcatagacacagagaagggtttacgccatgggacagccacgaaaatgtgatagcacttcagtttgactttaaaaaaaatactcttgtgcattgttcaattgtttttataatacagtgatcacatttatttagtgattggaaaagaggttatattttagttttggaaaattaaataagtgtagatgtgaaaacaaaataaataaaaattctcagaggtatgtttatctaaagtgagccagattcacgtgcattcaaaactcctctcctaggaaaacctcccctgattgtcttcttcaactgttaggaaatagtgttgaggtgttgccacaccctgaggcccattctcccctatgctgtgtgttagaagtctgtctcttgctctgaagaattcaggtttgtcccatgggtttacattttatttatctcttaatgcttacatcctctgatgcctctgacattttaccttgtctttggacaaattttcacaatgtttaaaaggtatgctttaaaaagatatgctcttacataacttttctgtgcccggctattctcccatatgttaaaggaggcctcctttccctggaccaccatgaccacccgagggacttgcaggttgccagggcatcacctgtcatgttcttttatggaaagaatctggactggagcacatgagctcaagaaaaatctcggacttaaaattcagtctacaagagaaactgacattgtcttggacctgtgtaatgggcaaagttcagagtctctggggtacagggaagtaggtcagggagtggggaaactgaggcctgttgtgggacttctcagcccaccctaattagaggggttgagccaaccccccaataaataccttctcatgtgacagcatgggacgtgagatgggaaaggcccatcagctaaatgcgagccacaccgtgaaggatgccccatgccatgcccaagggtttgggttttctcctaggaggggtgaagagctgctgaagcttttgagccctgtagatgcttagatgtgtgttgggccactccgttggcccccaggagagcagatggggaagggtgagcctaggcttagaagaccagcacagagtcctgagcttggggatgtgcacacaccatcaccccctcctttttttttgtttaagagagactttattttagggcattttaaattcacagcaaaattgagtggaaggtagagagatttcccatatgccccttaaccctacacacacacagcctccccactctcattatcctacacgtcatcatcacccagagtccatagtctgtgttggggctcactcttggtgttgtacattctatgcatttggacagatatatgacatgtatccaccatggccgtgtcatatagaatggttttactgccctaagcatcctctgtgatccatccatttattctccctccctgtgaacctggcaaccactgatttttttactgtctccatagttttgccttttctagaatgtcatgtggttggaatcatataatatgtagctttttcagattggcttctttcacttaataatacacatttaaggttcttcccaggcttttcatgagttgatagttcatttccttttagcgctgaatagcattttattgtccggaggtaccacagtttatttagccagtcacctacattgtgatggttaatttgaggtgccaacttgacaggattaaggaatacctaaaaacttggtagtgtagtattttggagtatatccttccagaggaatttagcatttgagcctgagtgaattaggtggagaaggttcaccctcagtgtgcatgggcaacatccgatctgctggggacccagacacgacaaaaacaggaaaggtgactgtgtcgatctgtctgctggaactgggatacaccctgcctctcctgaccctggacaattccaggcttgtcagcctttggattccagaacttacaccagtgccccaccctcaatccatcccctggttctcaggcctttggccacggactgggaatttcaccaacaccttcgctggttctgcagccttcagacctggactgaaccatgctgctagtgtgccagggtcttcagcttgaagatggcctgttgtgggacttctcagccaaccctaattagaagggttgagccaaccctcaagtaaatcccttcccatgtgtctatctagctatgcatattctattagttctgtctctctggagaaccctggctaatacagattttggtaccaggagtggttctagaggaaacaattttaatttttggttttggagtttctggaattggctctctaacctgtttagaactaaaaatgctggagactctacttctaatagcgcagagagcactgatagatcacgtgcttactgtttgtaaatatgcaaaataaatacatccatactcctaattcaccacttataaaaggcaaataatttagagactttatacctgatacgtttgaacatttgaggaaaaccaaagaatatgccacgttcattggttgctcctagcacggctaaaccaagtgatgagagaaaggggtgagctccggtattcacataaatgacctgagtttctacatgtgccctgggtcagaatcttctctcccgtagccacagggctgaaattgctgaaaatcaaacacaagcctttgtccctcgattgactgaattaccgcgaaagctgagtctcagctttgcggaaatttgggatggggaaacgtggggagaccttgatgaggctagagacattgagctcagaaattctgatgagtcttatttgctaaaagtgacctcccctatcccagaaataaatcccacctctgcctgaggggattaactctgatgggactcacagcctcccctgagccagttgccaggcaagacagtgctgaatctctggacccgcccccaccaaccctctttccttctagacctactgtaactagactgaagtcccagcagcccctacaggtgaggtacacagtgtgccccatgaggaggtggttacactctaaaagaactactttggttttctaatttatacaagcagaaattcggggaacatgtgtgggagtggatattaagggtatggactaacagagacataaagggagccgactgaatttattgatatggacccactgagcagagattctgcagttaatgctgcagcgcagggaatttggggaggggctcagtttgttttattggttggctgaaatgtgggtcaaaagatggtccaccatgactgagttggagatgcctgatctcctttggtttactttaaaggaagggatccagaggcttagggaggctggaatgctagagtggatttgtcacttaaaaccaactcccacacactaggagggtccagaagactggtctttcaccaaaaccttgagaaacagatttctggggggagtgccagcatccctgaagagctccgcgattgctcttctctgtaggctggactttacagtgggaacctcagtcactcaattaggaaacttaaatgctatggggataattggatcctggggtggcaggggccaagtggcagcactcaactgtcaaaggcaaggtggatgtagttactgtaaggtacagcagagcaaagcaacaatcaaaatggtctgactcgtgtagatctatggcactggctagttaatcacgtgtttctaaaagtgaaatagggtcgggcaaagtggctcaagcctgtagtcccagcactttgggaggccgaggcgggcagatcactaggtcaggagatagagaccatcctggctaacacagtgaaaccccatctctactaaaaatacaaaaaaattagccgggcatcgtggcgggcgcctatagtcccagcttcttgagaggctgaggcaggagaatggcttgcagtgagccaagattgcgccactgcactccagcctgggtgacagagggagactccatctcaaaaaaataaaaataaataaataaataaagtgaaatagataggaagcctactaaattcttacctgatctgtataagcataaaactttcaggtcaactgcacaaaagtccaactcaaatcataaaaactgagagtcgtggctcctcaatgaattcccagacttgagccagtttatagacccagaaccccttgggtgaaggagaggctgggttccctccaggaaggactctggtagactacaaagaacgtatactgttcatctttctcccatctttccctaaagggatctatggccttttaccatggtaactgtgtgctggggagagggaaataaacagacaatttgggaactactggacactggctctgaactgacattgattccaggagacctgaaatgtcaccatggccccccagttagtgtggggcttatggatgtcaggtgatcaatagagttttatctcagatccaatttacagtggactcagggggtccctgaccccatccagcggtcattcccccagttccagatgcataattggagtacacatactcagcagctggcagaatcctcacattggctccctgacctgtggagtgaaggcaattatgatggaaaagggtaaatggaagctattagtgctatctctacctacaaaaatagtaaatcaaaaacatgacctggagggactgcagagattagtgccaccatcaaggacttgaaagatgcaggaatggtgattcccaccttcaactctcctatttggcctgtgcagaagacagatagctcttggagaatgacagtgaattatgataagcttaaccaagtggtgacgccaattgcagcagctgtaccagttgtgatttcattgcttgaggaaattaacacatctcctgatacctggtgtgcagctattgatctggcaaatgcctttttctccatccttgtccagaaggcccacctgaagcagtttgccttcagctgacaaggctagcaatataccttcactctcccacctcaggggtatatcagttctctagcctatgtcacaattgttttcaggaatcttgatcacttttcccttcaataagatatcacactggtccactatattgatgactgaaggaacttatgtatacagtgatccatttccaagacaaagtgccttaaatcagcttaggtcagcaaaactacagaagaaacaggatgtaatatgcccctgcttagatagccaatgcctacttgttgtgcccaccccgccccccctagcccttagttgccctcacccaaaccaaaaaaagtttagtctaagatgaaagtttattagcctgcaaaatagctccctttttctattcttatcagcctgcccagctacttaggtcataagtcaaatacttaaagagttaagtattttacttataagctcaggggagctgacaaggattgcagtgcattgtgggctgcaacaaaatgcagcaagacaaccctaataaaaatcacctacagcccctacccaacaatcaataggcgatgtccaggaagattgtaaacccatagtactcagcctatagggaaccgggggagggacctacacactaggggataaagtgcttgttaaaactgtgctgggtgtgcctgcttatcagacactcaatcttgcaagactgtcattaaaagtctcacttttgctattccttgggtctctgagtccattctttgggtttggttgggtaagtttatttctcacaatgacaatatgctgattgggcctagagagcatgaagtagcaaccactctggacttataggttagacattcgtatatcagagaatgggaaataaattgagcaaaatttaggtgcctcctacctcagtgaaatttctaggggtccagtggtatggggcatgtcaagatatcccttctgaagtgaaggataagttgttgtatctggcccctcctacaattaaaaaagtgcctagtgaaactatttggattttgtaagcaacacatttctcctttgggtgtgttactccagcccatctaccaagtgacctgaaaagctgctagttttgagtgtgtcagaacaggagaaggctctgcaatagatccaggctgctgggcaagctgctctgccccttgggccatatgacccagcagacccagtggtgcttgcggtgtcagtggccgatagggatgctgtttggagccttcggcaggcccctataggtaaatcacggtgaaggctcttaggatttttgagaaaggcccggccactacctgcaggtaaccactatccttttgagagacaactcttggcctgctgctatatcttagtagaaatgaaacacttgattatgggccaccaagttaccatgcaatctgaactgctcatcatgaactgggtgtgatctgacccaccaaaccataaagatgggcatgcacagcagcacactatgataaaatggaagcggtgtatatatgtgattgggccagaaaaggtcctggaggcataagtaagttacatgaagaagtggcccaaatacttatggttcctactcctgctatgctgccttctctctcttgcctgcaccccatggtcatttgccagaggaagagaagaccaggacctgctttataggtggttctgcatgatatgtaggcaccaccaggaagtggacagctgcagcactatagcccctttctgggatgtccctgaatgacagtggtgaagggaaatcttctcaatgggcaaaacactgggcaatgtacctggttgtgcactttgcttggaaggagaaatggccagatgtgcaattatatactgattcatgggctatagccagtgatttggccagaaggtcagggacttggaaggaacatgattagaaaattggtgacaaagaaatttgaggaagaggtatgtggatagactgctctgagctggcaaaggaagtgatatttgtgttccatatcaatgcctaccaaagggtgacctcagcagaggaggactgaaataatcaggggtagaaaatgacctgttctgtgaatatttgtcagcctctttccccagccacccctgtcatggactcgtgatctaagtggtcatggtggcagggatggaggctgttcatgggctcaacaacatggacttctacttaccaatgccaacctggctacagccaccactgagtgaccactctgcctgctgcagagatcaacatggggtccccagtatggcaccattctttggggtgatctgccagcctcctggtggcaggttgattacaatgcagcaccttcattgtgaaaggggcagtgttttttccttactggaatagacactctggatatggatatgcttcttccacagctaccatctgtggactcatagattgccttatcaaccatcacggcattctgcacagcattgcttctgaccaaggaattcacccaaagccaaagaagtgcagcagtgggctcatgctcgtggaattcactagtcttaccatgttcttcatcaccttgaagcagccagtttggtagaacagtgaaatggccttttgaagtcacagttacagcaccagctaggtgacaatgccttgcaggctgaggcaaggttcttcagaaggttgtaggtgctctaaatcagtgtccaacatatgatgctctttctcctatagccgggattcacaggtccaggaatcaagggtcaggaatggaaatggcactactcaccattactcctagtgacccactagcaaaatttttgcttcccgttcccatgactgttagctctgctggcctagaggtcttagttccagagtgaggagtgcttccgccaggagacacaacaatgattccactcaagcagaagataaggatgccacttggccccttggggttcctcatgcctctgagtcaacaggctaagcagggagttacggtgttggctgcggtgactgatccagactaccaatgggaaattggactactactccatagtggaggcaaggaagatcttctggaatacagaagatcccctagggtgcctcttagtactaccataccctgtgattaaggtcaattggaagttacaacaaaccaatctaggcaggacaagtaatagcccaggctcttcaggaatgaaggtttaggtaactccagataaagaaccatgactatcatatgatgcagagaataaatacatgtgtacatacatacatacatacatacatacatacatacatatatacatacacacataaaatcatgaccagtgaggtgcttatcgaaggcaaagagaatacagaatgggtagtagaagacggcagtttatcaataccagctatgcccatgtaaccagttacagaaacagactgttatgagtatttcctccctattttgttaagaatatgtccatgtgtatagatgcatatagtaagcaaagatctttattttcattcctctcttattcttttatcatgtaacaagattttctgaacttatattagtatttaagtgttgttaattttgtatcatagtatttaagtcatgagatataaggagaagagtaaacatcatgcaaggactttttctgaagaaggaattatattcatcagttttcacactactgtaaaaaactacctgagactgggtaatttatgaagaaaagaggtttaattgactaacagttctgcaggcttaacaggaagcatgactgggaagcctcagaaaacttacaatcatggaggaagatgaaggggaagcaagaaccttcttcacgtggtggcaggaaagagagagagaaggggaaagtgtcaccttttaaaccatcagatctcgtgagaactcactcactatcatgagaacagcaagggggaaatccatcccatgatccagttacttcccaccaagcccctcctccaatttgacatgagatttgggcagtacacaaatcgaaactatatcagggattaatgtgtttttggttgtacgcaggatagctgcatcctgtcaggtgaaactattaccttattattatcttatctggaaattaagtatgatttcaggagttgcatatatataggtgccaagttgacaaggggtgggcttgtgatggttaattttaggagtcgactatattaaagaatatctaaaaacctggtaaaacattattttggggtgttgtctgagggtatttccagaggagattagcatttgagtccaagtggactagatgagaagccacactgccagcatctcagggactccaccttgcaaacggcttctcaagggactcctcagccacaactgtgtgaaccaatctccctaataaatcccctctcatatgtctatatattcattgcattgatttttgtttctctggagaatcctgactaatacagacatcttggttgcttccaagtttgggcaattaagaataaagctgctatatacatacatatgtgggtttttgtgtggacataagttttcacctcatttgggcaaatgccaaggagtgcaattgctggatcatatgttaagtgtgtttagttttataagaaaccactaaactatcttccaaagcagctgtaccattttgcatttgaagcaataaatgagagttcctgttgctccaaatccttaccagcatttggtgttgtcagtgttccggattttggctattacaataggtgggtagtggtgtcatattgttgctttaattagcaattccctaaagacagatgatgatgagcatctttatgtatgcttattttccatccatatgttttctttgttgatgtgtctgttctggtctttgcctgtttttttaatgggattgtttattttcttattgtgagttttaagagttttttttgtacattattgataaaaatcctttatcagatatgttctttgcaaatgttttctctccagtctgtggcttgtcttctcattaccttgacagtgtcttctgcagagcaggaagtttttaatcattatgaggttcaatgtatcaattctttcttttatgggtcatgtttttggtattgtatctaaaaagtcatccccttatccaaggtcatctagatttttttctgttattttctagagttttataattttgcatcttacatttaggtctatgatctattttgagttaattttttgtgaagggtataaagcctgtgtgtagattcattttttgcatatggatttacagttgtcccagcaccgtatgttgaaaagaccatcaatctttctgcctttgtattgccttcactcctttgttaaacatagttgaccatatttatgtgggtctatttctcttccattctgttggattttttccttcagatttgttacatagataatcatgtaatctgtgaacaagaatatttgcatttttacttcttaatctgtataccttttattttcttgtctcattgcaacagccatgacttcctgtatgatatcagaaaggctctccattctgttccaatgatgttgattattttactgaagctttatagttaagtcttggggtcaggtagtgtcagtcctccaactctgttcttctccttcaatattgcgttggctattgtgagtcttttgcctctctgtataaactttagtatcagtttgttgacatccacaaaataacttggtggagattttgactgggattgcattgaatctgtagatcaagtgaaagaactgacatcctgacagttttaaatttgtttatccgtgaacacggaatgtctctccatttatttagttgttatttgatttctttcatcagagttttaatatagatcttgtacatattttgttaggtttatgcctaggtatttcatatttagggatgctaatataaataggattgtgtttttaattttaaatttcacttgttcacttctggcatacagaaaagtgactaactttttgtattaactttataccctgaaacattactatagttacctattagttccaggaggtttttttgttttgtttttattcttccaggtttgttacatagataatcatgtaatctgtgaacaaaaatatttgcattttttacttcttaacctgtatatcttttattttattttcttgtctctgcaataaccataacctcccatatgatatcagaaaggagtgtcaagagaggacatccttgcctcgttcctgatcttagtgggaaaatgtctagtttcttaccattaacgtatgacattagctgtaggtttttcatagctgtcctttatcaacttgaggaaatttccttctattccttgtttgttgagagttttcatcatgagtcagttttgaattttgtcacgtgcttttttttgcatctgttgatataatcttgtgattttcttctttagcctgttgatgtgatggattacattaattgattttcaaatttgaatcaaccttttatacatgggataaatcccacttggttatggtgaatacttatttttatacattttggatttgatttgctaatattttgttgaagattatgcagctaaattcatgagagatattggtcaatagtgttgttgttttttttcttggaatgcctttggttttggtattgagggtaatatgggcatcatacaatgagtgttccctatgcttctatcttttggaagagatcatagaaaattgatttaatttatttcttaaatatttggtagaattcaccagtgactcatctgtgcctggtcctttctgttttagaagattattaattatttatttattttcttcaatagatataattctattcaacttgcctttttttttttttttttttcctgagacggagtttcactcttgttgcccaggctggagtgcaatggcacgatctcagctcactacaacctcctcctcctgggttcaagcgattctcccatctcggcctcccgaatagctgggattacaggcatgcgccaccacaccccgctaatttcgtatttttagtagagatggtgtttctccatgttggtcaggctggtctcgaactcccaacctcaagtgatccgcctgccttagcctcccaaagtgctgggattataggcatgagccaccacgcccggctgaaattgcctattttctttttttaaacagaaatccaggttaacaacaagttgggacttgaggggagtggagaaaaacagtgcaagagtttcctaaaagcttacccagttcatgcattcactcattcattcatttgtacattcattcatttggtaattcactcaatagcctcctactgaacccatttaccagaaataagtcctgcctcaagatgatagcatatgcctttcttatgccttcctgggcattttcttgaattacttttttgtggttgtttttaatgtgtaaatatacatccccaataactcagactcattaatttatgtttgtaataattgttgaaataagtttgacagaaggtgacctttctgaatttgcaaagaaagtgattgccaggcaaatttaaagcagaagtccattgtggggaagatgttgtgtccaaagccacacacttttcaacgagctttaagcactttctccatagtgttgagggcacaactgtggagtcagaccttccccagtatgatcctgggtcagctacttcccagcagggtgacttaggattgtctccttgtctctttaattctcctttccctgactgtaaagtgcaaataatatgacccaccttgcaggctgcttgtgagggtccatgagatattatgaagcttctagcacagtgcttggcagggcactgatgggagctcttacctttattattaattcattcaaagggagatttactgatactctctttagtgctgtaagagatgtgacgatatcaaagctagtgcctgtcctcttggagaatgtaactctttggggaagctaggacagatgcagattattactatgtaagatgggaagtaataaaagacctgggaaaacaagtgaatggcagacgcaggtcagaaaggagaccaatccctccagcttgggcatgcttgctggcatgcgggtgccccagggactcgagatcctgttgctgaatgatcagatggtggcatctggggtggacactgaaggacgaggctggctgtgtgtgtgacagcagacctttctccctgctgcctgtgctgtgctttcatactgcagacacccagcaagtgagcgctgagttagattccagagagagagttcaagaaaatgcatagcagcagacaaacccctctatcgttaatagcaaagatttcttatttgttcatttgcaagatcctttgtcaaattcttctggccttgggccctttgatcacttctgtgtcctgtgcacttaagtctggtggattccttcttgcccctcagcactcagctctggcagccgtgcctcccccgagtccggtctgaccgttccctgcagctttacctggttctctgtgagcctgagcacttgggctgctccatctttcaacagccccaggagttggtctgagtcatcttggacctcctagctcaataaatgtgtaaacttgccatagcccagatcttgctctgatttatcagcatttagccattagtccaaatacgtgaggcttttcaaaaaatacagaaacagccgggcgcggtggctcacgcctgtaatcccagcactttgggaggccgaggtgggcggatcactaggtcaggagatcaagaccatcctaacatggtgaaaccccgtctctactaaaaatacaaaaaaaattagccgggcgtggtggtggtcacctgtagtcccagctactcaggaggctgaggcaggagaatggcgtgaacccgggaggcagagcttgcaatgagctgagatagcaccactgcagtccagcctgggccaaagacccagactctgtcaaaaaaaaaaaaaaatacagaaacaagtagttcccactcacccttcctctctgctgttagtgtgcattcagacgatgccaaggaacgggcttctctgggcagagccggggctttgcgaggggtctttctcagccacacgtgtccagcatgtatgtggccttctcctggggcatagaagtacttatttcagaccacacaaatgaaaacgattaacattcagcaaaatatcccagctgcagtctaattatttcctatggttgttggtgacagcgtcccctttttggagcagggttggccggaggcaggatgagaacagtgtgggcttgttggggaggtgttgtaaggcacccagcacagagaattgaaaagccttctttagaagcttctctctggggcagggtgggggtgggagaggaaacggtaaagacccacagtggcctgggctcttctctccagcaagatgcaacaggacagggaaggagggcccccaggaggcccctggggtggcctggaacttgagaatgaactgatgctggcaggcacagccttcttcacttcttcacaagggtgggccccgccttagactgctgggtgacacctgtgcataagcgctccaccccccagcgagagaaagcgggccgctaggactggctgtcctttactgtgtgactctccccagcgtcttgtctgtcccttcctacctgtcactgcacttggtagtcacatatttatttgtaccttggtgtatttattcctgtctcccccacaagactgcaggcagcactgagaacccaggctgtcttgctcatgctctgccgccaggactagccgggccgggtacccctggggtactccccaggtgcctgtggaatgaatgaatgagtggatagatgagtgagtgaaagaatatatgaaattggttctgcaggatgtgcggcctgcagttaccactcaggagatgctggctccagggtaggagagagactccatttttctctgggtattactgggctgttttaatttcatcaaacaataggaatttattacctgttttcttaagctctaaataacaaagggaagcacagtaagtattctgttcttcttctttgccagggcccagaggttgagggaggagcagcccatggtgctgggtgccagtggggggtgaggaagaggctgcagaggtccctgctgcctggacacgtggctctagcccctaatggaggggcagccccaggtaccactccctgaacctgcgggaaggctggcacagacggcatgtgagtgcggcacatgccaggccacggggctcctttcttgtaactctcaaggccccatgaggcaggccaggtgaccgaggctcaaagggcaaagtcacttgttctgatggctcagctgcaggacagtgactgtagggccagtgctctgtgcacccgcagctgctccgctggacacagacctccccggggaagcagagaccaccctgcatctgaacggggaggggtcgatggagtgtgccctggtgacatgggctctgcagggcggggtcaggcctcccgctcagttctgcatcagtacttgcaccacagaggcctcaatgaagccgagggagctcaggggagcaggagtctaagccggaggccctcagcggggcggctgtgccctccccaacgcccaggacatcagcagtgtctggagacaggttggattgttatggccaaggagggagcagtgtcattggcagttagtgggcagaggccggggatgcagcggaacagcctacagtaccaggcagtccccagcccccgcaatgtccacggtgctgaggtggagaaatcctgggcttaggtcacgcccaggtgcacaggaaggatcttgcatcccaggaaccaggggctgggagagaggagagctgggctgggggctctggaggtcccaagttggggtgcccttggattcctatgtagaggtggaaacctgggtctataggccttgactaggggtctctggctgagaaacactggctgaggctgcaggaaagatgtgcctctgaggagcaggcctgtggaggccagacactgaggcagctcggtccccctggggcctatggaaatgcaggtaacccatgatatcctagggtcctgctcccacccaagagagggctggggtgagtgatcacagctgagggcctgcaactgtatggacccattctccacaccacacacagaccagcagaaacatctggcatgttcccccagcacccctgcatctcactaactcccattcctccctctcctgtcagccaaggccactccctacaggatgccttccctgactgctgctctgggtcaggttgcatcttctcaaagaactgtatgtaatttacatcctttgttaatatgagcatgtgattaactggcttctgggtggatgcatcctggtgtcttcagagcccaacacatagtagatgcccagaaagtggtttttaaatgattatgctttgtctttgataaataacataggcgtctttacagtgtgtataggcagagccgagttgagacatcgaaagtgggcagggaaattgtctccaatcctggagttgtggggaagcaccgtaggcgagcctgtttcctccgatctcacctggggtggagtgtgtgtgtgtgtgtgcatgcatgcgtgcgtgcacacacacgtgcacacacgtgtggaacccacggggctggggatagtctctagcagtgtgagacaaccagcacaccatcctggtgctgagcacagctcagtgtccctgtctgggaagcccatgacccttggggcagaagttctgcacagggagtccgcagtgggccaaggcagtggtcttcagcccatttgcagagctaccttgaccctctgtgcctgagataaaaaagatggtttctccttcatggagaaggagttcagttttattaatactttcttaaagtgagccacaggattagtgctggcccccaggttttgaaatcttccatctgatgttcagcctctttcctgttgagaccactccttgcaaagatgattgctcttgaagttgaacccagcataactgcagtccgtcgccccctcccagctggcagagtatcctttgctttcattttatttattgtttatttatttatttatttttgagacagagtcttgctctgttgcccaggctggagtgcattggcatgatcttggctcactgcaacttctgcctcccgggttccagcaattctcctgcctcagcctccagagtagctgggattacaggagcccaccaccgcgcccagctagtttttgaatttttggtagagattgtggtttcaccatgttggccaggctggtctcaaactcctgacctcaggtgatccgcccaccttggcctcccaaagtgctgggattacaggcatgagccaccatacccggcctcctttgcttttatcaaatgtaaacttctaggaatatattttcataatgtgtttctagccgtccttaagaattaatgaagttcttagttccatttttttttttttcgcccttgactaactgtggacaattctcaattgtaagggaaagatttacttctgtattaataagtctcactgtctccgaaactcttcacattcaaagggcactcgcattacctggcaattactctttctcgatgagacacaggttggtgatatttgggaatatgactttggggaatacaaactctgtttaagtaatgattttgaaaaaaatctctctgcaccagtgaatgccatgaacagtttcattttcagtaagcgcttgcagccagttgtctagttgtcagaagccgagctcatcagcgggctcctggtgtcggcgttctgctgctggaaggtcagggagttccctggaccccacggaagaagctgtctccatgaaggacagggaccgtgccgtgaaccttttattctcccactgacatggtatgaaggcatttcggttttaagcatcttttttcctaatttctcagaaccctttgaagatcttgcacttgacttctaaaattcagtccaattccttaataagtacatcattattacaagttggcttttttacttcttaagaagcatctttaatttgaacttccataaccatatttttgtaattatcaaatggcaggaaatagatgctgactcccgtaattttatcattctaaatgtcacaagaatggcaaggatctttaaaagacaaaagaagttcttcagttaggaattcccatggtaaccaatatagctcaatcttttttttttttctttctgtggctattttaagtatttaaattgatttacatccctgtggaaatgtgaaaaaagcctggatcccatatacatccattccacctggagtttgggggttttgttataagccatgttcttcttttgattccttgccagtgtctgcagtggaaggtcagggttcgtccctaagcaaaagccttttaagtttccttaacttaggaagaagtcgtgatttacctttgtattccttttgcagtggatattaaaacataaacatataaattatctcattttgaattgctcttttactttatgttgaacaatgaatataaatgtattttagcggccagatgttagaggataacactggcttaatttgtgctgagtgtggagccccttctgggtgcggtgagggtctgagcctgtgaacctcgtatctgcccagctcctgctggacagcatcagagatctgctggctcagggtccagccagtctccacctctttctggagcagaacactgcacaccaaattcactggtactgcagccacaaccctggacacctggacatgtgcattttcccatcttctgccacactggccctttgcctgacctcctcctggccttcagtctgaggtccgctgggggccatgaatgcctctgcaaagttgcaggtcctgatggtgggctatgggtgctcccagctgagcacagtcatgactcccacactcactgtacaaggaagtgaacacagtaagtgtggatctaccagatcttcactaagcctcagggcaaccctaaagagaggacaggcatgtttatcctcatttcatccatgaggaagtggaagctcaaagaggtcctatgctgcatctggggtcagtgtggttttctgcctctgggaccccctacccctgaggccagtgtccatccccctcagcaggtgtgtggagccctccggacacaacatcttgctaggaattgcatgacagttactgatcatttatcttttaaaatgggtcaataggctgaaggaaagatgggaagtttctcagatatcacatattggcagatattacagccaaaatacacatagagggaaacattttggggaaggttgtggccatgataaaaatcagtatttattgagcctgtgatatgtggcagacaccatgaagggtgcattgtagacattaccacatacaccccatgcagccatagggtgaccagccacctacttagccagggactgaggggtttgctgggatgggggattttagttttaaaacagggatggtcctgggcaaagcaaggtaagttggtcaccccacccgccaccccaaggttaagatggacttaccttcattttgtaaatagaaaacccatggctcagagagttgaggtaactgcccaggctgctctgatggcaagaggtgcagctgaggcctggaccaacacctgtcagaacccaaagccacattttttgaccacgacacctttgtcatggccatgaagagcagtccctcctgcgaaggaagtctcaaggtcatccatatgaccaacaccttgaaaagatttaggtgataaaattgagattcttaattcttttgaaaaaaatgattaaaatcagaaaattattttcagataaagtttatgccaaaagatacaaaggcttgctctgtaagagataaagcgttttgtggcaaacaaacaaacacttcaagggcaaagtcaggccaaccgtcctgctctctgccatccggtaagtctctccatgctcatggtcatttgtcgcccagtgtgaagactgctaagggcagatgacatgctcagtgtttctcagttgaccgtattttttaattgagtttttagacaaattaaattaatgaatttgtttgcttattcatgtagagtaaaaagcaatacccactctactccaaaatagtagctcaagctatgattttgcaaattgctatggtttggctgtgtccccacccaaatctcatcttgacttgcagctcccataatccccacgtgtcatgggagggaccctgtgggaggtaattgaatcatggcaacaggtttttcccgtgctgttctcgtgatagtgaataaattctcacgagatctgatggttttataaagggcagttcccctgcacacacactcttgcctgctgccatataaagatatgcttttgctcctccttcaccttccaccatgattgtgaggccttcccagccatgtggaactgtgagtccattaaacctctttttctttataaattacccagtctagggtatttcttcatagcagtatgaaaatggactaacacacaaattatattaaaatgcagcatccttacagaaaagtctgcaccagtcataagcacacagagctctgaactttcaagtgagccagcacccagatcaaggaatgtgacaggcccattcccccagcccaatccccttattgctcttccagtcccttcatgcctcccttccaggggtaaccattgtccccacttctgacaccatagattagtttcgcctgctttcgaactttctataagctggatcatagattatatcgaagtatttttatgtttattgaatacctactatgtgcaaggccttgcctagctattcaatgaatctaaatgacttggttttgaatgcctcccaggttgctctataggtgtgtgtgtgtgtgtgtgtgtgtgtgtgtgtgtgtgtccctggatataggtcacacagttgtcttagccgtcagccctcaaaaggcaggttcacatggtggccagctcacatcctgtgtgtgttcaggaaggcactgctgccatgcaaatgtcatttaaatgtggcatatctatgaattcaacagggtaatttggttcctatgttgttggaaagttatctgaatgctgaagtcaactgcactccaggcttgagtaaacaagtcaggacaatgaagagaggcagttgctgttaggtttggtggggggaggggaaggtgactttcacaagtcctcttgtacaagggccaccgtgtgaaacctgatgtccagtgaagacacagcaatccaaatagatcactgccttccagccccagggtctacgcaacaaaaagccttccccattgaggccatctggcgagttctgcctccaatgtcctgggagcagtcagccagagctcagggcagggggcctccccagcctccattctgcagatcgttctttctgggcaattcatgctcattcccacacagaaaggtttgaggcaatttgtggaaggagcgttgtatcaggatggtttctaattcaggtggattaaggtatggatgctgtcttcactgctcacttgtcaaataactgaactcctgacttctttgaactctgggcttctaatctgtgaagtcagaataataataactgggtagttgtgagcattaagtttaaaaatatgttgtggcagcactttgcatgagcagaccatagtataaatgccagctccctttccctggagataagaagtgattgtgattgtgcgttaagttctgttttaagcttcctagctgacaaggcaaaaatgaaacacaatggcttatattgttctagtctgattcaaagaatgtccctagtttttcaggaaatataaacactttcctggtgttacattctaagacaactttattttgcaagcctaaatgattcagtcagggtttccaacaatggttttcaccccaaaagatggagaattcccctttctatttccatagcatgtatcaatcctttaaaaaatactaatctggggttatgatagtgcaacactaaattcggaaggcgaacgccatggagctggaattgcattctgttgctccatttctttcaccgtctctttttactggacatcttatgataagaacgtaaatggaaagggcaagcctggagtgacaaagggaatcctccattcactccatacagtgctgttgctgggctcgggacgtggagagggcaggggccgtgaagacatcctggtgcctttgcagggtcgtcatgaggataaaggggatgggccaggtgtgacagctcaggtttataatcccagcactttgggaggctgagactggcagatcacttgaggccaagagtcccagactaccctggccaatatggtgaaaccctctctctactaaaaatacaaaaattagctgggcatggtggtgcacacctgtaatcccagctactcgagaggctgaggcacaagaattgcttgaacttgggaggcagaggttgcaatgagctgagattgggccactgcactccaccctggcaacagagcgatactctgcctaaaaagaaaaaaaaaaaggggggcggggggatgatgcagacatgagctttgtgtgtgttcttgtcatatcacacgtaaaggagacggtcagactggggctcttttctgttacaaaattatacatctggagaatcatggtgaacgtgaactctgtgtgagatgctggcacgtgcctccggcacttcatgaaggaggctgttggttctggagcatccgtggctttgctcctcaggctgctggccatggggaccctgagtcacaagctcttcagaaactctggtgctggggaggcaaagctgtctcctcctctctccctttgcacagaaattccagaatttggattcttttgtgacctcgagctgaatgttcaaaatgttgaagctgagttctttttttgtttttcatataagtacttacacacacgcgtaaaaaaaagagccagtaaccaagggcttttcccaggcccccagtggcctggaagtccagcccacactcccagactccagctgggagacacaagagggagggaacttgggtgggggacaccccacaatgatgaggtccttaggagactccagggcctccaggaacccagctgccacccaagagcaccctaaatctcagaagctagccaatccaatggaaggacggtaaggtcttttcagtgtaagcacagctttgcggatgaaagggttgtatttgcagaattcgttggaatcagagctccaagcctagaatgtcctcccaccctctacccaccccaggactggattcctgtcctgtaaggccaactgagctctcctccctcattacctgtccccaggtacagaccttcccctcacaccattggtaactcatctgttgcgttttttcatgtatcctgaagcacctacacagtgggcattcaataaatatctgctgagccgtggattacacagtagagccaccgatgaaatgatgcctacgaacctcctacaaaactcctgttttattatatcaaactctgaaaactgacagtttggactattcttttgttaatcctacaatgagttaagctgactaatggacactcaagtttccatcatggctgcgctcacaaaaaagatcacctgatattttccccttgttactatggaagattttaggtggatcatattcctattcgtatcataggaatcatttactggggaagggtcacttcatttcaattatgttttgaatgattagtcatttcaaaaataaaccatccctccaggttaaatttatctgggactcatggttggataagcttctatgtcattaaaattaattgatgtagcaaaatcagctgcagtcattatcctgtcccatatctgtggcagacccaggaaggtgctcactgtctacacacagcggcagaggtctctgtgtcctttgtcccatgcagagaccctgcctgtaagtctcactgaactgaaagttagatcgggagtctccatatgacctgactgagagcttcatgttgtcaagtgtccatacgaggctaatgtgacactcacgctggggctggcgcattggaacaaatggttcacttcaaacattcacatcccaaaaaggaccccagtgggcatagggagcccagcccaagacgaagtatcagggagctgttgggagcatgggtcttacgtaagagacctggagtagggtggggattggcacccccctggagggcgtctccatgttctcgatgcctctgtttcctcctctggaaggcagggagggcccttcttagctctccacactatgtggaggagtcggtgagatgaagcctgcccagtgttttcactcggtgccgccctgaggtgcgagagccccatgcatggtgccctggccttgctgtcctcgcaggtactagcgcattccacttcctttagagaatgagcactggagtaaggaaggtcagatgtgtgcccatcccggcctgcagcgagtgcttgcccctcacaaggcagttcctggggaaagcctccagcgtgcgcgatttgcagtttagtttaaacggaaggaggtttattagaatcttggcagctctagaagaagactcttaactctccattggctacagatcccggccagccatgtgggtggctccctcaggccggtcagatgttcctgctgggatggccatgggcaccaggagagctttcccttagcactttcagacctcagacctcctggcagattcaggtgcagcattgaacttcatgccgttttcctaaattcaacttccggagcttttaccttttcagagacccagcctagtttatagaagcccagcagaaacttttcttcaatttgctcgtgactcttagatgagtttggagccgggtgggaagatggtatttcagcatcctaggcaggggcacatcagatgaactgtgcacttcgaagccatgatgctgcctgaccatgagggtaacatgggggaggaggaatccttgtcagtatcctgtctgtggttggctgggcagtgaccacttcccctgaccgctgcctggggactgctcctccacacacaacttatctcccaggaagcaattccaggcccaggcaccaagggcccagggttttcctaatgttgaggacattattgcccagtggcatagaacatgggatttggagtcagatctggctggatttgatttctggctcaacctcttattgaccacgtgacttcacgcatgttacttcagggtggccgtgaggatcagacaggatcctgtatgtacagtgtctagcacgtgcctcaaggaccatttgtcctcataaatgtgattattaggtagcaaggcagtgctacatgtggccccacccacggccaaccccacggtgcgaagagcagcctttctgtccttacctgggctttaccctcctctatttccaagctcgtccacccaacattctctcactggatcattaccatgatgccgtggatcatcagcaagtatcggtgacctcatgtgtagtggccattgaagcacagagaggtggaggggctgaccaaggtcacccagtgggcagggatctggctggagagcacacgctgctgcttctccctcccttggactgtggattacaggagagaccagtagccatgcaggtggcaggtgggaggcctagggttctgcaggtatgagcagacacacatcagccccctctaccaccggcctttaatgtctccacatcacacagaggcctgggactgtccagctgtgtggtgcagcggctcgagcacatacactttgggatgattccttgagttgtaaagaaagaaaataaatctccttgagaatgatggatgcactgcttagcagtggaagaggagagctgacctttctaggtagacaaaacagaatgcctgtcaaacattacacttttgttgcagtatattgttccagaatggagcactgcatatttttgctataatgctgtttttgtttctaaatgaatactgcagtgttcttttatgtggaggagggattcattactggttcacagaactttttcagtggtggggaaaaaataggtgcttgtcctcagcaatgcgtatcaagcctagacacatttctgttaggagcacagtgatttgtgaattccctgacagcttaccttttattatgagctctattttaaatagagacatgtagcttttcctcttcaagacaatataacagatacctagaatcttctctgttatctctcatttgttttcaagctagcctcttttttgtgcattttatagggattcttatttatgaactagtttaaatggttcagatcagccagttgttgtggggtggaggatcattaaaacaataatatttacaaatgtttcagcaaagtggctagtcgattggcagatttttcattcatcagctttcctaattttcagaatcttcctgaggaatatctaacaaacgtctctgctctgagtaccatattcctgaaaaaaaaaaattgatctgttttcaggatgggtctccccttttctcaggctaatatattttcttagtctctcgctaatatcctctgcatgcctgtgaaggatgcatcattttctgatagctctgcgcctgaacgccacctctgtcgatggcaacgcttcacccaggcccccaagccaagcccacggtcacgcttagcttccttcccttttccccacttctcacatgcctccaaagcttgtgagctctgcttcctctattgttaaatggctgccccctccccggccgcccaccactgcccttgagaacacctgttcatcctgcatctggaccacaggtgtggcctcattagtcaggaatccacagtccacacttagcatggtcctgccccttattcttctctgcagccatcacagtgacctactttgcagagagcagaatgggaatgcccaggcttgcagacttctgcagcccccttgtggcttcagcccaaagccaaaggtctctgcagcattccgggatctgcccaccacacatcccaggctcacagcagtgatttattgggtccaggccttatactgagttctgcgtccggtccttacctcctttactaacccagcaggaaattttattatctctatttcgcagatgaagaaacttaggcttaaaactgttgtgacttgttccaaggtcaaagccaggatccaacttatttgccatttccagaatgttccatacactctctggcttcccggtttttgtttatgtgttcttcctgggaattttcttcttgagaaagatgacctaactttagccaaacacaggttagtgttctgcttccatctttcatggcccctgggcttgccacgacctagcactgctcacccctgcatttcaacgatctgtttataagcctgtcctctggacttcttttagggtaaggaggctgaggttctgtccatgtattttttttaatctctaaatcctaagcaattggcacagaaatagcacatagtagatgctcactaatgttaaatggctggtttagcatattgatgtactcattcaactagtattttatgagtttctggtacatgcacagaacttttcatatactgaagatgttcaagctagagatagcaggttttctattcagaaatcaccaaattagccacagatgctctgtctttggggagggtctgcacctgccttcgtcatctgtaaaatgtggatcatacctccccacctcatagggcagctgaaatggcagcaaatatccttaatgttaatccagagtcctactgtactgtgttgttgtagagacgtgaagacacataaagatggaaggggcgggcggggaacagctaggcaccaggagggatacaaccagggcagcccagagggctcagaaaagatgtcaaggagagagtaggacttgacctactggatcacctggcgtctagtgggtttgggtgagactgaggctttgtgggacagggagcagcatgagggaagcagggggactgtgctgtgttggagccccgtgagacacggttgtgtgagtgagcggcctctagaaggtggatcaccgtggtgtctgcccgacttgatgctcttcctgtgagcttccaaacctgttaagcaatccagagggctgtttaccaacacacgctactcttcttatccgtgccaatctcatcagcaaacatgctatttcccttattcgtgaataataattattattatttcataataaaatgatctttctgaataattatttaatgccagagctcatccattctgccttagtgaatattgctttgccctggcctctggccacaggttaccgtggacagccgagtgtcaagatcatcaccaaccaggcccttcacacacatgcttagcctcatcgcagacctagggtcacctggtgaaaggcaggtgtgtttccaattggcacgctgaaaaagaaaacctgaaaacactgatttaacatatctgagtattagagatcaatcattttctctggggctcactgaacaggaattagctcaagaaaaacatctttagcgtgttcagttaccattagcgtcaggcacccagcaagcttgtcctgaatacatttcagagtccgagcccaattggaggagcgaggcaggtagaggggagtaaaacaggccttgttttcgaggactgctgctctgaagtcccactgcactacccagtgtggggagtccttgtggcaagctaccctatatctctgagcctcagttccccctctgcaaagtgggtctaataacaaactcctcatatgagtattgagaggatgcagtgatccctgttgggtgcttggcggggagtctggcatgaagttcctgtccttacagtggctgtttcttctgcaagggtcttctcttggtggcagttaacaccgtccatccctgtcctcaaacactagatcccctgccaagaagctactcatagggcaaagatttttttaaaaaacatgacagtgactgtcagtatctgagaagtgtccatgcccagcgccaagaggtgaacaaattcatagctctcccctctggggcttggagggcacccctacacctcggaagggagcctggaatcaaattgccatgtttccaaatttgatactgataataatggcatagacagacgaggcagtatggaaaccagcagacactcgtacttgcaaaggaggatttgtctggctttctaatttgcatgtttcctaatccaaagagggtgcctcaaccgtggagtaattagagagtgtgtgccttggtcgggggaggagggagtttgagaggcaggcagggatgcacgtggatctgtgctgagcagctgcccatgaataatttctaaaagttgccaaatatttagaagtcctgctcgtctgtcatagactggcaaaaaggatggttgagaaaaatgggtctttttcttcccccaggcttagcaacattgttttaaagatttcatctccttttcaaggtggtttaacgctgtggggaaggcaggataagattctactaaactccttcatgtcagggagggctccctcccccaagttcagtagctccttctgtcctgtctttcatgcattagaaaatatagacattaacagctttactgttgcctggaaggtaaacaaggaagctgtgagtatgatatcagtgcgaatctgaagcctagatgattcagaaacgttttgctttcttccatcatttgaattacccactggaagcagaatgaagttgtccgatagaatgcagagacagccgcaggggccgtggctgcggttctgtccattccccactacacttaattaaagtgatgaggtctctgttatttttttatttggagtaatgatcctgctctttgaccagtccatctaaagtaatggtcatagtcatggtgcttaagcgttactgtgattgagggtgaatgacagttcttgctaccccagtgccctaattgatcaggacagctcagtgtcttttgggaaaaactggagtccaagagaaggcagccaggattgaggaacttggctccgggagctggtcattcacagtaagactgcttcaaggtcagtatctctggcatttaactgcatgaagtggagttttattccatatcctttctttataaaattttaatacatcatgaaaagttcacattttagatatactcatagaaacatgtatgtaaacatgttaaaataaaatgtaaatgcacttccaaaaaattcttcaaggttgctactcgtttaaaagactagcttggagatacgcatgctacacaagccactcagcaacctacattctgcccactcgaatgcttgtggcgccttaagccaaggttgtgtgtaaaaggcattttccttttgtccgagtttggaaagattggccagttctgttcatctcagaagtaatttgaatgtttcccttaaatggaatagttccgagggactgattctaaatgctgtagttttattttcttctttgcaaggttaggatgcacaataacgtcgctccgtctttccatatcttgtgagtaatcaggacccaacccagcagcctggccacttcctgtgggcgccctcactggcctgaccttgcctgcccctgccccccttcctaagggtggatttcccctgttccgatgttgtcaccatggctttttcccagggccttgtcgggctccctccctgcttctgttccctggaagcagagtggctgcagctgtgagctccagggactcaccagccaaagctccagccaaagctccaactctgtggccaggctggtccaggcctctgtgggccaggctacttccaaagacccacttctcccacagtaaatatatggccatcccggggctttcctttctggaggagagaacataatggagcatcctctaggacaggaaaatattttaaggaataatccagtatcattattgccatctgcagaatgcctttcctacaccattcctacactaaaggaattacagcttcactgggaggaaggagatatagacaaaagaagcaaaacaaaacaaaaacaaaaaaaaacaagcagacaagcctgggtatagtggctcacgcctgtaatgccagcactttgggaggccaaggtggctgggtcgcttgaccccaggagttcgagaccagtctgggcaacatcaggagaccctgtctctacaaaaaatacaaaattagtgggacatggtggcgcacacctgtagtcccagctactcggggggtggctgaggcagaaagacggcttgagcccaggacatcaagcctacagtgagatatgatcgtgccactgaacttcagcctggactatagagtgggaccctctctcagtttaagaaaaaaaataaaataaaacaagcagacaaacatacattgcaatgtaggatggcttcctccgggaggcctagccaggagtgggttcagcagccccgggccggcattgccgctgccctgtctcctcgctctgtgatgctggtccagagcctcaccaagcctcagtgtcctcatctttaaagtgggtctcaccggccgggcttggtaactcatgcctgtaatcccagctctttgggaggccgaggagggtggatcacttgaggtcaggagttgaagaccagcctggccaacatggtgaaactccatctttactaaatatacaaaaattagccaggtgtggtggtgtgtgcctgtaatcccagctactcaggaggctgaggcaggagaatcacttgaacccaggaggcaggggctgcagtgagccaagatcccgctactgcactccagcctgggttacagagcgagactccatctcaaaaaataaataaacaaataaaaatttaaaaataataaagtgggtcttgccttacctactaggatagttgttagatgtgggagagatgccataagcagtgcccagggcactccacagcccgagaaatctaagctcatctctccttccccttgctggaggacaacaacgttcaagagaacaacaggaagctttcccatgagtggaaggatgttgtgtcagcttctcactgaccccacttcacggatctgtctgctcaaaccaacctccccaacccccagcttttaaatgtgcctctttcacagtttccaacttgttgctcaatattacagtagccatcagcgtcgcagtgttggcctaatggaagtcttcacttggtaaaggtttactttagagaacattatgattggtttttgctctaattctttaagaaactgatagttgtggtttcctactatcatgtgtgagccacaaaccaataaaatacctgatctatttgtagtacaaaaattagcggaatgtggtagtgcacacctgtagtcccagctacttgggaggctgaggcaagaggactgcttgagcccaggaggttgaggctgcagtgagccatgattgcaccgctgcacaataaggcaagagttcagtgtattcttgccttattttttaaaaatcagctgcaagtttaatttcaagttatgatttgtgtttgattggggttgtcaatcgtgcaaccattggcaaagtgccaacaggttctaccaaaattggtcttttcccatgtggttttagtctggtttgactcaagcctgctgccactgtctgcagccctgctccagaaacttctgcatggcattgggttttattttacattaaaataaatcagcattgcattggaagattgatcatttctatcaatcctcttgagatattggaggtataacttccattcctggcttcttcccttgcttggccccagtcaaacatcacgagtcactctttttagcatcttttctggttcattttggggagtggaaaggatggactcttgtaataagttattcctacaaaaaagcagttcccctatctgcagtgatcctcaagtaatcatttcttgcccaaaaaccaatgccctctcagtcccctgccatcatgctctatgtaatactgggttccactgcaccctggaaaccctctgtgctttaatatgttactcaacagattttccccggttgttttatatagctgccccagtaggtgctttggcattatatagactatcttttaaattttatttataattgttgtttccatatacaaatccttttttttttttttgtattagtaactttataccttgtgaccttaactcatatattgcctctagtggctatttttgaatcctttgggattttctacatagactattatgtcatttgagaataaagttttatttttttcttttcaatgcttatgtcttttaattctttttcctaacttattgcattggctatgatttccaggataatgttaaataggagtgggacaaatccttgctttgttcccagtgctggggagaggtattaagtctttcaccattaagaataatttgagctgtagatttgcccaaatagacttaaaattccttagttaccagaagtattttttctgcttcttgggcttcaaacacacctcatgggtcagcttttaaatgtataacaatatacatttaaacaattttttaaaaatataacaatttataacaacttttaattgtgtgctgtagagtctttatttgatatgatttaaaatatctagttttgtcaggactaagatagcaagtagtctattttagtggaaaacatcatgtactgcattcactatttaaaaatagtaacaatcaaaacatgcttccacccttcaccagtgcagacgtgcagtgacgactgagcaaggtctcattcacaggtagatagcagcctaatcaataatcccagtgtgaggctgccatgctgaccactcagaacaaaacaagcgagctaaattcatccactgaagaatgggtcaacaaaatgtggtatatccatgcaatggaatattattcagccattaaaaggaatgaagtactggtacatgctacaatagggataaatctcaaaaaataatactattataagtgaaagaacccagtcacagaaggccacgtatagtgtgagtccatttatatgaaatactcagaataggccaatccataaagacagaaagtagattggtgttgccaggggctggggacaggatggagcttgggagctagtgagctaatagtgggtgtggggtttctttttgaatgatgaaaatgttctaatattagattacattgatggtttcaagctgggtgaatatactaaaaaccattgaactgcacactttaaacaagtgaactttatcgtatgaaaattatgtctcaataaaactgtttttttttaaaaaataaaataacatgagagctaatttaatgggaggctgggcatggaggctcatgcctgtaatcccagcactttgtgagcccgaggcgagcagatcacttgaggccaggagttcgagactagcctggccaacatggtgaaaccctgtctctactaaaaatataaaaattagcagggcgtggtggcacatacctgtagtcctggctactcaggaggctgaggcaggagaatcgcttgaactgggaggcgaagtttgcagtgagctgagatcactccattgcactccagcctgggcgacagagcaagactcactccgtctcaaaaaaaaaaaaaattaattaattaatggaattgatcaggctagataaagattaagctagaaagctggagaaagtgagctgccccagaccaggttgtaaaaggctcttctggggggaggatgggctaaaggaactacttgaaggggagggaaccctataggccacagcagaggcagctgactgatggagcatcttgggggctaggtcagtggccacaccccaccatcaactgatcattagaggttgtattaaagtgtggggtaggacatggttaaaagctgatgattgcagccaaaagaagtatcatctagataaagaggtctccagaagaccctgaacaattaagcaaaaatgggatagtcgactagtgctgtacagaaggaaatggctagaattagcagccaactcctgttaaccagaaaactgactttttttttcttctttcttcttcagactatagccttcactttccctcggtgagtacaaaactttttaaaaattatttttgatgtacagataactagtttaaaataatatgtgggatttaattattagaagagcattgttttcttgagaaagaaagccatatggtcagtgtgttatatttgaattttatgccttaccttgcttgagcaggctcatatcacacctttttgttacatcatttgcgtgtgtgtgtgtgtgtgtgtgtgtgtgtgtgtgtgtgtgtgaaatttggctcagctgcttggtctcctgaaatgtattgttgaggacacaatttctacatgctttcctttcactgacgtgtcgttgcttttaaaagggcctagccttatctcaatggcacacatctgaccagcgttcaggagcgcaggggcacataattgcattgatggtaactgcttcttaaattgatctgctctagagaaaactgctgaaaaggagcttctccggattgcatgctcagtaaaagcagtgtcagtggtgtcgcataattttatgccttttgggtccaggcaatggcaaaattccatgaccttgatgtccttagaaaaagaaaactttaaacaacagagcccttttgcagtctacttaatcaatttaatgctgttaaatttcctaaacaaatcgaaggaagaaaatctaagagctttcgcagtgtctgcagcgtgcaggtgagtggcacgtggacttctgggtgtgcttgcttttagcgggcaccatgataccggtcaccaggaagctgtatttcctaaatgtattgccttgtttgaaaggcactgactccgttctgaaatcattttcaggtcaatggatcaattattgttccctggctggccctcttttttaggcagggtgccctttgtggcactgttccatctgggcataaatgcttatggtcgttcttggtgtgatgaaatgcattttgactgtgtggattgtttctttctttcttgataaggaatgttaaataaaagtaatgattgattaagctagagaagagggatgccttcgtttgccacctaccaagttaattttgttgtctatgttctccattgcatcattataagacgttgttgattctgaatggggaactcttcttgggtaaatgtcagcggtgggatcaaagagaaggatcgttactggaatcatattatgggatggaggattaactatatgaaatctacaatccctgatagcctcagaatcctatacaaggggtgttggctggtcgatactggtttgaacgcttattaaatgtggctttcttcaaaagcaatgcagctgcgtgctttatcgaatgcaaatgtgcctaccgtgctagaatgaaatatttgagtgtcagatcccacagatatagctccttttgaaatacagggctcagattatgatttgttacaagtgtgtgacctttgcaccccttgtctctccactcagtactggcccctccgcaccccattttgccttctgccgcttccaaaatttcctattcctcccagcaagttgacctgctccccatcagtgagtcagcagctcccgcagctgagtgatcctgtgacaggtgcttccggggctcctccacctcccggccttgtctcaaaaccggcaagagattcagaagtaggtgccccacccgaaggtcactgaacgtgtgtgcaggagctgacttcctgtcctccctgcatccaccacgggtagttggcggccagagccgcttcagggggcacctcccttttcctggccccacccttcctttgcctccatttctttctttcttttttttttttttttttttgagacagagttttcctcttgttacccaggctgacgtgtgatggcgtgatctcggctcactgcaacctctgcctcccgggttcaagcgattctcctgcctcagcctcccgagtagctgggattacagacaagcaccaccatgtctggctaattttgtatttttagtagagacggggtttctgcatattggtcaggctggtctcgaactcccgacctcaggtgatctgcccacctcagcctcccacagcgctgggattacaagcatgagccaccgcgcctggccctgcctccatttcagtgggaaagtaaagctctgcttcaggaagatcctgagttcctagattaagggtacttctgattccagaaatctttctcttgacacgaggaaagggtatttgaagcctgggtctcctatctgtgctgccttgtctgctgcctttgtttgggacctgcccactgggccactgatatggtttggctctgtgtccccacccaaatctcatcttgaattgtactcccctaattcccacgtgttgtgggagataattgaatcatgggggcggttcccccatactgttctcgtggtagtgaataagtctcacgagatctgatggttttttaaggggtttccacttaacttcttcctcattctcttctcttgtctgtgccatgtgagacgtgcctttcaccttcagccatgattgtgaggcctccccagccatgtggaactgtgagtccgataggcctctttcttttgtaaattgcccagtcttgggtatgtctttatcagcatcatgaaaatggactatcacagctgccaaggtagaataccagactgatctattagcctgttgcattttcactgctaagcatttgagacgtgtacagagcttcaggatatctattaatggaagggaacttattgctcacttgtgggattcagagacgtgcttttgagtcagttagaactaaaaagatagtccaccttgggcagggcggctaagcccactaaggctggagatggtgccacagccaccgcccacactgggcagctgactgcagaggaacctagtcacacgtgctggatgaacacatcagcctgtgcaagccttcttttctctcatctatccatcacttcctaggatgtgatgtgacatttaagtgagatggcccaaagtaagcatttaataaaagctagctatgggctgggcgtggtggctcatgcctgtaatcccagaactttgggaggccaaggcaggtggatcatttgagatcaggagttcaagaccagcctggccaacatggtgaaaccccgtctctactaaaaatacaaaaattagccgggtggtagtggcacacacctgtaatcccagctacttgggaggctgaggcaggagaatcgcttgagtctgggaggtggaggttgcagtgagccaggattgtgccactgcactccagcctccatgacagagtgagaccctgtctcaaaaaataaaaataaaaataaataaaagctagctgtggttatcactgctactcttattatgatcatcatcgcagaatcatggcaactgtttccaacttttaaggaaggcatttaagtggattgtaacatatatgctggtactatagtgggtatcaaaaggagttccctttactagcagtattccaatttttcacttaaaaaaactatattaatatttgacatctaggccgggtgcggtggctcacacctgtaatcccagcactttgggaggccgaggtgggaggatcacctggggccaggagttcaagaccagcctggccaacatggtgaaagcttgtatctactaaaaataaaaaaattagctgagtgtggtggcgggtgcctgtaatcccagctacttgagaggctgaggcatgagaattgcttaaaccttggaggcggaggttgcagcgagccaagatcacaccactgcactccagcctgggtgaaagagcaagactctgtctcaaaaattaaaaaaaaattgacatccaaaaaaatgagtcaagtcttttttcctgttaagctggattaggaagggtctcttgagtggagtcacccattcccatgttttcctttcctttttgtttgtctttaacaatagatttatgaaaataacactgtagttcgtatcagtgatccaatatgaccaaagttccttcaactggagaaagcttgcaactctgtgggtctctcgcatagtatttaatgcaatggaatttcagaattaagaatttataaaagacgtatcgttaaggtttaaatattttctccagactctttgaggtcttatattgttatacctaagggtatccactagtaagtaaaattaaatgattctgtaacttgactatttcactttggttcatggaaatacccaaggaataaaagaaaacaaaattaacgtgtgaatattcagcaactgtgagtttcaattttaaaatgtacttaggcttcccttcactcgcttgttcttctttagtaatctctgtttcctcctctgtcacttttaagaagtgtgattttttctcttctgactcatttgtgtttcttgatgttgatttgaatggctaccatgacaggaaaaaaaaatctgttcgttttaaaataaagctcagggttctgacaggcagggggaccaccccaagctggtttctggctgcatccgctggggtgacgtttccagcagggcaggagctatatgccccgggccggccttcagaatcccagaaacaagtgagaagcctggtgggcagagcacatataaaattaaaactttctaagacatgtgattcctatttcccttcttcttcttatactggggttttttatgagcaccaatggattccacgggtttctatccaagccacagcgacaaattattctcctggtgactgaattagaagttgacagtaagttggaagaatgcctttggatctgacatgacaaacagattaatccttcatttatttccatttccagtcctccctccccccggaaagtttccacattctttcaagaagtgcctcctaaaagagcctagtaggtaatcctttgtcacagagcaaaggcatgggcgttaatcagtcacagtttcccctaagaggaaataggaacacagatttcctatataaactcatgtctcgtcaccgtgttcacagcagacccagttcaaagttggaattcttctagaaatcctttctcagctgtcagggaaacatggattctccaaggccaggatggtttcccagcgtcttcccaggaagacaagaggtaacggggagctcagaggggtctttcttgtctctttggttttgtgtcatggttgcttctaatataatttctaaagtacctaaaaatcaggctaaagaatattaccaggaaggcgtcgttaaatgaaaatttggcaagcctcttagttacctttatccagctctgtgcaggaaatctgggatctatgcttcaggaaaccatttccagtccaccacgcctcagtcgatagtgcactggggatggcatggaggcattgtctgtttgggcctggtgggttgtcagacagccaagaatccaaggtgaggttcacctttttagaaaaacagtccctaacaattgctggggacataggccggctcgatgacatgggaaaatcccacccattgtaaattagacattttgtatctttgtaatcaaaactgccctcttcatttttcatttgaagtggcattttaattaagtgacacacagagcaagtgtgagggacctggactgcaggccaatgagcaggtcttcaaggagcttgtcttcaggctcctgaggccagggccggcactctgtgtgtaagggtttcctgcctgtgcagttcatgttgcgatattgtcggctgcagatgagtgccaactgcctaaggggtggcatcattcatattgggagaaagaagacaagaaatgtataaatgcttttgggtgtatgagttacagagagatttctcagacccggcggtgcatcaggatgaggtctcgtgccaaatgcaagggccgtgggtggaccagtcctacctgatacaggaactcaccctcaactgcagcaccatcccctgggggtggccccctgtggattgagcaacaggaaaggagatcacattctgctcaaaaatgaccatttgcagtgactttttttcttttctttttttttttttttttttgagactgaatcttgctctatcactcaggctggagttcgttggcacaatctgggctcttcgcaaactttgcctccagggttcaagcgattctcctgcctctacctcctgagtagctgggattacaggcacctgtcaccatgcccagctaatttttgtgtttttagtagagatgggttttgccatattggccaggctggtctcgaactcctgacctcaggtgatccgcccaccttggtctcccaaagtgctgggattacaggcatgagccaccctacctggcctgcggtgactttttaaagctgctcatacacatgaacataaagatggaaacaacagatgcggggaactccaaaagggaagagggagggaggagagcaagggttgaaagactctctattgggtactatgttcactattggggtgataggttcaatagaagctcaaagcccagcattgtgcaatgtacccatgtaagaaacctgcacacatactctcgaatctacaataacattttttaaaactgcttattatgttgcagagtttggtaaaaattatttaaaatggtaagaaaggaaggatctctgtggaactactgaacaaaaacatgtcatgtaatagattttctgggtagcttttctccttgttttgaatagaaatgagcataagcaaaaaaacgaaaactggatttgtgtttattttatgatgagggctgtttcagttctaacaacttttaaaaaatatgttgatagtatttaactgatttcaaacaactctaccaagattcatctgctatcagaaatggtgcagatttcatgttcaagtgctataacatatacatatagatagcttcttaaagctatatatattatattttacatatacatattatatatttaaagctatcatgttagcatgttgctgctgagaaatgaatttttttaaatctcatgtttgtgaaattattatttagaaaaacaggatttgctctatttcctgtttttccagataacttacatgccttatgcttaaacaaaaattgcatttctgataagacacatacatatggagcacttatttacaaagcatgctcaaattcatgatctcaagtgggccccctaaacatgcagaggcagcagtccaggcatgccttgtcacctctcccttaaagaggggaaaacagagacccaggcaggctggataagtgagttgtaggacagtggaaaggaagtgccagtccccagcggtgctctctcggactgaaacgctgctcctctccaactgaaaaaacagtcggctctcattaccaccaccaaaggtgaagttcagacctgccaaaagaaaaacttttccgttatgaaaaatattatttgacagttatgcaagtgacagagaacattaacaaataagaacatatcctgtggattctgcaaacggtttgctggttgataattatattttggaacagttttatgatcacttgagttgatcttggccagagttcccaattatttctgcatgttatctgctgaccaaatccagctttctaatctcttttcacacacttggcatgtttccttccagcacagccagtaatttgttcattcgctcactgagggtttatcgggtttatcaggtatctcttgcaagcttataattgtgcaagatattgaaaaatgaagtggttgattctagttctaatttcaggaaatattgcttagagagccaatgtttgagtcctttctctactaagtataaatgtgtgttttgcccaggagcctagaaaaatgattgattagaaagattttagttctggaaagttgaatggcctctgtaatcccagcactttaggaggctgaggtggttggatcacttgaggtcaggagtttgagagcagcctggccagtatggtaaaacctcatctccactaaaaatacaaaaattagccaggcatggtggcacgtgcctgtaatctcagctactcaggaggcagaggcaggagaatcacttgaacccaggaggcggaggttgcagtgagccgagactgcaccgttgcactccagcctgggtgacagagcatgactccacctcaaaaaaaaaaaaaaaaaaaaaagaaagaaaagaaaagaaagcggagtgacttagacttgtcacttcatcactctgagtcacaccttcttggtctaaaacataaagggctgggtgagaattgtttaaagacatcttcaagacctagagttctatgctctagggtggtgctggtctggatttggagttagcaacagaaatccccagaaagtaactactgcatgtgtcggccatgacggggaccagccgcactcatccccttgtcttgcaggatgtgtctgcatgttttggggtgaatgttttgtcatcttcctatgagaaagcagcactggcctaggggcttcctcctcctgcctcgctcatttgtttttgctctctcactgctctccaaaagaagcaagaacatttttttaaaaagcattttattaatggaaaatttttatttgaaatatggtcatttcctttttataatgttgcttctattcaggaaggaaaagctgagaatccgtgggctttgattttaaaactcaaacagaaatgttagttaataaaacaaaggtatttttcccactgttctgagggccattctggcaaatggttccgggggccgcgtgatggtttatgttgctagaagaagtgattcactgggaaggtgaaccacttcatctcgtcagaaaccatggaaaccctcgatatcactctgttcccctcttaggggagttctcaacagaagacacactggaaagtggatgtcaccgggatgaagttagaaatcccaaaagtagactatttgagcaagatcgaattcaaaggcatcactattttcaaaacctgtttttggcttccttccaatctgaagccaacaccgacattctgttatgctgtttcccgttgtgggcggactgacttaatttttaatgagtgattcactacccacggctgtatattttccgcctgctgagctgagaattaagtcattttcctttaaaaaattctttctgataccagctgagtaatgtggttctcagtgtttgagacaaggaggcatgttaatatcacttaagagagggtgtttaacctgacctaggctcttaagacacggcaagcatgacgccatcatgaatggaatgctctctagaaaaggagcctgtctcattcagctttcaaaaacatttctttctataaaaatattttccaaagttgtgcaattttttgaaacattttctgggggtaatacaaatccaatccatttgggaagtcgcgtgggaggtttcctaatgggaagcctggtaatatcaggtgtttccctacagtgttcgcagggtctcacccctgtccactctgatgactctgactggcctctgtccgtccagcagggatctggcaatcctttctgatccataatctttattagaatagcaccggaggtacttgtggccaccagagctatgtagctcctgtcttgggccccaccccttaccaaactgttccctctcctgggcccctcaacaccactccttcaggatgactctgaagcacaggccgccctatctttacagtgagtccctcctgcctgtcctgagcactgaccagtgagcccaccgggtgagcagctgaagctcctccctgtgcccagcacctcggggcccccaggatcagattttacccagtagaacccaagaacctagaggatgggaaacacagctagcaaggaggtggcaggaggggtgcagatcagaggggtgagaaagggaggctggaggggcaggtggagcactcaggagttagtcatggggaaaacaacatgaccagaaaaggagcagaggtggggcctgtgaggctcaggtaccgaatgtctgtgctaagcaaccacgtgggggtcagcaggtcacccctcttctaaggacatccgtctccctagctgctcagtggagtcacctctgcaaggctctagggttctcgccatccaggttgggccctcagaagaaactgaaaagttctcgcacgaaattcatagcacactgaaaatacaaagggtggcagctgggggtctctttctttgggaatgatttgctcttagcccagcatcttgggcctcagctcggggcttcctgaagccgagcttccctggtgaagaggccccctattcagggcagctgctcaccaacgctcctgggcctcggggcactcaggaaacacccgagttaccctcagggccagaccgcaggcctggagcctctccttttgctgatgcacctgcatcagaaagccagacacctgctctagaaacctaggtaccaggtctaaaatccaaatacagcatcaggagcccaaatacctggttagaatccaagtccccattcagaaaactaagtatcatgcaacaaatccaaatacctcttcagaaactcaaatatgtggtcagaaagccaggtgcgtgcccaggcatttgtaataaccgcgtaagtgggagtcttcatttttctccagaacaagtagcccctttctgaaattcatcataaagctaaaaaataaataagaattgttttatttcagtgaggctgtagtgcatttttttaatgtgtctgctgtgtatatcacagtgtgcctgggatatctgaaaaagtttgaagttgccccggtcacagaatctagggtcctgattgaaaatgagcagagggttgaggaaatagactcactgaaattgcgtattcaggtctatgaaagacagagaactacatcaggggtgtcacagcaacccaggggctcctcttctaagtttcttaaatccccaggaaagggttttcttctaagacgtggttagaaggaaatgaactactaatcttgagaaatgaaaagtcaagttcggagaatggcctgacaattcgcgctatcagagagtgcatgttttctctaactcccattttctactttatccataatttatatgatgtgtgacacataacttgaagctaatgcgtggtggggccaccgcagagggacagaaaacagttgatgtggaagccatgcctgactgtcacagacggggatggggggccagggtctcagctcctggagggggcgagggagcccattgtcctgggatccccagggttcacccagcatgcatcgggtctcaggaacctgcgttgctgatggatatgtgaatgaggggatgaacaggcgtggaagaagggggagtagggagaagacagttgtatctttgcattcttcacagagcctggggcagcacttagcacaaacagggctggctacttaaaacccggggtcactcgttcaaaaggcaggaaaaaaagctttttcctttcttcgtgttctctctctggatctttcatagtggtgtgtgtgtgtgtgtgtgcgtgtgtgtgtgtgaccttctatttaatgtcacattcccccaggcactaggatactctcactgccttcaactgggcaggtagaaggcatccatccagcccccatcctccctgttgaggagttgggggagtctcagttgctggtcggggcaggaagtggacagtcaagaacccagcgggatggagcagaaggtgggccaggtgtgagttgaggggtcataccccggcacatgtcccattgccccaccagagttccctcacaaaacacaagttcagagataaaattcttatgacatatgaaggtagcagttacagagcattaaaccccaagcatgaggggctctcttctgagtgagggcctgagcactgctctgctctcgtgttcccgaagcccaccctgagtacgagaagtgagggtcagagccctgcagggtcctgcccatggtgactggcgaaggactttcttttccgggggagcacagccagagctctcttctctgattccatcattagtttctggaaaggccttccctgcccactctatctgatgttacctatccaacttccagccccaagttactttctgtacccccgttttactttatattgtcattaaagcacttaccagtatttggaactgtggtatttacccattttctcacccgcaagaacgcaggccccataggatagggatcccatccctctgtccccttgcacaagtgctggcatgagagaggcacccagtgaattttaggtgcatgaatgaatagtctttacgtgactttgtatccttttaggagcaagctggatataaataaatacatgaagtcaactaacacctccaagtggctcagtcaattttcagtgatttttctctgtccaacaccagactggcttcagtgatttctttcactgatgtggccacatttgttttgttttattttgtggccatgcagcccgatgtcgcaggggctgttggtcctcctgactccctgctggaacgtaagggagagcctcattccaggtctgcactggccccaaactgggccaagactgcacatttcagttcactttgggtttctgatagcagttctctccccagcccccatccctaggattcctaagagctaagggatgtacccaccaatccaagccacctactagcattgaacttctgtgtcttgaaaagggattttgttttgaaccagtcactgggagatgcgttctaaggtgagggcttccgggtcaataaacctgtaggtgtcgcccaccaaaaaaagtaaatctgacctcagcccagtcccatggtggtgcctattccatcagggcagagagtcagcagctggcaagagaggctcaacccccaccactcctgagagccaggaggccctggcattcaaactcaggcttcacagatccacacatctttgttctcagagaggagaacccaaagtgtgtctcccagtgagttctgaggatgagggaattatgaaaggcaccgtgtccacttgtgagataaaccgacccacctggttcctcatagtgggaggtggctcaagtgtcaagtggcccatgctacctggtcttcttggttttcttccatccgcgtccctggagatggttatgtgctttgcttcttactaaatgtatccagaagccaaggcaagctaggaggaggagtgggatctggatctgcagatccagaatagcaccgcaaaattacccagctggggcttagctgactgtccagaccctgacggtggggtttagaattggatagcatccaagcctttctgtgatttcatcagcaccatcatgcaataggttgtttaaacaccgatatgaaaacctgttttccaacgatgctgcaacatttgattaattcttctggatgggatatcaaggtggctaaagcaagcattgcttgcagttatgatgaatttgctgagtgtcagaggaaaattgtcaggccacaaactaatcattcttaggtgcctcatttagcattctgtccatgtttataccaactctacacgagcacacacacacacacacacacacacacacacacgcacgcaaagtattggagaccaccttgaactaaatggatttgacctctatttgtaaagggtgcattagcgtgaaatggaggggggctttctttgagggttcagcaagccatctagcctacatgctgctgtcttagatgccagccttggtcttaaagagcctggtttctaagggaaaattcatttcatggacatcttgttgccaggaatcagtgtgattcacttttcatttcaggatgatgttgagtcctctgtgttattcccagtgtggacgtggagtagtgactgatgtctaattatttggaagggagagagcttctctaagaaggacatgcaatgtcagaagcttccgttgcttggcaacacgtaactttacctatgtttcaccaaaggcagtttaaagggctaaagatgcccattcaggcaatagtagattacaaggaagatctcgaaagctggcccgtcaaaatcgctttccaccatagaaataaacacctaagagagggtttgggacgtgagtggttgcgtgttgcacaccagcaaacagggtgagaaagaaggcagtggtgccatttttccagaatcatactgtgtacatgccctgttaaagttgattcaaattgctcattccttacagtaaaacctccaacctgagactccccttcagaatgtatgcccctgcagcttccccagtttttatgactgtggcacctcactggaacccatgccagccccttcccatccagctagtatgcctcccggcattcggcttcagcctccagccttgtttcatgtcagcagctgagctcatgaaaactcttctttccaatgaaggaggaggctgagaggtgcaccaatggctggatccaggcacacgcaggctccgagttcacagaagcagggccatcatgtcccggctgttgggagttcctgtgaatgttgcaactctcatttctttttgaagtgggtgtgtaagaagcatgtctgttcattgtggcagcctgagttttggaggccccacaacattctttcctatgtggaaagagttttctttttaattcagaaacaacaaaccttctcagcctcaaaccaggccaatcttacagtgtcatggcaaaacaaagaaagaaagaaaaagaagaaattaaaacatgcaaagcaaaaaacaaatagaaaaaaaaagaacacagtgcaacagtcaaaatcaggttaacttaatgaatttgggccgaacttagatttaacatttccttgaagtttggctcccataaatcacctgtctttttcaaacagcagcctctgaagtacaatccagattatttctccccttttgatcaatgcttttcccgggaatcaaacccagctcttaagagcatttataatgaaagctaaagacagctgaccactaactggccctgaacacccaggtaaactctgagtttgcccacaaggaaaagttatggcgtccacatgctccaaaggggactccatgttcaaggtaatctcccaagtccctctgctgctgctggctgtgaggcatcaacatcctcccccaacctggactcaggggctgcagcctcagaggaccagcgtcgtggagttcaagttcagttcagcacttgaatagtacatccttcaggaagccagggcactgaagactgaaaagcagaatcctaacattcatgtatattttaaagccagagttcacaaatcggagaccttaggaattaatataagtacatgtacatgtatttgtggctatgtgtactagttacaaataagagacaagaaaggagatatacaaataggagaacaaagcatattcaaaaatgaatatggcaaaagttcaaatgaatgtgaactaacaacaaaaaaatatagtttctcatttctaggcatattacttctgctttttcaaaaccatttctcaagttattgataataatacgaattctgtccttaaattgctcagtgaaggagagtaaattaatgtattttaagatgatctttctgttttgaagtcctgctgcttccgtcacatttacaccagcagacacggacacttccatctgtccccattcgtcatcactgtgagatgaagctaagagttcgattcactaagttttatatagccagtaccatgtgaaacacaggatttgatttaagtgtgtggtgtgactgcaatgggaagcagtcgaacacacagcagcaatactgcaccatcaccatgacaaccagtggaaataatggccccctttgggaataaaagagatctcaaatcctgttgcagtcagtagcgctggctgtctgcagtagagatggttttcaatgattgtttcagaaattccttatcaacagaaaatgaacgagacggggagttttcagaaaataaatggatttgcagacaacttaacgtcatcatcgaagtcaccaaagaaagcaaagcagcgactggggaagctgaggctgaagtagcaagggcatgccacccaggctgctattccagtcgctgtgaaagcctggtcactgtctttgccacgcagaacacatctctgggaatgctgtcgatctatgcctccatcttttaaaggctttccatttctgtctggattttttctttatgggaggcagaaataatagtgaattgaatgaaatggagtatataagtgtctagcacagtccttggcacatagaaagtgctctgtaaacagtacttcgcatcaaattcttgggatgaatgaaagtgagtgcctggcgctgcacataagaaaagacgtggcagcaaaccatcctcccgacatgacgtgctgcgttggtgcgtttacgtccctgttccttctgaaagactcaaaattgtgttggagctactggtgcacctttgtaagacctgcactaacgaccgcgccactgggaagggcacttctttatgaagtcgccgtattcccagggagactggtcaacccatacaatgtgaaaaaaaaagaagctatgtgaaaaaatattcccctttgtcatagagaaggagaacttggtgaggctgggcagctgcaggagcctcagtttttcagataacagaacagagtagaccttgagggtccatttccctttttgttttaggaagtgctgtagaagggggcaaagagatctttgatatgagaaggtatttcttcaaagcagcatcgtgcctgaaatagcattgttctttacgctgagagaggaggaaatgggaatttggtgttcaacgggtttaaagtttcagttacaagaaatggacaagtccgagagctctgctgtgccacatcatgcctaaagtcaccgagactgtgttgtacacttttttgttaaaaggtaaatctcatgttaaatgttgttactacaattaaaaagaagccctaggctgtgtttgcagaaaggcactaaccatacatgtgctcaagagcccttttgtgttttcttcacttcatgttgtagttcaatttagttttgctttgcaaggatcctcggaaagtgtatcatcagcaatgacttttattttagaactaaatgtgaaccacggggggaatcatgatcgacattgctcgttgtcctttcttcctccctgtgggctgtgaatccaactcgtcctgggagcagggcgctctcgccttctgttcctctccgagctcagtgcacagaagcattctgcttacccagctgggactgctgactgcaccatcgagcacgcaggttgcactgttctcagcagctacactggattatatgccgcctttcttactgactgattatgaacttaaaacaaattcactctgctgctgtgaattatacatttatttttaagcatttatttcaactcgagatgagcggtctctcctgtaaatttctccctgctggatctttgctctggtttctggtgacatagtgtgagtgccggcagccgcgagcctcagaaggaaaattacaaagggaatactcagtaaatgatgtattgcctttcgcatcagtagcctgcttggaaatgttcaaattatcagcccaggagactccagtgctgtggacatgggtctgaacgaattgatcacctaggggctactgagaacgcggtgctctgtaagtatccatggatgcacagcgcagacgcggaaagggatccccagaatgttccttctgttgctggtttagattgtgaggaaaaaatgcattttccatgagtctttaacttagtattaaatgttgttactggttgataatttaggaatgtcacagcagttctattccttgtcaactctgcttgagcctctctcccggtttctaaaatgtaacatccattagttggtggggtaacagtttggttcaaatgattttctgcctgttttatgtcttcattaaattgatacttaacgtataagtaatccctctttcacctaaaataaaagagctaaattttctttaagcaaacagaacacccttagaattttggacccgcacccttcaactaacatgaaaatgttagattcattttgggaaatattttatatggaagctgactaaccaatgatttgaaatgtcacaacacaaaattggaggggagattttctctagctgcgtttcttttctctctctctctttttttagaagcatactgcttatagtttaagaccatatcctagtgaatgaatagcccaagttctcagagactgtatatttgataaatgctcaaattgtttttaaattgctaatgctcccatgtgggtaagctcagatatattatagctcacctatgaattggttttgatacttatgatgagaacagacttcctgctgagaataaatattagatttcattctatgtggtacttcgtttaaatcagtttatccctgaaccttggtttctttcctacaaagtgaaggaggccctacaagctcttgcactgcactcagaggaagagtcgtccctggcactgtaagaattccaccaagcctcactcacttgggcaactgcccagaagggctcagcagtgttcagacacctcatgtctcagtaggtgaccgtgcccttggacaggaagctaccactccagggccgtgctctctccattcagagtcatggatagtaggagagtctttataaatctgaactctgcatgtaccatcatgagtggctacagttcactctcaacatttgctgtattttgttcacactaagagagaatatttggctatttcatattagagaaactaggctaaaacaactaatcacgttttacctgtaatgtgaaagggcttccattaccttctaatcaacttatgttccatcccagttactctgtgtgcagagagatctgagtgtgtgtgtgtgtgcatgtgtgtgtgtgtactatgtgtgggacatcacatggaaaccagggaaggaatgcatttatagaagtgggaagttctcaggaagtcttcagctttggatctgctttcacactgagcacttggtgtcagtgtgctggagaaaggctgatagtaagtaaagtgtaggcacggctagggcgccggaactttccttgcatagtattttatcccagtcacgcactgggggattgtggacattttgtcagacccccattttaatggaaatttaaatccttccacccccggaattgcttcttcggggctgttcagtgctgctgtgtggttcagtggcctgccctcgtccccaagagtgcctcctacttcaaaaccccaggccgacccccagtacagtgcacaggcaggggctggcagctgctcaggtcccctgttcctggaggaaactcctcatagaggcaaattaaggattatgccctcgcttttctgacctaggggtctgccatgggatgatgacatgttctgggagataacgtatgggaaggttcttagtattaggagctgcggaaaccaggatgctgtggaaaccagccacatgttagctgggcaaacgtaagaaagcacgctgtcatctggagtaaggggcactggaaacatcaccggggccctatgggttgagaaggctgtgggctcacctggtttctcctctctcccgtccctcatcagtaaaccccatgaccctttggcagcagcaggggagatggcagtgttgcctctcgcctgatctctgcaggctggagggctgttctgtcgctggctctgctcgtacttggtagagcacagccactgcctcgggagcacagatccaggccgtgccgaggcaggactgtgagccccagttgtggggacctccggggcagccacggtctcagccgtcgcccccgctgacagccagagcctatgtgtctgtggagggaagagcaaagctgtcagcatgagtatgtgcatattcagctgaccctggactgctcctgcctgtttatttctgctgtgacctcagggtgctgcgggcatcgagggggaacaaggccatcaggaggaagtgggcaaggacaaaattatttccggggagtttgtataaaggtggtcatttgcaggggagctgcctagggagggggcagggaagacactgctgttcaggaggaagtcagtagtggggagcaccctctatctaattccaggggccctaccaatcagaacaggtgccaaggaaggaagatgcagcagtgcgtaggtaggcaggaaaagtcagtgataccagaaggatggatacaggaagggccagggcctgctcctggctgctggagccctgcgtggcacatggcgcaggctcaggcagccatgtctgaatggctgaaaatgatctgatgaatgatgcaatgtcaattgtctttgcagtgtttgggagtcacatatctttttttttttttttttttttttttttttttttttttgagacagggtcttgctatgttacccaggctggagtgcagtggtgcgatcacggctcttgacctcctgggcttaagcagtcttcccacctcagcctctcaagtagctgggaccacaggcgtgcaccaccataccggactaattatttttattttttgtaaagatggggtctcactttgtcacccaggcgagtcttgaactcctggctcaagtgatccccctgccttggcttcccaagatgctgggattacaggcatgagccactgtgcctggcctgggagtcatatgtcttatggatgttgggttttgtgcctctgcagatatgcatgcacagatagaccatttacagtcctgaagagcagagtgggctgtggctcagacactgggttcaaatcctggcttgtcacttccttcctgacctcgggaggtgacttgactgctgtaaggtagtatcctcctcattggtgatgagagacatagctgagatgattcttataaaccactcagcacacgcctgcacgtgagtgggccccatagagtgtagctgctgctgttgcggctgtttccctctctgtgttctcctaatcacactgggtggttgtcattttttatttcactgggaaaaaaaaatttaagtcagctgtactttgggaaaagagatgttaaaaaatgttttccaatacattaaaaatattagcactgttgattcttttagaatcatgctgtctaactcttactttgaaactatactctgttacattcatcctggtgatactgtttaacaaaaaggaaaaagaaaaaaaagaaagtagcataataatatccataaacagtttttaagtgtgaaataatcctgaatagctagaaaattcagaggacattttgcaccatgcatttgtggcagttcacgcgcggacatcaggctctggaattctgccagtgttttaaaattagaactaaagaaatgcgccccaacttaattagtataaatgttgccgtgcaacttctaataatgagtgtaatttgcaatacatgctcaccatgtaatttagcacaatggggcccgcagactgtaggactagtgtttaatcaagagaaatgattaatacagaaaggaacaaagtttagcaattggaagcctttgcaggggcctcatggatgcccattcggatgctcccatttatatcaccccattggcaacaccccacccattcccaccccagcgcgtctgccaggaccacagagaggaagccatgtcatctacttagctgtaggacaatttacacatgagtgggtgcctcagtttccttctctgtaaaatgaaggtgataatggtcctaacctcttattgttggggggattaaatgggtcaatgatgtgaagcacttagaaagggcaaaatgtgctgcgtgcaacaaatgttaggtgttaataacgatgatactacccttatgatcatctttcaagagtcattctccatggggatcttggatgccaggctgaggagggtgcccttcatcccaaagcgcagtacatgttggtcctggggtgatttatctttattgaggtaaaattcacataacataaaattcaccgttttgtacattcacagtgttgggcagccatcacctctacttagatacaacatattttcatcatccaaaatgaaacctgaacccactaagcaatcaatctccatttcccccttctcccatgctggcaaccgccaatcagctgtccatctgtattgatttacatattctggatattttccataaccatccagtatgggaccttttgtgtctggtttctttcatgtagcatttgttttgaggttcacctgttcggtagcatgtatcaatatttcattcctttttaggctgaatcatatctcattgagttactgtgttttgtttactcattcatcagtggatgaacttaggttagtttctgccttttggctatgatgaattatgtgctattacgataatcatggatgagtttttctttgaacacattttcagttcttttggatatatacctaggagtggaatttaaacatcattttatggttaaattttgaggaaccattaaatctttccgtggtggctgcaccatttgaattcttaccagcaatgtctgggggttctaattcttctacatccgtgccaacactgggcttccttttatttttttctaaattatagccatcctaagagatgtgaagtggagtgtgattatggtttgggtttgcatttctttaatgaatagtgatggtgggcatcttttcatgcactttctggctgtttgaatgacttctttggagaagcgtctattcaagtccttggcctgttttttgggtttatttgtctttttgttgttgagttgtaagagtactttatgcattctggctactacgtccttacgacataaatcatttgaaaatacgttttcacattctgtaggtcatcttttcactttgctgacgctgtcttctgatgcacaaaagtcctgagatgattctagacagagactgagcatagtgatgaaaaacattgcatcataaagggaaagactccttcctctcttcgttctcttccacccttccgtatacccagagagaaaggctcagcttggtgctggtgtggccttaacacccattcaatccttgctcatctacctttttcacaaagagagaacaggctctaacccaagagccttacacagggccagccaggaaattcatagtctgtcttccccatcttcactggtgatactcggcctcctatttatactaatgacaaagagttttaacataatgtatttgatgtctgaaaaaatgggtgagtgatatgataaggcttgtgagggtgctaagagaatagcagagtgtgagaactgctgctggacacctctgggcgtccccgagcaggtgacagatgggggatgcccaaggaccctcagtctggtgatgggatgtggggcagagggaggacgctgggcaggccggccataagccagcagtttactggaaatgtaatggctaaaacaatagaggtgtgtggtcccgcagttccggaggctgcaagtctgaagccaaggcgtcggcagggctgtgctccctttcaaggctctgggagggattcttccttccctcctccagagtccattgggtcccgcagtccctggcattccccagcttgtggttgcctccctccagcttccacctccatcttcatgtggccttcctctcagtatacatgtgtgtgccacctcctcttcttataaagacaccagtcattggattagggtccaccctaatccatgatgacctcatcttaactaactacatctgcaatgaccctatttctaaataaggtcacattctgaggttccagatggtcatgaatttgggggacactgtcaacccccagtactgcattgtttgcagctggctgatgacccattcccactgaaggtcttgaggcatgactgacaccactgagtttcccaaaaccaacctggtgcatccactttcactctgaagttctgacctaaattggcattggcaccatcagggtccctgggtatttcacagaagagcggggaagcagattttcacagacaaggttcaacctgagcacaggaattgtacgcctaatcacctaatcaacttgtaggtgcctctcaggtacttaacgttgacttgaaaatcattgcttaatttaggtctgtttcctgtctgaactaaatagagtccaggaggtttatagtctttcatgcttgctaaattgtgttttacttggcttccaactaaaaatgagagatcaaactatacttacgctatgttgatacagagttccgtctttattgcaaacttcaccacgtggagctggaagacgtctgagttcagggatcatgtttttgtctttgtttgcctgactctgccagacctggggtacagcagcaaggagacccataaacatttggggggataaaagaatagagaaaacatagtccgggtgaatgaaggggcctttcctgcaccccagagatggtgtcaagtcccacaaaagactcttttataccagaagaaaagtatttctgtcatggctctgccataaagtattctctctcctccttccaacctccccattgtcactgaatttcaaataaaatgatactctttgctcaaaaacgacaccagagtttgatacagctcaagtacaactccacggctcctagtcaggtcaactcttatttgacctgaacattgctaagcacaattgaataaactcaaactctcactgcctatttcctattttcccattacctgattttctagaagattgtgacatatatttattttgaggtttctctctctctcttttttttaagtaaccaagcaaggggttctctctcaaaagacatacaattctgtggctctacattgataaatatttgaggggatgtggctgagtgtttatagagacctgggtgggggaaaaatactgctcaactccaaccttttttcataaaccttttggctagttcattcactccttgaacaaatatttatgaagcacttattttaatgacgcaagggtttttccaggtaggccagaattaaaattaagttctgagtggagatgtttagaatccacaaaccacacagtattaacctctgttagtgccaaaataatccatggatattatttgggtagggggaggagttaaagtaaatatttccaatggatttctggaatttacaaaatgtgtgtccatttatcctgaaaggggatgtttgtctgtttcaatagagcttcccatacttgtcccgataaaatcttacaatgtttcagaataattcccacgtcttatgagctatgaccctggagatgtgggtttctgaagtctggttgctttcacactgctgggatttgaattcaggctctgccgttttaccagctgagaagccttaggcagattacttaacctctctgatcctcagtgtgcacagctggaaggcaggcaccatgagagtcttgtactgtctcagagagctgctttgggatcagatgtgttctgtagagaaagcgccatgaagacatcatgccctgatcagcatccgcatcttgacttttgctcatctgtgcaacttgctcggcttctgtacatgattgtctctgaaataagagggcaccttctgcctcacacaccttgttcctacattgataaaccctcctacatcccaatactgacttagggatggaaccccgagcttctcatcacacctgctgcttacaggggttgcactggggctggtagtcagcttcctccaggaagaaggcatctctcaccactggggaggcacaggaggcaccacgccacgtgtgcctgtctgcagagtcccctgcccagggtacatgcccctgggaaaactgcccaagggaaacagtgcatccaccatctctgtctgcagccccgctggctcgtccgccctcgcctccctccctccctttcaccctccctccctctctccctcattccctccctccctccctccaggtgcctttgcagcctcagggcattcacactgcacctctggcctcctcctggagcttctggaaggtgagataaagtccctgggatgccaagctctctctggacctgaaaaagcctcccatcaaggagagaaatcctagagatgcttagcaaaagtttcagtagccagggataatgtcaaacagccagtcccgggtttctaaacccagttgatcaatatcccctttattctagtaggtcaggggactctggagttgggggtggtatttcctgggagacagccagcctgggctagtggagcatcccgcctttgcttttggtatgggaaggagcccgacacacctggtctttctcaactcctgcaagtcacgtcttcccagaatctactgtggtaattccggccaggactcaaggatgtaagaagtaggagacgcaggatcaggggcacagagctgccagccctttctcagaagcacatcagctgaataaatgcaccaaagcctccaggcatccagcctgtgtcctcatcctgccagggcccctgctcctcctgtcactgatgaatgcagccagtcagtcaatcaacaaacctgcatgaattcagtgcccagtgctaagtcccagaagtgacagagagacagagagaggagtctctggtctggctggggctgggttactttgtttcctgcaaatcttaccaggagtgcaccttgtcagtcaggggttgcttggaaactcagtcctggaggcccggccagctacagtgtcacctccaacatggtgctggctgtcaccgcaagccacagaagcaccttttctcctggcagctgggagctgcagcatccaggaggggtctcactgtccagcagcctgcacattctggaggttcatatctttgggactgaaaattggggtcccctttaaatttccgtctcctgctcaggaatgtcagtgcataataagagaaataaaatggattttgttgccttctcgtttggttgcctttgagccaacatattttaaagtgtttgtttacattccagaaaagggtaaaacatcaacggcaggtgagcttgatgttctgttaattcctgagagatcagggatgttttcttgtgttcgtgaacaactcggggagagctgaagggtgcggcaggttctgtgagacttggtctctgcttcagggcacggagctgacatttgcaaaggccccctgtgtgccagccagtgccaagggcatgcttcttccctgtggagcttcccccgaggtgcagcgaggtcactattgtgcctgttttactctgagccggaaagagctggcccagagctacacggctggtggactggagctcgggtcgtcctgaacttgtggcccaggctctttgcagtgcgtgtgctgcaaaggacagccctgaaagatgctgaaaagaattcagtcatttaggttttaaaacttgggctttcccagctccttcattgtttaaatatcaccaatgaaatggaggaaatttatttagcacataattgtagtctcgtttcatatgaagacaacaacatttctctcttgtttctgagtggtggcctcacccccctccatggccaggagtcagggggacatatgagtcaccgtccaagccctgacccttatgagagaaagcaggcaagactgataattgcactgagcaatgaccatgagctgagatggtctggaactcacaggggcacgtggtcaccctatttatagaacatcttacagtgatgagggccattcattcattcattcactcactcacttattcatccctcaagtccttctgggcagcccctgggtgccaggccctttgctggccgtaaaccaggccgacttgtccccagcccttccagcctggatacctgagtgggggtggtttgtagcccctctgttctcacaccagagattacttcccattttataggttgccaaatgtacatgagcgcccacacacagaaaatggcagagcccagggactctgaagcctggtggttgaggtatgcccagcctttgctaaagacagcagagaatagcttggcttggggaattgaaattttttcttgcctgagaaatatgagaactcctccctttgccggggcacagatcccatcaccaccaggtcagggaagagaccatgcggttcccagcccagtgctctctgcctttttctctctcctacttccttctcctctcaactttagatccccagccacaaccgtcaaaccctgttccttttcttttcttttcttttcttttcttttcttttcttttcttttctttccttttgagatggagtctcgctctgtcgcccaggctggagggcagtgagggcagtggcgcgatcttggcttactacaagctccgcctcccgggttcacaccattctcctgcctcagcctcctgagtagctgggactacaggtgcccgcggacacgcccggctaattttttgtatttttagtcgagatggggtttcaccgtgttagccaggatggtcttgatctcctgacttcgtgatccacctgcctcggcctcccaaagtgctgggattacaggcgtgagccactgctcccggccttaaaccctggttccttaaacagctggtaacccatccattaagcggaaggcaggcagaaggttcgtgtcccaggagccacccgtcttcctgaattgccacccctgtcctaagcatgacacttcatttgtacatactttgagttatagtctccagaataattaattttaatatttatggaagaatcgattccgtgggaagaaagccaatttcctgaacatggcatgcggtttaatgggtaattctctctgtcattctaggcaagaaaagtccatttgggtgttaaatttcttggctggggtagagataacagacccacagattgtaaaattccaggctgggacatgggtgtgaggatgacttttgggaattcagatgaatcttagaatgctagttcctaattaatcactgtaacacggcgagtgaatggagaggaaaagctatttaagaacaaatgtttgggcattggtgaagtggttacgtttctagagtgatcctgcaaggatactggttgaggtgactgggggaccccggaggaggcgcgggttaggaaggttctgagcgtggcctgggcgcaccaggcgggtgcgcggtagacgttcctgtggcttacccgacggtgccaccttctggtcacctctggcatagcacccgggcttcaagcttggcgcatgcgcggaagcgtccccgtagcccgaccccatccctaatgcttccaaattcaggtgctaagttgagcccataaaatgcaaagttataacaccatttcactagaaattccttttaggacactcctgtcactctatgcttcctctatctgtcctgttgggctgtgagcatcctaggggctcccccctccaccccaagagggtgccatggctggtgcccagccaagctcagcaactgtgggtgcctcacacgtgccagttcacaggcggtggcctaaacacctggaatgtggctgtcactgtccccctcccaccagctcagagcagaacctgggccagagagtccacctgtgtcctcacccatcctaagggcggaaactgtcccaagatcagagaagggaaaaacctctaagagacagatgacatgcccagggtgacaagatagacagcccaggtgtgaacccaggtccacctgtctcctggctcagagtgacaagattgtgagcctgggtgtgaacccaggtccacctgtctcctggctcagagtgacaagattgtgagcccgggtgtgaacccaggtccacctgtctcctggctcagagtgacaagattgtgagcccaggtgtgaatccaggtccacctgtctcctgtgttggggctcttttcagagtgctggccgcctcctggagcctcccaatgcaactcacctttgcaactcaagggtgattttagggcaataaaatgctccaggatagatctgaccttgtggtcactcaagagtccagcaacttcctggccaataaaccaaacttctccttgccctgcacctgtgaggtctggcccatcctccccatgagcctgagctagtaaatatccagtgtcacctgctacccttgctttgctgagaagccccagatgaagctcctcaccatggcagcgaggcacccacccagccttgggagagaggctcatgccatcagctttgtcctaacagctgtgctgttgaacgcctgggtttctgctcagctcaggagtcattaaggtgcacctgctacttgctaggcatggacgaggcatcatttcctgtccttgggagcttcacaagagcagaggtgggggctagctctggtggcatattcagcacctaggacacctatgtcaaatacaaggattccagtaatttttcaaaaggttggcaccagccactccccacaaccctaccttgaacccactgaagtctttttcaggggagtgtggtctcccagttagactagaaatccctggaaggcagcaaggttgtcgaatctggttaattaaacaaggcctccttccctgctctgtatccagaagacagtcagtaaatatttgttaaacaaataaacacatttctattactggctcttggtcagtaaaatcagcatttctttaaaaattcatgagcgccgtagtgcttcccactgtgagagcaagccatgttatgaaattggcatgaattttctattaaagagaagagtctatttaataaaatactcccaaagggtagctttttgggttactgctgtgctgcattataattttctataattacatgctgttgactgtagtttaccaacagagtgtcagtaaagaaaaacgtaaccctggaactgtattacttcaagtgtgtagccttaccaaatggaaactcatcggttagctgggtcgagggtggcaaagagagtgttctccgaagtcgtgagtagaggaaacataaagttgccctgcagctctcaatgtgtgcggtcaaaacaaatagctggggggatctgttaggaaacctgttgggataatctagaaatggagggagaatcctatcttggagcgccactgggggctgctagtagaaaggagtttgatggacagcagctttaaggggcagcacctgctgagaaaagcaatcctttaaacggtcacatggtagacagaggagggatgtgctatgatctgaatgtctgtgtccccccccagatttatatgctgaaatctgatcaccaatgtgctgggttgaggaggcggggccttgggaggtgattaggtcatgaaggtggagccctcttgaatgggattagtgcccttacaaaataggctccaggggacctatttgttgaggacacagcaaaaaggtcttgaacaggcagagggccctccccagacaccacatccgctggcaccttcatcctggacttctcagctttcagaactgtgaggagcttctgttgtttacccagtctaagatattttgttatggcagctgggctgactaagacagtgtgtagagggagtttcccgaaggcagggctgtgtttgttgtgttcacctttgaatgcaagtaggtgccagtaagtagtggaatgcatgcatgtgtgcgtgcaggaggctaccacggaattagacaagacccaatgtgcccctacctctgccattctcttgaagtgtgcctcaggccaaattgcctgtcctctctgggtcgtggtcatctctgttctagatgaggattctgatcctccgtcccagactggctggcaaagcaggagatgcaccaccctaacaagtctgatgaggaccagggctttggcagggaccccagctccacaggtcccctccaggcactggcagagggtgcgtcccctcacagctggggtgtccagggaggttgtgaagcagcttctctgagttataaaatatggagcgttctctccttagttgcaatttggggtcaaatgttctcccaaacaaccggaggagcagttataaataatgcatgcacattgtttggcccaattaacattcttcagaagatgggggcaattctttgaagtctcttccttcaaacggcgtgtatcgagggggacattgacacacccctgttgggctgggccaagcttttgccctaattagatatttacacaagagacccatgcgaggagggcggatttatttttaataaaagaagattaaaatctgtgtatctgcagcatattaaggaagactgaaccaaattaaaaattctcccacaactttgaatagtgggggcctggaaaaggaagcatttcacttggaatttactaaaagttgttgctttgagtcaggaagaaaaaaaatcagcaatcccccatcccccgtctcaaccccagcttcaccttggcttgttggcgctcccagtgagggcactgggggtggtcagcggagccctctgcatttgacgagccactataaacccaaaccctgtggctgggcgtccatttgtggaactgcagccccaggctggtaggaaggagacagtggagatgacatggctgggaatgggatggggtgggggccatgctgacactctgcaagctcagggaccagcccagagggtcacacaagagtgcatgccacacccggtcttcccgagagaagacagatctccagactttcatgtggactcctctgattctaaatatttcaaactaatttaaaacctttgtaaacatatatacttcctcttccaacctagaaattccacttacggttgttgaccccaaatacatgaagaaatgtgtccacaaaaagacctgtacccaagcattcatagcaaagacctgtacccaagcattcatagcagctttgtttataagagcaaactaactgaaacaactcaaatgtgtatcaagaggagaatggggccgggcctcgtggctcacgcctataatcccagcactttgggaggtcaaggcagaagaatcgcttaagctcaggagttgaagaccagcctggacaacataggaagacctcttttttttttttgagatggagtctcgctctgtcgcccaggctgaagtgcagtggcgtgatctcggctcactgcaagctccgcctcctgggttcacaccattctcctgcctcagcctcctgagtagctgggactacaggcacccgccaccacacctagctaattttttgtatttttagtagagatggggtttcaccgtgttagccaggatggtcttgatctcctgacttcgtgatccacccgcctcagcctcccaaagtgctgggattacgtgagctaccacgtccagctgagctcatcttataattaaaaattggccaggtatggtgccacatgcctgtagtcccagctactcacttgggccctggaattcgaggttacagtgagctatgatcttgcaactgcactgcaacctgggggacagagcaagaccctgtctctaaaactaataaataaaaggggagagctggagagcagatggcagcacaatcacatgactgtatgctgttctgcagtccaaaggaatgggctgcggtgcccatgagaaagcggatgggccttgaagcctgtggcactagagaagctttaggacacgagctgcgtgtttccacttagaggaagctcaagggcaggcaagggcatcttctctggaggtggagatcagaatggtgtgggcttcctggggttgcaggaggggactttttggtagactgaaaagggacaagaaggaggtgtctaggatagtgacagatgttcctccaggggaccagggcatggaccatatgggggtctgcagcagacatttgcccaaactcatcataccgtaggcttaaggcccatgcacttaactctatgtaattacagcttttcaaaaaatgtaaaactttcaaaacacagaacaggtcaaacccagtaatacaggtcacatttagtcagcgggacccaatttgcagcttctgttacagtaaatttgtactcttatgttaaccaggcacccccagcccccaggtggagaaacatccttcaccaccagccatccatgtggtgagaacctaaaactactctaaaagcatgtctatttttttagaccagtgccactatgcctggccaattttttttttttttttacttttttagagatggagtcttgctatgttgcccaggctggtgaggagacagctcagggtccagggaacagcgtgggccaggctgacggcggtgagcagagggcagttcagtggcctgggaacagcacccaggtggccgagatgtgtcggtccctgggaggttggcacagctcagaggggaccacagggacagccagaaaggtctcatcccaccaaatcactgcccacccagtggccccagtcatgctactgttttggatttgttggtatgacttgccttttcacacaggtgagaggaacagactcaccttgtttagcccaacgtggctgaggcacagcagcttcctgccttcctgccggggagacctgtggccgctgtggagccggtgccgccctggccttggctccctctgccccagaccaaatactcaggggtcctgtgaggtccctcccactcctcctttaccttagaagagtttaggaaatgtgggtggaagatgaggcacatcttggaaagtgtaaggcagacctcgtcatgcacacgcattgagcacctgctgtatgcgagtcagactgcagaactgaggggtcagaatgctgggaggagacagcccagcatccagtgagcagcgtgggccaggctgatggcagtgaaaagagggcagttcggtggcctgggaacagcacccaggtggctgagaggtgctggtccctggagaggttggcatggctcagaggggaatggatcaaaagagtttggcggggcaagtggggcgggcattaagaacattaagaagagctgagagctatagaaattggaggtaagaggcctggcacgcttgctggatgggccagtgtgcacattgtgagggctgctgcccgagctggcctcaaagaggagtctgggttcaactccaagggcagtaggacagggtgagaattttttttctttctcacagacattttttagaggagtttcaggatcacagcaaaatggagtggaaggtacagaatttccatatgccctccccacacaggcacagccttccccaccgacaacatcctgaaatggaatgggacatttggtaattggatgacctactctgggacaacgcagtcacccagagtccatactctatgccactctcagcctcggtggccacattctgtgggtttggacagatttccggtgaccaggacccgccaatgtagtatccaaggggcttcactgccctaaaaattctctgtgcaccgaggcttttggaggcgcagaggctttcactctgaaagctgcagggggcagttagaggggtcaggctggaggctgtggtacaggaggtgggtaggcctcctcccccagcctggctcagacatccactgggcgtccttccacaaaacgaccttctcggagctccactcggactccactcgggcatcctgcatgcttcgcatcacacacagggacaggtgagggataaagtacccggcaggagcccacaggggctgtgacacaggagggtctggcacaaactcgctgggcttctcacgcccacaacagatccagtgcctgccctgcagagcccagcagcccgggctgagagtggaaattcccagagcaactgagaggggtgcctgttccggaggtggtaggagctttggggctgggggcgtgggaggtaggagctttggggcggggggcatgagagtgacttctgaaactcggcatcctctgaaacctcacctctaaatcgcacggcacattccttcttgactccagctgggccagaggctccctcccggctgctggatgctgctcctagcagtgctcatcacaggcaccagacccccaggggctggatggacccatctgcacagcagttccaactgcatctcacacacgttaccatcatctcgcagatctgtgtcatcctgacgcttaaatttcctgtcttgtggtccaatcaaaataggtcttactgacccagccaaaatctatattcgtgaggctttgacgatgcagatgtttcctgctattcctagagcaggaggcggtgttggccgaggccctcgccaacagctgttcccagaggaggggggctgagagaaactcggagggaggagaggccgcctccccacagcctccatctcctctgcgaccagggaccctcactctcctctgctgaagcctaaagctgctcccggccagttctcccagatcagcaccctcaggaaacctcaagagttgaaaaccaagacagccagaaaatgggggcttctcagcctagtggggagtactgagccctgggctctccaaccctccaagccactccagtccccatctctcacactctctgcatctctgtctctctctctctcacacacacacacacactcacacagcacatacagatagacacacagatacacagtgtttcagcccatttctacagaaatgactaatgtgcacactgcaccagggaaacaccacattgaaatcaaggactccagtgggtcaggagccgagggtgcatccacattgaaatcaaggactctaataggtcataagctaagggtgtattattttaccccaggcattggggcatcccctggtccctgggccccctctcccctcccagccccagacaggatcgtgcagcggttccatcaggacttgggaagctgagaggcccgacttccaatcccagccctgccccttgctcactgcatgacttgggcccattgcttggcctttctgagcctcagtttccccagctgggggagtgaagatggcagttcccgcctcataaggttgtgaggacaagacaagaaaacgcgggtggatcgtgtaggctgacgcccggcactcagaagatctccacgcgatgctgtttttgcggggtcactgccctcctctcatcccagcgtggcctcagccaccaggaggcctagaactctgggaaaggagcagtcaggaccggacatggctctaggcttttcttgtttctctaaaatactcagagtccagggtccttcgtgggatctgccttgagtagccagagctggcacaaagggaatgggaaccctgaacacgggtttgccattccggggctctctggtggtgtgtcctatgtcagcccattcattcattcattcattcattaagcacgtgttcatcagctgccccctgagcatccaccatgtgcattaaggttctccccaggcctctgttgggcggggagctcagcctgaagccaggctgtgagtgggatgggagggggtgccagacagggtcctgcgagaggtgttgggcccacagcacccacggtgcctctcccagaggacccctgcccctctcatctgggcagtgaggatggttggggtggacggcctggctttgcccaagcccagagcaatggtcccggtacagtgattgttattaagttctcagagaaatgcccagagtccctcaggttggtcgacatgtgttccttggactcctcaaaggctttgccagagggtggaggctgtgtgtgtgtgtgtgtgtgtgtgcacgcgcgcacgtgtgtgtgtgtgtgtgtgcgtgcacatgcacgctggacacagggctgtggcgtgattgtcagatcggaggcagagtaattgtgctcaaagcaagaataattaataagcccacacttttccacagcagaccttcttattaaagtatcagaatttaagcagcccaatgttgtacatatggatgtaaatgagatacacattgtccatttgaacagctctaactttgtttctgtctcaagtggtacatctgcactaacacaaatccattttttacttgactttgatttgctgattaatattatgacgctgagttcaggtatatgatcaggttacaaatataatcagtttttcactgaatggctgttatttggtttccaataacagaaacctgtttaaggatatttcaacaaaaaaacaaacaactgctgagtcttactgtggctcagtatttgggttttagtacaagagtaactagataaccctggacgtattctcttcttggtttatattttttaaaaagaaaagaaaaacagctcggttttgacattaaggaaatggccggtagcttctggtttctctgggcagccctgctgggaaatgctggtccttactcttctgctcactgccctcctggtgtcccagtggtgggataatccagagcccacctcagacaagttctaacccagatgagctcacagtactgccatcctgctgcatcctgagcgggcagtgggtttccagcgtggggccccattgtgggtgatggaatttagaggaagcggtgtgatttgataggggtggttcagccatagctcagccctgtgggtacagtgtcttttttttttctttttttttttcttgagatggagtctcactctgtcgcccaggctggagtgcagtggcgcaatctcagctcactgcaagctccgcctcccaggttcacgccattctgcctcagcctcccgagtagctgggactacaggtgcccgcccccacacccggctaatttttttgtatttttagcagagatggagtttcgccgtgttagccaggatggtctcgatctcctgacctcgtgatccacctgccttggcctcccaaagtgctgggactacagacgcgagccaccgcgcccggccgagtgtctttttttcaggtgggatggtggtgaactcaataagagataattttgcacatcaggctagaccctgcatctgaagtggcagagggcattcattctccaggtgccacacaggcctctgaagggactgtgacctcctgggggcaggggtgtgctccccgacctcctgcgagcctccttagcactgagcaaaccgcccccctccccgtgttcagggatattcgctgaaatgagccaggtgggatttaatggactctgagtacaactgtgtagcctcaggttgccttcagcatccaggccctcggggcaccgccatgctccccagaacatgagctctcagaaaatgcagtctgtacccagcattagcacaatcagagccctggtgagaaggtgaaactactgtccttggcctccaaagcatagaatgcccccaagccaatgagtggtcatgaaatttcccacctcctgttcttctcaccagtcagtcagagacagcgcctgctgtgagactgtgccccggagtctcaccacagcaggtcacgggaccacaggtgcctgctatgaaggagggaagacaggccaaaaggcacagaaaaaataatcttgcagttctaagtttcttgctgtatctcttaccacgtgcttcccgtgaggctgcttttggctggagggtcagcttctttgactctgatggcgagtcttcattcctgttcgaggctttgatttccagtagttggccgcaattttcaggactgatcctagagtatgcattcaattattgatcatcaatagtttattgtcccatcactgcgtctcccctggacgcggtaatggaagtattggtttattcatggatcctgtttgaaggacgtgacaaattgattttctgacacagcccagataaagtcaaatgaaagtcacaaggtagaagaggtggtcttgacctgatcatcacattgaaagtgtctttaccatgttcggaagccctggagaatctcacagcaccacccaggcccctcctgagtcggcagcactgagtccccaggtgcacctggcaccccgctccctgagaaccactcagacaggacctgagacaatgcccagtgaggaaacaaggcagggccagggctgcccccaggccttgtgaaagtgggagctgtgcttctcacaaccttcctgtccctgaagggggaatcccaaggccccactgggcattgtccaggcgttttgtgaagctcgggtccagaacaggagttgcctggcagctttggaatcgacggcccagggcctggcctgccccgtgctgactcccccagtgagagggaggcacacacctccctgtgctcctcctggtcaccctcccacagtttcactggtgctgctgctctggactctgccctgcacctccagaggcccctccagaaagtgcaagaccatgagagggccctgactcacaggagctcatgttcaagttcagagagaaaagcagacagggaggcacacagcgcagtggatggccagccatcatggccagcggagacagctagcagggcttcccaggcgggcacaggggcctgggaggggcgctggaggcaggtgtgatgccagagcggagtcctgtgaacaagggccaggagatgacggtcgattggaaagaaaccgccatcgctgctccaaagtcacccttggaattgtagtaattttgacaggctgtggcagctgaacatcttcattaatgtgaagacaatcatgaagacaatgaaaatgactctgaccacctgtcccagggagtagagagcgccatctgcatttgctacagtggcccccgtccgtggttccacttccacagtttcagttacccacggtcaactgtggcccaaaatacatgagtagagtgaacaagatagtttgagagagaagagagagaccacattcacgtgacttccatgacagtatattgttagaattgttctattttattattgttgttaatctcttactgtgcctgatttataaaataaactttatgtaggtatacatgtataggaaaaagcatagtatatatacattatataggaaaaaggatagtatatatacatagtatgtataggaaaaaggatagtatatatacatagtatgtataggaaaaaagatagtatatatacatagtacgtataggaaaaagcatagtatatatacatagtacgtatagaaaacggcatagtaactggtctaaaataggtgagtagagtacaagatactttgagagagaagagagagagcacgtttacatgacttccatgacagcatattgttagaattgttctattttattattgttgttgttaatctcttacagtgcccaatttggaaaataaactttacgtaggtacatatgtataggaaaaagcatagtatagtatatctgtggcttcaggggctcttggaccgtatcccgcacagataagcggggaactgctctacataaatcgatggtgctttccattagagccacggcagtttctaaaggaaaaagtaacaagactgcaaaagtattgagggcagtgtccttggtgtctgtgaattgtagtttacttttgtctgtggaggttaaccttgctgtcttcagggcacgctttcgtgatcataaatagtctactctgtaataatattttggtcttggtaagatccaaagactatcccaaacacaattatcttgtcactgcttaggtggcaatttttaatgagtaaaggattcaaccggtatcttggtattggtggtattagtggtatatattatttgaaatgacaatcagctactacaaggatgggaaaaattaattattggtaatctttgaaactcatgaagaattgtgagacattgtacattgttctcaaatttgagattaggtaaggaaacctttgtctttcatctttggttttatgtgacccccattcaggaacgaagagttatcactttaaggctctcaagggcttccttgctttgttctttcaagacaagaaggataatttttctctatagggaggaaatgtatacagtaaaagtctagctatatatagtccattctgtcttatttggccccagctattattgtaaggttcacttctttaaatgggtaattagccattgggcttgttggttgttgattaaatgaattcaagtatggctttagggtctgagaaagcgtgctttcgagaaggttccttgtgtctccagctatctgtgggtctgtcatctttattggaatccagatagacttaattctcacaggataagctctttactttttattcattttcaagaggacactatgcgtaattggaattacgcaaattagaggattagagtttggcacacaggaaattgaaattatcccttaacagacttgctgactcgccagcgttcttaccggtccttggcctgggtggagtgctgttgcgtcctcgggcagaccctgtgcctgtgctggtcccacccctcagcatctgggcctgcccagtccaagccaggcagggagccccaggaggaagcccatcaggggacttcctgcggctttaaaatttcttttgctttttaaaaaattatttgataaaataaagtttggtgtataatccacaggtgtgcacttggggcttctgcagtgtttatataagaaagcattgagcagtggctcacacctatatttccagcactttgggaggccaagacaggaggatcacccgaggccaggagtttgggatcagcctgggcaatatagcaagaacccatctctacaaaaaatataaatgaaattaggcaagcatgatggtgcataactgtagttccagctactcaggaggctgaggtgggaggatggcttgagcccaggagtttgaggctgcaatgagccatgatcatgccactgcactccagcctgggtgacagaatgagatcctgtctcaaaaaaaaaaaaaaaaaaaaaaagaacagaaaaaagcactgaaatgaattatgaagcattgttaactcttagacataatggattttttaaattcttttatgacaaattgccacttgctgttactctgccagcatcagggaatggggtcagtgctgtaactccccagtgaggacccacggcttcagcttctctgtcttagctttgctggagcttcttcttcactcaaggaaaccccatctgcagattccaagcctcccactggagaggtgaggtcaggacgtggtcatttgagtatggaaatagcgatagattttattgccccactttacagacgagtaaactgcagacatcacctccgcagtcatccacacagtcaggagtaggcctgggcgtcagagccggaccctgtgcccccatcctgagctcctcccagaccctgcagcttctccatgcagcctcatgtggagggagcccagtgccattcttcatccccccaggaactccccaaaccctggggcctccctgcctgcactggattcgggtctgagggcagcactgcccacagagccctacatgtcgggctggactttgccaccaggacctgctccaggggacccagggaagcagcccctccccagctcccagcagcctcctgggtcaccccctttcccagggcactgtctgttccccagggacctcagatgcctctgaacacacctggcttgtgtgtatagggtgcagggggcatgtttatatggggagggagacaaatatttttgtccatggctgcctttctttggcagccagagccaggtacttccagaagtacaatctcccagggtgtgctcagaattcactttccagcaccccgagagtctcccatgtatctccagaaaccccaaaacaacccatccctggcaggccaagcatgcaggcatggtgggtggctatgtggcctgactctgcacggcccaggtgggccccaggctgcagaagaaagaacacagcacttcctccctcaaacccccagcgcctctctggttggccagccaggtgcggtgggcaaatgcttcaccatctctgacctccctgtcctcatccatctgtgttccaaggcaaaggggcactgcctgtctgggctactgtcaggggagggtctggcgagaggccccctgcacgcttgccagccagcacaaagcagtatttatcccttcctcctttccacctggctggcacctgacctctccccagtctgtcttcagtgaaggaagatatagaacaacgcccacttccagtgggtcagctcgggcttgtccggagcatggctggtcccctgcccagacccctggaggacactgtggacccattcaggtccctgcagtgatagccagaccagggacaccaccaagcccctcatccttatttctctgcctgtttcccctcccactttttctctcttaacactggacttctttgccagtcttttagtagtgtttcagtgattttacatttagacataggcaattacttttgttcaaaacattcagctccaaccttcaccaactgagataaaaagcaagccctttgcatccttttctctttaacaagcaaagctgtctccaaagaggccagctcaccgtggctacatctgaaacatgctgagtcaacaaatttttaccattaagggttttttattgttattctttaaatctttcatccatgtaagtatattcacacttgaagcttggaaaatgtctggattttgtttctccttcagaaaacatactttgagcttttgtttttctccatgtgtaacaaataatcattttgccctatgaatcatattgtcctgatttcatagaaaacaggtgcagtattagatacagacgcagagagggtcaacccacagctgtggttttcatgtctgtgggcaggacttatagtagtccctatttcctagagagggtattagcaggcaatgaggtgacgcttaggttagaagcgcttagcacagggcctggtgtgtactcagtgcccagaaaggttgatttgtattattagcaattattctgagaaattaactctggccttgctggcctgtgcttcctgcagtgagaggcctgatggagcccagtggtctgtgtggctgtctgcttcctagcactctccctcctcggctgcttttgtggggcctgcatggtggagggatgcctagcaccccagaattcagcccctactgaccagcaccccacaggaggcaagtcaaagtgaggctctgagctgtaggttaaaggaggtgcttgtccactggccaggaatgccatttccctgctgtaatcgtccttgttgaggctgttctgctctcgggtttatcagaagataaaggggtcagcctccgcctcctggcctttgctttccttcctaatccagaaacatgtgggtcagcgcctgcatgaccaggatggatactggaagagtgggctatggagcctttggaaaggtatccgggcatcatttccacaccagcattcgtgttatttctccgtgcatgaatggatgggccttgaagagcccgaaaaagcaggctgagctctcctgaaaggggataatccttggtctttctctttttgccaaaagatcctggataaatgaaccgagagagaccccaatctggaccagagatggtgcccctgacgtttgagcccacctggctgaaagagggcaggtgacagtcccaggacacccacaggaggctactcaggagcctgtattttgggcagaaacaaagaagctgagaggcacagtgaggtgggacaccagcacgccgagggtctccacctccccaaggcaggacaggccatggcagaggcttcaggggagggttccggctttggctgggggcgtgggagtctccagaggcagcgagcccaggacactgatggtgcagaccactggatttctggagaacactgtacttcgccagcctgcggcagacacattattcacagaactttctccaaaggaggttagccatgtttgtcaggctcagggagaagtgaggaggcctccttaccatcaattaaatatgggatccggatcaccctgcgcctgtctcctccccagtcctttctgcgacctcaggtggcctcactcctggcaggcagaagggactcgggtcacacacagccacactcggggattaggacctgaaggatatcattgagcccgcgcccgccccaggctggaattgctgggccgtgatctcctccatcttttctttctccagctgcctctgaaggtcacagaatctgcatttcttaggaaaaatggggcaaaggtgagatggtacacagcacacctggccctggctcagccccccaatgtggacaggttgcagaaggccactgcctcgctgcaggaggctggccctcagcgcccagcagaagcggcagccccatctgtccccgagaccccaccccctcaggcacagctgagcagcccgccggggcccagggttccttggtcttgggttctgatgccttcattcccagaacacgaagccaaggccaggagtcgggatctgctcccaggaagaagcctccatccctgcggagggcttgagaccctctgggatgcgacccagtccccacccatggccactcacttccaccctgccccggcccagccctggccaccccagacgtctggccctcgtcccgaatgctgtcccctctggcctcagctgtcctcctcgaggagcgctatcctgctccgtctctgctcagggtccccccagaggcctcccgctgggatttgtctccactttgcagttgccttttgccccacatgccccgtcctgtggttatttaccttatgggggcatcctccctcttgattgcgactcctcggaggcagataagagtgggtggctttcatcttccagctcccaactcccagcccactgggctgctgtgctgtggtgcacgtgagtcatcagatgccccaggcggaaggctcgtgggggctcagggccccagctgggggcagagcacctcaagaatcctctcaactggctgcaggctcctagttcttggtgacttagtggatggaagacccacctacaggtttccaagctgctgcctttctctaacacatgggcgggtcctggctcagcactattgttctccaccagccctgacccagcacccactgtgagccaggcctgtgggtgtcagcactaaagaccagaagagtcgccagcccatggggcgtctgaagcaggtggcacaattgaccctctcagcccagacaccacccagggccttctccctgacctcggctacagcagatgagaaggacactgtgggagaaccgagtcaggatgcccaaactggatgacttccctgagggcggtgcactgctgaaggcccgtgggtctccattctctgggataatgccagactccagcagaagctttagtctatttctgaatagagaaaatctgctgaaaaacgtctattttccagctgaataaacaagcctttcaagaagaggaggatgaagaaaagacggcatgtgtccaagttgagctgagacggactgtaagcccaggcctgcctaaggcccccacagtgggcatcaccagggctggggtcttaggggccgccagcttcctccacccacctgcctgcacagacagctcctgggcatcaatgcctcagcagtcagatccgcccaggctgcttctgaaagagttttgtagagagattgctcttctgggggcggggacaggacacttatctccatccccccaccaatcatcacaggaggatgaaaaacctcaaagaaagacccagggggcagacacaggcacagatcccagtggagatgcaggctccggaattgcaggctcctttttggaaacggcagaggctccccatgtgggcttccctcacccctccgaggccttcccaggaagacttggtggcagagaccgtgtccactcagcaaacaccaccgttcgctttgctgagtttgcagtttcccgcagcatggctgctgccctggcgaacaacggggccacgccgttacaataaccagcccttgcctgatgagttacagtttacaagactcactcatttatgatttcgtgggattctcttgtttcactcattgggaagcaggcgcagagaggttaagtgttctggaaaaggccctcctctgggggtagagggaggggaaagagagagagagagaaaggatgagagttaatgagaaagagaggcagagagagacgtcgaaccagctgggtcttcaggctccgtggcccgggcccttcagaaagtgccgtgagttgatgtgctggagagaatccacgtgtcagtccccgacaccttatttcttcatggcgattcctttaaaaacacccgtggagcacctcctctgtggcagctctgagaaggcaccagggtagagacctgcatgagagttggcctctgctttgagcagtgccgcgtgcatggataatcctgccggcaaggtcttcctggacatctctgaacactgagggttggcgttgccaggggagactcctggtcgcccgcgtcctattcaggttctgtgggttcccagggccctgtgctctctgtcccctgcaggcctccctcacctccccagcctccttggaccggctgactccctttctccccaggcagccctccagacccctctagacaggggcaggcaccctgcagggcgcccctaccacatcgtgtgcttcccccgcctaacacatggcagttgcattctagttattgaagtcttttctctctctggctgaatcgcaagctttggaagtggggaggtgggaggaggggccaagggcagtgttggtcttgtttagttgtctctccacatcctggcagtcaactcttagtaaccaatgctgaatgaatgcatggatgaatacataagccaaccagcaggtactcttgagtctacagtctccctcaactggcagctgatggatttctttgaaatcaaaagtgtagcgtaaacctacccacagaagtttagtccagtcatcatcacttttggaggtctgatccagggtgggctgtactcagcgaggcctggggtgcagctggccagcgtctcctctgtcagccacacccctgagcggaggctccgtggaggggctgcaggcctcagctccgggtgccctcttctcagtgggagacagcgggagggctgtgctgtttccaacattcccaacacttccattgtttcacaccattactgccgagccccaagggcggatgggtggagccatgttgtgtgggccctgctgttttcctggccagggtgttttgggcagtggttctgtttcctatattgatgtttcccacgaccctgccaggtagcctctgataaagcacatctcttctgagtgagcaatggcctttgggggccatgaagtacctgtgagtgtgactctgccagatgacgggcaggtgcagccatcagtcggacctgagcaaggctacattccttacctggctcaggtgtgaatcttcagggcagcaaaatgtggatgagactgagtaaggggagaatcaatatgtgttttgttgagagaaggaaaaatatgagcaggtctcatgacagaggggtcagagatctaaatcttggagggccccagggctcagttccaaccacgccaccccttctctctgcattctctccagggggccgtatttccctcttcaacagagacagccctattttctgcttgctatccctgtgtcctgtccactttcacatttgctccatatttttcatcttagagcaattgaaaattaaacctttatttttttgaaacagagtctctttccatcacccaggctggagtgcagtggcgcaatctcggctcactgcaacctccgcctcctgggttcaagcaattctcctgcctcagcctcctgagtagctgggattacaggcgcctgccaccacgtctggctaatttttgtatttttagtagagatggggttataccatattggccacagtggtcttgaactcttgacctcaggtgatccacctgccttggcctcccaaagtgctaggattacaggcatgagctaccatgcctggctgaaaattaaacttttaatgttgaaatgatacttgcattaacagaatccaggaaggttctgtagactgtccacttgtgcctccaagcttctgctgtgctctcctcataggatggagacacctgtgtagggaacaggctctctttaatcatgactaaatccggaagatgacagccaccgtcttctctctcttcaccaccctttcctaacacaatgtcagtaacacttacctttcaaggacaacaggcaggttagaagagaaaaagtgcccttggagaagagtgggttagaaccaactcccgttggcctcccagtgtggcgggaggaggcttcagggttgggactcttgctggccacttgtggcaccagccagttgagctgcggcccacaccatggcctgtgagatgccatggtcatcagcacctatcagaaacatgagaaattacagggtagaggattttgaaatatttgcaggaagtcagtacaacttgtaaaaaacaaacagtctgaactctcttcttccatgggcttgggcatttatcatatggtgtgaatctgcaactctatttaaattatgtttcaatagaactttctgaatttgccttaaacttgtgggtcaggaataattagaaaaggcatatcaagcactgatttttttccagaaagttgatgataatgttggtataatttgtaatttatacaaaatacttataaacatttactaactacctcgggggctgtaatggtgtcgctgaccacgtagaagccaggaagaaataaaaagctactaagagatcatcagtattcactaagcggctgcttgaagaaccctgcacctgaggatgataaattgaaggtgctttgcaaactgctaacaaagcatgacttttcacttagctcaaacgactattctcagctaattttgctcatcttcaattccaggtttcactgtgtgcttacagaaggtgaagtgatagctgttagtgctcccaagggtagaagaatttcacgaaccatcagatgatgccagtgttacatcagtgtcactttcaagcaggaattttcaaacaaaatcagtccattcccatagcagttttattttatttttttattcatttcatggaatcccaatacaacttggatgttcctcctgtcaaaggcaagaccttgacttaattactaatgtcacaggaaaggcagtggaagagtcagcatcatgataaaattatttgtgggggaattatgaatatgattatataagtttgggtaaagctcggcatttgtagtaaatgtattgtacttggaattgattatggtgtacacatcatttatttacttatttatttattcaagatggagtgtcgctctgtcacccaggctagagtgcagtggcacaatctcggctcattgcaacctccgcctcccgggttcaagcgattctcctgtctcagcctcctgagcagctgggattacaggcgtgcaccacatcacccatctaatttttgtatttttagtagaggcggggttttgccatgttggccaggatggtctcgaactcctgacctcaagtgatctgcctgcctcggcctcccaaaatgctgggagtacaggcataagccactgcacccagcctcacagcatttataattgtgtccaaattggacatctcatcagttaaaatagaagctgttgatgtcaaaatttataaatagttacatgtacacagagtaactaaactgcaaattcctggtaattaggctgatgttgaatttttaaaacactgccgctttgtagtatgcactttctctctcctgctcatcatcaatcagattctagaaatgttcgggccttcaaagaattattttgtaaataaacctaagtgtcctaaaaagatggtgaatctttctgcaaacaaggccctaagtgtgagtttagtttagttccaaactagttggaaatatattatgggaggattcaataaatggagcacccaaaaccctcccctccacaccaccccccccaccgacacacacacagaactcttggcttttgaaacttttatagagttaaaattattgaaaacaaaggttatggctgggcatggtggtggctcacacctgtaatcccagcactttgggaggtcagggcgggaggattgcttgagtctaggagtttgagactagcctgggcaacatagtgagaccccccccatctttacgaaaaaaagtcttaaattttgccagtcatggtggtgcatccctgtagtcccagctacttgggaggctgagatgggaggatcccttgagcccaggaggttgaaactgcagtgagccagtctggcaacagagtgagactctgcctcaaacaaacaaatgaggttatgaacaggaagacactaaaacttatttctacaaaaacaaggaagcaataaagtgaattaaatgatgagagctcacaaaatgctaatctaaaaaaatgcaggcaattttattttaaaaattaaaacatgaataagaacattgaaaaatatattgaatgaaacatattaggaacagataaggctaagaaattgatcagagcctgtgaatgtgtgctgagaataagtattattttacgttctcattttattttcagataatcaatacaaaaaggtttttttcttttatttttatttgttgttgttgttgttgacacaaggtctggctctgttgtccaggctggagtgcagtggcatgatctcggctattgcaacctccgcttcccaagctcaagtgatcctcccacctcagtccagcaagtagctgggaccacaggcacccatcaccgcgcccagctaattttttgtattttttgtagagacagggttttgccatattgcccaggctggtctcaaactcctgagctcaaagcgatctgtcggcctgggcatccaaaaaaagttttcttaaaaacatatatatgtatatatatacacacacacacacacaaaatatgttattttgttttcagaaagattgtttaatttttaaaaatattttgtgaatagaactacttaatagcctcccagcttaataaaatcaaatcattggtcaataaatgttttagagaagcatatattttgaatcattttagccccctcctttgctctccaagaagtcctggtttggatgagaaattacatgggacccattccaggggctctctttaagtgcggtggctttgaaacctgagtatggctcccaaatcattgtctcctgcttccttgagaagaaccctcatctgtttcttagttgaaagccaaacagctgcaggttttgaccaggtaggtgatgggttcgggctgcagagagaaaacactaccctcttggagctgcagggggcagtgtgggtcagtccactgctggacatcctgggccagctggaaagggcactttcgggtttggtagggccagaggggtcagctgggcaactcctgctcagaggcactggcccagggatggccccttgccctcaaatgccctgaaagctccaattattgaggtgccggagagaaagtcagggactgggaaacggggacacaagacagcccctacactctggggtctcctgggctggttgagacgtccaaaacccaaatcaccttacacagggccagacagtgtgatgccaggaaggactccatctcaggtcgctcaacctcaaaatctggtggggtcaagtaaagggtgaggagagagaggggaccactccaaaagaggaggcagggatgttgggaagtggacggggatcaggaaaatccctttcctaccctgggtgtgtacctcgacctgccttctgccctcccccgagcccagccaccttttcccagtctcctgccgagctcccttcctcgggccctagtgcagtgacttcctgcagctcgccaccttgaccctggccttcctccccaataacccccacaaggagtgcctcccccacacgctgcacttgcagcccacctccccttctgacaaggccccagaccccagatgcaactcctacagacagtctccccaggaatgttccactgtcctctctaatgcaacctttcatcttcagtaacaacaggacattgctcataggatttcggtgagggttgatgaggtcatttatctcagaacctcaggtagcttatcccgcttcataggagcccagtaactgggagcttatattattgccttatgcctccagctaaaaatcatccttatccccaccctagagcccccattgtccttgtctcagctaatgaatgatgctccctcccaccccagctcccaagatggaaaccccgatacctttgttgccccacttatcacccacatcccactgaccaccactgccagggaccctacctttgaaatccttctaaactccctccctcctttccattcccattgcctccggcttggtctttcttaatccattgcttagaccaatacgtctcaaatattaatgagcagaggaaccaccgaggaatcttcataaaaggtgggaggtctgggatggtcctgagactctgcattcctcacatgctcccaggtgatgctcgggctcctggaccactctccggccgcaaaggctatcctgtgtcaggagctgacgcgcagcatccagggctccctttggaaactttcctggcggctcatgactcgtagccaagattttcaaacttcagtctatataaagccattattccccagatactcggatttcttccatctggagttgggcacaggagtgggcatcattccttagtgcccccaggagactctaataatgtagattgttaatggaaaatgagcccttagggccaagccaaatctagcatagcattgaggacctacccaattcctcttcagacttttcctcctcactcatcttctctcacatgccagagaactgtgaactgcctggacagtgggttctgtctgggctcccttcactcctctgtcccctggcctgcgtctccctcgccttccgtcctgaatgactcactcactgcactgcatttctggggaaggtttcattaactgccaagaggaatcattgctccctcacctttgctccttggcacatggccttgtctccagagcatgggagtggggtatcagggtggctgttcttctgcctgcccttcctcctcaatgagctactttagggctgaaacaagactatattctttgcatctctggggcttacctggctgcctgacctaagaacttgcccagtgaagaatattaaaaagacatcagatggaataaaaagacagatttataatgaagtagatagcaggttgactgttgaggggagtaaatatacaacctgctttatcataaataaactccttttctcatctcagttctagaaatcacatgcaatttgtgctctacatactcagcatttggtaggctcacaacagaaactgatgaaatacaaatccttaaaattctgcatttttttatatgagtctcgctctattgcccaggctggagtgcagtggtgcgatctcggctcactacaagctccgcctcctgggttcaagcaattctctgcctcagcctcctgagtggctgggattacaggtgcctgccaccatgtccggctaatttttttttttttttttttttttgtatttttagtagagacagggtttcctgggcaggctagtcttgaactcctgaacttatgatccacccgcctcggcctctcaaaatactgggattacaggcatgagccaccgcgtctggcccagcatgattttaatcactgtttcaagcatcatttggaccaattcatgtctgtgctcaccaggtcacttatgcaagcccactgagagtgaatggccagggactggcaagttgtcattggccttaaaactattgtggaagttagtgtggtgattccttaaggatctagaactagaaataccatttgacccagccatcccattactgggtatatacccaaaggattataaatcatgctgctatcaagacacatgcacacagatgtttattgcggcactattcacaatagcaaagacttggaaccaacccaaatgtccaacaatgagagattggattaagaaaatgtggcacatatacaccatggaatactatgcagccataaaaagggatgagttcatgtcctttgtagggacatggatgaagctagaaaccatcattctgagcaaactatcacaaggacaggaaaccaaacaccacaggttctcactcataggtgggaattgaacaatgagaacacttggacacaggaaggggaacatcacacaccggggcctgtcgtggggtggggggaggggggagggatagcaataggagatatacctagtgtaaatgacgagttagtgggtgcagcacaccaacatggcacatgtatacatatgtaacaaaactgcacgttgtgcacatgtaccctagaacttaaagtataaatatatatatacatataaaaaatggggacagacccgggctccagctggtggacaggctttgaattctgcaggttgtcgcccattgctggtcatgtcaggaaaatcacctgtggctctcctgagcacttccggacctcagtgtctaacagatgaacaaaatgatctttgtctttctcaagggtgtcggcctcctcttagatcactgagcatttggagaggagggttattaaattaatgcatgagataagaaaatgtttcaactaggaattctgcatctctggaattgtattaaaccaattaatactccccaatcaattactctgtaatttactcaagtatattaaaagtattgtggcagtgtgcaataccagatttttaaaaacagtgtgtgcctctgtgggcagagctgcctgtcgtagaagggccagggcagcatgcagcgggggtgagggcttctgtctccagggttcctgggttcaaatcacagttcctccatatcctccagcctgactttgggccagttctctatccttcgggaacctctgtttctcagccctgtggggttattgtgcggataatacagatataaaatacccagcatatagtaggtgctcatggaattatttgtgtatgtggctcattttacaaatctaaaatgtgtgttttggaaataaaaaaaagagggaagaagtaaaagtgctgattgccaccatcgtcgttgttctgggctaattgttataatatcctcataatggtttcatcttaatagtggtgattactgaaacacaattaggatttttcatgctcattcagatgtcttctctaattacaagcattagtgtttaacatgtgagcagccggggaatggatgtttcttggcctcagttctgaaatgggtccatttgcatccctgagggaggcctcaggcaggagggctgctggtggggacaggctgaccttggacggggttcagggggtggggagtgggggggccacacctccactgcatgtctgcattgtcaacagcaaatcccattcagcaggtgccactgcccacctgcacctgccagtgagcctctagtgaggggtttccacagcaccaagcagtcactgccacatttagcgatgctctgatgttttggcaaaatggagtgccaggtgaagatctgaggtgatggcccacaggtcctcagcatgaaagagcctttttctgtgctgagccactttacaatctgggtctgcccctgggcattcccaccccctacccccaataaatacctgcatcccagggtactcaattggctctgtcaagatgagtggaccgtggctccatcctcgcagaggcagcttacctccacttcacctgagagtttagacatggccatggtcagatgcaagggtcaagctggagccaggcagagcatgctggagccagaccaagcatgtaggagctagaccgagtgtgctggagtcagactgagtgtgctggagcagcgaagaacagagaacttggctgcagctgggtgatgaggctgcatgaactaggccctgaaaggatcgaggtttcccaggcagagtgaagggggatgaagaaaccgattgaaaagcagtgtcttgctgggtgtgaagaggacagcgtggccagagaacagggcttgaatgggaggggtagagttagcagggcgagtggagggtggatggtagagggtccaggatgccaggctaggtcacccagatgtgatttctgctaagtggttccagggagggcaggggtctgggatgagatcaccctgggttccagtccctgatctgcccccactaaccatcaaccaagcaggtcctttgagtcctggttccctcctttgagtgaatgtccacagtcagtgactgtatccccctggtgttgagaagcctcagtgagatgatccgtgaaagtgacaggcaaagttcttggctcagagaaagccctgaaatgaagttggctcttctcctcatcatcactgcctggcaccggagggtcccacagctgctgctggatgacctctgagcctgtccctgcctctctgcaggtccaccatggagcccttgtgtccactcctgctggtgggttttagcttgccgctcgccagggctctcaggggcaacgagaccactgccgacagcaacgagacaaccacgacctcaggtaaggacccctttccctggctgcctcccctactacctcctcctaagctcctgggaccattcagagggtgataaaggggcttagggtaagagaagaatggtgtggctgaatttcttagtgtgcttttaatagtttgtgcctctaaaaacaggcaggctaaataagctagtttagcatttggttgttcagcaaagcttagtgttctggaggtgacccatctacaaccccagctgagagtcagggtcagccttggctggaggcaggagtgcaaaagggttaaaaatcccccaaaatccccttgtgcagaaaaccaaccatgaacatacatggatgaggcgatgtgacagttaatgataggaactaaagaattattcttaggccgggtgcggtggctcatacctgtaatcccagcactttgggaggccgaggtgggcagatcacgaggtcaagagattgagaccatcttggccaacacggtgaaaccctgtctctactaaaagtacaaaaattagctgggcgtggtggtgggcgcctgtagtcccagccactcgggaggctgaggcaggagaatcacttcaacctgggaggcggaggttgcagtgagctgagattgtgccactgcactcttgcctggcaacagacagagactacgtctcaaaaaaaaaaaaaaaaaaaaaaaaagaattattcttaatattttaggtcagatattggcgtcatggttagttttatttttaaaaagaagagtccttgccttttgacatgcctgctgaagggttcacgagtgaactgtgatgtctgggctttatgcagaaaagagcaagagggaagtgggggtgggggcttgtgaacaaagtcacatcaagagccccacatgattccctaaccctggacaaatgtcctgcaaaccctggacaaatgtcctgcaaatgtaccactggccccaggaggaggaaagagtaaagatttctggtgacagaacctctagcaaggagagcagttctgagcgcccacctgtctttttcctcttcatgttgaaacctcagagaaatccaaaaccccaaaaatgagagaccagaagctgcccgcaaatattaacttgcctctttgcaagccagcctttcaggggcaggtcactggtggttcaaagagttatttcaacccacaatcccgacttttcttcccaacgctaatcgactggccacgtagaaacatctgacaaactgcccggatgtaccctcacatccaggagccacaatggcagaacatccgtttgcgcaacagtggggaagaatctgcccgagctctgcagacagtccctcgggtggcactgtgggcaccggcaggatgacgtccactccacatctgaggcttttcagtttatttgaaaacaaaattattgggctcacatattgccatagacatggggtgttacttgcaggaggaggacactgtcatgtgacctcagcacccagagcagaactaaatgtgaaactcacaccttggtccagcctctgccagggcctctgcgtgcttagcaaggctgctggtccacgagcaagagggaaatgggcctcgggttataaggcgcagacgggctcgtttgacctagaattgtggaggggaacctgaccctgtgaagtgccctttaaatcttcatctgacccctggaagaagtatgaaggaccatttcagatgataacacgtttccacagtgtgccaaaataataggaagctgtttcatttgttcatcgaacaaaatatattcagtactcatgcagacaaattcttttacagaaggaattgaaggtaaatgtttacaaattatgtaataaaatgttatctataaaaaataatacgtaaaagaacacagtcgaaaaatgagtagagaatatgaccaggtagaatacaaatatctaattatgtttcactaatcatgaaaagatgctcaactctctagaaattagagaaatgcaagttaaaatgacattatcattttacctatcagatggaccaaaatgcaaagggttggcaaaatatcccagagaagagaaactggctccatcgcacattatgggcagcattccgcaacctttgtggcaccagggactggtttaatggaagacagtttttccgtggaattggggtggggggtggtttggggatgattcaagtgcattatatttattttgtactttatttctattatgattacattgtaacatataatgaaataattatacaactcaccatcatgtagaatcagtgggagccctgagcttgtgttcctgcaactagacggcccctctgggggtgatgggagacagtgacagatcaccaggcatcaggttctcttaaggagcacgcaacctagatccctggcatgcgcagttcacaatagggctcacggtcctgtaagaatctaatgccgctgctgatctgacggggggcggcgctcaggcgataatgcctgctcacctgccactcacctcctgctgtgtggcccagttcctaacaggccacagactgatatccatctgtgtcctgggggagttagagacctccgctgtggatgaatgtagaagttggacacttggggctgccatcttggcaattgtgttaaaatgtaacaggcgtacaggctaacacagaagctgtcctacagaaacacttgcgagacacaaagatatatatgtgtgtgtgtgtgtgttgcagaattgtaggtaatagcagaaaagttggaaacaacttaaatgtccatcctattttggtctatccaaaaagtggaatccaagcagctattaaacagaatgaggaagcttatctgggctgacatggaaactctccaagacagacagccgtaccagaaaagcaagctggagaactgccccgtggtgcatttcatctgtttctttcatatttaatgtggctatatggggacgtgtcttaaaattggtagcacttcacaattataatcagtatcattttttttctttcttagtggttcttaaaataacgatactttttacagtccgtgaagctgtagatgaaacgagaggagtagtatggtctatttatttcccaaacctgcatatttatgaacgtaaatttatagaagaaaatgtcaggaaagatacgctttgaaatcttaattatgtttaactctggcaaagagaacaggttcctgaacattgaaaattttaggccggagtgagttgggaccttaaaccattgtctgcacacctgtccttgagtgagccacatcttttcagtatggacttgctccgcctctagggcccctggggaaggagggcttggaatgaaggtgggagaggaaaagagggatggagataagataggggacacagtgagagcagctgtgccctggggcagaactgaggtagtccctggaagaccccggctcttggccagagctcaggatcagcttcctgagcagagggtgctgggtggctacactccagccaaggttgccattctctttgtctcctccaaagtcacattcaagcctaaggttttcaactttcaggatcagattctatgtaagcaaacacacacacacacaagttgcaagtgtatgtatgtataaaattggtgtgaacatacacacacttcactttgggcaatagctagacagtcatacaggaagaaggctgcttcaggatgaccaccctggggactggccaccctgacctctgtcccaccccgtcagagccacaataacatgaaatgaaaataaactgtgacaggaatttcttctcagatgactccctgactcctggctggaccacattggtgcccagaggacatatttttgttttcttcctggtccacaccatttaaaaaaaatgtttttaaattgaatttcttaccaatatttaaaaatcagaaaatgtttacataaaaatatgaatttcttctttaggaaagtgggaagaactaggcactgtgggcaccccttgtgcttggccccagtgggctggaactcagaagtagccatccctgcaggcaggacatggggagtcccaactctcgcccccacctggcctccccacaccacccagggatgttcttacctgccccttgaaggccattgggtttctgatcctgatctctggcctcctgaacagaggtagctactaagaaacaccaggaacagagctggggaagccactggccctgtgacccatcccttctgcccgtgtgcacggcccctttctgggcttgggtggttcagccccgtcttctatggaagtacccatgtctgtgaccgcagccttcactacaggcaggaacgtggaggccagacccacggagggctggtggggcccaggtgctccctcgtgtctctctctgatgagaggaccccatgagttggcttggactaaacttagtagtcaccatctctgcagaagctgagcccagagcctgacctctgtgtgatgcagaagtgggaagtttcattgcctggggccccgggagatgagggcagatgtacctgggtacattcgagagggaggagggggaccgaggcccaagctttctggctgagtctggagtgtgagcagcagccaggccctccgtggggctgggctccaggctgcccctccctccgtgtcaggggtcttcccactcagcctgtcggagttgctccagggccctggcagagtggagaggggtgctctctggagctgcaagtgtggaagcctcaggccagctcacttccccagggaagctctgctgtgggcaggttggaggtcaaaggcgggggactgtggcagccacacccagccactcatcctagcaccaaccgaaccttgtgtggggccagaaccagctcctctgggccagggtcacagacacctgagtgtgggggctgcagaggacagccaggctgccaggctactgacactgctagccagatgggggaggcacggaccccacctggaaggtgggcacttagtgggtgggccaggtgaacagtgccctctctcacggctggggccacctccgcatcacagaccagctgtttcctctgtgaacttcaacatctaatggtcacccacgtccctaaaacttcatcatttatgagaaacgatggtgcggaccactcatagggtgccagagtcagtgccaggcctgggagggtaggggatgagggaggaaaagccacaccttccctggggagtttataacctagggtgcaaggcggaaggcatgagggtcccttttagaatccagatgaggggtgtgaggtgagcggagcctctggggctgcggagccctcccagtgcccggagcaccctcgggaggccgtgccagccctcaggaggaagcgcatggaccctgcgctgggctcagggcaagtgattggtgcttctgggaacttaaagagttggaagcccggtcgttgccttcgaggggcttccccagaaacccgggagccaaggcagtcatgctgaggcctgttcactcatggcagagcgaggtttgcgctttgaggcatccagggcccctctgggtccacaagagaggagtccagtgggcttcttggaggtgggggcctttgagattgtcctcgaggagtgggaacattttggtgatgagggggctgtgtggccagcgggggaggagcaggaacaaagtgcgacgttgggagagtgggtggggtccaaaggtggcagcgaggccaccagtgtcccttggaatttgggctgatgagccaggggtgggtcctgagcagcaacaaagccacttagctgggcctggggctgtgggtctctttctgggtgaggaaggtcaacctgtcaggtacaaaggacacagtaagaggccggtggtggctgggcactcggcctctgacaggtggaatcgggacctggaggctctgaaaggtggggtcacagtgcgccagggccacaggcctctgcccagccccagggccaggccgcctccctgtgcctgagctcctgcccaagcccaaggggccctaagagctgagctacgccgagtcagcctcccgcgctgtcctaggaccctgcagtggacaccggccccttcacccttctccagcttcttagcaggagagccaggcagccaggccgtgtgggggtctggggaactcacccctgtcatgcctgccccagggattttggacaagtcacctcacccactcctcagtcccggattctttctctgagtcaacagcaacaactaaagtcgcttctctgttacctcgtggggccttttcaggatgatggatccactttacaaaatatagtttggggttctggtgtccaggaagaagggcttatggagggagactcttttgcaaactgtgaagtgaggtcaggggttagggtctttctgcttctcctgcaggccctccggacccgggcgcctcccagccgctgctggcctggctgctactgccgctgctgctcctcctcctcgtgctccttctcgccgcctacttcttcaggtaggagtgtcccggggcactgacttgccccaaccagctcagaggaggaaatgagccttctgatgctgtgggcgtgggctgtgcagcagagggcagctgagaggctgggtggctgggcctgggagacacacagaggccaggccttagcgcggctcagccatgagcaacaggagtagcttttcccggctcacctggtgggtatcactgtgccgagcgctgttggctctcggggaacctagaagtgtggaaacctaacgcatcctgtgtctctaaggttcaggaagcagaggaaagctgtggtcagcaccagcgacaagaagatgcccaacggaatcttggaggagcaaggtacagaagctgctctctggctggggcttgggggaaaatgtgttcttttttagcagacactgaggtgctttctgcctttaacgttcaaagtaaattttgaggtgtgccttgccactttcatctcactctacctggtatgatgctgtcatttagaatttcactatttcaaaactttaaggatcatttttctctcgggtgtgacatggacgtttgagacatttttgtttgtggctttttttagagagactgttcatatcctgtcgttttcacagggtgttgacatgtaacacttttcagtgaggccttacaatcattctcagataagactgtctggctcagaaactattcagctgggtttgtactgctttaggatccagctggaaaaagagactcgctctcctgaagtgcttcatgtttacagagcatcagagtcagctgggagcatttccaacttttccatccgaagtacagtttcgaacttgttgtcgctacttacaaaggaacattgtttttctctccatacatagagatgagatgaccgagaaacgtgtgtaaggcagaggccacaaaaatattgcttcacaccccagtgactacaacttcctggagtcagcttcctacctaactgtagtcagttctatggtttgggggaacacacgtgccgtcagactaaaatagactcttaaaacggacccttagatcattaaaagaatatcaccaacaaggacccagctgaaatgtagcctcccagcgctcgcgggacccgagacctggttagacgataatgtggcaccaggtgctgtcttcctgccctcgttccagtccccggttccctgccctgaatgtttcaatagcaggaacggaagggaggagggtgcggctagtgagtagcagggaagctcagcatcactcattctgatcccatctgtcacttggaggaatgagtcttgggtgaagtttttggctgggcttcagaatggctctgcaggtggggacaggcccttttgcaagtgtgacagctgggttatcatgttgaccttgtcaatatcagctcattctccaggaagagaaagacatgtccataactgttcttgcctctcactccgaacttggggaaggaatgtgtccaagaactcccttataacgcgtacttctccttctcaatgccccttgtcaccctccccagacaagggtggttttcccttggagcttatggggtttgaggaaccaggaatatttgttatgaagaagaaaaggctgagagagaacgttctgagtttgctcccagaagccgtaattactctttcttatttccaaacctggaaagctgagctggagagtgaggccgtcttgaatcctttttggaaggagcaggggctggagggaaggagaggtgggagggtgggtggatgagtggcctcgggagggggacaggctgtctccaatgggggagagcaggtgacactgggcagagggacgcccgtcacttgtaaatgatgtggatgcctcacaggaggcgagtgtttggatacagctgctcgggacccggcttagcccagctatgcgatttgagagaactgttccagctccagcctggtcatttcttcaggaatgtcgttggtcagcttggttactcagtcgcctatccaggaatgtggctaaatggcccccatgtttcttttgatcccacagagcagcaaagggtgatgctgctcagcaggtcaccctcagggcccaagaagtattttcccatccccgtggagcacctggaggaggagatccgtatcagatccgccgacgactgcaagcagtttcgggaggagttcaacgtgagtgtggggagggctctctgctgggtgccctgtggtggagacctgtgaaatccagtgttagagttcaggatgaattatcccaaagactcagaacccttgcatcagttatgacactgtcccatgaatgggcgtgtgaggaaacctggtgtgtggtgcagtgccctttgccaatgcttatttcctcatttataagatgagacagttgagttagaggtttgtgaagcctccttacacctccccactctgagtgtcaagttctaaaatcaactgtggcagaaacactgttatctcgataacacagaaggctaggacctgagcgcggcacggtatgacaatatcatggggaaagtaacagagccccaaaccatcctttcatggcttctgagagctggagcacctaatcagagtaggcgatgttcctcttctgaaccaccatctcggcattaatgaagcagctgtgagctgccttgagagaacactagctatatctgcttgtgtatcaacatgggtgggtgggtgggtggatggatggataagtgggagggcagagggatggatggatggatgggtggatggatggatggatggatgatggatggatgagtgggagggcagatggatggatgaatgaatgcatggatggatggatgagtgggagggcggatggatggatggatggatggatggatggatggatggatggatggagtgggagggcatatggatggatgagtgggagggcagatggatggatggatggatgggtggatggatgatggatggatgagtgggagggcagatggatggatggatggatggatgggcagatagagaagtagacaaacagatatgaggaagtattgtcttggattttgctattcagtatttatccttggtcgtgttttgaattttatgcatcttcaccttcttgcatgtggctgtgctaagtgttgatcttaaagaggatagattttcagcaggattgctaactggtcctggctatgcctgttgcgtgctggccctggcacatcagttaatttccttgaagctgtcttccaccacctaggaaggggcaatggagatgggcctgtcttaattaccttgaagttggaatgggaggctcagacaatggattttgtaaacaacagtgttgtgcagttgttatatctgaagatggcatgaattctgctgcagaaatgagactttacccagaagcaacgtgtgaaggcatgggaaggtggtggcccatgtaaactgtacattgcttgttttttggtgcaagcctcctctgcctttatcttacttcctttttattgtttccaaatgtgtccccttttttggagaaattaggtcatattgaaagacagcttgaagcactattgtgttgcaatctctcagcctgacaacagaccttaattctcattttcaaaataactatccaagttttcagctagcagatcccctcccccacccttctcatcccatttccccttgcacttttgtttccccaagtgacaggggctgggcttgctgatgggggtgtctgagtcagaaccagctcaaaagagggggccactcccagggagggagtccaggaatagggtggggcaggtgagacactcaccccaggcacataatttaagacagcaccaaaagctcagcagtgggataaattatattttaacataacacttaaaatatcaaaattatcagcaccacaggaaaggtaacaaatacaatatcaaaatgaatacaggcagtccctggtgaagaaaataacacgaccataaaggaagacaggcttgggccgactgattttccctgttgccctgggccctgatacatacggcttgtcaccacacagctactcatcctgccattagttaaaagtttcatattttggtcatcatagattttgtgccttgatttggacttttaaaaatattgcattcgaatattatttcccctggtttctgaattttgggggcaccccattaaattttgcacccagaggaagttcctcattctcttctgccactgggtggcccttggctgaacattggagtccttgtgctcagtgacaggcagggtagagacgcggtggaggggaagaacactggacccaaagtcaggatcacggagcctgatcctgtgtctggggaaccctgggggagctgcttttttgtcatatttttgttttatttgtttgtttgagatggggtctcgctgtgtctcccaggctggagtgcaggggcctgatcacagttcactgaagactcaacctcccagctcaagtgatcctcccacctcagcctcccaagtagctgggactacaggcatgtgccaccatgcccactggcttttttactttttttgtagagatggggtcccactgtgatgtccaggctgtgcttttctgctcatctgtccagtgaagggctggccatgtcctgagtccacgttgatgcagcacccgtcctctgagagcactggagcctgccatggccagctctctcttgggtcctcagagggacatgctcccaaccccatctttcttttctcttgccgtctgatctgagctcctctccccaaaatcatgaaagggagacaccagagtctcaaagtggccgatccagttgagttttgacatgtgtgcattgccaattcaaagtgcaggcaaacttggtggggatgaaaaggatttgatattggggtcagtacagtgaatgctggagtcggcctgagtgtttgtcccggctcagagagcttgggcaattaacctctgcctgtttcctcatctatgaaatgaggatactccctgccccccataggattgtgataacatggacatgaggttggataacctggacatgcaactgaatccttacgagaccgcactctgcaatctccaccctgcactggtagaacaccatcctaggaaagtctgcttccaggccccagagtaagtcctggaaagtgagcaccactgggccaaggaaaatcccccgcttcagacaggctcgtccctacctccatcctagcaagttatgtcaagccaagtgcctaggactaacacactgatacgtgctgtaaaagtgcagagggaggtgtcaaccgcaaagcttccagcacaacaaacaaaccagaaccattccctggagctccatagagcagctggggcctcttgcagaactgcccacacctagcttcttgtacactctcctgctgcctgcccgacactaccgagcggagagagaaaggaacgtggctcatgagttcattgaaatgaaatctctaggccgggtgtggtggctcatgcctgtactctcagcactttgagaggccaaggtgggcagatcatttgagcccagtagttccagaccagcctgtccaacatggtgaaaccccatctctaccaaaaaactacaaaaattagctaggcgtggtggtgtgcacctgtaatcccagctactcaggaggctgaggtgggagaatcacttgaacccgggaggcagaggttgcagtgagtcgagatcgcgccactgccctccagcctaggtaacagagtgagaccctgtctcaaaaataaaaatgttaaaaaaggaaagaaatgaaatgaaatctcacctcaaacagacaaaccacaaacaatttggacaatttacataatgggacagccaatcacacaatatttattggccagttactatgagcaagccccaagcaagacactgcagggctcatccaggtgtgcagacccctccaggtgctctcctttcccctgggccacctgagcagctcagagatttgagaccccagtagactgacagccagagaaagggctgcagagtccatgtgggtggttattctgtgcacttggatccaggatacatacgtgtgcggtgcacagggcccgatgcccctccccacttcaactttgaccctctgtgcatctgggtggctacgccccatctccatggtctggatctctgcagctgccaggaacacgcaaccgtggggtgtcagagcttggtccatcctcgccatccaatccctccagggagccttctcaatttcttttaatttatttatttatttatttattttttgagagagagtctctctctgtcccccaggctggagtgcagtggtgaaatcttggctcacggcaacctccgcctcccaggttcaagcgattttactgcctcagcctcccaagtagctgagattacaggcgcccaccaccatgcccagctaatttttgtatttttagtagagacggggtttcaccatgttggccaggctggtcttgaactcctgacctcaggtgatccacccgcctcgacttccaaagtgctgggattacaggtgtgagtcaccacacctggcctaatttatcttttaacagactttattttagagcagttttagctatacagaataattgcacagaaagtacagagttcccatatgcccccttcctcccctgtgagcagtttctcctattattaacttcctgcatttgtgtgtttcaactgctgcaattgatgaaccaacactgctctgttgttactagcagaagtccacacgtcagggtttgtgtgttgcgcattctgtgggttttgacgaatgcacagtatcatgtacccaccatccggtatcatgcctcccgtgtattttcagtgctggggccaaaaaaagcttttgcgggtagaaaaagagaaggctctagaattttaccacagatgtcaggcttagattaggtcaagaaggtcagtattaagatagccctgatgaagaatcaggaactacaggcggcgtaggtgctggtggctggggatgaggccaagaaccacctatcacccttgccacagtgccgccagacaggtccctccacataggccaccctcttgcctctgctttgggaccaacaaaccccatggcctggaagggcctcaacctgtggccatggggtttggtggccccaaagcagaggaaagagggcggcctacggtgccccaccatgagctccagctggtcagatctgagtctgcaattggagaagcttcctgccctcaggagaggaagactcgggacacctgtgggtggcaggtcaggctgtgctctgcattgtgagcaagcccaggcctgcacggcttggatccaaagctacgcagtgaggcctgctctgctctcctgggtagttgcagcgggacccctcacattctcctacatttgaagaagtgaacatcctcctggtagtctgggtgccgctctaggctcctgccccttgcttgctgggtgaccctggacaaatctataacacctcgctttggctttcagtttcctcacccataaaacggggaccatcctagtatcacctaatgataggtgagttgcaaaggctggggcagatgacccagctccttccaccgtacctggcgcactgagacactcagtgcatattcatgcatgttactggaatcccagtggccccgggtccagccgctggtcgggtgtaatgtcaaaggacaggactcagggcttcaagaggctcttcacactgtgcattgtggggagctcacctgagcagcgagcagacgcaggagacagctggagatgaccaggaatcagagatggcggctgcaggcggcagaaagaactagaatgcaggccaccagagttcacgctcggttatgctgctcagaggggataggggccccgcggcattccttgcacacctgacttccctgactgggacaaaggagttggttagcgtggtgcttcccaaagacgtgtgcactgaagccctggggatcatggtaatgttcgtgctgattctgtgggtctggggtgtggacagagcatctgcatttctaacaagcaccctcccgggagtgcccaagccactggtccacagagcacacctggagtcttaagcaagatcaggactgtatctaaggctgtttcccgccccttgcctcccaggaccgctctattcatccttcagccccaaagtcacagcatcccgctgtaaaccctccgcatgcctgtcctcgctcccccatctcccggttcacagttctgtcgtagtcagttcttaccaaatcataggcacctgctctaggccagggtccatgggccctgagataccatggaaatgcaaaggcaagtctccgccttcatgcagcataggatctggctgatgagaccatcactttggccacaccactgttcaccccacatgaaagagcaccctggggaaagccagggtctgtgtctccttcattctctgccccatgcctggtgcaaggtctgaggcagagacggactatggacagcaggtagtttgctccacgtgttttcattaattcctgtgctcagtagggaaactgacatgagcatggtgcttgaatccatcacatttcatactaatgctacattttcttccagtcattgccatctggacacatacaaggaacttttgaactggcaaataaagaagaaaacagagaaaaaaacagatatcccaacatccttcccagtaagattttattttatgttttgcatgatcaatggtgtttgcactaaactcagctttgccttgcagctccccgtgactgcaggccattcctggtgctcagaggccccaaatcagtctaactgctcagcatttgcacctaggactccagaacgttcctctttaggtgcccatctccctgcacaggccctgggtaccttggttgctcgtgggtctggctgccaagcctatgcttactgttgggacctgggctgcctggggcccagcgggtaagggtgctgcttcgtggccatgggtgccagctggcagggcccctcctgtggaactcaggcctggcacccagtctaggcctgccagtggccctgggcagctcccttccctcagactcagtgcttgtgaccacaagctgcaaatccctagagttgtctggggtcctggaccaatgacaaaaataatagaatacgaaatttgtaggtgggcagcagctttgtaggaaggctctgaatcgacctgatttaaccccagcagtacaagtgggcatgggctggggagcgtgggtgccaggaggtggcagggccagagagcagacggatgtgtgcgggggtgcgtctcggtgacctgctccagctggatgggtcgtgacctgggggcagctttcacagcctgtcttggtgccaagacaaaccctgtaggttctgaaccgcctttgtaagagctggcccgggccgggtgctgtggctcatgtctgtaatcccagcactctgggaggccgaggcaggtggatcacctgaagtcaggagttcaagaccagcctggccaagatggtgaaaccccgtctctactaaaaatacaaaatattagccgggcatggtggtggacgcctgtaattccagctacgtgggagactgaggcaggagaatctcttgaacctgggagacagaggttgccatgagccgagatcgcgccattgcactccagcctggggaacaagagcgaaactccatctcaaaaaaaaaaaaagatctggcccagcagcaggggggcggggcgtgcagttggggatccttcacctgggcccctcccatcatctgctttgcccatcaggctaacaaaatggcatcctcaaagggctccactaagttcaggtttaaatcccgaacctgcagcctccgcctggtccacaaagggagtccatcccaccctgccagggccttggtgggatggaggaaggcagcccatgtgggccccggaatgctgctccctgctgcatttttaggactagaattgagttcatctgctctgtatgtcagagcagctatggtctgtccgatctgtggtctgtccaaactgtgcactttttagaaataattctgtcccattttacatgcgtgggattgatatgtggttttcacaaaacccactgtgaacccatgatccaatgaaaactgggagaaattcatcaaatagtctttcattcaacacatatgtgtaggggtggccaaggcaacaccaggaagatgggtcaggaggtcatgtggtggccacacagctgagccttgacagtagctcagacatggctagatgctgcacactttggagaggatttgttgccaatgggtagaagatatcacagaaagaggggaggcaacttacatggcttgagcagctcaaaggaaaggaaggccatggcaggaggaggtctgagatctttggaataatgtgtgtcaaatttgggatatttagaaattagtttcactgcttaatttattaccttaattgagtggttttaagttgtgccttgatttgccaaccatatgtgttaagaaaccgctcagtgctaagcacattcctgaattcctcatttctctcaatcactaaaccttcatcccttaaaggtaacatcacattgttaattaagactatttagctgggacctctgtggaagcagaatcatggccaagtgtgggcattacctacacccagtccccttctcaaagtgtgatggcccagggcagagcagtggcctcacctgggagtcccccacctggaagaggccgggcccacccactgcaagtcttctctgagccacgttctcaagtcttctctgagccgcgttctccaggttgtgctgctggagtcagttggcatttcctccaagcctgaaagtgtagtcagattcagaatgggcttttctagattcccctgtaagatctttcccctgctcctggcaggagcaccacaccatgggaaccccagggcccacgcagctgcccgggactgggggaccaggacgtggcacttctcacatgggtggaaagatgggtttacagaatggtggcatggagacgctgtggcctggcaaggatcaatggggtggcatctggcattagccatcaggaagacttaaggctgaagggacattgggcagggagctctcagggctgctccacccgcccccagggtgacagcccatagtatcacttagggtgggactgagagtcacctgggggagaggagagaaggggcccaacttccccagcccctagtatcacttagggtgggactgagagtcacctgggggagaggagagaagggacccaacttccccagcccctggcaccttccctgcctttcccagtcttttaccagagtcataagatggtccttggctctgggcaggcatgtggccctggggagctctggggtcagaggtcaaggtgctttgcatgtcaggcaggcttgacttttgcctgtagaaagactatagaaagatggcaagctaggcctcttttctggaaaagtgccaacagctgataattttaggaaataatgttttgaatgtgaagtgtgactttttagaataaaaagacaggaagctcttagaaactgcaagattctaaatctaagcagaaggctatattttaccctgtgcttttctatagctgtcctcaaagcgtaaaccattccaaattattttcaactagtgttatatgtgttcagcagagctatttctgcctgggcattgccagtccctgagcaggagggtctcacagtgaggtctgcaggactgtaagtttggggtctgactccctggccaccctgtgtgggctgtgactgtctctcagagctatacccgccctttctctgctggcagcccgacagagctggctcaaccatcggaggtcgcaggccaccagcccgtggcccacctggcagccttccaggtgaaggtgagacaaacaaggcatgacctgggggctgcccggctccccatcacaaacgccacaaacaccacaaacacaacccaccctgatcagagactaagcagagaaagcagggagaggacctagagttactcagtaatgactcaggaaggagaccctaagcttctaccacatgccagactctgtgcccagtgcagcataaacgtcctcagaaccagcctggtcccagcctggccgagccggacgttcctgggaaaggttacaggaggagcagggccaggcccacagcacttttagaagcccatgaaaatgtcttcatttctcttcaaatcacaaacaaaacgtgcaaaacccattctggagtgcatcttttcactggcgaccaacccagtcctaagataaccttcttaatagttctatggaggaagctgcaaaggcagaagtgactacaacccacaaaagtcatgatggagccctgacgtgtgtgtacacacacactacatgcacacacacacacacaacacacacactacacacccaccgcacataacgcacacacatacacactccacacacacaccacacactccacacaaacatatcacacacataccacacacatacacaccacacacataccacacactacacacacacacacacacacactacacatacacaccaaacccacatgcacacattcacacagcatacacactacacacatgcacaccacacatacaccacacacactacacacatgcaccatacacaccacacacacaccacacattacacacatacacaaccacacacaccacacacacaatacacacacaccacactcactacacacacaccccacacacagagcactccacggtcagccttctaccccggaaaactgcttgtgcaacaggctggatctaccagacgctcctcactgtgctcgccttccagctctggggcagactccggtggtgggactgagggtcagagtccccaatcctagacccccacatccacttgtcctcatggggctggtgctcatttccctgggaagcctgggagagtggacccagttctcctgtcctggggctcttttaagtgcacccacacttggcaacactggggctgggcccagtgcagggttcataaaatacagcccttgcactgccaggggcctggcacattctgctattcttgctatgtaatgacccagcagcccccatcctggatgctgaaatcctacccttgtcgtcatgccacagctgagacagtcagtgttcaccaaagcaggtgcattctctctcaggacactagtgttctccatgggaacgctgaggaagaacatctctgccatccctggtgtcgctgtgaagctgcttttgcccttcccaggagctgacactgcagatgaagagacagcactgtgatttctggaagagacagcactgtgattcctggtcctaatatcttggctttgtttgggttttttttccagatgaccattctagggtgattctgagccaactggatggaattccctgttcagactacatcaatgcttcctacatagatgtaagtgggcagaggtttcttgttcccctggcccagctggggcatgagcagtaagcactttcttgtctggtgcccggagtgtaaattgtcacaacctttctgggcagtttggccacaagcacaaagggtacggaactgcccgtgctttacacactggcatcagcatctgggaatttgaattcaagagacagagatgatcacagaggatgttcactacagcattatgtatattcctgaccaaatagaacagggagcatgggtagttaaacagattgtggtaaaaccatgtgcagtggactattagaacttgatttgtattaatctttaaaagatcatttaatatcaattataaaattcccattaaaaatacaacataaaataaattgtacatgttcacccaattagaggacagaaatgtctgcatgtctgcttgtgggtgagtctgggaaaggtttggcagaaaaacagactagagaggggacgtatgaagtaggatttgttatttctggaattgctggtgagttttctttccttcttaaccttttcctgtgttttgcaaattttccatggtgaatatgtactgctttcctaacagcaacaccaacaaatcctttcagcaaagataattctgaaaaaatataaatctgatgttattttttctagggttacaaagagaagaataaattcatagcagctcaaggtaagctttttattaatttctcaacgtatttttggtaacgtgaatagctcttatttccttctctgtatgccaacaagaccaaggacttatactggatttgtgtgtgtgtgtttacacctaacagacacgttagatatccattcacacaacggatatgtgtagtcagagaaacacacacttattcccacacctatatggtgtgaacacagggcacgcaatggccccggctgccattccagcttgttaatacttgcgtcagataaccttgaatttctctcagctggctgagccctacatggctccccaagtttgggcgaggggttagtggggtgaatagcttttcagatcaaggccataactaagaatatacttcagctgtgaaaacgtgcccttcaccttgcacagggctaccttgtgtgttgagtactgaatccagcatcactccgttccaccagaggttgtcatctgcaggacctggtgtacgtgcggttagatatcagtggagctgaaaccagattcctcagacttcctcactcttaggccaggagtctttagggcaatgagctctgaaagtcagcgtccatcaaaatcacctgggaagttgttacaaatgcagaatctctaagaaaaaaggacacacatgtgcacacgcagaggccatcaggaatgtgcccagctggaaacctcaggaaacctgacgatcctggctgaagcagatgaaagcatgtgtttctctcttgccagcagacacccacagggagctagcccacggcaggacagcagcagggccaggggcttccttatctccccgcccatccccctcagcacgtagcacttagctcgggactgcaaaatagccgcagctcagacaggaagtggataggggcagaggactttctcctagtgacactttgtctgttgttcacatccccctcccgagggactttagctcatatcgtattggacagcaccatgctgagagtaagattttgacaaagaggaggggattgggaatgcagggtgaggctggccagtctctgcctctcgcaaggctttcaccagctctcccacacttggctgtagacccagccctcatctgaaccctgcttcttacctgtgaacgtcataactcaacctgggcccaattatcttgcccactgaggagttcctagctggagtgggtgagtggcaggacaggcagcccctaccggttccggaacgcttcagggcatgacgtgtgtgtccccaacaagcctgagaagggagtgaacagctgtccaggggccaacggccagggtgccggggctgggaccccaaagggacttcccttcatgccttttgacttcgaaattgtcctctacacacaggtcccaaacaggaaacggttaacgacttctggagaatggtctgggagcaaaagtctgcgaccatcgtcatgttaacaaacttgaaagaaaggaaagaggtgagttccaggcatctggccccggtatcacttcctttcagctgctgctgccgggctggggatgttcttaccacttccttttcctacttccctcccttcctcccactatcactgcagaaaaaaacccaaacacatgcagtggcttacagcattttcttcacgaaacaaatcccctgtgtccccagcatctcactgttatctaatctgtgataacacaatgacacaagagtttcctttttttcccaagtaagagtggcccccggctggccccatggctctgcctgcctgatgagagacatgaatcttagagttttaggatgttgagcgtgtcatgcggcagagctcaggctagaaatgaacatgatttcgcgcttattatccagactagagaaatgcttttggaaaacgttggtatattgaagaaatgttgatttgtcatccgaatatatttaattagctgtttctaattcaaaaaaggatgcatgtacgttataagaatttgaatcattgacaaagaagaaaagttgaactgattcgtttcctaatgtaacaagtactcattagcatctgctgtgctgggccagctctgtcccaggtgcaaggagtaggggctcaaattcagggctgagaagtggtggctgtggggagcctgggtgcgggggtggggttgagtgacaggcagggagccatgccataggcagtgagctcactctccagaatgaaagaagaactgggagcctgagggacacttaccacaggtggggcgaagttaggaggctgtgcttctggcgtgttctggcatgaaggcccggttcagtgggagccgtgtgtcctgcccggccctgctcagggcctgctgtgtcctgagctcctcagtcatctgctggcctggccccagcagttcccaggcattgtagcgaggactgtggggaagggctgccatttttagatgggagtaccaggccttggggagctcagctggggcagaagatgggtggcaggagcccacaggaagggcaggctgggaggtgctctggtccccgcagtgccagcttactaattgccacccaaaacacaggaagcagcctccagccaccgtttctggtcaggaataggatggaggctttcattctgctctggggggagctgaaacaggactgtggagggccacccctttgcccttgcaaatcttgcccttgtcatgctttgtcatggggaagagggaagagtgacagagggaagtaggggattcccagcccaggactcttcactctgcctgcgctgtagcggccatggaggaggacacccacgcgagggaaaaggaggctgacccttcatcaggagcctcagggtcccccgtgacccatggcccatagggaccagtcttcctggggttggtgctccaagcatgagaccagcacagaagcagtggggctcaaggacgccctcacttccctctttcagtcattctgctgaaagtgtcactggcattttctctccatcttcccccacaacctcctactggatttttccactcctgtgactcagtgctgggctgctgccgcgtcctcattctcaccaattcagatgaaatcatttctcaatagagttccctttttcttaattgctcacacttctgttgtggtttccttggcttccacatcctgggctctgcattgcaaaacacagctgccctggcatctcatgatcgggtgggagggaggcagagggagggggtataaaaccagccaccaacactgagcccttgagaggctgacacatgttctcagttcctactggagagccttcaaggtgagaagctcaggagggaccccagggctccccatagggccaagactcacccaaaccgggacatgtaggcatttgcaattcccctgggctgcatcagagatggcctgtgacccacagccccgcagccccgcccggagccaggtgtaaggagggggaagttgtcggtagccgtcataacacaatgatgctgctctctactaaataccagatgctgtttgtttcataagactgttaatcctggaggcttgaccacgagctgtgctgctcaggggccattccctggatatactctctgactactggcaggcttgttccattccagacatgtttctctggcagatgaagtagatcttttcccccttgaggtgggtcctcattcactcgcgaagtgatggaggacaccaccgggcaggtgctgcgggcggtgctgcaaaatgaaagggctcctggaacgtgtattttgggtcattcttgcttcagaggtcgggaaggtaaagaaatgggcctgtctgtagcaatgcaccctcttctgcagactccacggcagcctcgtccctgctagagaaaaatgaagatcgttagaccaatcataggttcatcactcttgaatggcagactggcagtggtgcaaattaactttgtccttagtgaactgagcaggaaaaacacaaattgctgaatccagtgggaaatgtctggggggagcaccgtccttgccctctagcctgagggtcacttggagttaggtgcccagctctgaaggctacaggctcagcataactggttgatggtcagttaataaaatgtatgggattcgtgaatgaatgaagaaatgagacattgttgctgtctacatcagaaagccttggggcatcctagtcatagtttcaaggctgctgttcaccagttacgcagcctcagtttccccattcaagaggtcgacacacgtgaacttgtttctcagctgtgggagctgtgtgactccagctggtgtgtgtgaggccttctgtagttgggtggggggatgtcatgatgcattgcacccacagatctatttgcttctattaaattgttccgtgcaggaaaagtgccatcagtactggcccgaccaaggctgctggacctatggaaacatccgggtgtgcgtggaggactgcgtggttttggtcgactacaccatccggaagttctgcatacagccagtaagcatctctagttgctgcccttccagaaagatcattttcagagagcatcatgcccagagttaacatccttctgcatttatgaagtgctttgctcccagcccggcactgtcgcagccctggctgagagggtgcagtggccaggctgtgataggcagacatgcaagagggccaagacaccagaggccggtgcacaagggtctgcaggcagtgctgggggctcctaattcctagtgactcctctgggcctctccaggaggaagaccagtcattggaggggcctgggggtgaggtcctccctcagtagcacagtgacatttcacacactgactgggaggtggggtttttttctaacactttccgaattctgtctgaaacctcctcgttattttccacacttgggttctgctagttccaccaccccaaaaggtttaagagagagatcagtaaaaatatgtgtatgcaaagcagatgaaatcaagatatagaagaaagtttaaaaccatagagaaaagacagaagaccattttgtcaggatttaaagatacagtcattggcataattaggtcacaaaataagtgtctgaggttccaagaagccacagcaaaaaaggaaaaggatgagctgattattcttatcctatcccctgctggcatctctcccagaaatgtgcacacaggcgcacacaggcactcacatgcacacgcacacacatactcccacacacaggcacacacatgcacacatgcacatacacccacacacaggcatacacatgcacacacacccacacacaggcacacacacgtacacctacccacacacaggcacgcacatgcacatactcccacacacatgcatgcacatgcacacgtacacccacacacaggcacacacatgcacacacacgcacacaaccgtacaatgcatgcacgttcacacacacatgtgcacacacaccccactcacatgcacacatgcacacattcacacatgtgcacacacatacacccacacacattcacatgcacacacatgcacacacgcacacacattcatgcacatgcatacatgtgcacacacacgcacccacacacgcacagatgcacacatgcacattcacacatgtgcacacacatacatccacacacattcacacacgcacacgtgcacacatgcacacattcacacatgtgcacacacacatacacgcacatgggtgcacacgtgtacgcacccacattcacacatgcacacacatgcatacatgtgcacacattcacacgtgcgcacatacacacattcacccacacatacatgcgcacacatgcacacatacacatgcatacatgtgcacacacatgcacacacatctacttccaaatacgctgtgtatcaccttccgtgatctgggcaagggaattaaactcatgggctctgggtccaaaagctgagagaggctctgagggcaggtctgggagactccaaagagcaggccgactgcatcatctgtcctcccaaatgtaagacagctttgcccaaggaaggatgtgctgttggtgattactcagcaccaccggcctgaggtgggactgtccaggcaagatgggccatggccgccctgcttgcacgggggtctgtgagcacacagaccaactttggggatgtgtctggctgcttgggccctgcctgacctgggcatagacggttgctgcgggccctgttctgtggccccaaaagcagcacatgttcagagtcggcaggggtaaggtccaggctgtctggctcaggcaggccctctgtggatgggcccctcccctacgcagccccagcacaggggagcccacggcggcgtcctcagaatgagatgctgggggagcagggggaggattgtttcacccactcttgtctccccgcgtcccccgcagcagctccccgacggctgcaaagcccccaggctggtctcacagctgcacttcaccagctggcccgacttcggagtgccttttacccccattgggatgctgaagttcctcaagaaagtaaagacgctcaaccccgtgcacgctgggcccatcgtggtccactgtaggtacgctgtgggggccacggggcgggaccctcaagggcaggccacagtgggatgactcatcctcatgggcaatgccaggggaccaatatcagggtgggcagaggtttgggcagggctgatgtgaacacaaatgtcagtgactgagccaggccccaagaaccctacaggagaatgttcagagagccccctcttcatcccagtcgataaaacccaccaaagccaggaggctccggttgatggtgggaatggagttctgggcaggtctgcttggccgccctcacctctccccgggaatcccaagtcacctctgggaaccaagatggaaatgagagaaagccccctggtttctacaggaagccccccaacaacacacaagtcgttatggaagagcacaccatgctcagatgggcccccacagcagagtcttccagaacacgctgtcctggagaccagggtccgaggaccaggctgcccctcgccagctgccacttcagcctcggtttcctcacctgtgatgggaaggacagcctcggcggagcctgtgactggcatctgggtgggctttgcacaacatgaagaattactcaaatgtacagccaaggccaccccacaaccccctgtccacagagagccccacttgttcctggcagtgattggcctcaccagggtatcactttgattagatactaagtttccatctgttacagacaggttcattctgagtatcacagcactgccagggacaaaagccacctgcacgcagtgatgcaggcgctctgcacctgtcatgcaggtgttcacgagaagccgtccaggaacccccgaagaacgggctttccccaagtctgagcaacactcgcttgctggggccaagcagaggctggaccccaccccacccaccctccagtgtagctcatactgggaagaatttgcagggtcagataacagtaatctgacagatgccacctcacttaatgccccgagtcagatgacttggtttgcccagtttgacagagagggaggctggataccagagctgttgcatagttgatctgaggtccctctgctcccagggggacagatctaggatgccaatcacacatataagaagaagacactgtttggtaagtgagaaaaattctccacacagcctaatccgaggcttactgcaccagctgttaatgtgcctcttagtagagtgtcggtggctatcagaggaccggcctgagatcactttactgagaccacagctccctctggtggctgcactgtagctttgctcctaattaaccaaaaaaacaaaaagttgcatccagtgacctgggtgcgcaggccccttcggggcctttcatttacttcgggaggagtgtgactcacgacgcagcatctttctctttccccaaagcgcgggcgtgggccggacgggcaccttcattgtgatcgatgccatgatggccatgatgcacgcggagcagaaggtggatgtgtttgaatttgtgtctcgaatccgtaatcagcgccctcagatggttcaaacggatgtgagtcatgccttcctcttgccctgatctagcttcccaaagcaaacccgatgccttcgccacacaggtgtgcagggccctggctctgatgggcacgtggcaaccagcctgggcctggcctggcttcgctccccatagccttccctgcccacgcgtgggacctcagggactccctcgtcctcatctttccctcgtatcctcatgtgagatggggcaatcctactcccccaaacggtgcatgtacacagtgcgtggcgtgctcaccaatgtgaggctctgctgctaccgttctccttactcaagggcataaatggtcgttatccgtggccggtactctgactcttgcctcacacctccttgtgttggcaaaaagagaaaaagaagaaagccgccctctttggtctgccaagctccacgtgggccaagactgcagggcagagttgagggtgtgggcacccctggcctctctgcagatgcagtacacgttcatctaccaagccttactcgagtactacctctacggggacacagagctggacgtgtcctccctggagaagcacctgcagaccatgcacggcaccaccacccacttcgacaagatcgggctggaggaggagttcagggtgagtacagctgaccctcctctccatcctggtgtaagcagccaagggcacgaggaaaacaggtgaccatttaaaggaagcctctttcaatcacttgccccttaactgacctcagaaaaagcaggggcaatacctgagccatgatttacaagggaagaaggatcaggtggctttcagagcctcatagcagccctactaggcacacatttgtatttcccaatgctaaggaggcttcctgggttggagagtggtttcctttgagcaggcgtccttgccagcagcactagtcctcggctgagcaatgtgctcaggagtgtcagaggtttaactgtgtcattatatccttctctgctgcagaaattgacaaatgtccggatcatgaaggagaacatgaggacgggcaacttgccggcaaacatgaagaaggccagggtcatccagatcatcccgtgtaaggcacccgtggcgtggcttgggcagggctggggcggggctggtgccggaggctttcatcctggagaagccattgaccgcttacccctgtgcaccagggtcaaaagcagggtgtcctctggcctcaggctaagggctcctgtgtgctgtaccaaatgtcaggtagaaaagacacctgtgaagcaccagctgacccagacagtcctgcatgggtctcaggcaagctgcttcggatatgagcttgcagatgcgggagggcagccgaggctgatttcaggggagggcttcacagaagctggagatttgcggcaggcacgacttgggattccttgtctcattccttgtctccccaagcctgggcagaggcccgcctcccccttacacagaggggcctgagccacccagccgaggccatgcaggtgaactcagctgagaccacctaggaaaaggctgtggccttgaggaggacaagaaatgggcctagggtagagtcaggacactggtctgccacccaggtcatgggagagggaaggaggacctggcaggtgtgggaggagtggaggcaaagctggcaagaagacagggccacatgcagtgggcaggggagtgggaatcgggaggctcaccaagtgggaaaaagagaaacttgagaaacccggccccaaagcagactccacagggatccgaggaactagggagggaggtgggagatgccccgcttgggaggagggtgtgcctcgttatagacttgtcctcgacagagtcacagctcaacaaacaccatctggaaacagctgggagcaggcctgctgcagcagggtctccccgagcactgcaggcgagtagagctccccggggtgctgccatcttcagactctggcccttggttttctggggccccggtgtcctgctggccactctagattgcttgcctggccacattccatttactccttcagcgcaatatttttagtgcttgtattttggtatgtattatatcgattcctgaaaaatgaaaatgataattgggtggttttcagagttttccttcaaaaagaaaaatacccctgagccactttaattatcgtctctcataacgtaaactttccaaagagttggctttacgtagtgattttattaataggatctatattttgcaagcttgtagcaatggattaaagttagcaaggtgggaccctttttgtaataactaaaggaagataatgctttgcagatgacttcaaccgagtgatcctttccatgaaaaggggtcaagaatacacagactacatcaacgcatccttcatagacgtacgtatgctggcctgggttgtgtttatgcagatgtgtttccttcacatgtgaccacagacttgattagcttgttttcacaatgagaaagaattgacatgtttccagaaactcagccatttttgttagcagaattacttgcttaaaaaaaaaagtggcaaggcacgatggctcacacctgtaatcccaacactttgggaagctaaggcgggcagatcacttggggtcaggagtttgaaactagcctggccaatatggcaaaacccgtctctactaaaaatacaaaaattagctgggcgtgatggcaggtgcctgtaatcccagctacttgggaagctgaggcaggagaattgcttgaatccgggaggctgaggttgcagtgagctgagattacaccactgtactccaacctgggcaacagagtgagagcgagaccccctctcaaaaaaaaaaaaaaaaaaaaaaaaaagtggactttggaaatagaaggacatagactcgctgctaagtaattggctcttttttcattctgcttttaatatatagttatttttcttttacatatctgttacacaccaattatatatccactgcctctgtgctctttctcttgtgtgatttctgcaatgtgtacccacgtggacaatacaattatgtgttcagggcacacagaggctgtcttcatgcacagtggagttcgcagaagaagggcaaaagggggaatggcagccaagtgtgtgttcatattgccatttgcagatgagaaaattgtaccagaatccggtgggctccatgctgatctagctgaaacacacagaaacatgttagaatctaagccaccaggtgctgttcaggtggccacaggcaactaccagttcataatacccatttgttgatgacctcaggggctgacggtggccgttatggtttcagataagcgagcttcttgacctgcttgtcaggtgtgtgcctgagtgctcgtgccaactggggtctggtgcagaccatggaggatgagagagttttcctcattaggccttaggctgtcctaaacttagggagcaggtaatggagccaggatggaaggaagtcagactctaacctctgcgcctccatttgaagggctaccgacagaaggactatttcatcgccacccaggggccactggcacacacggttgaggacttctggaggatgatctgggaatggaaatcccacactatcgtgatgctgacggaggtgcaggagagagagcaggtgaggagtgccgcccagcccggtccctccagggcagcctgtgcacctgaggcactgtccccgttcattacaaatgtcccacctgccatcactgcaaacacatgggtgagaccaagccaggaccactagggaaggagagagattggaaatgaaaaaggtttcatttcaccctttccctttttgggagtgaggtttaaccatagctacccgctgccctttatccttagcctacatgttatttaacctgccctgaaattctgccatttggtattggccaagatcataacctgttgaagaaaaagacatttattgacttttttcaagacatgaagacaacagtaaacccaaggtttaccaacaatgtacatctatctgggttctcccccaagacataactactgctgtgtatttgtgtttttatttctttgctctaaaaaggaacagctttattgtgatataattcacctataattcatgcaattcatctttttaaagggtacaattcagtgaattggagtatattcacagagttgtgcaaccaccaccactatctaatctgtgatcattttcatcaacccaaaaggaaacctcgtaccattaacagtcactccccattccttccttcccctaacccctggcaaccactaatctgcttcctgactatgggtttgtctattctggacattattataaatggaatcatacagtatgtggtcttccgtgattgggttctttcacttagcatcatgctttcaaggttcatctatgttgtagcatgaatcagtaaatcattcttttttactggcaaatagtatttgcttgtgtggatatactacactctgcatatccattcatcagttgatggacatttaggttgtttccactttggagctattataaatagtgctgctttgcttggtgcggttgctcacacctgtaatcccagccctttgggagtccaaggcgggcagatcacctgaggtcaggagttcgcggccagcctggccaacatggcaaaaccccgcttctactaaaaatacaaaaattagctgggcatggtggcgggaacctgcaatcccagctacttgggaggctgaggcagggagaatcgcttgaacctgggaggcagaggttgcattgagctgagatcgcgccattgcactccagcctgggggacagaacaagactctgtctcaaaaaaaaaaaaaaagtttcttgaacattcaggtagtagtttttgtatggacatgctttttcacttcccttgggtatacgcctaggagtgtatatactaggagcggaattgctaggtcacgtcataactctatgtcataaacttactgtttaacatttttgaggacctgccagactgttttcctcagtagctgccccattttacattcctaccagcaatatataaggttctaatttctccacatcctcaccaacattgtcattgtctttttgattaaagccatcctagtggatatgttctttgcttttttaaaacatcttttaaaatcacatctgtttatgcctacgtagactgttgtttagttttacttgtgtttgaactttatagaaagagcatcattacagcatgttgtcttctgagacttgcttcttccactcaaaattgtgcttttgatattctatgttgatgcttatgctgtggttcattcgtcatcattatttcattatattccactgcacaaactatatcctgcattagctcaggtattccttctcctgtcatgggcacttgagttggtttcatttcgttgttttacaaacagtgctacctgtacattctttttttaaaactttaagttctggagacaagtgcagaatgtatagttttgttacataggtatacatgtgcatggtagtttgctgcacctgtcaacccatcatctaggttttaagccctgtatgcattagctatttgtcctaatgctctccctcccctcaccctccaccccccaactggccccagtgtgtgttgttcccctccctgtgtccttgtgttctccctacctgtatattcttgtacatgtcttatgctgcactaggactaagatgtatcaaagagccaaaatgcttcattgaagagtatgtgaaagtttgaatttgcaagataattcctaattgctttccaaagtggacatcaacaatgtataagagatatcattcatctacatacctagtatggtattctcaaggtgtcctaattattgttaattgaaaattaacaaaatattgttcattgtataattattgtataaatactatctttttgtggctctgatttgcatcattctgattccaaggtcaaccccatcacttcatatatttgtggatgacatatttcctcttgtgtcaaatgcctgttcctgcctttcactgatttttctattgtttgtcctttcttattgatttataggagctctttacgtaatcttgatactaattctttattggttttatgtattgcaaacatcttattgcaatctgtggcgtgtcttttcactctgttaatgatgtttgttgatgaagagaagtgaatgttactgtaatccaatgtttcaaattgttgcatcgtgattggttagctgtttgggctcttgtctagtaatccttgtgtacctcaaagtgaaaagatatttcttcttccaagagtttcacagttttgttttttaccttaaaccttactccatctgggtgtatttttattaaggttcaaggccatgacacgtggaagtgcagggtgtacactgcccagttctaggaagagctgggggcactcacatcaaccctggtagggaatgagtcagggatccaatttcattttccccaggtagaaaaccaatttttccagccccacatattgaagaatctctcctttctgccatatgttaaggtttcatatatatatgtgtctgtttctagactctgttctgttccctgggtcaacaagcattaacaggcatttatattcatattcatatgtttttttttttggtcagatgaaatacattaactggtttgagaacaattctgtgttataaactcaaaatcgccagcagcaaattatttattacaagacttaaatttttattttatcccctaaggataaatgctaccagtattggccaaccgagggctcagttactcatggagaaataacgattgagataaagaatgataccctttcagaagccatcagtatacgagactttctggtcactctcaatcaggtattgttgaagtagctctttaaacgcttgtgaatttagtgaaccacgccctccctctgggttctgcttgtcattgaaccagaatgtggccctgcacctgtcctgctccttcacctagtcccattggctatgaatggccaatgggtttcagagacagtggcaggcctcctgtacaattcccatctccctagctgcctacctcccatttatctctcaggagaattacattagccattagctgtaagaggaagaacaaaggggactaatgaagccacaggggcctctctggggccctcctccaggtgccttctcagagccccccaactgtccccatttccactgggtccttggttctgctcatcctctctccttggcaaagaatgagaactcggggtcagctgtaggtaggatgactcacttgtacacaggtcttgatgctgagctccaaacagtcagtatcacgtaatgcacctgccactgtctcctccatgccagccacagagccaccctggccaccagactcagatgctgctcatttgagctctgaagggaaatgcagggccctggtttcttgagaaagtgattcccttctctccagcagagctgccaaggatcccgtaggggttgggcagtcctcacagggatccaggggatgtcctctggacggctcgctcccccttgcagaaggaagcacagccagccacctgccttcttctttaggacaagcaggtttcagccttctccttcaggctgcctcggtggggatgttggcagcacagaaggctcagggagggctcccagccttggccaatccctgagagggcagatggggctggggcctgttccccggcaggcaggcgacgctgagaccccctctcctccctgcagccccaggcccgccaggaggagcaggtccgagtagtgcgccagtttcacttccacggctggcctgagatcgggattcccgccgagggcaaaggcatgattgacctcatcgcagccgtgcagaagcagcagcagcagacaggcaaccaccccatcaccgtgcactgcaggtgagcccccagcccgaagccctccaggtggggtggacacaggctgcacccccccagtacccgcagctgtgggcagcagagcctggtcgctgcccctggccatggtattccctagagtctcctctctacctctccttacacacatgcacacacgacttcacttaagtacatacatgtcatcgtatcatacataagatgcatgtgtgtcatcttaacataaatgcagaatgcacttcagagggtggagttgggaaaccactcaaacacacatttccatgtgcttttgaagtcatcaaaaaattgccaggcagggctcggaggaacacactcctggacttcaagttcctgggtgggttttgagagactgccttactgggctgcattggcctacacccgccctctcccatgggctgggattcctggccatatgaggagtcccaactgggattgtagccacgctggtcctgcagcctcagcctggctcccaagtcaggcccaaaggcagagggacaccctgggcaggtgggacatgcttagtccgcaggtgaaggcgtgaaacacagcacgcccaggggcagatacgcaaatctggtcaagcccactggccacccacttgcctgccttctgtgcctctgtagttctgatcctgctgcacacttctgctggcagggacctcgacacccggcaaggttcttccaagcaggagcttggggtgtttctatccttgggtggtgctaatgtgctcctgaaactgcagttccatggcttgtctcacactggtggcgcccactctttgtgactgcctttcacccccacccactgtcccaccccttatagctggcagagctctctccctgcctggaccccaccggggtgcactcctggcctggccagtaggtggagcacacacctcggtttttggttttcctggtgccttccaagaaatcatttaggataaacttgaaatgagttccatagggcactggagatgcagaggtgaacaagatcccacctgtgagaacgcagatgcggggccgatcattgcggcaggacaggagccttggaataagcccacatgtgagattatggagcacagagggcagccaggcatctccagaagacctgctggaagaggccggtccaccccacctcctccagcccagtacacctgggctctcctacgagggcctgggctctgctcttgtggggtttatcagcaaatgaccatggtaaatggagatgactcaaatcggccttgctgggaagtgtcagttgactgttcctggttgtgaatgtccttaaactgcccctccctttcagcatcacctgtttcagaagacacccctggcacagtaaggagagggtatgccaagattacctccttcctagacctgaactgggagggtttgcatttctggcaagttcccaggtgccaactgtgctgctggtccagggaccacactttgagaaccattgccctacacaaggaagaggggaaatggtgatacttgcttaaaaacttgtcactggtcctgtctcattttataaaatatcggtctcctgtaattagggaggtgttttaggaattttacatttgctaaatcttgctcagaagttacactacatacaaatgatcaaaaaaaaattcagtcaaactcagatcattttcagcgattgttgcttcagtgctgatatgcaggggttggctgtcagctctccatttctcatccccaagtgcaagtcggatgatctgcattaagcgctttttctctgcagtgccggagctgggcgaacaggtacattcatagccctcagcaacattttggagcgagtaaaagccgagggacttttagatgtatttcaagctgtgaagagtttacgacttcagagaccacatatggtgcaaaccctggtaagaatctgaataaggatgttaccagaagagtggagaattatttcatgttgtatttgatgtatgtttcattaaaggaccttaagtacatgggggaggtgggaaggtaaaattcccaaggaaaagagccagctgcaatgtttcgcagtaaacctctgcagagccctccttccttgggtctgtgacgtgggtgtcttctgggcaagacccgggaggaggggcctgtccgccagtttcaatgtctgagaccactcggagagacgcactgaggataggagcttttgtgtcaaagtgaactattctacgagagtcctgtgtgcactgaaaaacatactcttatcaagttcaaaagcagcactgaaatttaaaatcctggtccttctcatttcaatgttatactcgtccatgtggcttgttttccctggcggggtctcactcagcctcaagataccgtccacgggctgcaggaatccctaatgtattagtagttttccccgtacattctttcatttcaaggactcctgctttctggttttcagaagcgattcctcctcaccaaatactatggctcgtgctgctaacccagcgagtacaattttaaagcctgttacaatgcatcctttttaccaaagcttcccacttcgcacctgtcctccctgcctgctcactctccacgctgacaatcctggctcacgcagcgccctgtgcatagagaggcaggaaggaggcccttctacgtccccctcacagcaaggcgcagcaggagcgaggacacctgagctgagggcgggtctgtccatccacacccaacgacctacagcaaagaccatctctctaagcagtagcatacagagcttcccctagtctttttagggactcacagctctcattaatctgcctggtctggccaatatattctatcttcgagagcatctgtcatccaggtgtcctcccgtctcagctgtgctcttcctgaccgcagcttgcccctccatgctccctgactgagaagctgccctcagatgttgtgtggctgtcatctgtgctctcttgcctggtaacagcttgacagagcactttgtagcctgacttttagagtaaagggtctagtccaatgcctggcccgtggcgagggctccagagcaagtggtgaatcagcaaacgaatgagggagaagagtcactaaataaccatcaggcgttgggcgcagtgcaaccaggtgccttacagatgtctttctcatttcatcttcaccatctgcttgcccttgtgcctgcttctgttcacaaggagtgttctgcaggtgcctccagttaggacggttgaggggcctgcctgagatcttcaggtattaagtgtcagaatgagattcaaactctatctgattccagaagggaaaggcttaactagtactgtatgctgccagatctaacatttggcagggagattttggcaaaaaaaaaacacaggcacaaaaatattattgagtaaagagatgattgctgacctgcaggttgatttagccctgcaggtgagcacagtggcccaagaaccagtttccaattgctgagcactcagggaagacttgcttccagcatcctaggtaggctttggttggaactgacatttcaaaggccacaagtacatctgttctcctggagtctgtggattgaccagaaatgtctgtctgtgacctgaggtgtggcttgtatcatccgagcacactccaaagtacacttcatccatcactggccccaatcacttgtactcgagactacgtgggatggtcagcagttagctgctgtgttagctatgaaaacagagtaattagtgatcgtctccatttccatcccagactgagccagctttgatattcagtcaatgttgctttaagttggaaaagtcaatgttgctttaagctgtttgcctctttaaagagagcctgtatatttattgagaattccttctaagaattcaattccctttccttctgaaggctgggtgatcagctgcgtgtcttagtagccagaagtgaggaccctacaatgtggtcttggctcttccaacaacttcatgcaaattcagaagcaaagtttcaggctcaccttttccttctctggaaaactcagctccttcccatcctggccagaccgggactcaggaagggctaagctagctgtttggcatctttccacccaaaagcatttagatatttgttcactgtgtgctgagaagagcccaagtgcaatgtgttggaaagaagcagggaggtgctgaggacagctcttggcctctccggctttccaagcccctgtcccggccccaaactcacatatcccacccaggggtctgggaggaccagcctatatataaaacaatgtcccttttgtacagtaaatgtaggctttaaaataatctagatatgcattttatttaacttttatagatcgctttatgaaatttatctggcaatgttagtactctgatttttttctctttcttttttttttttttttttttttttttttgagacagggtctcactctgtcacctaggctggagtgcagtggtgcagtggcacaatcttggctcactgcaatctgcctcctgggctcaaggaatcctcctgtctccgcttcccaagtagctgggactatgggtgtgcaccactatgcccagctaatttttgtatcttttttgatagagatggggtttcaccatgttgcccaggctggtctggaactcctgggctcaagtgatcctcctgcctcagcctctcaaagtgctcttattacagacgttgagccattgcacctggcctggttttttgtttttttttttaaattcaaaaatagttgttttccagtttcaatgtgaaagccacagacatttaactcagcccattaaatacactaagttgaaatgttaatacactaactgacacattctacgtagaaaaacagctttatttggaattctgcatgtgcctaaaaactaaattacgatgtgcataaaggtatatggtattttaatgaatagtcacacttatttatgtcttgtttttgagtatttctcctgattaatcaaatatatttttgggaatatcattttaatgtgtccttgtttgtcttttcaggaacagtatgaattctgctacaaagtggtacaagattttattgatatattttctgattatgctaatttcaaatgaagattcctgccttaaaatattttttaatttaatggtcagtatattttgtaaaaatcatgttaatttatttcatagttgacattaatatcttccctaatttctttgtatatattttgttatgccttaaaggccacctgctatacagttgttaaatcttaaatatgctttttaaaaattggaataatgtattaaggtcaaataatatcccataaaatatatatttctgctaatattagtaaatatcttaatttttcattagattcatatcatttaatttcacatattcaacacctttaaatgttgtaatcttaatatgcgaagtgtgcctctgcaagatactaacacaaagctcatgttaagaaaacagttgaggactcagaagtcagttcgaaaatgcactttcctaacagtgaattcacaaccctgaacagcagcatttttggaaggcaaactgttcgtgatggtacaatgtaaatggggacttctgtaaagttctcagtttcggtccatgtggtttatctttacattttaaagatcaaagaagtctttacaacctgaatccaggtctaaaacacactagagtagctggtgactataaataatattttaaaatgctgtgtctacaccatcaagactgtgtctacactatcttggctgaacgagaagagatgtaaatgctgggtggtcccgttgacccacggcgttgggtacaacaaaaccagccatcggagttacaccccaaagcaccatttgctgtccagctgcctgtcgtttggcccagaccaccctcagaaaaaaaccagctgcctctcccattctcccctcccgttctgccacagcggcctgggctggtccagtgctatgcctggaggctcaacacaaaacttcccatccaaacattcagatgaactgagcgtcttacacacgcagtacagaggagcacacattaggatagaaacagtagaataaccacgggcaattaaactttaaattttctgagcagcattttggtatttaaacatttcttgtaaaaagctgagacagtttgtaagaaaagaatccttaaaatctagatttataccattttttaaagtcccacctttcaatgtttaataaaacaaaaagagaaatccttaatctaaaagctaaattatttttgaatggaaatactactgagaccattgacactggataacagtaatgatcccattaccagataagattgactgacggggaaaaaaaaaaaaaaagaatggggtgtgaatgtaccaacactgaatcttacagcagttatctttctatggccattaggtacctagcagatgtgcacaatataaacaaaaagatatctggcctaccttactactaaaagcatttaacacgtgcatttttggtacttttttttttgttttctaaaagctacataaaggccttatttgacattttcactgataactgatcgcaccctcatgttgcagtgttcgtcccctattcattaacagtgtgtgcaaatgcaaccccaggacacattattgttctgatagatgctcacagggaatcaagcttctcgcccactccacggctctgagccccatccaagggcaagacttggtgcccagctggaaggacgaaagcacactttgtgaccgccatcctcaccagctgcatgcctgggctgcacactgctgaacgtgctcctctctccttctctgtacaatgattcagcatctcggcggaagaggaaaatggagctttttgaggctcgccaggtcccttttgttttcaccattaaaattccaaacccaaagcctttgtttgactgaaggagaagagagggaagtaagcttctgttcagcacctgaaccctagaaaaagagccagtttgctacgatgaaggtgacatttctctggtcatttatttgagagttcgaagtcaaagtcgaggggcaccggctttggttcatgtctaggagccctctgtcagaatccttgaagccctttaatggtctaactggcatctcttgtatcaagaagtacctttaaggtagaccttttcagggtgccctcaggaaagggccctgctcatgtttttttcctgtccccttagaccaaccccaggtgtccactgcaggggttctgcctgttcccaaactttttccattccaggaacaaaggagaagccactttccccaggacgcaagactctcccctccactgtccgggacagcgttcgccctttagcggggaggtcattacagcctcatggcctctaccaaggccccagatcacaggatctcctgggccttggagcacctcacgctgggggaatcaatccctgagggactcagaatcttctccgtgcaacctggaaagttcatctcttgtttccttcagtcaaagaaagtccattgtacataacaaaacagcccccaaacagcccagtgccgacaccattgttcctttcacactttcctttgttgcatgcagttgggttcaaatgccaaatagtgattagaagacgaccattctgatctgtgtgtgatctggtcactatgtgactgcctttacggtttctctccatgtgctatatgaatgaagaatgcataccagtgttttaaaaggtatttttatgtgtttttaaacacttttttaaatgagcctgacacctgtgtttcagcatttggagacatccccatgttattcttttaagtgtataattactgatacttttttgtttgtttgtttaactaagttgtgtttaacttatgtgcagtctttataatgtatgtatgttattacagtttcaactatcatattttctttgattacatttataatttgatcttgctctgattataatgccagtgaatgttgctgaactctttgtatatgcaaattgcaagatttaaaccattctgatgcaaggataaacctttactttgactaccagcctgtgtttttgtctttaaatctcttaatttcattcctctgcaaaaaaaaaaaaaaaaaa

SEQ ID NO: 11 - DNA, 5392 bp, Homo sapiens
agccggagctggagccgaggcggcggcgggacgcggccggccggacaaatttcctgctaggctgcggacagcgggcggcaggagccggcgcgagcggcttcaggaacccacggcctctgcgcgtccccgcgacccttcttcgcgcccggcgaagacagccgggcgccccggagggcggcgggcaggcgcccgggagatgcggagcctccgctgcagcgcgatctgcgcgaccagaccggcccccccgagactatagccttcactttccctcggtccaccatggagcccttgtgtccactcctgctggtgggttttagcttgccgctcgccagggctctcaggggcaacgagaccactgccgacagcaacgagacaaccacgacctcaggccctccggacccgggcgcctcccagccgctgctggcctggctgctactgccgctgctgctcctcctcctcgtgctccttctcgccgcctacttcttcaggttcaggaagcagaggaaagctgtggtcagcaccagcgacaagaagatgcccaacggaatcttggaggagcaagagcagcaaagggtgatgctgctcagcaggtcaccctcagggcccaagaagtattttcccatccccgtggagcacctggaggaggagatccgtatcagatccgccgacgactgcaagcagtttcgggaggagttcaactcattgccatctggacacatacaaggaacttttgaactggcaaataaagaagaaaacagagaaaaaaacagatatcccaacatccttcccaatgaccattctagggtgattctgagccaactggatggaattccctgttcagactacatcaatgcttcctacatagatggttacaaagagaagaataaattcatagcagctcaaggtcccaaacaggaaacggttaacgacttctggagaatggtctgggagcaaaagtctgcgaccatcgtcatgttaacaaacttgaaagaaaggaaagaggaaaagtgccatcagtactggcccgaccaaggctgctggacctatggaaacatccgggtgtgcgtggaggactgcgtggttttggtcgactacaccatccggaagttctgcatacagccacagctccccgacggctgcaaagcccccaggctggtctcacagctgcacttcaccagctggcccgacttcggagtgccttttacccccattgggatgctgaagttcctcaagaaagtaaagacgctcaaccccgtgcacgctgggcccatcgtggtccactgtagcgcgggcgtgggccggacgggcaccttcattgtgatcgatgccatgatggccatgatgcacgcggagcagaaggtggatgtgtttgaatttgtgtctcgaatccgtaatcagcgccctcagatggttcaaacggatatgcagtacacgttcatctaccaagccttactcgagtactacctctacggggacacagagctggacgtgtcctccctggagaagcacctgcagaccatgcacggcaccaccacccacttcgacaagatcgggctggaggaggagttcaggaaattgacaaatgtccggatcatgaaggagaacatgaggacgggcaacttgccggcaaacatgaagaaggccagggtcatccagatcatcccgtatgacttcaaccgagtgatcctttccatgaaaaggggtcaagaatacacagactacatcaacgcatccttcatagacggctaccgacagaaggactatttcatcgccacccaggggccactggcacacacggttgaggacttctggaggatgatctgggaatggaaatcccacactatcgtgatgctgacggaggtgcaggagagagagcaggataaatgctaccagtattggccaaccgagggctcagttactcatggagaaataacgattgagataaagaatgataccctttcagaagccatcagtatacgagactttctggtcactctcaatcagccccaggcccgccaggaggagcaggtccgagtagtgcgccagtttcacttccacggctggcctgagatcgggattcccgccgagggcaaaggcatgattgacctcatcgcagccgtgcagaagcagcagcagcagacaggcaaccaccccatcaccgtgcactgcagtgccggagctgggcgaacaggtacattcatagccctcagcaacattttggagcgagtaaaagccgagggacttttagatgtatttcaagctgtgaagagtttacgacttcagagaccacatatggtgcaaaccctggaacagtatgaattctgctacaaagtggtacaagattttattgatatattttctgattatgctaatttcaaatgaagattcctgccttaaaatattttttaatttaatggtcagtatattttgtaaaaatcatgttaatttatttcatagttgacattaatatcttccctaatttctttgtatatattttgttatgccttaaaggccacctgctatacagttgttaaatcttaaatatgctttttaaaaattggaataatgtattaaggtcaaataatatcccataaaatatatatttctgctaatattagtaaatatcttaatttttcattagattcatatcatttaatttcacatattcaacacctttaaatgttgtaatcttaatatgcgaagtgtgcctctgcaagatactaacacaaagctcatgttaagaaaacagttgaggactcagaagtcagttcgaaaatgcactttcctaacagtgaattcacaaccctgaacagcagcatttttggaaggcaaactgttcgtgatggtacaatgtaaatggggacttctgtaaagttctcagtttcggtccatgtggtttatctttacattttaaagatcaaagaagtctttacaacctgaatccaggtctaaaacacactagagtagctggtgactataaataatattttaaaatgctgtgtctacaccatcaagactgtgtctacactatcttggctgaacgagaagagatgtaaatgctgggtggtcccgttgacccacggcgttgggtacaacaaaaccagccatcggagttacaccccaaagcaccatttgctgtccagctgcctgtcgtttggcccagaccaccctcagaaaaaaaccagctgcctctcccattctcccctcccgttctgccacagcggcctgggctggtccagtgctatgcctggaggctcaacacaaaacttcccatccaaacattcagatgaactgagcgtcttacacacgcagtacagaggagcacacattaggatagaaacagtagaataaccacgggcaattaaactttaaattttctgagcagcattttggtatttaaacatttcttgtaaaaagctgagacagtttgtaagaaaagaatccttaaaatctagatttataccattttttaaagtcccacctttcaatgtttaataaaacaaaaagagaaatccttaatctaaaagctaaattatttttgaatggaaatactactgagaccattgacactggataacagtaatgatcccattaccagataagattgactgacggggaaaaaaaaaaaaaaagaatggggtgtgaatgtaccaacactgaatcttacagcagttatctttctatggccattaggtacctagcagatgtgcacaatataaacaaaaagatatctggcctaccttactactaaaagcatttaacacgtgcatttttggtacttttttttttgttttctaaaagctacataaaggccttatttgacattttcactgataactgatcgcaccctcatgttgcagtgttcgtcccctattcattaacagtgtgtgcaaatgcaaccccaggacacattattgttctgatagatgctcacagggaatcaagcttctcgcccactccacggctctgagccccatccaagggcaagacttggtgcccagctggaaggacgaaagcacactttgtgaccgccatcctcaccagctgcatgcctgggctgcacactgctgaacgtgctcctctctccttctctgtacaatgattcagcatctcggcggaagaggaaaatggagctttttgaggctcgccaggtcccttttgttttcaccattaaaattccaaacccaaagcctttgtttgactgaaggagaagagagggaagtaagcttctgttcagcacctgaaccctagaaaaagagccagtttgctacgatgaaggtgacatttctctggtcatttatttgagagttcgaagtcaaagtcgaggggcaccggctttggttcatgtctaggagccctctgtcagaatccttgaagccctttaatggtctaactggcatctcttgtatcaagaagtacctttaaggtagaccttttcagggtgccctcaggaaagggccctgctcatgtttttttcctgtccccttagaccaaccccaggtgtccactgcaggggttctgcctgttcccaaactttttccattccaggaacaaaggagaagccactttccccaggacgcaagactctcccctccactgtccgggacagcgttcgccctttagcggggaggtcattacagcctcatggcctctaccaaggccccagatcacaggatctcctgggccttggagcacctcacgctgggggaatcaatccctgagggactcagaatcttctccgtgcaacctggaaagttcatctcttgtttccttcagtcaaagaaagtccattgtacataacaaaacagcccccaaacagcccagtgccgacaccattgttcctttcacactttcctttgttgcatgcagttgggttcaaatgccaaatagtgattagaagacgaccattctgatctgtgtgtgatctggtcactatgtgactgcctttacggtttctctccatgtgctatatgaatgaagaatgcataccagtgttttaaaaggtatttttatgtgtttttaaacacttttttaaatgagcctgacacctgtgtttcagcatttggagacatccccatgttattcttttaagtgtataattactgatacttttttgtttgtttgtttaactaagttgtgtttaacttatgtgcagtctttataatgtatgtatgttattacagtttcaactatcatattttctttgattacatttataatttgatcttgctctgattataatgccagtgaatgttgctgaactctttgtatatgcaaattgcaagatttaaaccattctgatgcaaggataaacctttactttgactaccagcctgtgtttttgtctttaaatctcttaatttcattcctctgcaaaaaaaaaaaaaaaaaa

SEQ ID NO: 12 - protein, 700 aa, Homo sapiens
MEPLCPLLLVGFSLPLARALRGNETTADSNETTTTSGPPDPGASQPLLAWLLLPLLLLLLVLLLAAYFFRFRKQRKAVVSTSDKKMPNGILEEQEQQRVMLLSRSPSGPKKYFPIPVEHLEEEIRIRSADDCKQFREEFNSLPSGHIQGTFELANKEENREKNRYPNILPNDHSRVILSQLDGIPCSDYINASYIDGYKEKNKFIAAQGPKQETVNDFWRMVWEQKSATIVMLTNLKERKEEKCHQYWPDQGCWTYGNIRVCVEDCVVLVDYTIRKFCIQPQLPDGCKAPRLVSQLHFTSWPDFGVPFTPIGMLKFLKKVKTLNPVHAGPIVVHCSAGVGRTGTFIVIDAMMAMMHAEQKVDVFEFVSRIRNQRPQMVQTDMQYTFIYQALLEYYLYGDTELDVSSLEKHLQTMHGTTTHFDKIGLEEEFRKLTNVRIMKENMRTGNLPANMKKARVIQIIPYDFNRVILSMKRGQEYTDYINASFIDGYRQKDYFIATQGPLAHTVEDFWRMIWEWKSHTIVMLTEVQEREQDKCYQYWPTEGSVTHGEITIEIKNDTLSEAISIRDFLVTLNQPQARQEEQVRVVRQFHFHGWPEIGIPAEGKGMIDLIAAVQKQQQQTGNHPITVHCSAGAGRTGTFIALSNILERVKAEGLLDVFQAVKSLRLQRPHMVQTLEQYEFCYKVVQDFIDIFSDYANFK

SEQ ID NO: 13 - DNA, 5039 bp, Homo sapiens
ctgatgctgtgggcgtgggctgtgcagcagagggcagctgagaggctgggtggctgggcctgggagacacacagaggccaggccttagcgcggctcagccatgagcaacaggagtagcttttcccggctcacctggttcaggaagcagaggaaagctgtggtcagcaccagcgacaagaagatgcccaacggaatcttggaggagcaagagcagcaaagggtgatgctgctcagcaggtcaccctcagggcccaagaagtattttcccatccccgtggagcacctggaggaggagatccgtatcagatccgccgacgactgcaagcagtttcgggaggagttcaactcattgccatctggacacatacaaggaacttttgaactggcaaataaagaagaaaacagagaaaaaaacagatatcccaacatccttcccaatgaccattctagggtgattctgagccaactggatggaattccctgttcagactacatcaatgcttcctacatagatggttacaaagagaagaataaattcatagcagctcaaggtcccaaacaggaaacggttaacgacttctggagaatggtctgggagcaaaagtctgcgaccatcgtcatgttaacaaacttgaaagaaaggaaagaggaaaagtgccatcagtactggcccgaccaaggctgctggacctatggaaacatccgggtgtgcgtggaggactgcgtggttttggtcgactacaccatccggaagttctgcatacagccacagctccccgacggctgcaaagcccccaggctggtctcacagctgcacttcaccagctggcccgacttcggagtgccttttacccccattgggatgctgaagttcctcaagaaagtaaagacgctcaaccccgtgcacgctgggcccatcgtggtccactgtagcgcgggcgtgggccggacgggcaccttcattgtgatcgatgccatgatggccatgatgcacgcggagcagaaggtggatgtgtttgaatttgtgtctcgaatccgtaatcagcgccctcagatggttcaaacggatatgcagtacacgttcatctaccaagccttactcgagtactacctctacggggacacagagctggacgtgtcctccctggagaagcacctgcagaccatgcacggcaccaccacccacttcgacaagatcgggctggaggaggagttcaggaaattgacaaatgtccggatcatgaaggagaacatgaggacgggcaacttgccggcaaacatgaagaaggccagggtcatccagatcatcccgtatgacttcaaccgagtgatcctttccatgaaaaggggtcaagaatacacagactacatcaacgcatccttcatagacggctaccgacagaaggactatttcatcgccacccaggggccactggcacacacggttgaggacttctggaggatgatctgggaatggaaatcccacactatcgtgatgctgacggaggtgcaggagagagagcaggataaatgctaccagtattggccaaccgagggctcagttactcatggagaaataacgattgagataaagaatgataccctttcagaagccatcagtatacgagactttctggtcactctcaatcagccccaggcccgccaggaggagcaggtccgagtagtgcgccagtttcacttccacggctggcctgagatcgggattcccgccgagggcaaaggcatgattgacctcatcgcagccgtgcagaagcagcagcagcagacaggcaaccaccccatcaccgtgcactgcagtgccggagctgggcgaacaggtacattcatagccctcagcaacattttggagcgagtaaaagccgagggacttttagatgtatttcaagctgtgaagagtttacgacttcagagaccacatatggtgcaaaccctggaacagtatgaattctgctacaaagtggtacaagattttattgatatattttctgattatgctaatttcaaatgaagattcctgccttaaaatattttttaatttaatggtcagtatattttgtaaaaatcatgttaatttatttcatagttgacattaatatcttccctaatttctttgtatatattttgttatgccttaaaggccacctgctatacagttgttaaatcttaaatatgctttttaaaaattggaataatgtattaaggtcaaataatatcccataaaatatatatttctgctaatattagtaaatatcttaatttttcattagattcatatcatttaatttcacatattcaacacctttaaatgttgtaatcttaatatgcgaagtgtgcctctgcaagatactaacacaaagctcatgttaagaaaacagttgaggactcagaagtcagttcgaaaatgcactttcctaacagtgaattcacaaccctgaacagcagcatttttggaaggcaaactgttcgtgatggtacaatgtaaatggggacttctgtaaagttctcagtttcggtccatgtggtttatctttacattttaaagatcaaagaagtctttacaacctgaatccaggtctaaaacacactagagtagctggtgactataaataatattttaaaatgctgtgtctacaccatcaagactgtgtctacactatcttggctgaacgagaagagatgtaaatgctgggtggtcccgttgacccacggcgttgggtacaacaaaaccagccatcggagttacaccccaaagcaccatttgctgtccagctgcctgtcgtttggcccagaccaccctcagaaaaaaaccagctgcctctcccattctcccctcccgttctgccacagcggcctgggctggtccagtgctatgcctggaggctcaacacaaaacttcccatccaaacattcagatgaactgagcgtcttacacacgcagtacagaggagcacacattaggatagaaacagtagaataaccacgggcaattaaactttaaattttctgagcagcattttggtatttaaacatttcttgtaaaaagctgagacagtttgtaagaaaagaatccttaaaatctagatttataccattttttaaagtcccacctttcaatgtttaataaaacaaaaagagaaatccttaatctaaaagctaaattatttttgaatggaaatactactgagaccattgacactggataacagtaatgatcccattaccagataagattgactgacggggaaaaaaaaaaaaaaagaatggggtgtgaatgtaccaacactgaatcttacagcagttatctttctatggccattaggtacctagcagatgtgcacaatataaacaaaaagatatctggcctaccttactactaaaagcatttaacacgtgcatttttggtacttttttttttgttttctaaaagctacataaaggccttatttgacattttcactgataactgatcgcaccctcatgttgcagtgttcgtcccctattcattaacagtgtgtgcaaatgcaaccccaggacacattattgttctgatagatgctcacagggaatcaagcttctcgcccactccacggctctgagccccatccaagggcaagacttggtgcccagctggaaggacgaaagcacactttgtgaccgccatcctcaccagctgcatgcctgggctgcacactgctgaacgtgctcctctctccttctctgtacaatgattcagcatctcggcggaagaggaaaatggagctttttgaggctcgccaggtcccttttgttttcaccattaaaattccaaacccaaagcctttgtttgactgaaggagaagagagggaagtaagcttctgttcagcacctgaaccctagaaaaagagccagtttgctacgatgaaggtgacatttctctggtcatttatttgagagttcgaagtcaaagtcgaggggcaccggctttggttcatgtctaggagccctctgtcagaatccttgaagccctttaatggtctaactggcatctcttgtatcaagaagtacctttaaggtagaccttttcagggtgccctcaggaaagggccctgctcatgtttttttcctgtccccttagaccaaccccaggtgtccactgcaggggttctgcctgttcccaaactttttccattccaggaacaaaggagaagccactttccccaggacgcaagactctcccctccactgtccgggacagcgttcgccctttagcggggaggtcattacagcctcatggcctctaccaaggccccagatcacaggatctcctgggccttggagcacctcacgctgggggaatcaatccctgagggactcagaatcttctccgtgcaacctggaaagttcatctcttgtttccttcagtcaaagaaagtccattgtacataacaaaacagcccccaaacagcccagtgccgacaccattgttcctttcacactttcctttgttgcatgcagttgggttcaaatgccaaatagtgattagaagacgaccattctgatctgtgtgtgatctggtcactatgtgactgcctttacggtttctctccatgtgctatatgaatgaagaatgcataccagtgttttaaaaggtatttttatgtgtttttaaacacttttttaaatgagcctgacacctgtgtttcagcatttggagacatccccatgttattcttttaagtgtataattactgatacttttttgtttgtttgtttaactaagttgtgtttaacttatgtgcagtctttataatgtatgtatgttattacagtttcaactatcatattttctttgattacatttataatttgatcttgctctgattataatgccagtgaatgttgctgaactctttgtatatgcaaattgcaagatttaaaccattctgatgcaaggataaacctttactttgactaccagcctgtgtttttgtctttaaatctcttaatttcattcctctgcaaaaaaaaaaaaaaaaaa

SEQ ID NO: 14 - protein, 642 aa, Homo sapiens
MSNRSSFSRLTWFRKQRKAVVSTSDKKMPNGILEEQEQQRVMLLSRSPSGPKKYFPIPVEHLEEEIRIRSADDCKQFREEFNSLPSGHIQGTFELANKEENREKNRYPNILPNDHSRVILSQLDGIPCSDYINASYIDGYKEKNKFIAAQGPKQETVNDFWRMVWEQKSATIVMLTNLKERKEEKCHQYWPDQGCWTYGNIRVCVEDCVVLVDYTIRKFCIQPQLPDGCKAPRLVSQLHFTSWPDFGVPFTPIGMLKFLKKVKTLNPVHAGPIVVHCSAGVGRTGTFIVIDAMMAMMHAEQKVDVFEFVSRIRNQRPQMVQTDMQYTFIYQALLEYYLYGDTELDVSSLEKHLQTMHGTTTHFDKIGLEEEFRKLTNVRIMKENMRTGNLPANMKKARVIQIIPYDFNRVILSMKRGQEYTDYINASFIDGYRQKDYFIATQGPLAHTVEDFWRMIWEWKSHTIVMLTEVQEREQDKCYQYWPTEGSVTHGEITIEIKNDTLSEAISIRDFLVTLNQPQARQEEQVRVVRQFHFHGWPEIGIPAEGKGMIDLIAAVQKQQQQTGNHPITVHCSAGAGRTGTFIALSNILERVKAEGLLDVFQAVKSLRLQRPHMVQTLEQYEFCYKVVQDFIDIFSDYANFK